(19) United States
(12) Patent Application Publication
(10) Pub. No.: US 2025/0179013 A1
(43) Pub. Date: Jun. 5, 2025

(54) INHIBITORS OF RNA HELICASE DHX9 AND USES THEREOF

(71) Applicant: ACCENT THERAPEUTICS, INC., Lexington, MA (US)

(72) Inventors: Matthew H. Daniels, Somerville, MA (US); Kenneth W. Duncan, Westwood, MA (US); Brian Andrew Sparling, Saugus, MA (US); Young-tae Lee, Needham, MA (US); Jennifer Castro, Billerica, MA (US); Scott Ribich, Lexington, MA (US); Ernest Allen Sickmier, Needham, MA (US); Andrew Stewart Tasker, Simi Valley, CA (US); James Edward John Mills, Sheffield (GB)

(21) Appl. No.: 18/839,362

(22) PCT Filed: Feb. 17, 2023
(86) PCT No.: PCT/US2023/013303

§ 371 Date: Aug. 16, 2024

Related U.S. Application Data
(60) Provisional application No. 63/311,649, filed on Feb. 18, 2022.

(51) Int. Cl. C07C 311/32; A61K 31/166; A61K 31/381; A61K 31/40; A61K 31/402; A61K 31/4025; A61K 31/404; A61K 31/41; A61K 31/415; A61K 31/4155; A61K 31/416; A61K 31/4184; A61K 31/4245; A61K 31/426; A61K 31/427; A61K 31/437; A61K 31/4409; A61K 31/4418; A61K 31/4436; A61K 31/4439; A61K 31/451; A61K 31/4535; A61K 31/454; A61K 31/496; A61K 31/50; A61K 31/501; A61K 31/506; A61K 31/5377; C07D 207/337; C07D 207/34; C07D 211/34; C07D 213/76; C07D 213/81; C07D 231/14; C07D 237/20; C07D 257/04; C07D 271/10; C07D 277/30; C07D 277/56; C07D 333/24; C07D 333/34; C07D 333/38; C07D 401/04; C07D 401/14; C07D 403/04; C07D 405/04; C07D 405/06; C07D 409/04; C07D 409/12; C07D 417/04; C07D 417/12; C07D 471/04

(52) U.S. Cl. CPC C07C 311/32; A61K 31/166; A61K 31/381; A61K 31/40; A61K 31/402; A61K 31/4025; A61K 31/404; A61K 31/41; A61K 31/415; A61K 31/4155; A61K 31/416; A61K 31/4184; A61K 31/4245; A61K 31/426; A61K 31/427; A61K 31/437; A61K 31/4409; A61K 31/4418; A61K 31/4436; A61K 31/4439; A61K 31/451; A61K 31/4535; A61K 31/454; A61K 31/496; A61K 31/50; A61K 31/501; A61K 31/506; A61K 31/5377; C07D 207/337; C07D 207/34; C07D 211/34; C07D 213/76; C07D 213/81; C07D 231/14; C07D 237/20; C07D 257/04; C07D 271/10; C07D 277/30; C07D 277/56; C07D 333/24; C07D 333/34; C07D 333/38; C07D 401/04; C07D 401/14; C07D 403/04; C07D 405/04; C07D 405/06; C07D 409/04; C07D 409/12; C07D 417/04; C07D 417/12; C07D 471/04; C07C 2601/16

(57) ABSTRACT

Provided are compounds of the Formula (I): or pharmaceutically acceptable salts thereof, which are useful for the inhibition of DHX9 and in the treatment of a variety of DHX9 mediated conditions or diseases, such as cancer.

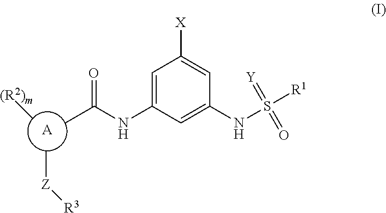

### RELATED APPLICATIONS

This application claims the benefit of the filing date, under 35 U.S.C. § 119(e), to U.S. Provisional Application No. 63/311,649, filed on Feb. 18, 2022. The entire contents of the foregoing application are expressly incorporated herein by reference.

### FIELD OF THE INVENTION

The present disclosure relates to inhibitors of RNA helicase DHX9, and pharmaceutically acceptable salts thereof, compositions of these compounds, processes for their preparation, and their use in the treatment of diseases.

### BACKGROUND OF THE INVENTION

DHX9, also known as RNA Helicase A (RHA) or Nuclear DNA Helicase II (NDH II), is a DExH-box RNA helicase, which shuttles between nucleus and cytoplasm, and can use all four NTPs to power cycles of directional movement from 3′ to 5′. Functionally, DHX9 can bind to and unwind or resolve dsDNA/RNA, ssDNA/RNA, DNA:RNA hybrids (such as R-loops), circular RNA, and DNA/RNA G quadruplexes. As such, DHX9 has regulatory roles in various RNA and DNA related cellular processes, such as transcription, translation, RNA splicing, editing, RNA transport and processing, microRNA genesis, and maintenance of genomic stability (Pan et al., 2021, Current Protein & Peptide Science (22), 29-40).

Due to its regulatory role in processes such as transcription and maintenance of genomic stability, DHX9 has shown to be a key regulator in a variety of cancer types (Gulliver et al., 2020, Future Science OA (2), FS0650). DHX9 has been shown to be involved in the regulation of genes associated with sustained proliferative signaling, evasion of growth suppressors, evasion of apoptosis, angiogenesis, and metastasis, all of which are hallmarks of cancer. Specifically, microsatellite instable cancers, such as Microsatellite Instable (MSI) colorectal cancer, and tumors with defective MisMatch Repair (MMR) exhibit a strong dependence on DHX9.

In addition to its role in cancer, DHX9 has been implicated in other diseases involving gene replication, translation or regulation. These diseases include viral infections and autoimmune disease.

Thus, there is a need for DHX9 inhibitors as potential therapeutic agents for treating diseases or disorders that are responsive to DHX9 inhibition.

### SUMMARY OF THE INVENTION

The present disclosure provides compounds that are DHX9 inhibitors. In a first aspect, the present disclosure relates to compounds having the Formula I:

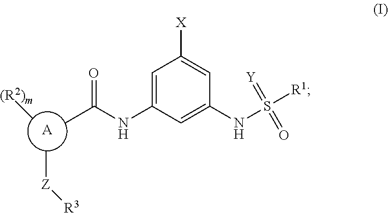

or a pharmaceutically acceptable salt thereof, wherein:
X is H, C₁₋₄alkyl, C₂₋₄haloalkyl, or C(O)R^{x};
R^{x} is H, —OH, —OC₁₋₄alkyl, or C₁₋₄alkyl;
Y is O or NR^{y};
R^{y} is H or C₁₋₄alkyl;
R¹ is C₁₋₄alkyl or C₃₋₆cycloalkyl; wherein the C₁₋₄alkyl or C₃₋₆cycloalkyl are each optionally and independently substituted with 1 to 3 halo;
Ring A is a C₃₋₆cycloalkyl, 4 to 6-membered monocyclic heterocyclyl, 6 to 10 membered bicyclic heterocyclyl, phenyl, 5 to 6-membered monocyclic heteroaryl, or 8 to 10-membered bicyclic heteroaryl;
m is 0 to 3;
each R² is independently selected from C₁₋₄alkyl, C₃₋₆cycloalkyl, halo, OR^{2a}, cyano, —NR^{2b}R^{2c}, —SO₂R^{2a}, —C(O)R^{2d}, and —C(O)NR^{2b}R^{2c}, wherein the C₁₋₄alkyl and C₃₋₆cycloalkyl are optionally substituted with 1 to 4 R^{2e}; or 2 R² together form oxo;
R^{2a} is H, C₁₋₄alkyl, C₁₋₄haloalkyl, C₁₋₄alkoxyC₁₋₄alkyl, or benzyl;
R^{2d} is H, C₁₋₄alkyl, C₃₋₆cycloalkyl; OR^{2a}, phenyl, 4 to 6-membered monocyclic heterocyclyl or 5 to 6-membered monocyclic heteroaryl, wherein the C₁₋₄alkyl is optionally substituted with 1 to 3 halo;
R^{2b} and R^{2e} are each independently selected from H, C₁₋₄alkyl, C₃₋₆cycloalkyl, phenyl, 4 to 6-membered monocyclic heterocyclyl and 5 to 6-membered monocyclic heteroaryl; or
R^{2b} and R^{2c}, together with the nitrogen to which they are attached form a 4 to 6 membered monocyclic heterocyclyl or 6 to 10 membered bicyclic heterocyclyl; wherein the 4 to 6 membered monocyclic heterocyclyl or 6 to 10 membered bicyclic heterocyclyl are each optionally substituted with 1 to 3 R^{2d};
each R^{2e} is independently selected from halo, cyano, NR^{2b}R^{2c}, OR^{2a}, phenyl, and 4 to 6 membered monocyclic heterocyclyl;
Z is a bond, —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³; Z^{a} is H or C₁₋₄alkyl;
provided when X is H and Z is a bond, ring A is phenyl, pyridinyl, thiophenyl, pyrrolyl, pyrazolyl, thiazolyl, imidazolyl, furanyl, or 1,4,5,6-tetrahydrocyclopenta[c]pyrazolyl;
R³ is C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl, wherein the C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl are each optionally and independently substituted with 1 to 4 R⁴;
each R⁴ is independently selected from C₁₋₄alkyl, halo, OR^{4a}, cyano, —NR^{4b}R^{4c}, —C(O)R^{4a}, —C(O)OR^{4a}, —C(O)NR^{4b}R^{4c}, —NR^{4b}C(O)R^{4a}—NR^{4b}C(O)OR^{4a}, —SR^{4a}, —SO₂R^{4a}, —SO₂NR^{4b}R^{4c}, —P(O)R^{4b}R^{4c}, phenyl, 5 to 6-membered monocyclic heteroaryl, 4 to 6-membered monocyclic heterocyclyl, and 6 to 10-membered bicyclic heterocyclyl, wherein the C₁₋₄alkyl is optionally substituted with 1 to 4 R^{4d}, and wherein the phenyl, 5 to 6-membered monocyclic heteroaryl, 4 to 6-membered monocyclic heterocyclyl and 6 to 10-membered bicyclic heterocyclyl are each optionally substituted with 1 to 3 R^{4e}; or two R⁴ together form oxo;
R^{4a} is H, C₁₋₄alkyl optionally substituted with 1 to 4 R^{4d}, C₃₋₆cycloalkyl, 4 to 6-membered monocyclic heterocyclyl, phenyl, or 5 to 6-membered monocyclic heteroaryl; wherein the C₃₋₆cycloalkyl, 4 to 6-membered monocyclic heterocyclyl, phenyl or 5 to 6-membered monocyclic heteroaryl are each optionally and independently substituted with 1 to 3 R^{4e};
R^{4b} and R^{4c} are each independently selected from H, phenyl, 4 to 6-membered monocyclic heterocyclyl, 5 to 6-membered monocyclic heteroaryl, and C₁₋₄alkyl optionally substituted with 1 to 4 R^{4d};
each R^{4d} is independently selected from halo, OR^{4f}, —C(O)C₁₋₄alkyl, —C(O)NR^{4b}R^{4c}, —C(O)C₁₋₄haloalkyl, phenyl, 4- to 6-membered monocyclic heterocyclyl, and 5 to 6-membered monocyclic heteroaryl, wherein the phenyl and 5 to 6-membered monocyclic heteroaryl are each optionally substituted with 1 to 3 R^{4g};
each R^{4e} is independently selected from halo, C₁₋₄alkyl optionally substituted with 1 to 3 halo, cyano, OR^{4f}, —NR^{4b}R^{4c}, —C(O)H, —C(O)C₁₋₄alkyl, —C₁₋₄alkylene-C(O)NR^{4b}R^{4c}, and —C(O)NR^{4b}R^{4c}; or two R^{4e} together form oxo;
R^{4f} is H, C₁₋₄alkyl, C₁₋₄haloalkyl, phenyl or 5 to 6-membered monocyclic heteroaryl, wherein the phenyl and 5 to 6-membered monocyclic heteroaryl are each optionally substituted with one to three halo;
each R^{4g} is independently selected from halo, OR^{4f}, C₁₋₄alkyl, halo, cyano, —NR^{4b}R^{4c}, —C(O)H, and —C(O)OR^{4f}. In some embodiments, the compound of Formula (I) is not any one of the compounds listed in Table I below.

Another aspect of the disclosure relates to pharmaceutical compositions comprising compounds of Formula (I) or pharmaceutically acceptable salts thereof, and a pharmaceutical carrier.

In yet another aspect, the present disclosure provides a method of treating a disease or disorder that is responsive to inhibition of DHX9 in a subject comprising administering to said subject an effective amount of at least one compound described herein or a pharmaceutically acceptable salt thereof. In some embodiments, the method is for the treatment of cancer.

Another aspect of the present disclosure relates to the use of at least one compound described herein or a pharmaceutically acceptable salt thereof, for the manufacture of a medicament for the treatment of a disease or disorder responsive to inhibition of DHX9. Also provided is a compound described herein or a pharmaceutically acceptable salt thereof for use in treating a disease or disorder responsive to inhibition of DHX9.

### DETAILED DESCRIPTION OF THE INVENTION

The present disclosure provides compounds and pharmaceutical compositions thereof that may be useful in the treatment of diseases or disorders through mediation of DHX9 function/activity. In some embodiments, the compounds of present disclosure are DHX9 inhibitors.

### Compounds and Compositions

In a first embodiment, the present disclosure provides a compound of Formula (I):

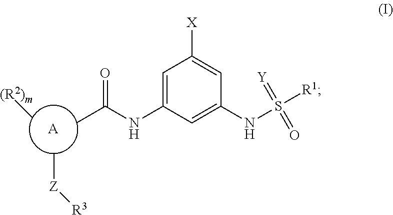

or a pharmaceutically acceptable salt thereof, wherein:
X is H, C₁₋₄alkyl, C₂₋₄haloalkyl, or C(O)R^{x};
R^{x} is H, —OH, —OC₁₋₄alkyl, or C₁₋₄alkyl;
Y is O or NR^{y};
R^{y} is H or C₁₋₄alkyl;
R¹ is C₁₋₄alkyl or C₃₋₆cycloalkyl; wherein the C₁₋₄alkyl or C₃₋₆cycloalkyl are each optionally and independently substituted with 1 to 3 halo;
Ring A is a C₃₋₆cycloalkyl, 4 to 6-membered monocyclic heterocyclyl, 6 to 10 membered bicyclic heterocyclyl, phenyl, 5 to 6-membered monocyclic heteroaryl, or 8 to 10-membered bicyclic heteroaryl;
m is 0 to 3;
each R² is independently selected from C₁₋₄alkyl, C₃₋₆cycloalkyl, halo, OR^{2a}, cyano, —NR^{2b}R^{2c}, —SO₂R^{2a}, —C(O)R^{2d}, and —C(O)NR^{2b}R^{2c}, wherein the C₁₋₄alkyl and C₃₋₆cycloalkyl are optionally substituted with 1 to 4 R^{2e}; or 2 R² together form oxo;
R^{2a} is H, C₁₋₄alkyl, C₁₋₄haloalkyl, C₁₋₄alkoxyC₁₋₄alkyl, or benzyl;
R^{2d} is H, C₁₋₄alkyl, C₃₋₆cycloalkyl; OR^{2a}, phenyl, 4 to 6-membered monocyclic heterocyclyl or 5 to 6-membered monocyclic heteroaryl, wherein the C₁₋₄alkyl is optionally substituted with 1 to 3 halo;
R^{2b} and R^{2e} are each independently selected from H, C₁₋₄alkyl, C₃₋₆cycloalkyl, phenyl, 4 to 6-membered monocyclic heterocyclyl and 5 to 6-membered monocyclic heteroaryl; or
R^{2b} and R^{2c}, together with the nitrogen to which they are attached form a 4 to 6 membered monocyclic heterocyclyl or 6 to 10 membered bicyclic heterocyclyl; wherein the 4 to 6 membered monocyclic heterocyclyl or 6 to 10 membered bicyclic heterocyclyl are each optionally substituted with 1 to 3 R^{2d};
each R^{2e} is independently selected from halo, cyano, NR^{2b}R^{2c}, OR^{2a}, phenyl, and 4 to 6 membered monocyclic heterocyclyl;
Z is a bond, —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³;
Z^{a} is H or C₁₋₄alkyl;
provided when X is H and Z is a bond, ring A is phenyl, pyridinyl, thiophenyl, pyrrolyl, pyrazolyl, thiazolyl, imidazolyl, furanyl, or 1,4,5,6-tetrahydrocyclopenta[c]pyrazolyl;
R³ is C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl, wherein the C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl are each optionally and independently substituted with 1 to 4 R⁴;
each R⁴ is independently selected from C₁₋₄alkyl, halo, OR^{4a}, cyano, —NR^{4b}R^{4c}, —C(O)R^{4a}, —C(O)OR^{4a}, —C(O)NR^{4b}R^{4c}, —NR^{4b}C(O)R^{4a}—NR^{4b}C(O)OR^{4a}, —SR^{4a}, —SO₂R^{4a}, —SO₂NR^{4b}R^{4c}, —P(O)R^{4b}R^{4c}, phenyl, 5 to 6-membered monocyclic heteroaryl, 4 to 6-membered monocyclic heterocyclyl, and 6 to 10-membered bicyclic heterocyclyl, wherein the C₁₋₄alkyl is optionally substituted with 1 to 4 R^{4d}, and wherein the phenyl, 5 to 6-membered monocyclic heteroaryl, 4 to 6-membered monocyclic heterocyclyl and 6 to 10-membered bicyclic heterocyclyl are each optionally substituted with 1 to 3 R^{4e}; or two R⁴ together form oxo;
R^{4a} is H, C₁₋₄alkyl optionally substituted with 1 to 4 R^{4d}, C₃₋₆cycloalkyl, 4 to 6-membered monocyclic heterocyclyl, phenyl, or 5 to 6-membered monocyclic heteroaryl; wherein the C₃₋₆cycloalkyl, 4 to 6-membered monocyclic heterocyclyl, phenyl or 5 to 6-membered monocyclic heteroaryl are each optionally and independently substituted with 1 to 3 R^{4e};
R^{4b} and R^{4c} are each independently selected from H, phenyl, 4 to 6-membered monocyclic heterocyclyl, 5 to 6-membered monocyclic heteroaryl, and C₁₋₄alkyl optionally substituted with 1 to 4 R^{4d};
each R^{4d} is independently selected from halo, OR^{4f}, —C(O)C₁₋₄alkyl, —C(O)NR^{4b}R^{4c}, —C(O)C₁₋₄haloalkyl, phenyl, and 5 to 6-membered monocyclic heteroaryl, wherein the phenyl and 5 to 6-membered monocyclic heteroaryl are each optionally substituted with 1 to 3 R^{4g};
each R^{4f} is independently selected from halo, C₁₋₄alkyl optionally substituted with 1 to 3 halo, cyano, OR^{4f}, —NR^{4b}R^{4c}, —C(O)H, —C(O)C₁₋₄alkyl, —C₁₋₄alkylene-C(O)NR^{4b}R^{4c}, and —C(O)NR^{4b}R^{4c}; or two R^{4e} together form oxo;
R^{4f} is H, C₁₋₄alkyl, C₁₋₄haloalkyl, phenyl or 5 to 6-membered monocyclic heteroaryl, wherein the phenyl and 5 to 6-membered monocyclic heteroaryl are each optionally substituted with one to three halo;
each R^{4g} is independently selected from halo, OR^{4f}, C₁₋₄alkyl, halo, cyano, —NR^{4b}R^{4c}, —C(O)H, and —C(O)OR^{4f}.

In some embodiments, the compound of Formula (I) is not any one of the compounds listed in Table I below.

In a second embodiment, for the compounds of Formula (I) described in the first aspect, or a pharmaceutically acceptable salt thereof, R¹ is —CH₃ or —CH₂CH₃; and the remaining variables are as described in the first aspect or the first embodiment.

In a third embodiment, for the compounds of Formula (I) described in the first aspect, or a pharmaceutically acceptable salt thereof, X is H; and the remaining variables are as described in the first aspect or the first or second embodiment.

In a fourth embodiment, for the compounds of Formula (I) described in the first aspect, or a pharmaceutically acceptable salt thereof, X is C₁₋₄alkyl, C₂₋₄haloalkyl, or C(O)R^{x}; and the remaining variables are as described in the first aspect or the first or second embodiment.

In a fifth embodiment, for the compounds of Formula (I) described in the first aspect, or a pharmaceutically acceptable salt thereof, X is —CH₃ or —C(O)H; and the remaining variables are as described in the first aspect or the first or second embodiment.

In a sixth embodiment, for the compounds of Formula (I) described in the first aspect, or a pharmaceutically acceptable salt thereof, ring A is phenyl, thiophenyl, pyrrolyl, pyrazoyl, furanyl, isothiazoyl, thiazoyl, imidazoyl, cyclobutyl, benzofuranyl, 2-oxo-2,3-dihydro-1H-benzo[d]imidazolyl, imidazo[1,2-a]pyridin-6-yl, 1,4,5,6-tetrahydrocyclopenta[c]pyrazolyl, 2-oxo-2,3-dihydro-1H-imidazolyl, indolizinyl, pyrrolo[1,2-a]pyrimidinyl, pyrrolo[1,2-c]pyrimidinyl, pyrrolo[1,2-a]pyrazinly, 5-oxo-5H-thiazolo[3,2-a]pyridinyl, thieno[3,2-b]pyridinyl, thieno[3,2-c]pyridinyl, thieno[2,3-c]pyridinyl, benzothiophenyl, thieno[3,2-d]pyrimidinyl, pyrrolo[1,2-b]pyridazinyl, pyrrolo[1,2-a]pyrazinyl, pyrrolo[1,2-a]pyrimidinyl, pyrrolo[1,2-b]pyridazinyl, pyrrolo[1,2-c]pyrimidinyl, 1-oxo-1,2-dihydropyrrolo[1,2-a]pyrazinyl, pyrrolo[2,1-f][1,2,4]triazinyl, 4,5,6,7-tetrahydrothieno[2,3-c]pyridinyl, 5,6-dihydro-4H-cyclopenta[b]thiophenyl, or 4,7-dihydro-5H-thieno[2,3-c]pyranyl; and the remaining variables are as described in first aspect or the first, second, third, fourth, or fifth embodiment.

In a seventh embodiment, for the compounds of Formula (I) described in the first aspect, or a pharmaceutically acceptable salt thereof, ring A is phenyl, pyridinyl, thiophenyl, pyrrolyl, pyrazolyl, thiazolyl, imidazolyl, furanyl, or 1,4,5,6-tetrahydrocyclopenta[c]pyrazolyl; and the remaining variables are as described in the first aspect or the first, second, third, fourth, or fifth embodiment.

In an eighth embodiment, for the compounds of Formula (I) described in the first aspect, or a pharmaceutically acceptable salt thereof, ring A is

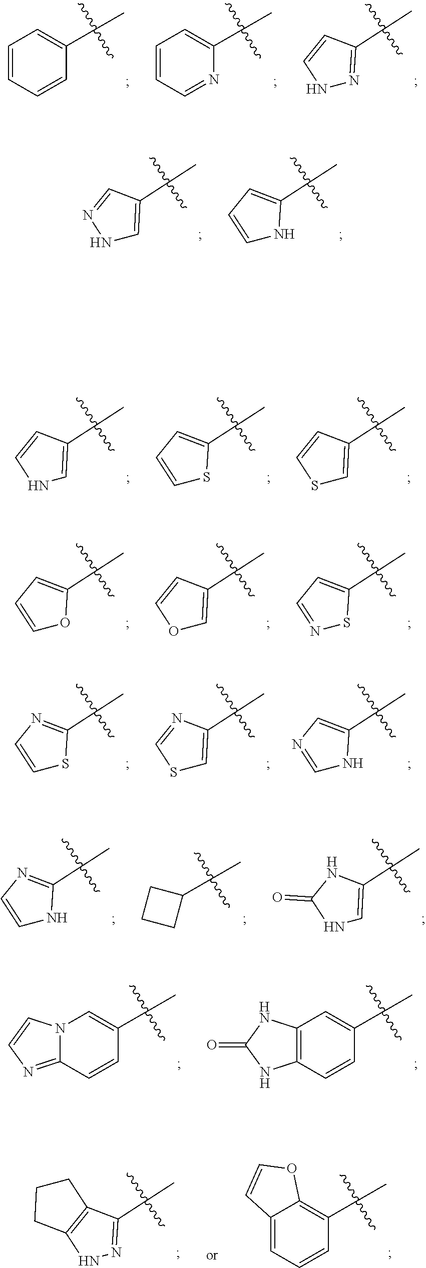

each of which is optionally substituted with 1 to 2 R²; and the remaining variables are as described in the first aspect or the first, second, third, fourth, or fifth embodiment.

In a ninth embodiment, for the compounds of Formula (I) described in the first aspect, or a pharmaceutically acceptable salt thereof, ring A is

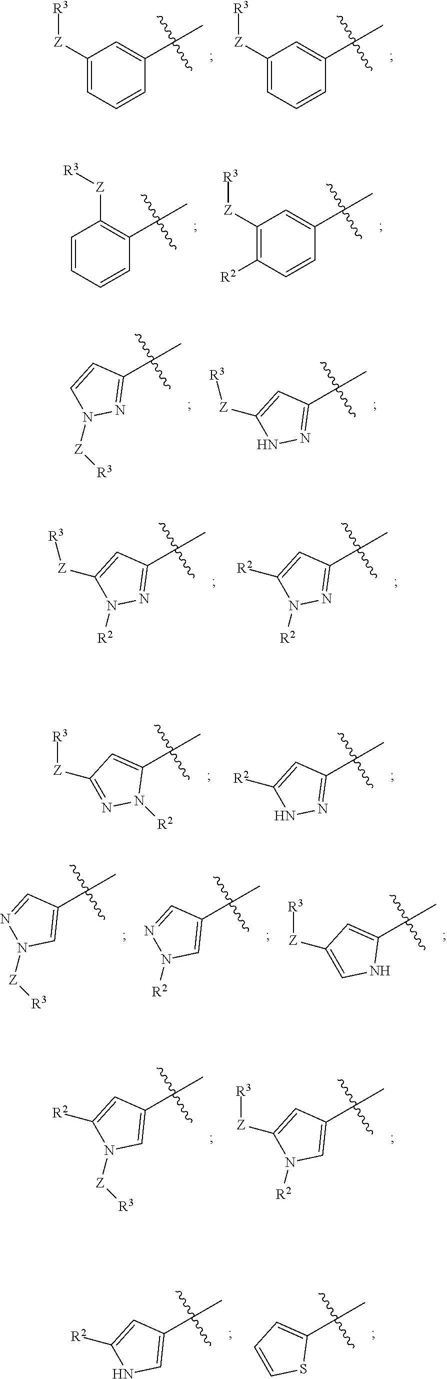

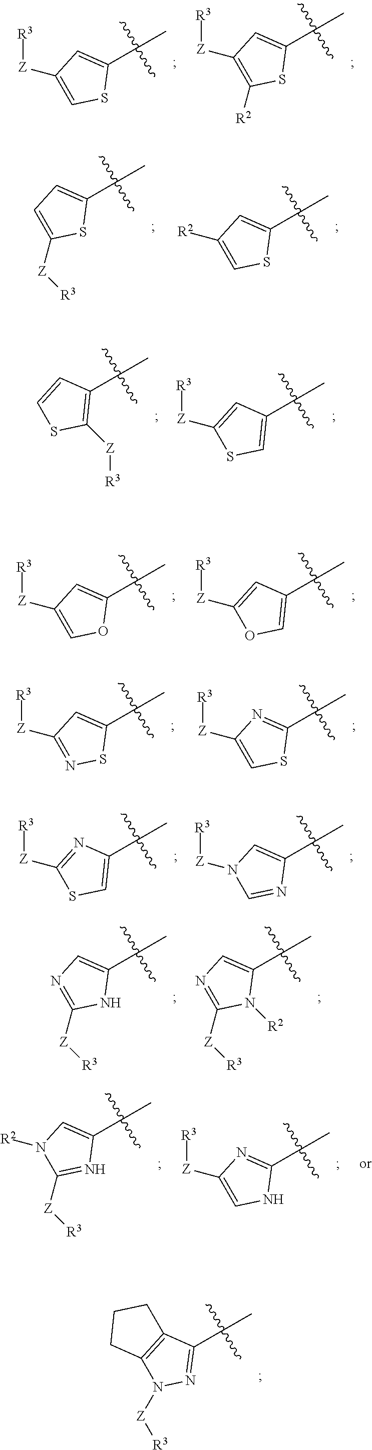

and the remaining variables are as described in the first aspect or the first, second, third, fourth, or fifth embodiment.

In a tenth embodiment, for the compounds of Formula (I) described in the first aspect, or a pharmaceutically acceptable salt thereof, Z is a bond; and the remaining variables are as described in the first aspect or the first, second, third, fourth, fifth, sixth, seventh, eighth, or ninth embodiment.

In an eleventh embodiment, for the compounds of Formula (I) described in the first aspect, or a pharmaceutically acceptable salt thereof, ring A is phenyl, thiophenyl, pyrazolyl, isothiazolyl, or imidazolyl; and the remaining variables are as described in the first aspect or the first, second, third, fourth, or fifth embodiment.

In a twelfth embodiment, for the compounds of Formula (I) described in the first aspect, or a pharmaceutically acceptable salt thereof, ring A is

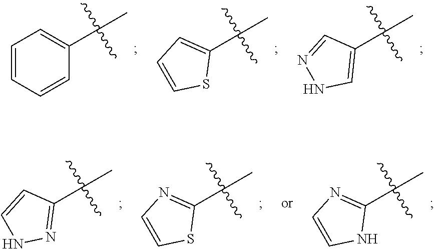 [SMILES: *c1ccccc1 ; *c1cccs1 ; *c1cn[nH]c1 ; *c1cc[nH]n1 ; *c1nccs1 ; *c1ncc[nH]1]

each of which is substituted with 0 to 1 R²; and the remaining variables are as described in the first aspect the first, second, third, fourth, or fifth embodiment.

In a thirteenth embodiment, for the compounds of Formula (I) described in the first aspect, or a pharmaceutically acceptable salt thereof, Z is a bond and ring A is

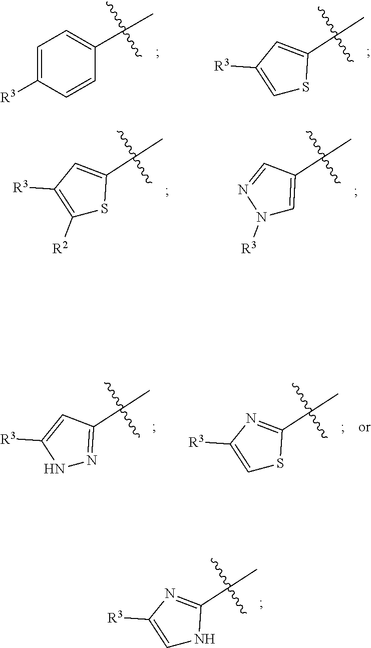

and the remaining variables are as described in the first aspect or the first, second, third, fourth, or fifth embodiment.

In a fourteenth embodiment, for the compounds of Formula (I) described in the first aspect, or a pharmaceutically acceptable salt thereof, Z is a bond and ring A is

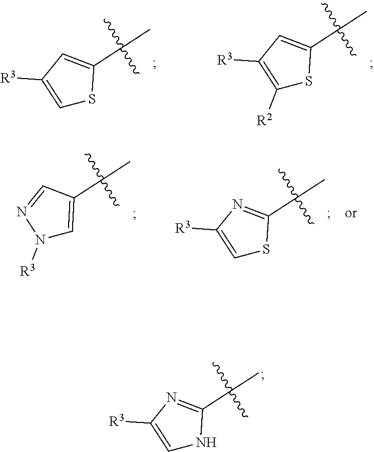

and the remaining variables are as described in the first aspect or the first, second, third, fourth, or fifth embodiment.

In a fifteenth embodiment, for the compounds of Formula (I) described in the first aspect, or a pharmaceutically acceptable salt thereof, Z is —CH₂—, —O—, —O—C₁₋₄alkylene-*, —C₁ 4alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³; and the remaining variables are as described in the first aspect or the first, second, third, fourth, fifth, sixth, seventh, eighth, ninth, eleventh, or twelfth embodiment.

In a sixteenth embodiment, for the compounds of Formula (I) described in the first aspect, or a pharmaceutically acceptable salt thereof, Z is —C(O)NH—* or —C(O)NH—C₁₋₃alkylene-*, wherein * indicates the attachment point to R³; and the remaining variables are as described in the first aspect or the first, second, third, fourth, fifth, sixth, seventh, eighth, ninth, eleventh, or twelfth embodiment.

In a seventeenth embodiment, for the compounds of Formula (I) described in the first aspect, or a pharmaceutically acceptable salt thereof, Z is C(O)NH—*, wherein * indicates the attachment point to R³; and the remaining variables are as described in the first aspect or the first, second, third, fourth, fifth, sixth, seventh, eighth, ninth, eleventh, or twelfth embodiment.

In an eighteenth embodiment, for the compounds of Formula (I) described in the first aspect, or a pharmaceutically acceptable salt thereof, ring A is

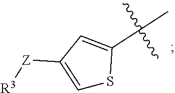

and the remaining variables are as described in the first aspect or the first, second, third, fourth, fifth, sixth, seventh, eighth, ninth, eleventh, twelfth, fifteenth, sixteenth, or seventeenth embodiment.

In a nineteenth embodiment, for the compounds of Formula (I) described in the first aspect, or a pharmaceutically acceptable salt thereof, R² is C₁₋₄alkyl, halo, cyano, —C(O)R^{2d}, or —C(O)NR^{2b}R^{2c}, wherein the C₁₋₄alkyl is optionally substituted with 1 to 4 R^{2e}; R^{2b} and R^{2e} are each independently selected from H or C₁₋₄alkyl optionally substituted with C₁₋₃alkoxy; R^{2d} is OH or C₁₋₃alkoxy; and R^{2e} is halo, —OH or C₁₋₃alkoxy; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, or eighteenth embodiment.

In a twentieth embodiment, for the compounds of Formula (I) described in the first aspect, or a pharmaceutically acceptable salt thereof, R² is —CH₃, —CF₃, —CH₂OH, halo, cyano, —C(O)OH, —C(O)NHCH₃, or

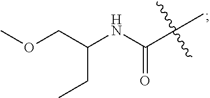 [SMILES: *C(=O)NC(CC)COC]

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, or eighteenth embodiment.

In a twenty-first embodiment, for the compounds of Formula (I) described in the first aspect, or a pharmaceutically acceptable salt thereof, R³ is cyclopropyl, cyclobutyl, cyclohexyl, piperidinyl, phenyl, pyrrolyl, pyrazolyl, pyridyl, pyrimidyl, pyrazinyl, pyrazinyl, thiophenyl, tetrahydropyranyl, thiazolyl, triazolyl, tetrazolyl, oxadiazolyl, 3-oxo-2,3-dihydro-1H-pyrazoly, benzamidazolyl, indazolyl, indoyl, 2,3-dihydrobenzofuranyl, imidazo[1,2-a]pyridinyl, 2,3-dihydro-1H-indenyl, 2-oxo-2,3-dihydro-1H-benzo[d]imidazolyl, and 2-azaspiro[3.3]heptanyl, or 2-oxaspiro[3.3]heptanyl, each of which is optionally substituted with 1 to 3 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-second embodiment, for the compounds of Formula (I) described in the first aspect, or a pharmaceutically acceptable salt thereof, R³ is

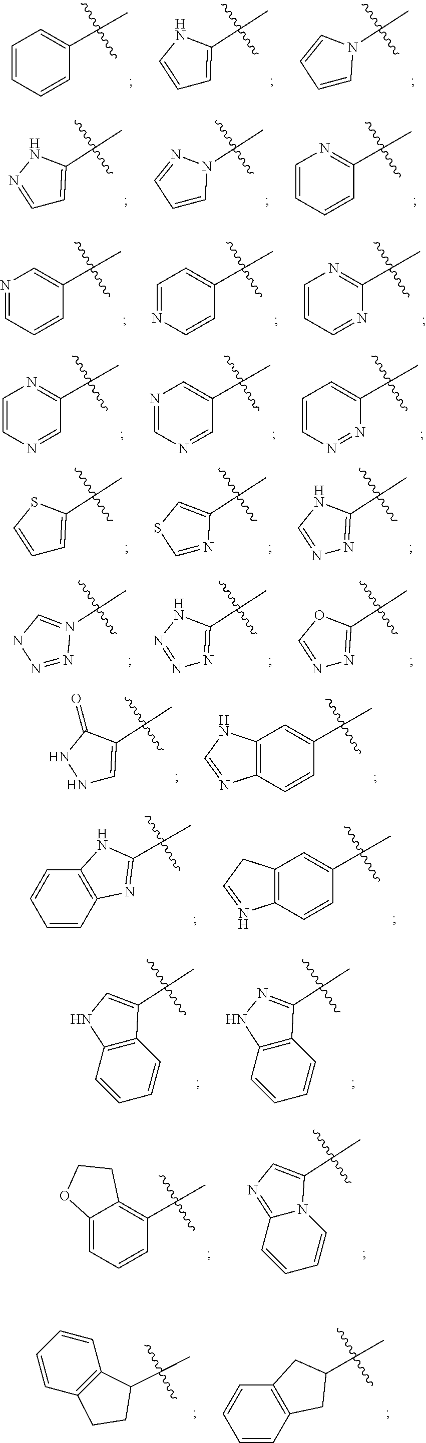

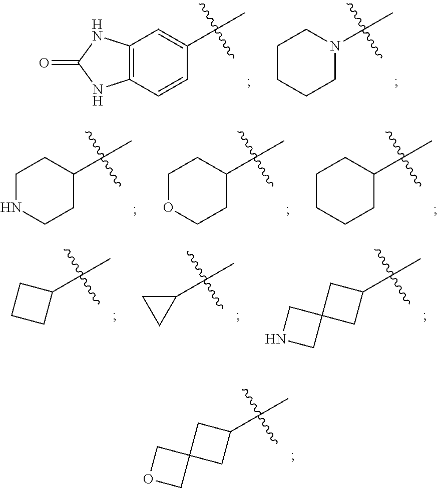

each of which is optionally substituted with 1 to 3 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-third embodiment, for the compounds of Formula (I) described in the first aspect, or a pharmaceutically acceptable salt thereof, R³ is

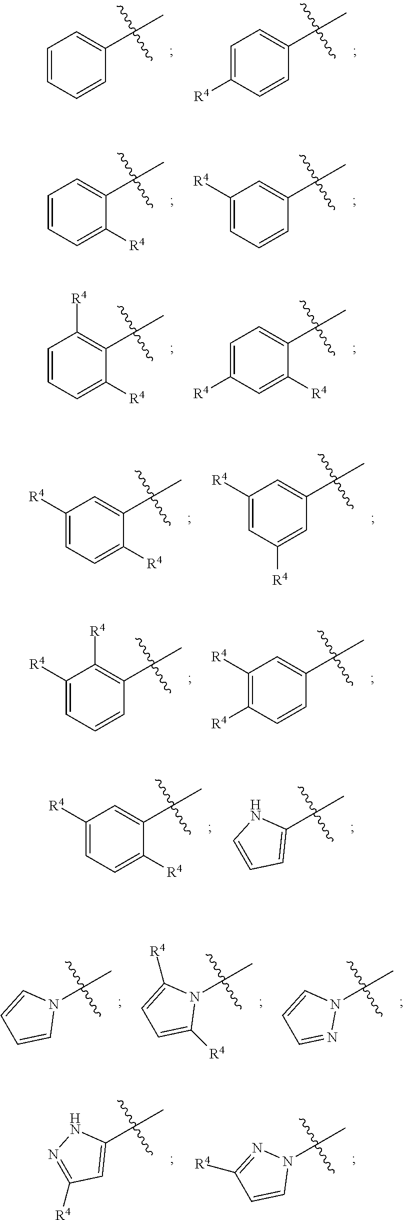

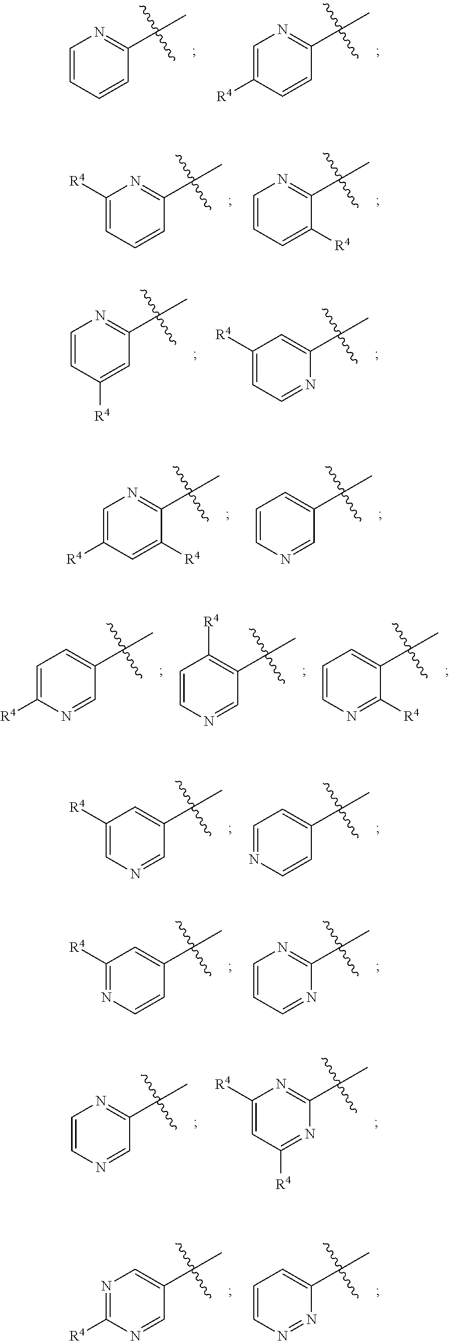

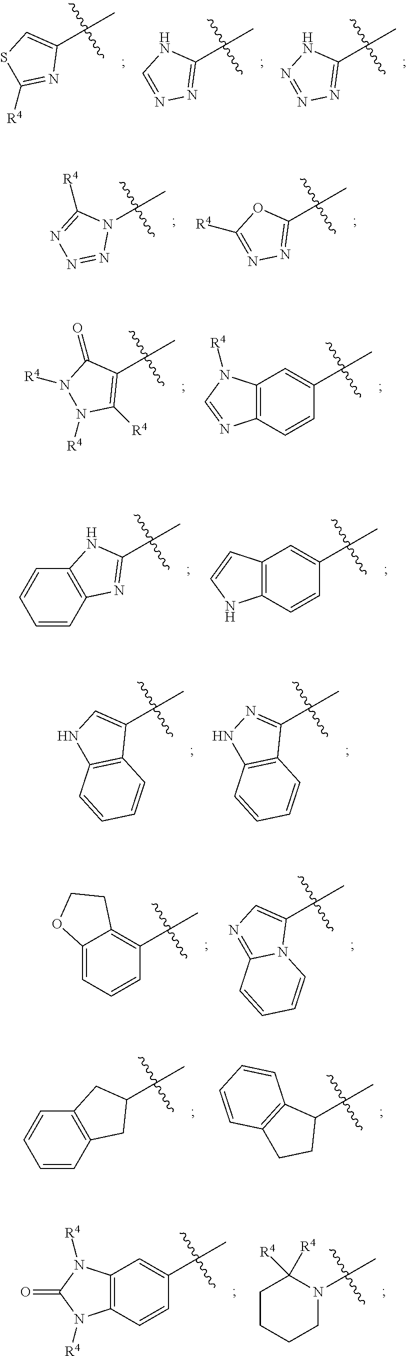

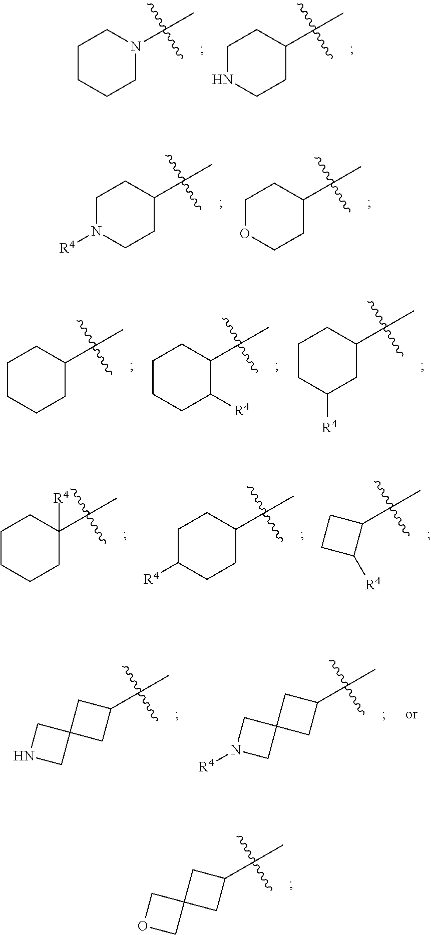

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-fourth embodiment, for the compounds of Formula (I) described in the first aspect, or a pharmaceutically acceptable salt thereof, Z is a bond, R³ is phenyl, pyridyl, pyrimidyl, pyrazinyl, thiophenyl, pyrrolyl, pyrazoyl, isothiazoyl, triazoyl, tetrazoyl, or oxadiazoyl, each of which is optionally substituted with 1 to 2 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-fifth embodiment, for the compounds of Formula (I) described in the first aspect, or a pharmaceutically acceptable salt thereof, Z is a bond, R³ is

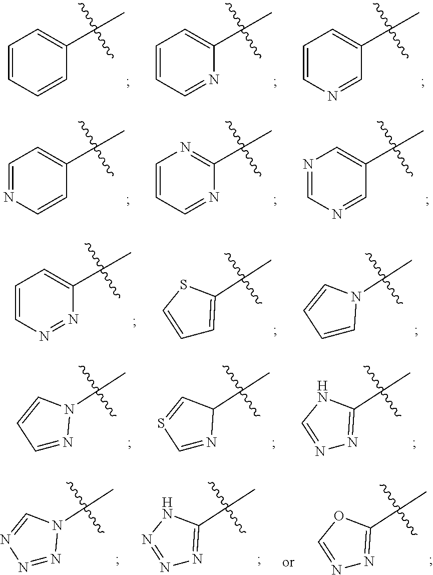

each of which is optionally substituted with 1 to 2 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-sixth embodiment, for the compounds of Formula (I) described in the first aspect, or a pharmaceutically acceptable salt thereof, Z is a bond, R³ is

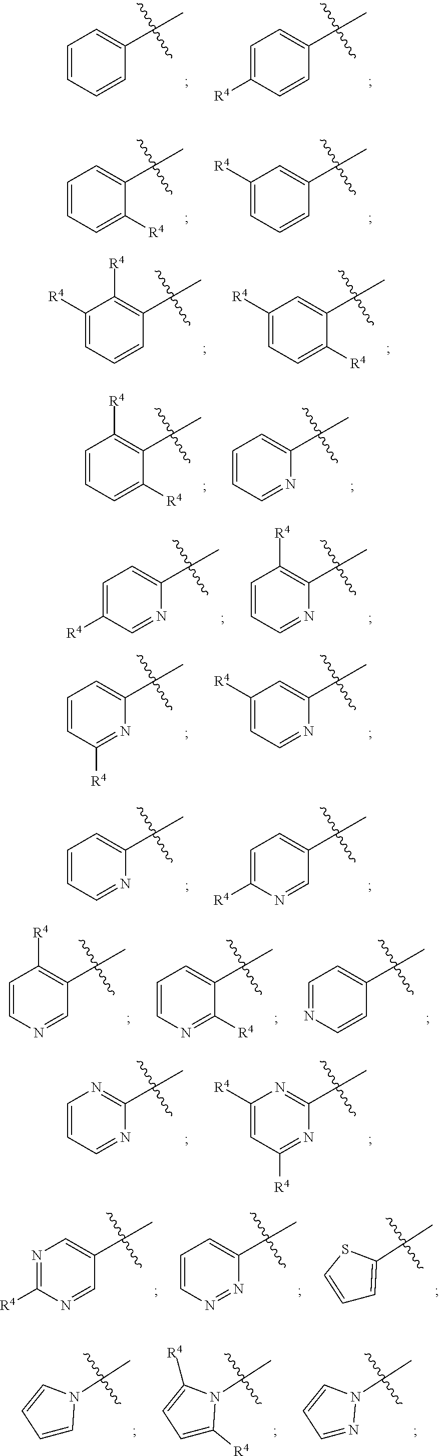

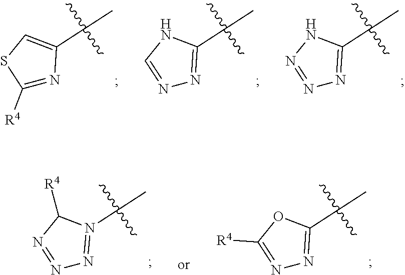

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-seventh embodiment, for the compounds of Formula (I) described in the first aspect, or a pharmaceutically acceptable salt thereof, Z is a bond, R³ is phenyl, pyridyl, or pyrimidyl, each of which is optionally substituted with 1 to 2 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-eighth embodiment, for the compounds of Formula (I) described in the first aspect, or a pharmaceutically acceptable salt thereof, Z is a bond, R³ is

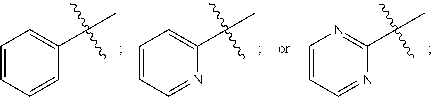 [SMILES: *c1ccccc1 ; *c1ccccn1 ; *c1ncccn1]

each of which is optionally substituted with 1 to 2 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-ninth embodiment, for the compounds of Formula (I) described in the first aspect, or a pharmaceutically acceptable salt thereof, Z is a bond, R³ is

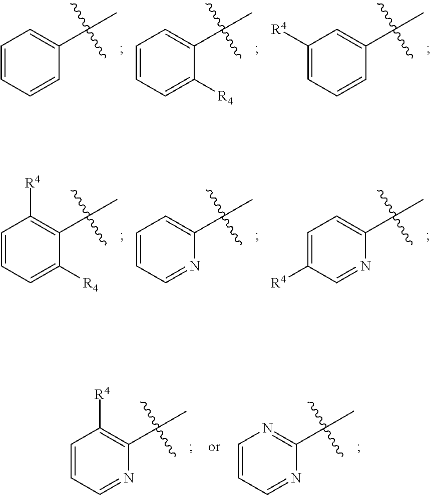

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a thirtieth embodiment, for the compounds of Formula (I) described in the first aspect, or a pharmaceutically acceptable salt thereof, Z is —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³, R³ is cyclopropyl, cyclohexyl, piperidinyl, phenyl, pyridyl, pyrazoyl, isothiazoyl, 2-oxo-2,3-dihydro-1H-pyrazoly, benzamidazoyl, indazoyl, indoyl, 2,3-dihydrobenzofuranyl, imidazo[1,2-a]pyridinyl, 2,3-dihydro-1H-indenyl, or 2-oxo-2,3-dihydro-1H-benzo[d]imidazolyl, each of which is optionally substituted with 1 to 3 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a thirty-first embodiment, for the compounds of Formula (I) described in the first aspect, or a pharmaceutically acceptable salt thereof, Z is —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³, R³ is

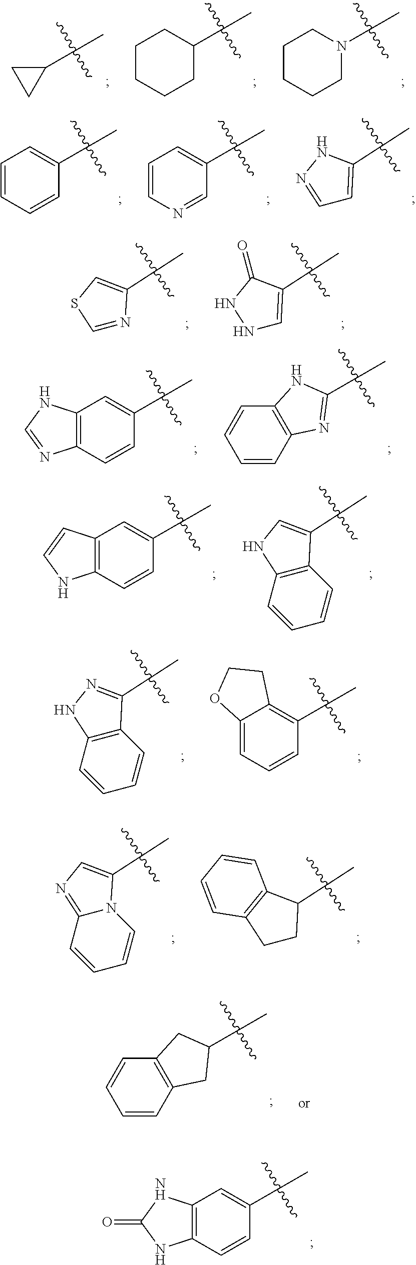

each of which is optionally substituted with 1 to 3 R⁴; and the remaining variables are as described in the first aspect of the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a thirty-second embodiment, for the compounds of Formula (I) described in the first aspect, or a pharmaceutically acceptable salt thereof, Z is —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³, R³ is

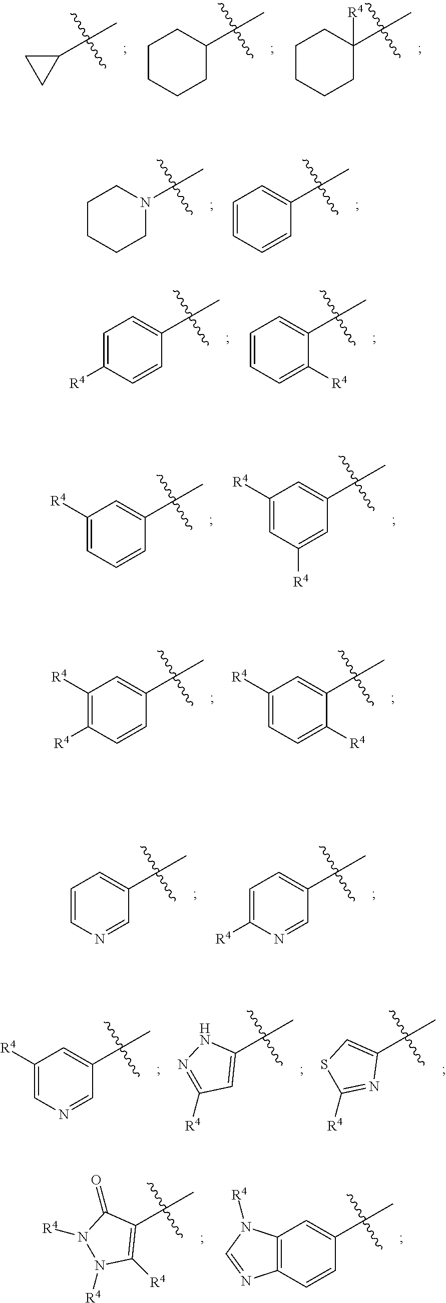

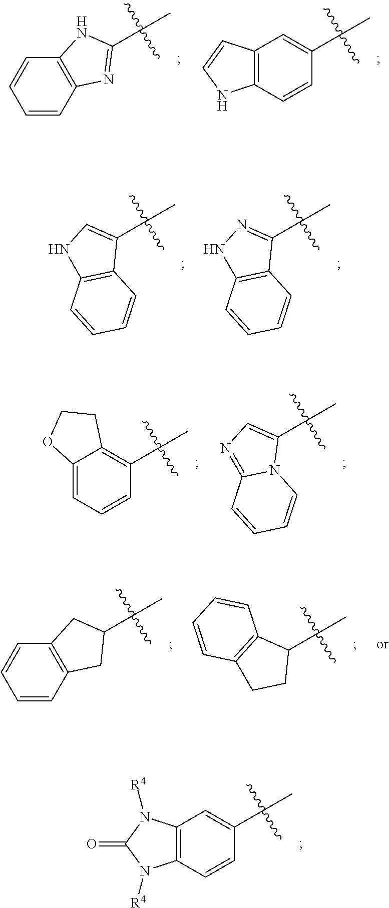

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a thirty-third embodiment, for the compounds of Formula (I) described in the first aspect, or a pharmaceutically acceptable salt thereof, Z is —C(O)NH—*, wherein * indicates the attachment point to R³, R³ is benzamidazolyl or 2-oxo-2,3-dihydro-1H-benzo[d]imidazolyl, each of which is optionally substituted with 1 to 2 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a thirty-fourth embodiment, for the compounds of Formula (I) described in the first aspect, or a pharmaceutically acceptable salt thereof, Z is —C(O)NH—*, wherein * indicates the attachment point to R³, R³ is

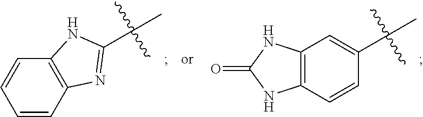 [SMILES: *c1nc2ccccc2[nH]1 ; *c1ccc2[nH]c(=O)[nH]c2c1]

each of which is optionally substituted with 1 to 2 R⁴; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a thirty-fifth embodiment, for the compounds of Formula (I) described in the first aspect, or a pharmaceutically acceptable salt thereof, Z is —C(O)NH—*, wherein * indicates the attachment point to R³, R³ is

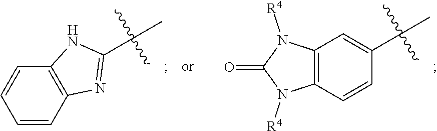

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a thirty-sixth embodiment, for the compounds of Formula (I) described in the first aspect, or a pharmaceutically acceptable salt thereof, R⁴ is independently selected from C₁₋₄alkyl, halo, OR^{4a}, cyano, —NR^{4b}R^{4c}, —C(O)OH, —C(O)NR^{4b}R^{4c}, —NR^{4b}C(O)R^{4a}, —NR^{4b}C(O)OR^{4a}, 4 to 6-membered monocyclic heterocyclyl, phenyl, and 4 to 6-membered monocyclic heteroaryl, wherein the C₁₋₄alkyl is optionally substituted with 1 to 3 substituents selected from OR^{4f}, cyano, or 4 to 6-membered monocyclic heterocyclyl; and wherein the 4 to 6-membered monocyclic heterocyclyl, phenyl, and to 6-membered monocyclic heteroaryl are each optionally substituted with 1 to 3 R^{4e}; or two R⁴ together form oxo; R^{4a} is H, phenyl, or C₁₋₄alkyl optionally substituted with 1 to 3 substituents independently selected from halo, phenyl and 4 to 6-membered monocyclic heterocyclyl; R^{4b} and R^{4c} are each independently selected from H and C₁₋₄alkyl; each R^{4e} is independently selected from C₁₋₄alkyl or —C(O)C₁₋₄alkyl; R^{4f} is H or C₁₋₄alkyl; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, or thirty-fifth embodiment. In an alternative thirty-sixth embodiment, for the compounds of Formula (I) described in the first aspect, or a pharmaceutically acceptable salt thereof, R⁴ is independently selected from C₁₋₄alkyl, halo, OR^{4a}, cyano, —NR^{4b}R^{4c}, —C(O)OH, —C(O)NR^{4b}R^{4c}, —NR^{4b}C(O)R^{4a}, —NR^{4b}C(O)OR^{4a}, 4 to 6-membered monocyclic heterocyclyl, phenyl, and 4 to 6-membered monocyclic heteroaryl, wherein the C₁₋₄alkyl is optionally substituted with 1 to 3 substituents selected from OR^{4f}, cyano, or 4 to 6-membered monocyclic heterocyclyl; and wherein the 4 to 6-membered monocyclic heterocyclyl, phenyl, and to 6-membered monocyclic heteroaryl are each optionally substituted with 1 to 3 R^{4e}; or two R⁴ together form oxo; R^{4a} is H, phenyl, or C₁₋₄alkyl optionally substituted with 1 to 3 substituents independently selected from halo, phenyl, 5 to 6-membered monocyclic heteroaryl, and 4 to 6-membered monocyclic heterocyclyl, wherein the phenyl, 5 to 6-membered monocyclic heteroaryl, and 4 to 6-membered monocyclic heterocyclyl are each optionally substituted with 1 to 3 R^{4g}; R^{4b} and R^{4c} are each independently selected from H and C₁₋₄alkyl; each R^{4e} is independently selected from C₁₋₄alkyl, halo, or —C(O)C₁₋₄alkyl; R^{4f} is H or C₁₋₄alkyl; and each R^{4g} is independently halo or —OH.

In a thirty-seventh embodiment, for the compounds of Formula (I) described in the first aspect, or a pharmaceutically acceptable salt thereof, R⁴ is

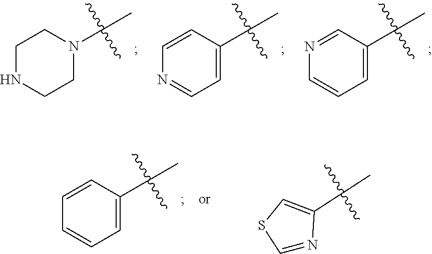 [SMILES: *N1CCNCC1 ; *c1ccncc1 ; *c1cccnc1 ; *c1ccccc1 ; *c1cscn1]

each of which is optionally substituted with 1 to 2 R^{4e}; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, or thirty-sixth embodiment or any alternative embodiments described therein. In an alternative thirty-seventh embodiment, for the compounds of Formula (I) described in the first aspect, or a pharmaceutically acceptable salt thereof, R⁴ is represented by the following structural formula:

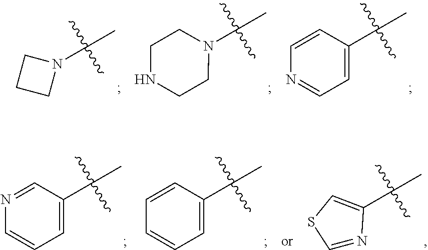 [SMILES: *N1CCC1 ; *N1CCNCC1 ; *c1ccncc1 ; *c1cccnc1 ; *c1ccccc1 ; *c1cscn1]

each of which is optionally substituted with 1 to 2 R^{4e}; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, or thirty-sixth embodiment or any alternative embodiments described therein.

In a thirty-eighth embodiment, for the compounds of Formula (I) described in the first aspect, or a pharmaceutically acceptable salt thereof, R⁴ is

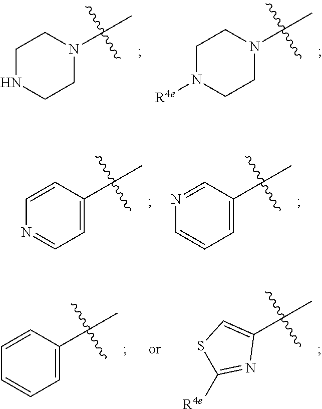

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, or thirty-sixth embodiment or any alternative embodiments described therein. In an alternative thirty-eighth embodiment, for the compounds of Formula (I) described in the first aspect, or a pharmaceutically acceptable salt thereof, R⁴ is represented by the following structural formula:

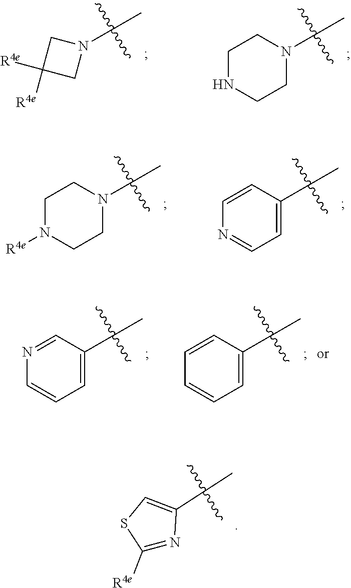

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, or thirty-sixth embodiment or any alternative embodiments described therein.

In a thirty-ninth embodiment, for the compounds of Formula (I) described in the first aspect, or a pharmaceutically acceptable salt thereof, R^{4e} is —CH₃ or —C(O)CH₃; and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, or thirty-eighth embodiment or any alternative embodiments described therein. In an alternative thirty-ninth embodiment, for the compounds of Formula (I) described in the first aspect, or a pharmaceutically acceptable salt thereof, R^{4e} is —F, —CH₃, or —C(O)CH₃; the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, or thirty-eighth embodiment or any alternative embodiments described therein.

In a fortieth embodiment, for the compounds of Formula (I) described in the first aspect, or a pharmaceutically acceptable salt thereof, R⁴ is —CH₃, —CH₂OCH₃, —CH₂CN, —CH₂CH₂OH, halo, cyano, —OH, —OCH₃, —OCH₂CH₃, —OCH(CH₃)₂, —OCF₃, —NH₂, —NHC(O)CH₃, —C(O)OH, —C(O)NH₂, —C(O)NHCH₃, —C(O)N(CH₃)₂, —C(O)NC(CH₃)₃,

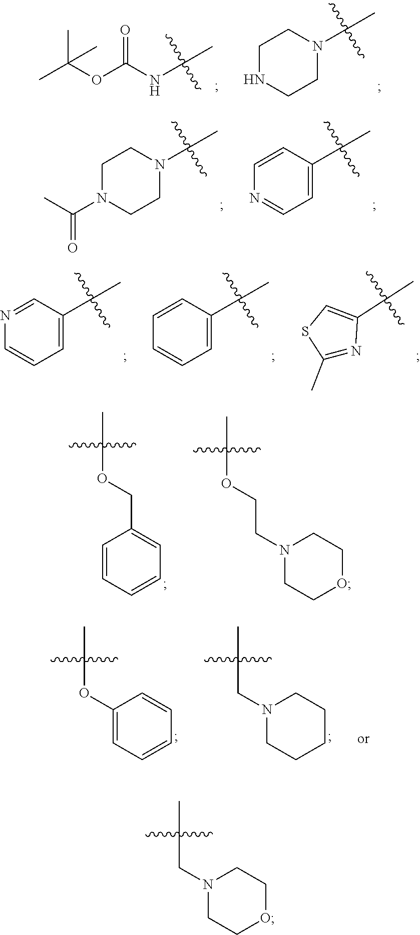

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, or thirty-ninth embodiment. In an alternative fortieth embodiment, for the compounds of Formula (I) described in the first aspect, or a pharmaceutically acceptable salt thereof, R⁴ is —CH₃, —CH₂OCH₃, —CH₂CN, —CH₂CH₂OH, halo, cyano, —OH, —OCH₃, —OCH₂CH₃, —OCH(CH₃)₂, —OCF₃, —NH₂, —NHC(O)CH₃, —C(O)OH, —C(O)NH₂, —C(O)NHCH₃, —C(O)N(CH₃)₂, —C(O)NC(CH₃)₃, or R⁴ is represented by the following formula:

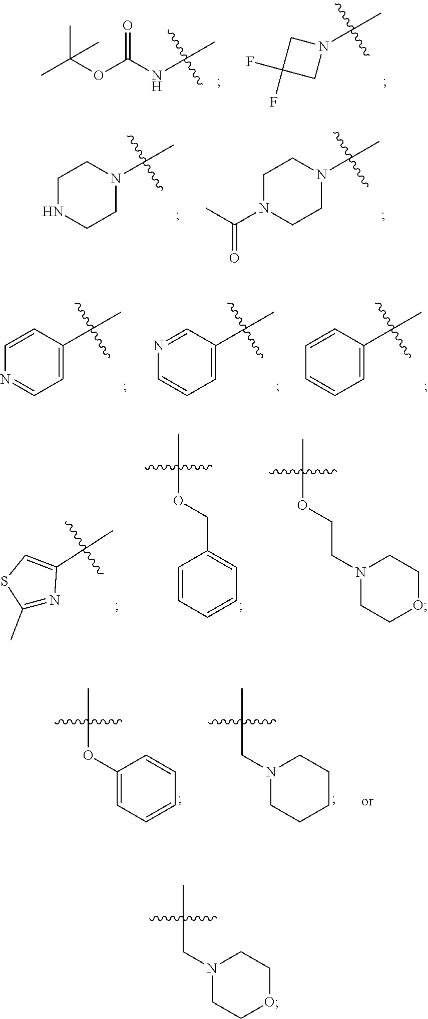

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, or thirty-ninth embodiment.

In a forty-first embodiment, for the compounds of Formula (I) described in the first aspect, or a pharmaceutically acceptable salt thereof, R⁴ is —CH₃, halo, cyano, —OH, —OCH₃, —OCH₂CH₃, —NH₂, —NHC(O)CH₃, —C(O)OH, —C(O)NH₂,

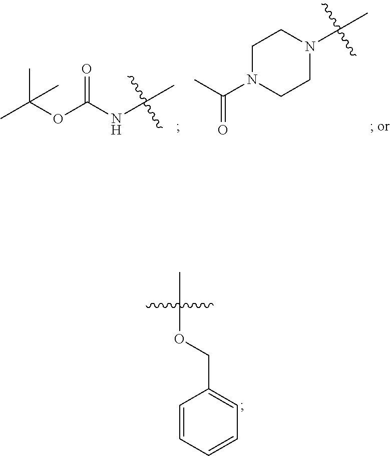

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, or thirty-ninth embodiment or any alternative embodiments described therein.

In a forty-second embodiment, for the compounds of Formula (I) described in the first aspect, or a pharmaceutically acceptable salt thereof, R⁴ is —CH₃, —NH₂, —OH, halo, or —NHC(O)CH₃,

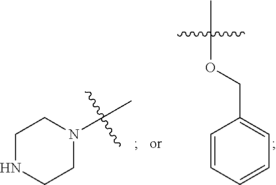 [SMILES: *N1CCNCC1 ; *OCc1ccccc1]

and the remaining variables are as described in the first aspect or the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, thirty-fourth, thirty-fifth, thirty-sixth, thirty-seventh, thirty-eighth, or thirty-ninth embodiment or any alternative embodiments describe therein.

In a forty-third embodiment, for the compounds of Formula (I) described in the first aspect, or a pharmaceutically acceptable salt thereof, Z is —CH₂—, —OCH₂—*, —S(O)₂, —S(O)₂N(H)—*, —N(H)—, —N(H)CH₂—*, —C(O)NH—*, or —C(O)NH—C₁₋₃alkylene-*, wherein * indicates the attachment point to R³, R⁴ is —CH₃, —CH₂OCH₃, —CH₂CN, —CH₂CH₂OH, halo, —OCH₃, —OCH(CH₃)₂, —OCF₃, C(O)NHCH₃, —C(O)N(CH₃)₂, —C(O)NC(CH₃)₃,

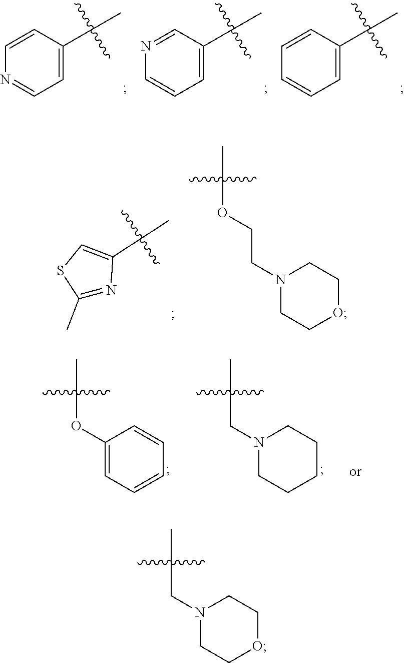

and the remaining variables are as described in the first aspect or the first, second, third, fourth, fifth, sixth, seventh, eighth, ninth, eleventh, twelfth, fifteenth, eighteenth, nineteenth, twentieth, twenty-first, twenty-second, twenty-third, thirtieth, thirty-first, thirty-second, thirty-sixth, thirty-seventh, thirty-eighth, or thirty-ninth embodiment or any alternative embodiments.

In a forty-fourth embodiment, for the compounds of Formula (I) described in the first aspect, or a pharmaceutically acceptable salt thereof, Z is —CH₂—, —OCH₂—*, —S(O)₂, —S(O)₂N(H)—*, —N(H)—, —N(H)CH₂—*, —C(O)NH—*, or —C(O)NH—C₁₋₃alkylene-*, wherein * indicates the attachment point to R³, R⁴ is —CH₃; and the remaining variables are as described in the first aspect or the first, second, third, fourth, fifth, sixth, seventh, eighth, ninth, eleventh, twelfth, fifteenth, eighteenth, nineteenth, twentieth, twenty-first, twenty-second, twenty-third, thirtieth, thirty-first, thirty-second, thirty-sixth, thirty-seventh, thirty-eighth, or thirty-ninth embodiment or any alternative embodiments described therein.

In a forty-fifth embodiment, the compound of the present disclosure is represented by Formula (IIA), (IIB) or (IIC):

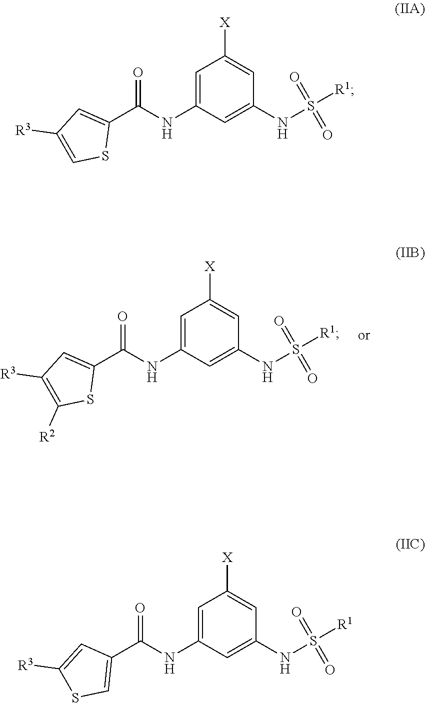

or a pharmaceutically acceptable salt thereof; where the variables X, R¹, R² and R³ depicted in Formula (IIA), (IIB) or (IIC) are as described in the first aspect or the first, second, third, fourth, fifth, nineteenth, twentieth, twenty-first, twenty-second, twenty-third, twenty-fourth, twenty-fifth, twenty-sixth, twenty-seventh, twenty-eighth, twenty-ninth, thirty-sixth, thirty-seventh, thirty-eighth, thirty-ninth, fortieth, forty-first, or forty-second embodiment or any alternative embodiments described therein.

In a forty-sixth embodiment, the compound of the present disclosure is represented by Formula (IIIA) or (IIIB):

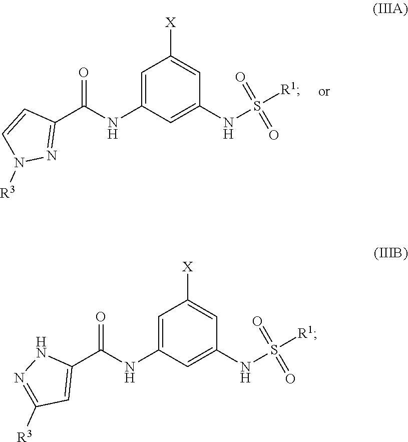

or a pharmaceutically acceptable salt thereof; where the variables X, R¹, and R³ depicted in Formula (IIIA) or (IIIB) are as described in the first aspect or the first, second, third, fourth, fifth, nineteenth, twentieth, twenty-first, twenty-second, twenty-third, twenty-fourth, twenty-fifth, twenty-sixth, twenty-seventh, twenty-eighth, twenty-ninth, thirty-sixth, thirty-seventh, thirty-eighth, thirty-ninth, fortieth, forty-first, or forty-second embodiment or any alternative embodiments described therein.

In a forty-seventh embodiment, the compound of the present disclosure is represented by Formula (IVA), (IVB), (IVC), or (IVD):

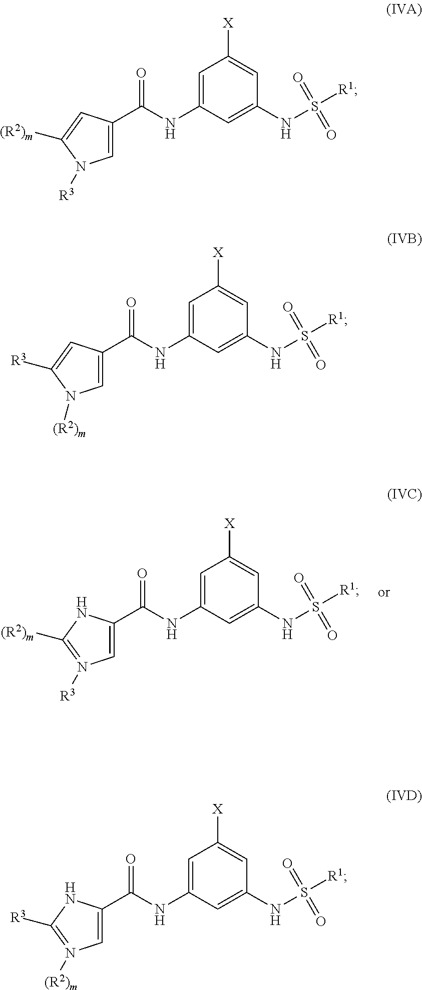

or a pharmaceutically acceptable salt thereof; where m is 0 or 1, and where the variables X, R¹, R² and R³ depicted in Formula (IVA), (IVB), (IVC), or (IVD) are as described in the first aspect or the first, second, third, fourth, fifth, nineteenth, twentieth, twenty-first, twenty-second, twenty-third, twenty-fourth, twenty-fifth, twenty-sixth, twenty-seventh, twenty-eighth, twenty-ninth, thirty-sixth, thirty-seventh, thirty-eighth, thirty-ninth, fortieth, forty-first, or forty-second embodiment or any alternative embodiments described therein.

In a forty-eighth embodiment, the compound of the present disclosure is represented by Formula (V):

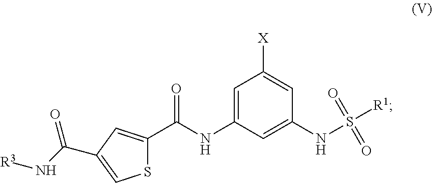

or a pharmaceutically acceptable salt thereof; where the variables X, R¹, and R³ depicted in Formula (V) are as described in the first aspect or the first, second, third, fourth, fifth, nineteenth, twentieth, twenty-first, twenty-second, twenty-third, thirtieth, thirty-first, thirty-second, thirty-third, thirty-fourth, thirty-fifth, thirty-sixth, thirty-seventh, thirty-eighth, thirty-ninth, fortieth, forty-third, or forty-fourth embodiment or any alternative embodiments described therein.

In a forty-ninth embodiment, the compound of the present disclosure is represented by Formula (VI):

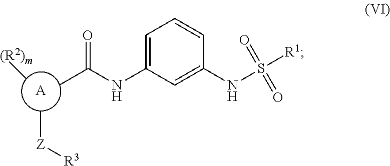

or a pharmaceutically acceptable salt thereof, wherein;
R¹ is C₁₋₂alkyl;
Ring A is selected from phenyl, thiophenyl, pyrazolyl, isothiazolyl, and imidazolyl;
m is 0 to 1;
each R² is independently selected from C₁₋₄alkyl and —C(O)OH;
Z is a bond or —C(O)NH—*, wherein * indicates the attachment point to R³;
R³ is selected from phenyl, pyridyl, pyrimidyl, benzimidazolyl and 2-oxo-2,3-dihydro-1H-benzo[d]imidazolyl, each of which is optionally substituted with 1 to 2 R⁴;
each R⁴ is independently selected from C₁₋₄alkyl, halo, —OH, —O-benzyl, cyano, —NR^{4b}R^{4c}, —C(O)NR^{4b}R^{4c}, or 4 to 6-membered monocyclic heterocyclyl;
R^{4b} and R^{4c} are each independently selected from H and C₁₋₄alkyl.

In a fiftieth embodiment, for the compounds of Formula (VI) described in the forty-ninth embodiment, or a pharmaceutically acceptable salt thereof, Z is a bond, ring A is

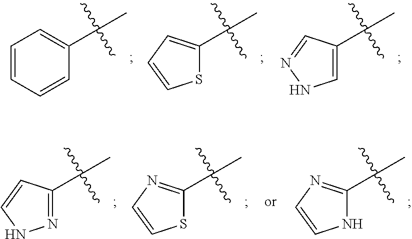 [SMILES: *c1ccccc1 ; *c1cccs1 ; *c1cn[nH]c1 ; *c1cc[nH]n1 ; *c1nccs1 ; *c1ncc[nH]1]

each of which is substituted with 1 R³ and optionally substituted with 1 R²; and the remaining variables are as described in the forty-ninth embodiment.

In a fifty-first embodiment, for the compounds of Formula (VI) described in the forty-ninth embodiment, or a pharmaceutically acceptable salt thereof, Z is a bond, ring A is

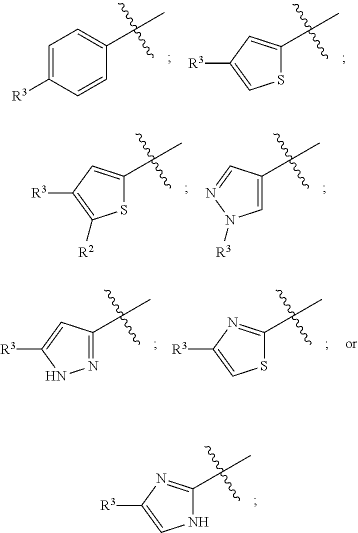

and the remaining variables are as described in the forty-ninth embodiment.

In a fifty-second embodiment, for the compounds of Formula (VI) described in the forty-ninth embodiment, or a pharmaceutically acceptable salt thereof, Z is a bond, ring A is

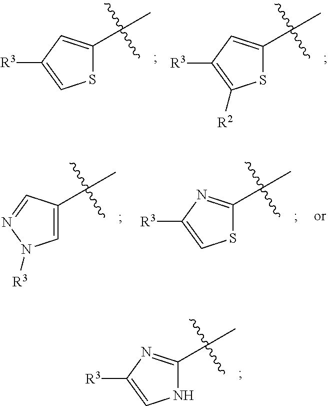

and the remaining variables are as described in the forty-ninth embodiment.

In a fifty-third embodiment, for the compounds of Formula (VI) described in the forty-ninth embodiment, or a pharmaceutically acceptable salt thereof, R² is —CH₃ or —C(O)OH; and the remaining variables are as described in the forty-ninth, fiftieth, fifty-first, or fifty-second embodiment.

In a fifty-fourth embodiment, for the compounds of Formula (VI) described in the forty-ninth embodiment, or a pharmaceutically acceptable salt thereof, R³ is phenyl, pyridyl, or pyrimidyl, each of which is optionally substituted with 1 to 2 R⁴; and the remaining variables are as described in the forty-ninth, fiftieth, fifty-first, fifty-second, or fifty-third embodiment.

In a fifty-fifth embodiment, for the compounds of Formula (VI) described in the forty-ninth embodiment, or a pharmaceutically acceptable salt thereof, R³ is

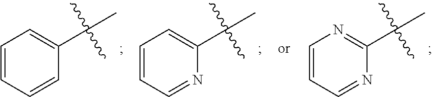 [SMILES: *c1ccccc1 ; *c1ccccn1 ; *c1ncccn1]

each of which is optionally substituted with 1 to 2 R⁴; and the remaining variables are as described in the forty-ninth, fiftieth, fifty-first, fifty-second, or fifty-third embodiment.

In a fifty-sixth embodiment, for the compounds of Formula (VI) described in the forty-ninth embodiment, or a pharmaceutically acceptable salt thereof, R³ is

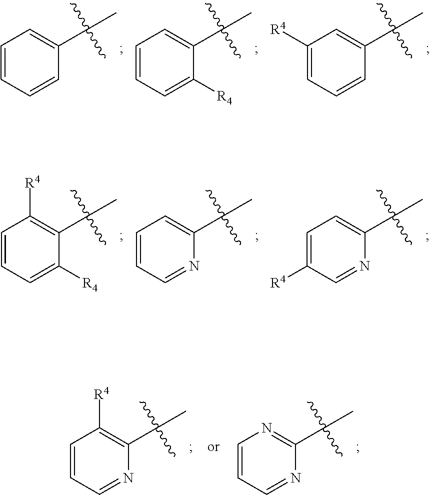

and the remaining variables are as described in the forty-ninth, fiftieth, fifty-first, fifty-second, or fifty-third embodiment.

In a fifty-seventh embodiment, for the compounds of Formula (VI) described in the forty-ninth embodiment, or a pharmaceutically acceptable salt thereof, R⁴ is —CH₃, —NH₂, —OH, halo, or —NHC(O)CH₃,

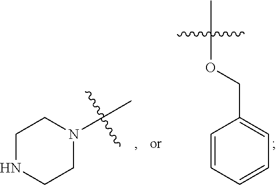 [SMILES: *N1CCNCC1 ; *OCc1ccccc1]

and the remaining variables are as described in the forty-ninth, fiftieth, fifty-first, fifty-second, fifty-third, fifty-fourth, fifty-fifth, or fifty-sixth embodiment.

In a fifty-eighth embodiment, for the compounds of Formula (VI) described in the forty-ninth embodiment, or a pharmaceutically acceptable salt thereof, Z is —C(O)NH—*, wherein * indicates the attachment point to R³, ring A is

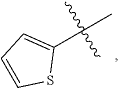 [SMILES: *c1cccs1]

which is substituted with 1 R³; and the remaining variables are as described in the forty-ninth embodiment.

In a fifty-ninth embodiment, for the compounds of Formula (VI) described in the forty-ninth embodiment, or a pharmaceutically acceptable salt thereof, Z is —C(O)NH—*, wherein * indicates the attachment point to R³, ring A is

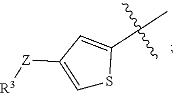

and the remaining variables are as described in the forty-ninth embodiment.

In a sixtieth embodiment, for the compounds of Formula (VI) described in the forty-ninth embodiment, or a pharmaceutically acceptable salt thereof, R³ is benzamidazolyl or 2-oxo-2,3-dihydro-1H-benzo[d]imidazolyl, each of which is optionally substituted with 1 to 2 R⁴; and the remaining variables are as described in the forty-ninth, fifth-eighth, or fifth-ninth embodiment.

In a sixty-first embodiment, for the compounds of Formula (VI) described in the forty-ninth embodiment, or a pharmaceutically acceptable salt thereof, R³ is

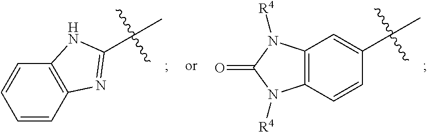

and the remaining variables are as described in the forty-ninth, fifth-eighth, or fifth-ninth embodiment.

In a sixty-second embodiment, for the compounds of Formula (VI) described in the forty-ninth embodiment, or a pharmaceutically acceptable salt thereof, R⁴ is —CH₃; and the remaining variables are as described in the forty-ninth, fifth-eighth, fifth-ninth, sixtieth, or sixty-first embodiment.

In a sixty-third embodiment, the present disclosure provides a compound described herein (e.g., a compound of any one of Examples 1-162), or a pharmaceutically acceptable salt thereof.

In a sixty-fourth embodiment, the present disclosure provides a compound selected from the group consisting of:
N-(3-(methylsulfonamido)phenyl)-1-phenyl-1H-pyrazole-4-carboxamide;
N-(3-(methylsulfonamido)phenyl)-4-phenylpicolinamide;
4-(benzyloxy)-N-(3-(methylsulfonamido)phenyl)benzamide;
3-(benzyloxy)-N-(3-(methylsulfonamido)phenyl)benzamide;
N-(3-(methylsulfonamido)phenyl)-5-phenylthiophene-2-carboxamide;
N-(3-(methylsulfonamido)phenyl)-4-phenylthiophene-2-carboxamide;
4-(5-methyl-1,3,4-oxadiazol-2-yl)-N-(3-(methylsulfonamido)phenyl)benzamide;
N-(3-(methylsulfonamido)phenyl)-4-(1H-tetrazol-5-yl)benzamide;
N-(3-(methylsulfonamido)phenyl)-2-(4H-1,2,4-triazol-3-yl)thiazole-4-carboxamide;
1-benzyl-N-(3-(methylsulfonamido)phenyl)-1H-pyrazole-4-carboxamide;
N-(3-(methylsulfonamido)phenyl)-[1,1′-biphenyl]-3-carboxamide;
N-(3-(methylsulfonamido)phenyl)-[1,1′-biphenyl]-4-carboxamide;
N-(3-(methylsulfonamido)phenyl)-4-(1H-pyrazol-1-yl)benzamide;
N-(3-(methylsulfonamido)phenyl)-3-(1H-pyrazol-1-yl)benzamide;
N-(3-(methylsulfonamido)phenyl)-1-(pyridin-2-yl)-1H-pyrazole-4-carboxamide;
N-(3-(methylsulfonamido)phenyl)-4-phenylthiazole-2-carboxamide;
N-(3-(methylsulfonamido)phenyl)-1-(pyrimidin-2-yl)-1H-pyrazole-4-carboxamide;
N-(3-(methylsulfonamido)phenyl)-4-(thiophen-2-yl)picolinamide;
4-(3-fluorophenyl)-N-(3-(methylsulfonamido)phenyl)thiophene-2-carboxamide;
N-(3-(methylsulfonamido)phenyl)-4-(o-tolyl)thiophene-2-carboxamide;
N-(3-(methylsulfonamido)phenyl)-4-(m-tolyl)thiophene-2-carboxamide;
N-(3-(methylsulfonamido)phenyl)-4-(p-tolyl)thiophene-2-carboxamide;
5-methyl-N-(3-(methylsulfonamido)phenyl)-4-phenylthiophene-2-carboxamide;
4-(3-carbamoylphenyl)-N-(3-(methylsulfonamido)phenyl)thiophene-2-carboxamide;
4-(3-cyanophenyl)-N-(3-(methylsulfonamido)phenyl)thiophene-2-carboxamide;
4-(2-fluorophenyl)-N-(3-(methylsulfonamido)phenyl)thiophene-2-carboxamide;
4-(4-fluorophenyl)-N-(3-(methylsulfonamido)phenyl)thiophene-2-carboxamide;
N-(3-(methylsulfonamido)phenyl)-4-(pyridin-3-yl)thiophene-2-carboxamide;
N-(3-(methylsulfonamido)phenyl)-4-(pyridin-4-yl)thiophene-2-carboxamide;
4-(3-methoxyphenyl)-N-(3-(methylsulfonamido)phenyl)thiophene-2-carboxamide;
4-(2-hydroxyphenyl)-N-(3-(methylsulfonamido)phenyl)thiophene-2-carboxamide;
4-(3-aminophenyl)-N-(3-(methylsulfonamido)phenyl)thiophene-2-carboxamide;
4-(2-methoxyphenyl)-N-(3-(methylsulfonamido)phenyl)thiophene-2-carboxamide;
4-(2-methylpyridin-3-yl)-N-(3-(methylsulfonamido)phenyl)thiophene-2-carboxamide;
4-(4-methylpyridin-3-yl)-N-(3-(methylsulfonamido)phenyl)thiophene-2-carboxamide;
N-(3-(methylsulfonamido)phenyl)-4-(pyrimidin-5-yl)thiophene-2-carboxamide;
4-(2-ethoxypyrimidin-5-yl)-N-(3-(methylsulfonamido)phenyl)thiophene-2-carboxamide;
5-(4-hydroxyphenyl)-N-(3-(methylsulfonamido)phenyl)-1H-pyrazole-3-carboxamide;
4-(6-methylpyridin-3-yl)-N-(3-(methylsulfonamido)phenyl)thiophene-2-carboxamide;
N-(3-(methylsulfonamido)phenyl)-4-(pyridazin-3-yl)thiophene-2-carboxamide;
4-(3-methoxy-2-methylphenyl)-N-(3-(methylsulfonamido)phenyl)thiophene-2-carboxamide;
4-(5-methoxy-2-methylphenyl)-N-(3-(methylsulfonamido)phenyl)thiophene-2-carboxamide;
4-(3-hydroxypyridin-2-yl)-N-(3-(methylsulfonamido)phenyl)thiophene-2-carboxamide;
4-(2-fluoro-6-hydroxyphenyl)-N-(3-(methylsulfonamido)phenyl)thiophene-2-carboxamide;
4-benzyl-N-(3-(methylsulfonamido)phenyl)thiophene-2-carboxamide; tert-butyl (3-(5-((3-(methylsulfonamido)phenyl)carbamoyl)thiophen-3-yl)phenyl)carbamate;
4-(3-hydroxyphenyl)-N-(3-(methylsulfonamido)phenyl)thiophene-2-carboxamide;
4-(3-hydroxy-2-methylphenyl)-N-(3-(methylsulfonamido)phenyl)thiophene-2-carboxamide;
4-(5-hydroxy-2-methylphenyl)-N-(3-(methylsulfonamido)phenyl)thiophene-2-carboxamide;
4-(2,6-difluorophenyl)-N-(3-(methylsulfonamido)phenyl)thiophene-2-carboxamide;
4-(2-(benzyloxy)phenyl)-N-(3-(methylsulfonamido)phenyl)thiophene-2-carboxamide;
5-(4-methoxyphenyl)-N-(3-(methylsulfonamido)phenyl)-1H-pyrazole-3-carboxamide;
5-(4-hydroxyphenyl)-1-methyl-N-(3-(methylsulfonamido)phenyl)-1H-pyrazole-3-carboxamide;
5-methyl-N-(3-(methylsulfonamido)phenyl)-4-(pyridin-2-yl)thiophene-2-carboxamide;
N-(3-(methylsulfonamido)phenyl)-3-(pyridin-2-yl)-1H-pyrazole-5-carboxamide;
N-(3-(methylsulfonamido)phenyl)-4-(pyridin-2-yl)thiophene-2-carboxamide;
4-(3-methylpyridin-2-yl)-N-(3-(methylsulfonamido)phenyl)thiophene-2-carboxamide;
N-(3-(methylsulfonamido)phenyl)-4-(pyrimidin-2-yl)thiophene-2-carboxamide;
4-(6-aminopyridin-2-yl)-N-(3-(methylsulfonamido)phenyl)thiophene-2-carboxamide;
4-(4-aminopyridin-2-yl)-N-(3-(methylsulfonamido)phenyl)thiophene-2-carboxamide;
2-(5-((3-(methylsulfonamido)phenyl)carbamoyl)thiophen-3-yl)isonicotinic acid;
4-(2-aminophenyl)-N-(3-(methylsulfonamido)phenyl)thiophene-2-carboxamide;
4-(3-aminopyridin-2-yl)-N-(3-(methylsulfonamido)phenyl)thiophene-2-carboxamide;
N-(3-(methylsulfonamido)phenyl)-4-(pyridin-2-yl)-1H-imidazole-2-carboxamide;
N-(3-(methylsulfonamido)phenyl)-4-(5-(piperazin-1-yl)pyridin-2-yl)thiophene-2-carboxamide;
2-(5-((3-(methylsulfonamido)phenyl)carbamoyl)thiophen-3-yl)isonicotinamide;
4-(2-acetamidophenyl)-N-(3-(methylsulfonamido)phenyl)thiophene-2-carboxamide;
4-(3-acetamidopyridin-2-yl)-N-(3-(methylsulfonamido)phenyl)thiophene-2-carboxamide;
4-(5-(4-acetylpiperazin-1-yl)pyridin-2-yl)-N-(3-(methylsulfonamido)phenyl)thiophene-2-carboxamide;
N-(3-(ethylsulfonamido)phenyl)-4-phenylthiophene-2-carboxamide;
N-(3-(ethylsulfonamido)phenyl)-4-(pyridin-2-yl)thiophene-2-carboxamide;
5-bromo-N-(3-(methylsulfonamido)phenyl)-4-phenylthiophene-2-carboxamide;
N-(3-(methylsulfonamido)phenyl)-4-phenyl-5-(trifluoromethyl)thiophene-2-carboxamide;
5-cyano-N-(3-(methylsulfonamido)phenyl)-4-phenylthiophene-2-carboxamide;
5-((3-(methylsulfonamido)phenyl)carbamoyl)-3-phenylthiophene-2-carboxylic acid;
N2-(3-(methylsulfonamido)phenyl)-N4-phenylthiophene-2,4-dicarboxamide;
N2-(3-(methylsulfonamido)phenyl)-N4-(2-(pyridin-3-yl)ethyl)thiophene-2,4-dicarboxamide;
N4-(3-chloro-4-methylphenyl)-N2-(3-(methylsulfonamido)phenyl)thiophene-2,4-dicarboxamide;
N4-((1-hydroxycyclohexyl)methyl)-N2-(3-(methylsulfonamido)phenyl)thiophene-2,4-dicarboxamide;
N4-(6-methoxypyridin-3-yl)-N2-(3-(methylsulfonamido)phenyl)thiophene-2,4-dicarboxamide;
N4-(1,5-dimethyl-3-oxo-2-phenyl-2,3-dihydro-1H-pyrazol-4-yl)-N2-(3-(methylsulfonamido)phenyl)thiophene-2,4-dicarboxamide;
N2-(3-(methylsulfonamido)phenyl)-N4-(4-(trifluoromethoxy)phenyl)thiophene-2,4-dicarboxamide;
N4-(5-fluoro-2-methoxyphenyl)-N2-(3-(methylsulfonamido)phenyl)thiophene-2,4-dicarboxamide;
N2-(3-(methylsulfonamido)phenyl)-N4-(1-phenylethyl)thiophene-2,4-dicarboxamide;
N4-((5-fluoropyridin-3-yl)methyl)-N2-(3-(methylsulfonamido)phenyl)thiophene-2,4-dicarboxamide;
N4-(2,3-dihydro-1H-inden-2-yl)-N2-(3-(methylsulfonamido)phenyl)thiophene-2,4-dicarboxamide;
N4-(4-chloro-3-methylphenyl)-N2-(3-(methylsulfonamido)phenyl)thiophene-2,4-dicarboxamide;
N4-(4-(dimethylcarbamoyl)phenyl)-N2-(3-(methylsulfonamido)phenyl)thiophene-2,4-dicarboxamide;
N2-(3-(methylsulfonamido)phenyl)-N4-(2-phenoxyphenyl)thiophene-2,4-dicarboxamide;
N2-(3-(methylsulfonamido)phenyl)-N4-(4-morpholinobenzyl)thiophene-2,4-dicarboxamide;
N2-(3-(methylsulfonamido)phenyl)-N4-(4-phenoxyphenyl)thiophene-2,4-dicarboxamide;
N4-(4-isopropoxyphenyl)-N2-(3-(methylsulfonamido)phenyl)thiophene-2,4-dicarboxamide;
N4-(cyclopropylmethyl)-N2-(3-(methylsulfonamido)phenyl)thiophene-2,4-dicarboxamide;
N2-(3-(methylsulfonamido)phenyl)-N4-(4-(morpholinomethyl)phenyl)thiophene-2,4-dicarboxamide;
N2-(3-(methylsulfonamido)phenyl)-N4-(3-(trifluoromethoxy)phenyl)thiophene-2,4-dicarboxamide;
N4-(3-isopropoxyphenyl)-N2-(3-(methylsulfonamido)phenyl)thiophene-2,4-dicarboxamide;
N2-(3-(methylsulfonamido)phenyl)-N4-(4-(2-morpholinoethoxy)phenyl)thiophene-2,4-dicarboxamide;
N4-(2,3-dihydrobenzofuran-4-yl)-N2-(3-(methylsulfonamido)phenyl)thiophene-2,4-dicarboxamide;
N2-(3-(methylsulfonamido)phenyl)-N4-(4-(morpholinomethyl)benzyl)thiophene-2,4-dicarboxamide;
N4-(3-chloro-5-methoxyphenyl)-N2-(3-(methylsulfonamido)phenyl)thiophene-2,4-dicarboxamide;
N4-(4-(tert-butylcarbamoyl)phenyl)-N2-(3-(methylsulfonamido)phenyl)thiophene-2,4-dicarboxamide;
N2-(3-(methylsulfonamido)phenyl)-N4-(4-(pyridin-4-yl)phenyl)thiophene-2,4-dicarboxamide;
N4-(imidazo[1,2-a]pyridin-3-yl)-N2-(3-(methylsulfonamido)phenyl)thiophene-2,4-dicarboxamide;
N4-(4-methoxyphenyl)-N2-(3-(methylsulfonamido)phenyl)thiophene-2,4-dicarboxamide;
N4-(1,3-dimethyl-2-oxo-2,3-dihydro-1H-benzo[d]imidazol-5-yl)-N2-(3-(methylsulfonamido)phenyl)thiophene-2,4-dicarboxamide;
N4-(1-methyl-1H-benzo[d]imidazol-6-yl)-N2-(3-(methylsulfonamido)phenyl)thiophene-2,4-dicarboxamide;
N2-(3-(methylsulfonamido)phenyl)-N4-(3-(pyridin-3-yl)phenyl)thiophene-2,4-dicarboxamide;
N4-(3-(methoxymethyl)phenyl)-N2-(3-(methylsulfonamido)phenyl)thiophene-2,4-dicarboxamide;
N4-(2-isopropoxyphenyl)-N2-(3-(methylsulfonamido)phenyl)thiophene-2,4-dicarboxamide;
N4-(2,3-dihydro-1H-inden-1-yl)-N2-(3-(methylsulfonamido)phenyl)thiophene-2,4-dicarboxamide;
N4-(4-chlorophenyl)-N2-(3-(methylsulfonamido)phenyl)thiophene-2,4-dicarboxamide;
N4-(2-(cyanomethyl)phenyl)-N2-(3-(methylsulfonamido)phenyl)thiophene-2,4-dicarboxamide;
N4-(4-(methylcarbamoyl)phenyl)-N2-(3-(methylsulfonamido)phenyl)thiophene-2,4-dicarboxamide;
N4-(2-(1H-indol-3-yl)ethyl)-N2-(3-(methylsulfonamido)phenyl)thiophene-2,4-dicarboxamide;
N4-((1H-benzo[d]imidazol-2-yl)methyl)-N2-(3-(methylsulfonamido)phenyl)thiophene-2,4-dicarboxamide;
N4-(2,5-dimethoxyphenethyl)-N2-(3-(methylsulfonamido)phenyl)thiophene-2,4-dicarboxamide;
N4-(2-(2-hydroxyethyl)phenyl)-N2-(3-(methylsulfonamido)phenyl)thiophene-2,4-dicarboxamide;
N4-((1H-indazol-3-yl)methyl)-N2-(3-(methylsulfonamido)phenyl)thiophene-2,4-dicarboxamide;
N4-((1H-indol-5-yl)methyl)-N2-(3-(methylsulfonamido)phenyl)thiophene-2,4-dicarboxamide;
N4-(1H-benzo[d]imidazol-2-yl)-N2-(3-(methylsulfonamido)phenyl)thiophene-2,4-dicarboxamide;
N2-(3-(methylsulfonamido)phenyl)-N4-(3-(2-methylthiazol-4-yl)phenyl)thiophene-2,4-dicarboxamide;
N2-(3-(methylsulfonamido)phenyl)-N4-(5-phenyl-1H-pyrazol-3-yl)thiophene-2,4-dicarboxamide;
N4-(4-(2-hydroxyethyl)phenyl)-N2-(3-(methylsulfonamido)phenyl)thiophene-2,4-dicarboxamide;
N2-(3-(methylsulfonamido)phenyl)-N4-(2-(piperidin-1-yl)ethyl)thiophene-2,4-dicarboxamide;
N2-(3-(methylsulfonamido)phenyl)-N4-(3-(piperidin-1-ylmethyl)phenyl)thiophene-2,4-dicarboxamide;
N2-methyl-N5-(3-(methylsulfonamido)phenyl)-3-phenylthiophene-2,5-dicarboxamide;
5-(hydroxymethyl)-N-(3-(methylsulfonamido)phenyl)-4-phenylthiophene-2-carboxamide;
1-benzyl-5-methyl-N-(3-(methylsulfonamido)phenyl)-1H-pyrazole-3-carboxamide;
4-(benzylamino)-N-(3-(methylsulfonamido)phenyl)thiophene-2-carboxamide;
N-(6-(methylsulfonamido)pyridin-2-yl)-4-phenylthiophene-2-carboxamide;
N-(3-(methylsulfonoamidimidamido)phenyl)-4-phenylthiophene-2-carboxamide;
N-(3-methyl-5-(methylsulfonamido)phenyl)-4-phenylthiophene-2-carboxamide;
N-(3-formyl-5-(methylsulfonamido)phenyl)-4-phenylthiophene-2-carboxamide;
5-(5-(3,3-difluoroazetidin-1-yl)pyridin-2-yl)-1-methyl-N-(3-(methylsulfonamido)phenyl)-1H-pyrrole-3-carboxamide;
5-(3,5-difluoropyridin-2-yl)-1-methyl-N-(3-(methylsulfonamido)phenyl)-1H-pyrrole-3-carboxamide; and
N4-(2-cyclopropylethyl)-N2-(3-(methylsulfonamido)phenyl)thiophene-2,4-dicarboxamide;
   or a pharmaceutically acceptable salt thereof.

In some embodiments, the compounds of Table I are excluded from the compounds of the present disclosure (e.g., compounds of Formula (I)).

TABLE I
Compound Name

N-(3-(methylsulfonamido)phenyl)-4-(1H-pyrrol-1-yl)benzamide
5-(2-methoxyphenyl)-N-(3-(methylsulfonamido)phenyl)-1H-pyrazole-3-carboxamide
N-(3-(methylsulfonamido)phenyl)-4-(tetrahydro-2H-pyran-4-yl)benzamide
N-(3-(methylsulfonamido)phenyl)-5-(thiophen-2-yl)-1H-pyrazole-3-carboxamide
5-(furan-2-yl)-N-(3-(methylsulfonamido)phenyl)-1H-pyrazole-3-carboxamide
N-(3-(methylsulfonamido)phenyl)-2-(pyrazin-2-yl)thiazole-4-carboxamide
4-methyl-N-(3-(methylsulfonamido)phenyl)-2-(tetrahydro-2H-pyran-3-yl)thiazole-5-
carboxamide
3-(4-fluorophenyl)-1-methyl-N-(3-(methylsulfonamido)phenyl)-1H-pyrazole-5-
carboxamide
N-(3-(methylsulfonamido)phenyl)-[1,1′-biphenyl]-2-carboxamide
N-(3-(methylsulfonamido)phenyl)-4-(pyrrolidin-1-yl)benzamide
3-fluoro-N-(3-(methylsulfonamido)phenyl)-[1,1′-biphenyl]-4-carboxamide
2-methyl-N-(3-(methylsulfonamido)phenyl)-6-phenylnicotinamide
N-(3-(methylsulfonamido)phenyl)-2-(1H-pyrazol-1-yl)benzamide
N-(3-(methylsulfonamido)phenyl)-3-(1H-tetrazol-1-yl)benzamide
5-(2-fluorophenyl)-N-(3-(methylsulfonamido)phenyl)thiophene-2-carboxamide
N-(3-(methylsulfonamido)phenyl)-2-(1H-pyrrol-1-yl)thiazole-4-carboxamide
1-methyl-N-(3-(methylsulfonamido)phenyl)-3-phenyl-1H-pyrazole-5-carboxamide
5-methyl-N-(3-(methylsulfonamido)phenyl)-1-(o-tolyl)-1H-pyrazole-4-carboxamide
2-(3-chlorophenyl)-N-(3-(methylsulfonamido)phenyl)thiazole-4-carboxamide
1-(4-bromophenyl)-5-methyl-N-(3-(methylsulfonamido)phenyl)-1H-pyrazole-4-
carboxamide
1-(3-chlorophenyl)-5-ethyl-N-(3-(methylsulfonamido)phenyl)-1H-pyrazole-4-carboxamide
5-ethyl-1-(4-fluorophenyl)-N-(3-(methylsulfonamido)phenyl)-1H-pyrazole-4-carboxamide
1-(3-methoxyphenyl)-2,5-dimethyl-N-(3-(methylsulfonamido)phenyl)-1H-pyrrole-3-
carboxamide
1-(3-chlorophenyl)-5-isopropyl-N-(3-(methylsulfonamido)phenyl)-1H-pyrazole-4-
carboxamide
1-(4-fluorophenyl)-5-isopropyl-N-(3-(methylsulfonamido)phenyl)-1H-pyrazole-4-
carboxamide
1-(4-fluorophenyl)-N-(3-(methylsulfonamido)phenyl)-2-(methylthio)-1H-imidazole-5-
carboxamide
N-(3-(methylsulfonamido)phenyl)-1-phenyl-1,4,5,6-tetrahydrocyclopenta[c]pyrazole-3-
carboxamide
2-(1H-indol-2-yl)-N-(3-(methylsulfonamido)phenyl)thiazole-4-carboxamide
3-(tert-butyl)-5-(2,4-dioxotetrahydropyrimidin-1(2H)-yl)-2-hydroxy-N-(3-
(methylsulfonamido)phenyl)benzamide
1-(3-chlorophenyl)-N-(3-(methylsulfonamido)phenyl)-5-(trifluoromethyl)-1H-pyrazole-4-
carboxamide
1-(4-chlorophenyl)-N-(3-(methylsulfonamido)phenyl)-5-(trifluoromethyl)-1H-pyrazole-4-
carboxamide
1-(2,3-dihydrobenzo[b][1,4]dioxin-6-yl)-2,5-dimethyl-N-(3-(methylsulfonamido)phenyl)-
1H-pyrrole-3-carboxamide
2-cyclobutyl-N-(3-(methylsulfonamido)phenyl)benzamide
N-(3-(methylsulfonamido)phenyl)-4-(2H-tetrazol-5-yl)benzamide
N-(3-(methylsulfonamido)phenyl)-2-(piperidin-1-yl)benzamide
N-(3-(methylsulfonamido)phenyl)-2-(pyrrolidin-1-yl)nicotinamide
4-(5-methyl-1H-tetrazol-1-yl)-N-(3-(methylsulfonamido)phenyl)benzamide
5-methyl-N-(3-(methylsulfonamido)phenyl)-2-(2-oxopyrrolidin-1-yl)benzamide
N-(3-(methylsulfonamido)phenyl)-5-phenyl-1H-pyrazole-3-carboxamide
2-cyclopropyl-N-(3-(methylsulfonamido)phenyl)thiazole-4-carboxamide
1-cyclohexyl-N-(3-(methylsulfonamido)phenyl)-1H-pyrazole-3-carboxamide
N-(3-(methylsulfonamido)phenyl)-2-phenylthiazole-4-carboxamide
N-(3-(methylsulfonamido)phenyl)-1-(piperidin-3-yl)-1H-pyrazole-3-carboxamide
5-methyl-N-(3-(methylsulfonamido)phenyl)-1-phenyl-1H-pyrazole-4-carboxamide
N-(3-(methylsulfonamido)phenyl)-1-(piperidin-3-yl)-1H-pyrazole-3-carboxamide
5-cyclopropyl-1-ethyl-N-(3-(methylsulfonamido)phenyl)-1H-pyrazole-4-carboxamide
3-(tert-butyl)-2-methoxy-N-(3-(methylsulfonamido)phenyl)-5-(2-oxo-1,2-dihydropyridin-
3-yl)benzamide
N-(3-(methylsulfonamido)phenyl)-2-(o-tolyl)thiazole-4-carboxamide
1-cyclopropyl-2,5-dimethyl-N-(3-(methylsulfonamido)phenyl)-1H-pyrrole-3-carboxamide
3-cyclohexyl-1-methyl-N-(3-(methylsulfonamido)phenyl)-1H-pyrazole-4-carboxamide
5-(4-fluorophenyl)-N-(3-(methylsulfonamido)phenyl)-1H-pyrazole-3-carboxamide
N-(3-(methylsulfonamido)phenyl)-2-(1H-1,2,4-triazol-5-yl)thiazole-4-carboxamide
N-(3-(methylsulfonamido)phenyl)-5-(2-methylthiazol-4-yl)furan-3-carboxamide
5-cyclopentyl-1-methyl-N-(3-(methylsulfonamido)phenyl)-1H-pyrazole-3-carboxamide
N-(3-(methylsulfonamido)phenyl)-2-(piperidin-1-yl)thiazole-4-carboxamide
2-cyclopentyl-4-methyl-N-(3-(methylsulfonamido)phenyl)thiazole-5-carboxamide
2-(4-fluorophenyl)-N-(3-(methylsulfonamido)phenyl)thiazole-4-carboxamide
1-(2,6-difluorophenyl)-N-(3-(methylsulfonamido)phenyl)-1H-pyrazole-3-carboxamide
5-methyl-N-(3-(methylsulfonamido)phenyl)-1-(tetrahydro-2H-pyran-4-yl)-1H-pyrazole-4-
carboxamide
2-(2,5-dimethyl-1H-pyrrol-1-yl)-N-(3-(methylsulfonamido)phenyl)thiophene-3-
carboxamide
1-(4-methoxyphenyl)-5-methyl-N-(3-(methylsulfonamido)phenyl)-1H-pyrazole-3-
carboxamide
1-cyclohexyl-2,5-dimethyl-N-(3-(methylsulfonamido)phenyl)-1H-pyrrole-3-carboxamide
3-(3-methoxyphenyl)-1-methyl-N-(3-(methylsulfonamido)phenyl)-1H-pyrazole-5-
carboxamide
2-(2,4-dichlorophenyl)-N-(3-(methylsulfonamido)phenyl)thiazole-4-carboxamide
1-(4,6-dimethylpyrimidin-2-yl)-5-methyl-N-(3-(methylsulfonamido)phenyl)-1H-pyrazole-
3-carboxamide
N-(3-(methylsulfonamido)phenyl)-5-(3,4,5-trimethoxyphenyl)-1H-pyrazole-3-carboxamide
3-(2,5-dimethoxyphenyl)-1-methyl-N-(3-(methylsulfonamido)phenyl)-1H-pyrazole-5-
carboxamide
3-(4-(2-cyclopropyl-1-methyl-1H-imidazol-4-yl)-1H-1,2,3-triazol-1-yl)-4-methyl-N-(3-
(methylsulfonamido)phenyl)benzamide
2-(1-methyl-1H-indol-2-yl)-N-(3-(methylsulfonamido)phenyl)thiazole-4-carboxamide
2-(1-ethyl-1H-indol-2-yl)-N-(3-(methylsulfonamido)phenyl)thiazole-4-carboxamide
1-(2-chloro-5-(trifluoromethyl)phenyl)-N-(3-(methylsulfonamido)phenyl)-1,4,5,6-
tetrahydrocyclopenta[c]pyrazole-3-carboxamide
6-(3-bromophenyl)-2-methyl-N-(3-(methylsulfonamido)phenyl)nicotinamide
N-(3-(methylsulfonamido)phenyl)-3-nitro-4-(pyrrolidin-1-yl)benzamide
N-(3-(methylsulfonamido)phenyl)-2-(methylthio)-1-phenyl-1H-imidazole-5-carboxamide
2′-methyl-5′-(5-methyl-1,3,4-oxadiazol-2-yl)-N-(3-(methylsulfonamido)phenyl)-[1,1′-
biphenyl]-4-carboxamide
5-((3-(methylsulfonamido)phenyl)carbamoyl)-1-phenyl-1H-pyrazole-3-carboxylic acid
5-(N,N-dimethylsulfamoyl)-N-(3-(methylsulfonamido)phenyl)-2-morpholinobenzamide
N-(3-(methylsulfonamido)phenyl)-5-(5-phenyl-4-((pyridin-2-ylmethyl)amino)phthalazin-1-
yl)nicotinamide
2,5-dimethyl-N-(3-(methylsulfonamido)phenyl)-1-(2-(trifluoromethyl)phenyl)-1H-pyrrole-
3-carboxamide
(1R,5S,6r)-3-(6-(isopentyloxy)-5-((3-(methylsulfonamido)phenyl)carbamoyl)pyridin-2-yl)-
3-azabicyclo[3.1.0]hexane-6-carboxylic acid
ethyl (1R,5S,6r)-3-(6-(isopentyloxy)-5-((3-(methylsulfonamido)phenyl)carbamoyl)pyridin-
2-yl)-3-azabicyclo[3.1.0]hexane-6-carboxylate
5-cyclopropyl-1-ethyl-N-(3-(propylsulfonamido)phenyl)-1H-pyrazole-3-carboxamide
1-(piperidin-4-yl)-N-(3-(propylsulfonamido)phenyl)-1H-pyrrole-2-carboxamide
N-(3-(cyclopropanesulfonamido)phenyl)-5-cyclopropyl-1-ethyl-1H-pyrazole-3-
carboxamide
N-(3-(cyclopropanesulfonamido)phenyl)-4-cyclopropyl-1H-pyrrole-3-carboxamide
1-(piperidin-3-yl)-N-(3-(propylsulfonamido)phenyl)-1H-pyrazole-3-carboxamide
1-phenyl-5-((3-(propylsulfonamido)phenyl)carbamoyl)-1H-pyrazole-3-carboxylic acid
1-(piperidin-4-yl)-N-(3-(propylsulfonamido)phenyl)-1H-pyrrole-2-carboxamide
N-(3-(methylsulfonamido)phenyl)-3-(tetrazolidin-1-yl)benzamide
5-methyl-N-(3-(methylsulfonamido)phenyl)-1-(tetrahydro-2H-pyran-4-yl)-1H-pyrazole-3-
carboxamide
N-(3-(cyclopropanesulfonamido)phenyl)-2-cyclopropylfuran-3-carboxamide
3-chloro-6-(3,5-dimethyl-1H-pyrazol-1-yl)-N-(3-(methylsulfonamido)phenyl)picolinamide
N-(3-(methylsulfonamido)phenyl)-1-(tetrahydrofuran-2-carbonyl)piperidine-3-
carboxamide
N-(3-(methylsulfonamido)phenyl)-1-(5-nitrofuran-2-carbonyl)azetidine-3-carboxamide
N-(3-(methylsulfonamido)phenyl)-1-(pyrazine-2-carbonyl)azetidine-3-carboxamide
1-(benzo[d][1,3]dioxole-5-carbonyl)-N-(3-(methylsulfonamido)phenyl)azetidine-3-
carboxamide
1-(3,4-difluorobenzoyl)-N-(3-(methylsulfonamido)phenyl)azetidine-3-carboxamide
N-(3-(methylsulfonamido)phenyl)-2-(thiophene-2-carbonyl)benzamide
1-(furan-2-carbonyl)-N-(3-(methylsulfonamido)phenyl)piperidine-3-carboxamide
N-(3-(cyclopropanesulfonamido)phenyl)-4-(3-fluorobenzoyl)piperazine-1-carboxamide
N-(3-(methylsulfonamido)phenyl)-1-(tetrahydrothiophene-2-carbonyl)piperidine-3-
carboxamide
N-(3-(methylsulfonamido)phenyl)-1-(thiophene-2-carbonyl)azetidine-3-carboxamide
1-(1H-indole-3-carbonyl)-N-(3-(methylsulfonamido)phenyl)azetidine-3-carboxamide
1-(5-methylisoxazole-3-carbonyl)-N-(3-(methylsulfonamido)phenyl)azetidine-3-
carboxamide
N-(3-(methylsulfonamido)phenyl)-1-(pyrrolidine-1-carbonyl)piperidine-3-carboxamide
2-(4-methylbenzoyl)-N-(3-(methylsulfonamido)phenyl)benzamide
N-(3-(methylsulfonamido)phenyl)-1-(thiophene-2-carbonyl)piperidine-3-carboxamide
N4-(3-(methylsulfonamido)phenyl)-N1-(thiophen-2-yl)piperidine-1,4-dicarboxamide
N1-cyclopropyl-N4-(3-(methylsulfonamido)phenyl)piperidine-1,4-dicarboxamide
3-iodo-N1-(2-methyl-4-(perfluoropropan-2-yl)phenyl)-N2-(3-
(methylsulfonamido)phenyl)phthalamide
N1-cyclopropyl-N4-(3-(methylsulfonamido)phenyl)terephthalamide
3-iodo-N1-(2-methyl-4-(perfluoropropan-2-yl)phenyl)-N2-(3-
((trifluoromethyl)sulfonamido)phenyl)phthalamide
N3-(3-(methylsulfonamido)phenyl)-N1-(thiophen-2-yl)piperidine-1,3-dicarboxamide
N1-cyclopropyl-N3-(3-(methylsulfonamido)phenyl)piperidine-1,3-dicarboxamide
N-(3-(methylsulfonamido)phenyl)-3-(phenoxymethyl)furan-2-carboxamide
2-((2,4-dimethoxyphenyl)amino)-N-(3-(methylsulfonamido)phenyl)thiazolidine-4-
carboxamide
2-((3,4-dimethoxyphenyl)amino)-N-(3-(methylsulfonamido)phenyl)thiazolidine-4-
carboxamide
4-((5-chloro-4-(1-isopropyl-1H-pyrazol-4-yl)pyrimidin-2-yl)amino)-3-methoxy-N-(3-
(methylsulfonamido)phenyl)benzamide
3-((6-methylpyridazin-3-yl)amino)-N-(3-(methylsulfonamido)phenyl)benzamide
3-((1-cyclopentyl-2,4-dimethyl-3-oxo-1,2,3,4-tetrahydropyrido[3,4-b]pyrazin-7-yl)amino)-
N-(3-(methylsulfonamido)phenyl)benzamide
3-((2,4-dimethyl-3-oxo-1-(tetrahydro-2H-pyran-4-yl)-1,2,3,4-tetrahydropyrido[3,4-
b]pyrazin-7-yl)amino)-N-(3-(methylsulfonamido)phenyl)benzamide
N-(3-(methylsulfonamido)phenyl)-2-(pyridin-3-ylamino)thiazole-4-carboxamide
N-(3-(methylsulfonamido)phenyl)-2-(pyrimidin-2-ylamino)thiazole-4-carboxamide
2-((4,6-dimethylpyrimidin-2-yl)amino)-N-(3-(methylsulfonamido)phenyl)thiazole-4-
carboxamide
N-(3-(methylsulfonamido)phenyl)-2-((4-(trifluoromethoxy)phenyl)amino)thiazole-4-
carboxamide
2-((2,4-dimethoxyphenyl)amino)-N-(3-(methylsulfonamido)phenyl)thiazole-4-carboxamide
2-((4-fluorophenyl)amino)-N-(3-(methylsulfonamido)phenyl)benzamide
1-ethyl-N-(3-(methylsulfonamido)phenyl)-4-((tetrahydro-2H-pyran-4-yl)amino)-1H-
pyrazolo[3,4-b]pyridine-5-carboxamide
2-((4,6-dimethylpyrimidin-2-yl)amino)-N-(3-(methylsulfonamido)phenyl)thiazolidine-4-
carboxamide
2-((4-ethoxyphenyl)amino)-N-(3-(methylsulfonamido)phenyl)thiazolidine-4-carboxamide
N-(3-(methylsulfonamido)phenyl)-2-((4-(trifluoromethoxy)phenyl)amino)thiazolidine-4-
carboxamide
2-(cyclopropylamino)-N-(3-(methylsulfonamido)phenyl)thiazole-4-carboxamide
4-methyl-N-(3-(methylsulfonamido)phenyl)-3-((4-(pyridin-3-yl)-5H-pyrrolo[3,2-
d]pyrimidin-2-yl)amino)benzamide
3-((1-isopropyl-2,4-dimethyl-3-oxo-1,2,3,4-tetrahydropyrido[3,4-b]pyrazin-7-yl)amino)-N-
(3-(methylsulfonamido)phenyl)benzamide
N-(3-(methylsulfonamido)phenyl)-2-(pyridin-2-ylamino)thiazole-4-carboxamide
2-((6-methylpyridin-2-yl)amino)-N-(3-(methylsulfonamido)phenyl)thiazole-4-carboxamide
N-(3-(methylsulfonamido)phenyl)-3-(pyrimidin-2-ylamino)benzamide
2-((3,4-dimethoxyphenyl)amino)-N-(3-(methylsulfonamido)phenyl)thiazole-4-carboxamide
2-((4-ethoxyphenyl)amino)-N-(3-(methylsulfonamido)phenyl)thiazole-4-carboxamide
2-ethyl-3-(methyl((1-methyl-1H-imidazol-2-yl)methyl)amino)-N-(3-
(methylsulfonamido)phenyl)imidazo[1,2-a]pyridine-6-carboxamide
3-((3-bromobenzyl)(ethyl)amino)-2-ethyl-N-(3-(methylsulfonamido)phenyl)imidazo[1,2-
a]pyridine-6-carboxamide
2-ethyl-3-(methyl(3-phenylpropyl)amino)-N-(3-(methylsulfonamido)phenyl)imidazo[1,2-
a]pyridine-6-carboxamide
4-((4-acetylphenyl)sulfonamido)-N-(3-(methylsulfonamido)phenyl)benzamide
N-(3-(methylsulfonamido)phenyl)-2-((tetrahydrofuran-3-yl)oxy)nicotinamide
N-(3-(methylsulfonamido)phenyl)-4-(thiazol-2-yloxy)benzamide
N-(3-(methylsulfonamido)phenyl)-2-((tetrahydrothiophen-3-yl)oxy)nicotinamide
5-chloro-N-(3-(methylsulfonamido)phenyl)-6-((tetrahydro-2H-pyran-4-
yl)oxy)nicotinamide
N-(3-(methylsulfonamido)phenyl)-2-((tetrahydrofuran-3-yl)oxy)isonicotinamide
N-(3-(methylsulfonamido)phenyl)-3-(pyridin-2-yloxy)benzamide
4-(cyclopentyloxy)-N-(3-(methylsulfonamido)phenyl)benzamide
N-(3-(methylsulfonamido)phenyl)-2-((tetrahydrothiophen-3-yl)oxy)isonicotinamide
2-(cyclopentyloxy)-N-(3-(methylsulfonamido)phenyl)isonicotinamide
N-(3-(methylsulfonamido)phenyl)-6-((tetrahydrothiophen-3-yl)oxy)nicotinamide
N-(3-(methylsulfonamido)phenyl)-6-((tetrahydro-2H-pyran-4-yl)oxy)nicotinamide
N-(3-(methylsulfonamido)phenyl)-6-((tetrahydrofuran-3-yl)oxy)nicotinamide
5-chloro-N-(3-(methylsulfonamido)phenyl)-6-((tetrahydrofuran-3-yl)oxy)nicotinamide
2-(2,4-dichlorophenoxy)-N-(3-(methylsulfonamido)phenyl)benzamide
4-(tert-butyl)-2-(2-chloro-4-fluorophenoxy)-N-(3-(methylsulfonamido)phenyl)benzamide
6-(benzyloxy)-N-(3-(methylsulfonamido)phenyl)pyrimidine-4-carboxamide
8-((2,6-difluorobenzyl)oxy)-2,6-dimethyl-N-(3-(methylsulfonamido)phenyl)imidazo[1,2-
a]pyridine-3-carboxamide
3-((2-methylbenzyl)oxy)-N-(3-(methylsulfonamido)phenyl)azetidine-1-carboxamide
N-(3-(methylsulfonamido)phenyl)-6-(1-phenylethoxy)nicotinamide
3-ethoxy-N-(3-(methylsulfonamido)phenyl)-4-(pyridin-3-ylmethoxy)benzamide
4-((3,5-dimethylisoxazol-4-yl)methoxy)-N-(3-(methylsulfonamido)phenyl)benzamide
N-(3-(methylsulfonamido)phenyl)-4-((2-methylthiazol-4-yl)methoxy)benzamide
N-(3-(methylsulfonamido)phenyl)-6-((tetrahydrofuran-2-yl)methoxy)nicotinamide
3-(benzyloxy)-N-(3-(methylsulfonamido)phenyl)isoxazole-5-carboxamide
2-(benzyloxy)-N-(3-(methylsulfonamido)phenyl)-4-(3-(pyrrolidin-1-yl)propoxy)benzamide
N-(3-(methylsulfonamido)phenyl)-4-((4-methylthiazol-2-yl)methoxy)benzamide
N-(3-(methylsulfonamido)phenyl)-3-(thiophen-2-ylmethoxy)benzamide
N-(3-(methylsulfonamido)phenyl)-4-(quinolin-2-ylmethoxy)benzamide
3-((3,5-dimethylisoxazol-4-yl)methoxy)-N-(3-(methylsulfonamido)phenyl)benzamide
5-((3,4-dihydroisoquinolin-2(1H)-yl)sulfonyl)-N-(3-(methylsulfonamido)phenyl)thiophene-
3-carboxamide
N-(3-(methylsulfonamido)phenyl)-5-(morpholinosulfonyl)thiophene-3-carboxamide
N-(3-(methylsulfonamido)phenyl)-1-(phenylsulfonyl)pyrrolidine-2-carboxamide
N-(3-(methylsulfonamido)phenyl)-1-(phenylsulfonyl)pyrrolidine-2-carboxamide
2-methyl-N-(3-(methylsulfonamido)phenyl)-5-(piperidin-1-ylsulfonyl)benzamide
1-(cyclopropylsulfonyl)-N-(3-(methylsulfonamido)phenyl)azetidine-3-carboxamide
1-((3,5-dimethylisoxazol-4-yl)sulfonyl)-N-(3-(methylsulfonamido)phenyl)piperidine-4-
carboxamide
N-(3-(methylsulfonamido)phenyl)-1-(phenylsulfonyl)pyrrolidine-2-carboxamide
4-ethyl-N-(3-(methylsulfonamido)phenyl)-3-(morpholinosulfonyl)benzamide
1-((3,5-dimethylisoxazolidin-4-yl)sulfonyl)-N-(3-(methylsulfonamido)phenyl)piperidine-4-
carboxamide
N-(3-(methylsulfonamido)phenyl)-4-(N-(pyridin-4-yl)sulfamoyl)benzamide
4-(N-cyclohexyl-N-methylsulfamoyl)-N-(3-(methylsulfonamido)phenyl)benzamide
N-(3-(methylsulfonamido)phenyl)-4-(N-(p-tolyl)sulfamoyl)benzamide
3-(N-(4-fluorophenyl)sulfamoyl)-N-(3-(methylsulfonamido)phenyl)benzamide
4-(N-ethyl-N-phenylsulfamoyl)-N-(3-(methylsulfonamido)phenyl)benzamide
5-(N-cyclohexylsulfamoyl)-2-methyl-N-(3-(methylsulfonamido)phenyl)benzamide
5-(N-cyclopropylsulfamoyl)-2-methyl-N-(3-(methylsulfonamido)phenyl)benzamide
3-(N-(4-methoxyphenyl)-N-methylsulfamoyl)-N-(3-
(methylsulfonamido)phenyl)benzamide
2-methyl-N-(3-(methylsulfonamido)phenyl)-5-(N-(m-tolyl)sulfamoyl)benzamide
4-(N-(2-methoxyphenyl)sulfamoyl)-N-(3-(methylsulfonamido)phenyl)benzamide
N3-(3-(methylsulfonamido)phenyl)-N1-(thiophen-2-ylmethyl)piperidine-1,3-
dicarboxamide
4-benzyl-N-(3-(methylsulfonamido)phenyl)-5-oxomorpholine-3-carboxamide
1-((6-methyl-1H-benzo[d]imidazol-2-yl)methyl)-N-(3-
(methylsulfonamido)phenyl)piperidine-4-carboxamide
1-((1H-benzo[d]imidazol-2-yl)methyl)-N-(3-(methylsulfonamido)phenyl)piperidine-4-
carboxamide
2-benzyl-N-(3-(methylsulfonamido)phenyl)benzamide
2-benzyl-4-methyl-N-(3-(methylsulfonamido)phenyl)thiazolidine-5-carboxamide
1-((4-methoxynaphthalen-1-yl)methyl)-2-methyl-N-(3-
(methylsulfonamido)phenyl)azetidine-2-carboxamide
1-(2-(difluoromethoxy)benzyl)-2-methyl-N-(3-(methylsulfonamido)phenyl)azetidine-2-
carboxamide
5-((3,5-dimethyl-1H-pyrazol-1-yl)methyl)-N-(3-(methylsulfonamido)phenyl)furan-2-
carboxamide
N-(3-(methylsulfonamido)phenyl)-2,4-dioxo-3-(thiophen-2-ylmethyl)-1,2,3,4-
tetrahydropyrimidine-5-carboxamide
3-(benzo[d][1,3]dioxol-5-ylmethyl)-N-(3-(methylsulfonamido)phenyl)-2,4-dioxo-1,2,3,4-
tetrahydropyrimidine-5-carboxamide
3-(2-chlorobenzyl)-N-(3-(methylsulfonamido)phenyl)-2,4-dioxo-1,2,3,4-
tetrahydropyrimidine-5-carboxamide
N-(3-(methylsulfonamido)phenyl)-1-((3-(pyridin-4-yl)-1,2,4-oxadiazol-5-
yl)methyl)piperidine-3-carboxamide
N-(3-(methylsulfonamido)phenyl)-1-((3-(pyrimidin-2-yl)-1,2,4-oxadiazol-5-
yl)methyl)piperidine-3-carboxamide
3-((1H-imidazol-1-yl)methyl)-N-(3-(methylsulfonamido)phenyl)piperidine-1-carboxamide
4-benzyl-N-(3-(propylsulfonamido)phenyl)piperazine-1-carboxamide
1-benzyl-N-(3-(methylsulfonamido)phenyl)-6-oxo-1,6-dihydropyridazine-3-carboxamide
4-((1H-imidazol-1-yl)methyl)-N-(3-(methylsulfonamido)phenyl)benzamide
2-benzyl-4-methyl-N-(3-(methylsulfonamido)phenyl)thiazole-5-carboxamide
2-(1-(4-isopropylbenzyl)-2-((3-(methylsulfonamido)phenyl)carbamoyl)-1H-indol-3-
yl)acetic acid
4-(imidazolidin-1-ylmethyl)-N-(3-(methylsulfonamido)phenyl)benzamide
1-(2,3-dimethoxybenzyl)-2-methyl-N-(3-(methylsulfonamido)phenyl)azetidine-2-
carboxamide
2-methyl-N-(3-(methylsulfonamido)phenyl)-1-(4-(methylthio)benzyl)azetidine-2-
carboxamide
1-(cyclobutylmethyl)-N-(3-(propylsulfonamido)phenyl)-1H-pyrazole-5-carboxamide
3-(furan-2-ylmethyl)-N-(3-(methylsulfonamido)phenyl)-2,4-dioxo-1,2,3,4-
tetrahydropyrimidine-5-carboxamide
1-(4-methylbenzyl)-N-(3-(methylsulfonamido)phenyl)-6-oxo-1,6-dihydropyridazine-3-
carboxamide
3-(2-fluorobenzyl)-N-(3-(methylsulfonamido)phenyl)-2,4-dioxo-1,2,3,4-
tetrahydropyrimidine-5-carboxamide
N-(3-(methylsulfonamido)phenyl)-1-((3-(pyridin-3-yl)-1,2,4-oxadiazol-5-
yl)methyl)piperidine-3-carboxamide
N-(3-(methylsulfonamido)phenyl)-1-((3-(thiophen-2-yl)-1,2,4-oxadiazol-5-
yl)methyl)piperidine-3-carboxamide
2-ethyl-3-(3-methoxy-N-methylbenzamido)-N-(3-(methylsulfonamido)phenyl)imidazo[1,2-
a]pyridine-6-carboxamide
4-(cyclopropanecarboxamido)-N-(3-(methylsulfonamido)phenyl)cyclohexane-1-
carboxamide
2-benzamido-N-(3-(methylsulfonamido)phenyl)oxazole-4-carboxamide
N-(2-chloro-5-((3-(methylsulfonamido)phenyl)carbamoyl)phenyl)thiophene-2-
carboxamide
N-(2-chloro-5-((3-(methylsulfonamido)phenyl)carbamoyl)phenyl)tetrahydrothiophene-2-
carboxamide
2-(cyclopropanecarboxamido)-N-(3-(methylsulfonamido)phenyl)oxazole-4-carboxamide
5-methyl-N-(4-((3-(methylsulfonamido)phenyl)carbamoyl)oxazol-2-yl)isoxazole-3-
carboxamide
2-(furan-2-carboxamido)-N-(3-(methylsulfonamido)phenyl)oxazole-4-carboxamide
N-(3-(methylsulfonamido)phenyl)-2-(thiophene-2-carboxamido)oxazole-4-carboxamide
3-(2-methylcyclopropane-1-carboxamido)-N-(3-(methylsulfonamido)phenyl)benzamide

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed. In one embodiment, the present disclosure provides deuterated compounds described herein or a pharmaceutically acceptable salt thereof.

Pharmaceutically acceptable solvates in accordance with the disclosure include those wherein the solvent of crystallization may be isotopically substituted, e.g. D₂O, d₆-acetone, d₆-DMSO.

It will be recognized by those skilled in the art that the compounds of the present disclosure may contain chiral centers and as such may exist in different stereoisomeric forms.

As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

The present disclosure also provides a pharmaceutical composition comprising a compound described herein (e.g., a compound according to any one of the preceding embodiments), or a pharmaceutically acceptable salt thereof, and one or more pharmaceutically acceptable carriers.

### Methods of Use

The compounds described herein have DHX9 inhibitory activity. As used herein, “DHX9 inhibitory activity” refers to the ability of a compound or composition to induce a detectable decrease in DHX9 activity in vivo or in vitro (e.g., at least 10% decrease in DHX9 activity as measured by a given assay such as the bioassay described in the examples and known in the art).

In certain embodiments, the present disclosure provides a method of treating a disease or disorder responsive to inhibition of DHX9 activity (referred herein as “DHX9 mediated disease or disorder”) in a subject in need of the treatment. The method comprises administering to the subject a compound described herein (e.g., a compound described in any one of the first to sixty-fourth embodiments) or a pharmaceutically acceptable salt thereof or a pharmaceutical composition thereof.

In certain embodiments, the present disclosure provides the use of a compound described herein (e.g., a compound described in any one of the first to sixty-fourth embodiments) or a pharmaceutically acceptable salt thereof or a pharmaceutical composition comprising a compound described herein or a pharmaceutically acceptable salt thereof for the manufacture of a medicament for the treatment of a DHX9 mediated disorder or disease in a subject in need of the treatment.

In certain embodiments, the present disclosure provides a compound described herein (e.g., a compound described in any one of the first to sixty-fourth embodiments) or a pharmaceutically acceptable salt thereof or a pharmaceutical composition comprising a compound described herein or a pharmaceutically acceptable salt thereof for use in the treatment of a DHX9 mediated disorder or disease in a subject in need of the treatment.

In certain embodiments, the DHX9 mediated disease or disorder is selected from cancer, viral infections, and autoimmune disease.

In some embodiments, the present disclosure provides a method of treating cancer. In some embodiments, the cancer is selected from colorectal, endometrial, ovarian, gastric, hematopoietic, breast, brain, skin, lung, blood, prostate, head and neck, pancreatic, bladder, bone, soft-tissue, kidney, and liver cancer. In some embodiments the cancer is selected from colorectal, endometrial, ovarian, hematopoietic, and gastric cancer. In some embodiments, the cancer is colorectal cancer. In some embodiments, the cancer is Ewing's sarcoma.

In some embodiments, the cancer is a microsatellite instable (MSI) cancer. In some embodiments, the cancer is MSI-high cancer. In other embodiments, the cancer is MSI-low cancer. MSI is determined by PCR analysis of 5 different nucleotide repeats, which is dependent on the cancer type. MSI-low cancers are characterized by instability at only 1 of the 5 sites; while MSI-high cancers are characterized by instability at 2 or more of the 5 sites (G. Yang et al. Correlations between microsatellite instability and the biological behavior of tumors. Journal of Cancer Research and Clinical Oncology, 2019).

MSI-high cancer is additionally characterized by defective mismatch repair (dMMR) (M. Lorenzi, et al. Epidemiology of Microsatellite Instability High (MSI-H) and Deficient Mismatch Repair (dMMR) in Solid Tumors: A Structured Literature Review. Journal of Oncology, Volume 2020, Article ID 1897929). For example, dMMR in colorectal cancer can be determined by MLH1 promoter hypermethylation, rendering MLH1 inactive, which comprises about 80-90% of MSI-high colorectal cancers. MSI-high colorectal cancer with inactivated MLH1 is also named sporadic MSI-high colorectal cancer. Alternatively, dMMR can be determined by immunohistochemistry mutation status of MLH1, MSH2, MSH6, MSH3, PMS1 and/or PMS2 mismatch repair (MMR) proteins. In MSI-high colorectal cancer, MLH1 and MSH2 are the 2 predominantly mutated MMR proteins. They are mutated in about 10-20% of MSI-high colorectal cancers. MSI-high colorectal cancers with these mutations are also known as Lynch Syndrome cancers.

In some embodiments, the cancer is a MSI cancer and/or has mutations or defects in DNA mis-match repair (MMR), and/or mutations or defects in RNA splicing and the kinetochore complex.

The compounds, or pharmaceutically acceptable salts thereof described herein may be used to decrease the expression or activity of DHX9, or to otherwise affect the properties and/or behavior of DHX9 in a cell.

One embodiment of the present disclosure includes a method of decreasing the expression or activity of DHX9, or to otherwise affect the properties and/or behavior of DHX9 in a subject comprising administering to said subject an effective amount of at least one compound described herein, or a pharmaceutically acceptable salt thereof.

In certain embodiments, the present disclosure relates to the aforementioned methods, wherein said subject is a mammal.

In certain embodiments, the present disclosure relates to the aforementioned methods, wherein said subject is a primate.

In certain embodiments, the present disclosure relates to the aforementioned methods, wherein said subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 g and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally.

In certain embodiments, the present disclosure relates to the aforementioned methods, wherein said compound is administered intramuscularly, intravenously, subcutaneously, orally, pulmonary, rectally, intrathecally, topically or intranasally.

In certain embodiments, the present disclosure relates to the aforementioned methods, wherein said compound is administered systemically.

The compounds of the present disclosure are typically used as a pharmaceutical composition (e.g., a compound of the present disclosure and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present disclosure and a solvent (i.e., solvate) or water (i.e., hydrate).

The formulations may be prepared using conventional dissolution and mixing procedures. For example, the bulk drug substance (i.e., compound of the present disclosure or stabilized form of the compound (e.g., complex with a cyclodextrin derivative or other known complexation agent)) is dissolved in a suitable solvent in the presence of one or more of the excipients described above. The compound of the present disclosure is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations.

### Definitions

As used herein, a “patient,” “subject” or “individual” are used interchangeably and refer to either a human or non-human animal. The term includes mammals such as humans. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In some embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present disclosure to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein the term “cancer” has the meaning normally accepted in the art. The term can broadly refer to abnormal cell growth.

As used herein, a subject is “in need of” a treatment if such subject would benefit biologically, medically or in quality of life from such treatment (in some embodiments, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkylene” refers to a fully saturated branched or unbranched divalent hydrocarbon radical. The term “C₁₋₄alkylene” refers to an alkylene having 1 to 4 carbon atoms. The terms “C₁₋₃alkylene” and “C₁₋₂alkylene” are to be construed accordingly. Representative examples of “C₁₋₄alkylene” include, but are not limited to, methylene, ethylene, n-propylene, iso-propylene, n-butylene, sec-butylene, iso-butylene, and tert-butylene.

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. In some embodiments, alkoxy groups have about 1-4 carbons, and in some embodiments about 1-2 carbons. The term “C₁₋₂ alkoxy” is to be construed accordingly.

As used herein, the term “C₁₋₄ alkoxyC₁₋₄ alkyl” refers to a C₁₋₄ alkyl group as defined herein, wherein at least of the hydrogen atoms is replaced by an C₁₋₄ alkoxy. The C₁₋₄alkoxyC₁₋₄ alkyl group is connected through the rest of the molecule described herein through the alkyl group.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or two fused ring system containing 6 to 10 carbon atoms. Examples include phenyl, indanyl, tetrahydronaphthalene, and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, tetrazinyl, and the like. Bi-cyclic heteroaryls include groups in which a monocyclic heteroaryl ring is fused to one or more aryl or heteroaryl rings. Non-limiting examples include indolyl, indazoyl, benzofuranyl, benzimidazolyl, and imidazo[1,2-a]pyridine.

The term “carbocyclic ring” or “carbocyclyl” refers to a 4- to 12-membered saturated or partially unsaturated hydrocarbon ring and may exist as a single ring, bicyclic ring (including fused, spiral or bridged carbocyclic rings) or a spiral ring. Bi-cyclic carbocyclyl groups include, e.g., unsaturated carbocyclic radicals fused to another unsaturated carbocyclic radical, cycloalkyl, or aryl, such as, for example, cyclohexyl, cyclohexenyl, 2,3-dihydroindenyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, piperdinyl, piperazinyl, tetrahydropyranyl, morpholinyl, and pyrrolidinyl. Bi-cyclic heterocyclyl groups include, e.g., unsaturated heterocyclic radicals fused to another unsaturated heterocyclic radical, cycloalkyl, aryl, or heteroaryl ring, such as, for example, 2-oxo-2,3-dihydro-1H-benzo[d]imidazolyl, 1,4,5,6-tetrahydrocyclopenta[c]pyrazolyl, 4,5,6,7-tetrahydrothieno[2,3-c]pyridinyl, 5,6-dihydro-4H-cyclopenta[b]thiophenyl, and 4,7-dihydro-5H-thieno[2,3-c]pyranyl. In some embodiments, the heterocyclyl group is a 4 to 6 membered monocyclic heterocyclyl group. In some embodiments, the heterocyclyl group is a 8 to 10 membered bicyclic heterocyclyl group.

As used herein the term “spiral” ring means a two-ring system wherein both rings share one common atom. Examples of spiral rings include 5-oxaspiro[2.3]hexane, oxaspiro[2.4]heptanyl, 5-oxaspiro[2.4]heptanyl, 4-oxaspiro[2.4]heptane, 4-oxaspiro[2.5]octanyl, 6-oxaspiro[2.5]octanyl, oxaspiro[2.5]octanyl, oxaspiro[3.4]octanyl, oxaspiro[bicyclo[2.1.1]hexane-2,3′-oxetan]-1-yl, oxaspiro[bicyclo[3.2.0]heptane-6,1′-cyclobutan]-7-yl, 2,6-diazaspiro[3.3]heptanyl, -oxa-6-azaspiro[3.3]heptane, 2,2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. bicyclo[1.1.1]pentane, bicyclo [2.2.1]heptane and bicyclo [3.2.1]octane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

As used herein, the term “a,” “an,” “the” and similar terms used in the context of the present disclosure (especially in the context of the claims) are to be construed to cover both the singular and plural unless otherwise indicated herein or clearly contradicted by the context. The use of any and all examples, or exemplary language (e.g. “such as”) provided herein is intended merely to better illuminate the disclosure and does not pose a limitation on the scope of the disclosure otherwise claimed.

It is also possible that the intermediates and compounds of the present disclosure may exist in different tautomeric forms, and all such forms are embraced within the scope of the disclosure. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form. In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions.

### EXEMPLIFICATION

### Abbreviation:

PE=petroleum ether
EtOAc=EA=ethyl acetate
ESI=electrospray ionisation
MeOH=methanol
EtOH=ethanol
DCM=dichloromethane
DIAD=Diisopropyl azodicarboxylate
DMF=dimethylformamide
DPPA=Diphenylphosphoryl azide
EDCI=1-Ethyl-3-(3-dimethylaminopropyl)carbodiimide
HATU=Hexafluorophosphate Azabenzotriazole Tetramethyl Uronium
HCl=hydrochloric acid
H₂O=water
NBS=N-bromosuccinimide
LCMS=liquid chromatography mass spectrometry
HPLC=high pressure liquid chromatography
THF=tetrahydrofuran
MeCN=ACN=acetonitrile
DMSO=dimetylsulfoxide
AcOH=acetic acid
CDI=carbonyldiimidazole
TFA=trifluoroacetic acid
FA=formic acid
DIPEA=diisopropylethyl amine
TLC=Thin Layer Chromatography
N2=Nitrogen
KOH=Potassium Hydroxide
NH₄HCO₃=Ammonium Bicarbonate
t-BuOH=tert-butanol
NH₄Cl=ammonium chloride
t-BuOK=potassium tert-butoxide
NaH=sodium hydride
Xantphos=4,5-Bis(diphenylphosphino)-9,9-dimethylxanthene
Na₂SO₄=sodium sulfate
K₂CO₃=potassium carbonate
NaHCO₃=sodium bicarbonate
Pd(OAc)₂=Palladium (II) Acetate
NaOH=Sodium Hydroxide
TCFH=N′-tetramethylformamidinium hexafluorophosphate
NMI=1-Methylimidazole
XantPhosPdG₃=[(4,5-Bis(diphenylphosphino)-9,9-dimethylxanthene)-2-(2′-amino-1,1′-biphenyl)]palladium(II) methanesulfonate
Pd/C=Palladium on Carbon
Pd(dppf)Cl₂ DCM=[1,1′-Bis(diphenylphosphino)ferrocene]dichloropalladium(II), complex with dichloromethane
Pd(dppf)Cl₂=[1,1′-Bis(diphenylphosphino)ferrocene]dichloropalladium(II)
Pd(PPh₃)₄=Tetrakis(triphenylphosphine)palladium(0)
Pd(PPh₃)Cl₂=Bis(triphenylphosphine)palladium(II) dichloride
PPh₃=triphenylphosphine
AlMe₃═Al(CH₃)₃=trimethylaluminum
Et₂O=diethyl ether
C₂Cl₆=hexachloroethane
PEPPSI IHept-Cl=(SP-4-1)-[1,3-Bis[2,6-bis(1-propylbutyl)phenyl]-4,5-dichloro-1,3-dihydro-2H-imidazol-2-ylidene]dichloro(3-chloropyridine-xN)palladium (ACI)
Br₂=bromine
B₂pin₂=bis(pinacoloto)diboron

### General Methods

1. ¹H NMR Spectra were Recorded on:
NMR10 Bruker AVANCE III HD 300 MHz
NMR16 Bruker AVANCE III HD 300 MHz
NMR19 Bruker AVANCE III HD 400 MHz
NMR24 Bruker AVANCE NEO 400 MHz
NMR30 Bruker AVANCE NEO 400 MHz

### 2. LCMS Measurement was Run on SHIMADZU LCMS-2020 Using the Follow Conditions:

Method A: Mobile Phase: A: Water (0.05% TFA) B: Acetonitrile (0.05% TFA); Gradient Phase: 5% B to 100% B within 2.0 min, 100% B with 0.7 min (total runtime: 2.8 min); Flow Rate: 1.5 mL/min; Column: HALO C18, 3.0*30 mm, 2.0 μm; Column Temperature: 40° C. Detectors: AD2 ELSD, PDA (220 nm and 254 nm), ESI.
Method B: Mobile Phase: A: Water (0.1% FA) B: Acetonitrile (0.1% FA); Gradient Phase: 5% B to 100% B within 2.0 min, 100% B with 0.7 min (total runtime: 2.8 min); Flow Rate: 1.5 mL/min; Column: HALO C18, 3.0*30 mm, 2.0 μm; Column Temperature: 40° C. Detectors: AD2 ELSD, PDA (220 nm and 254 nm), ESI.
Method C: Mobile Phase: A: Water (5 mM NH₄HCO₃) B: Acetonitrile; Gradient Phase: 10% B to 95% B within 2.0 min, 100% B with 0.6 min (total runtime: 2.8 min); Flow Rate: 1.5 mL/min; Column: Poroshell HPH—C18, 3.0*50 mm, 4.0 μm; Column Temperature: 40° C. Detectors: AD2 ELSD, PDA (220 nm and 254 nm), ESI.

The observed molecular ion for all compounds listed below is for [M+H]⁺, unless otherwise indicated.

### Preparation of Intermediates

### Intermediate A: N-(3-amino-5-bromophenyl) methanesulfonamide

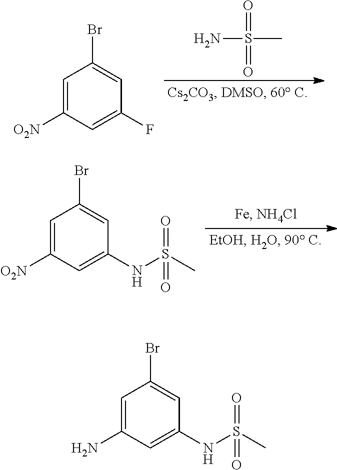

Step 1: A mixture of 1-bromo-3-fluoro-5-nitrobenzene (50.00 g, 227.27 mmol, 1.00 equiv) and methanesulfonamide (21.62 g, 227.27 mmol, 1.00 equiv) and Cs₂CO₃ (222.15 g, 681.83 mmol, 3.00 equiv) in 500 mL of DMSO, the resulted solution was stirred for 1 hour at 60° C. The mixture was cooled then quenched with 1000 mL of water. The aqueous layer was extracted with 3×500 mL of ethyl acetate. Dried over anhydrous Na₂SO₄ and concentrated and the residue was purified onto silica gel column eluted with 35% of ethyl acetate in petroleum ether to afford N-(3-bromo-5-nitrophenyl)methanesulfonamide (35.2 g, 52.5%) as a yellow solid. LCMS (ESI) [M+H]⁺: 295

Step 2: A mixture of N-(3-bromo-5-nitrophenyl)methanesulfonamide (35.2 g, 119.28 mmol, 1.00 equiv) and Fe (66.61 g, 1192.78 mmol, 10 equiv) and NH₄Cl (63.80 g, 1192.78 mmol, 10.00 equiv) in 200 mL of ethanol and 50 mL of water and this was stirred for 2 hours at 90° C. The resulting mixture was filtered, the filter cake was washed with 6×50 mL of methanol. The filtrate was concentrated under reduced pressure. The residue was purified onto silica gel column eluted with 50% of ethyl acetate in petroleum ether to afford N-(3-amino-5-bromophenyl)methanesulfonamide (25.1 g, 81.2%) as a yellow solid. LCMS (ESI) [M+H]⁺: 265. ¹H NMR (300 MHz, DMSO-d₆) δ 9.63 (s, 1H), 6.58-6.36 (m, 3H), 5.53 (s, 2H), 2.98 (s, 3H).

### Intermediate B: 1-methyl-5-(4,4,5,5-tetramethyl-1,3,2-dioxaborolan-2-yl)pyrrole-3-carboxylate

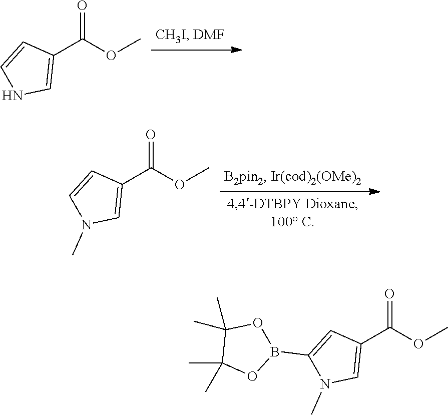

Step 1: To a stirred solution of methyl 1H-pyrrole-3-carboxylate (100 g, 800 mmol, 1 equiv) and NaH (60%) (64 g, 1.6 mol, 2 equiv) in DMF (1 L) was added iodomethane (227.2 g, 1.6 mol, 2 equiv) dropwise at 0° C. The resulting mixture was stirred for additional 3 hours at room temperature. The reaction was quenched by the addition of water (5 L) at 0° C. The resulting mixture was extracted with EtOAc (3×5 L). The combined organic layers were washed with brine (3×5 L), dried over anhydrous Na₂SO₄. After filtration, the filtrate was concentrated under reduced pressure. The residue was purified by silica gel column chromatography, eluted with PE/EA (5:1) to afford methyl 1-methylpyrrole-3-carboxylate (95 g, 85.6%) as a white solid

LCMS (ESI) [M+H]⁺: 140

Step 2: A solution of methyl 1-methylpyrrole-3-carboxylate (95 g, 683.5 mmol, 1 equiv), B₂Pin₂ (138.9 g, 546.7 mmol, 0.8 equiv), Di-mu-methoxobis(1,5-cyclooctadiene)diiridium(I) (6.7 g, 10.3 mmol, 0.015 equiv) and 4,4′-Di-tert-butyl-2,2′-bipyridine (5.49 g, 20.5 mmol, 0.03 equiv) in 1,4-dioxane (1 L) was stirred for 3 hours at 100° C. under nitrogen atmosphere. The mixture was concentrated under reduced pressure. The residue was purified by silica gel column chromatography, eluted with PE/EA (2/1). The residue was triturated with PE for 12 hours at room temperature and filtered to afford 1-methyl-5-(4,4,5,5-tetramethyl-1,3,2-dioxaborolan-2-yl)pyrrole-3-carboxylate (105 g, 57.8% yield) as a white solid.

LCMS (ESI) [M+H]⁺: 266

¹H NMR (300 MHz, Chloroform-d) δ 7.37 (d, J=1.6 Hz, 1H), 7.21 (d, J=1.6 Hz, 1H), 3.82 (s, 3H), 3.78 (s, 3H), 1.31 (s, 12H).

### Example 1: 1-(cyclohex-2-en-1-yl)-N-(5-(methylsulfonamido)cyclohexa-1,5-dien-1-yl)-1H-pyrazole-4-carboxamide

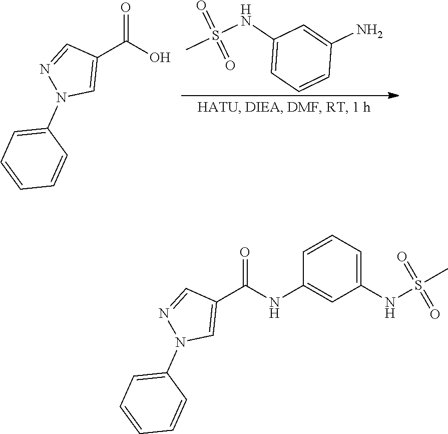

Into an 8-mL sealed tube, was placed N-(3-aminophenyl)methanesulfonamide (100.0 mg, 0.54 mmol, 1.00 equiv) and 1-phenyl-1H-pyrazole-4-carboxylic acid (100 mg, 0.54 mmol, 1.00 equiv), DIEA (109.1 mg, 1.62 mmol, 3.00 equiv) in 4 mL of DMF, to this was added HATU (266.7 mg, 0.7 mmol, 1.30 equiv) and this was stirred for 1 h at room temperature. The mixture was then quenched with 10 mL of water and extracted with 3×20 mL of ethyl acetate and dried over anhydrous Na₂SO₄. After filtration, the filtrate was concentrated under reduced pressure. The residue was purified onto silica gel column chromatography, eluted with 50% of ethyl acetate in petroleum ether to afford N-(3-methanesulfonamidophenyl)-1-phenyl-1H-pyrazole-4-carboxamide (28.8 mg, 14.9%) as a white solid. LCMS (ESI) [M+H]⁺: 357.09. ¹H NMR (300 MHz, DMSO-d6) δ 10.05 (s, 1H), 9.80 (s, 1H), 9.11 (s, 1H), 8.32 (s, 1H), 7.90 (d, J=8.0 Hz, 2H), 7.68 (s, 1H), 7.55 (q, J=6.9, 6.1 Hz, 3H), 7.40 (t, J=7.4 Hz, 1H), 7.31 (t, J=8.1 Hz, 1H), 6.93 (d, J=8.0 Hz, 1H), 3.02 (s, 3H).

The compounds listed in the following table were prepared using a procedure similar to that described for Example 1:

| | | MS | |
|---|---|---|---|
| | Example | (ESI) | |
| Structure | No. | [M + H]⁺ | ¹H NMR |
| 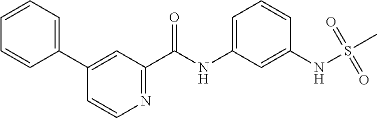 [SMILES: CS(=O)(=O)Nc1cccc(NC(=O)c2cc(-c3ccccc3)ccn2)c1] | 2 | 368 | ¹H NMR (400 MHz, DMSO-d6) δ 10.72 (s, 1H), 9.81 (s, 1H), 8.80 (d, J = 5.1 Hz, 1H), 8.41 (d, J = 1.9 Hz, 1H), 8.01 (dd, J = 5.1, 1.9 Hz, 1H), 7.91 (dt, J = 5.2, 1.6 Hz, 3H), 7.65 − 7.52 (m, 4H), 7.32 (t, J = 8.1 Hz, 1H), 6.98 - 6.93 (m, 1H), 3.05 (s, 3H). |
| 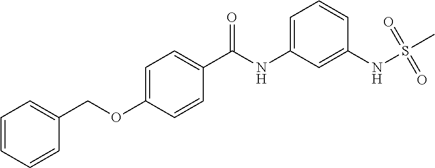 [SMILES: CS(=O)(=O)Nc1cccc(NC(=O)c2ccc(OCc3ccccc3)cc2)c1] | 3 | 397 | ¹H NMR (400 MHz, DMSO-d6) δ 10.14 (s, 1H), 9.73 (s, 1H), 7.98 − 7.92 (m, 2H), 7.73 (t, J = 2.1 Hz, 1H), 7.49 (ddd, J = 13.4, 7.8, 1.6 Hz, 3H), 7.44 − 7.38 (m, 2H), 7.37 − 7.31 (m, 1H), 7.27 (t, J = 8.1 Hz, 1H), 7.17 − 7.10 (m, 2H), 6.92 (ddd, J = 8.1, 2.2, 1.0 Hz, 1H), 5.21 (s, 2H), 3.00 (s, 3H). |
| 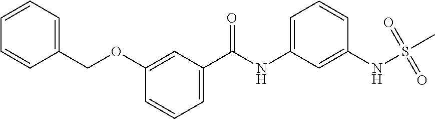 [SMILES: CS(=O)(=O)Nc1cccc(NC(=O)c2cccc(OCc3ccccc3)c2)c1] | 4 | 397 | ¹H NMR (400 MHz, DMSO-d6) δ 10.26 (s, 1H), 9.77 (s, 1H), 7.74 (t, J = 2.1 Hz, 1H), 7.59 − 7.22 (m, 11H), 6.94 (dd, J = 8.0, 2.1 Hz, 1H), 5.19 (s, 2H), 3.01 (s, 3H). |
| 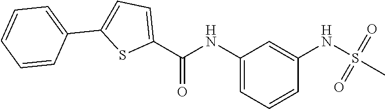 [SMILES: CS(=O)(=O)Nc1cccc(NC(=O)c2ccc(-c3ccccc3)s2)c1] | 5 | 373 | ¹H NMR (300 MHz, DMSO-d6) δ 10.32 (s, 1H), 9.81 (s, 1H), 8.05 (d, J = 4.0 Hz, 1H), 7.84 − 7.72 (m, 2H), 7.68 (t, J = 2.1 Hz, 1H), 7.63 (d, J = 4.0 Hz, 1H), 7.56 − 7.43 (m, 3H), 7.43 − 7.36 (m, 1H), 7.31 (t, J = 8.1 Hz, 1H), 7.00 − 6.90 (m, 1H), 3.02 (s, 3H). |
| 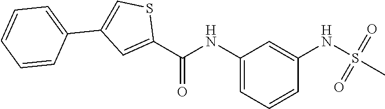 [SMILES: CS(=O)(=O)Nc1cccc(NC(=O)c2cc(-c3ccccc3)cs2)c1] | 6 | 373 | ¹H NMR (400 MHz, DMSO-d6) δ 10.34 (s, 1H), 9.82 (s, 1H), 8.49 (d, J = 1.5 Hz, 1H), 8.19 (d, J = 1.4 Hz, 1H), 7.79 − 7.72 (m, 2H), 7.69 (t, J = 2.1 Hz, 1H), 7.55 − 7.44 (m, 3H), 7.39 − 7.29 (m, 2H), 6.95 (ddd, J = 8.0, 2.2, 0.9 Hz, 1H), 3.02 (s, 3H). |
| 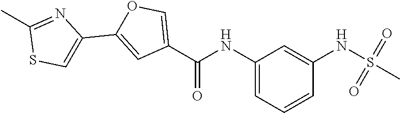 | 7 | 378 | |
| 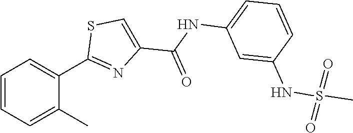 [SMILES: Cc1ccccc1-c1nc(C(=O)Nc2cccc(NS(C)(=O)=O)c2)cs1] | 8 | 388 | |
| 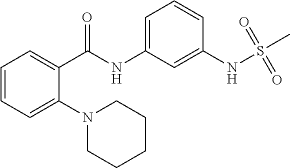 [SMILES: CS(=O)(=O)Nc1cccc(NC(=O)c2ccccc2N2CCCCC2)c1] | 9 | 374 | |
| 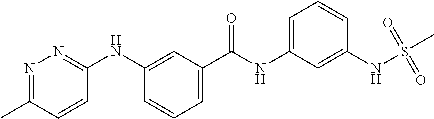 | 10 | 398 | |
| 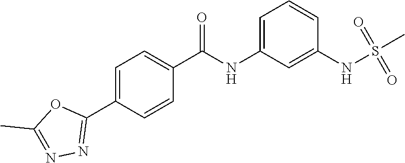 | 11 | 373 | |
| 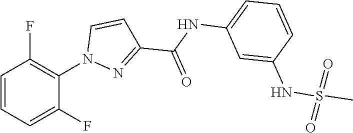 [SMILES: CS(=O)(=O)Nc1cccc(NC(=O)c2ccn(-c3c(F)cccc3F)n2)c1] | 12 | 393 | |
| 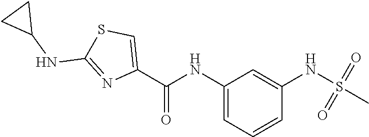 [SMILES: CS(=O)(=O)Nc1cccc(NC(=O)c2csc(NC3CC3)n2)c1] | 13 | 353 | |
| 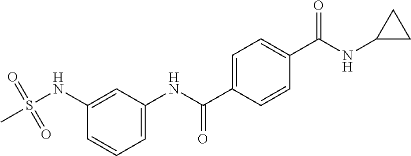 [SMILES: CS(=O)(=O)Nc1cccc(NC(=O)c2ccc(C(=O)NC3CC3)cc2)c1] | 14 | 374 | |
| 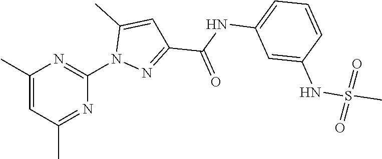 | 15 | | |
| 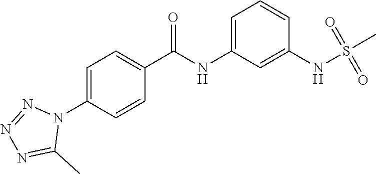 [SMILES: Cc1nnnn1-c1ccc(C(=O)Nc2cccc(NS(C)(=O)=O)c2)cc1] | 16 | 325 | |
| 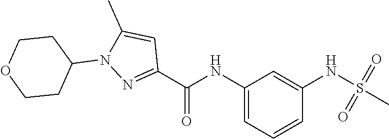 [SMILES: Cc1cc(C(=O)Nc2cccc(NS(C)(=O)=O)c2)nn1C1CCOCC1] | 17 | 379 | |
| 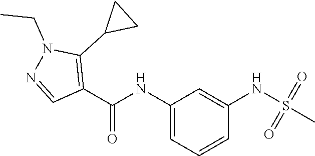 [SMILES: CCn1ncc(C(=O)Nc2cccc(NS(C)(=O)=O)c2)c1C1CC1] | 18 | | |
| 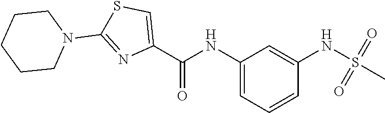 [SMILES: CS(=O)(=O)Nc1cccc(NC(=O)c2csc(N3CCCCC3)n2)c1] | 19 | 381 | |
| 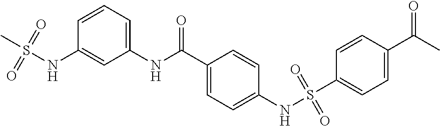 [SMILES: CC(=O)c1ccc(S(=O)(=O)Nc2ccc(C(=O)Nc3cccc(NS(C)(=O)=O)c3)cc2)cc1] | 20 | 488 | |
| 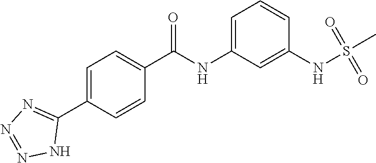 [SMILES: CS(=O)(=O)Nc1cccc(NC(=O)c2ccc(-c3nnn[nH]3)cc2)c1] | 21 | 359 | |
| 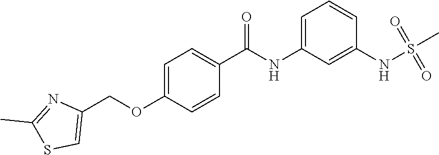 | 22 | 418 | |
| 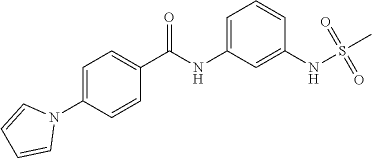 [SMILES: CS(=O)(=O)Nc1cccc(NC(=O)c2ccc(-n3cccc3)cc2)c1] | 23 | 356 | |
| 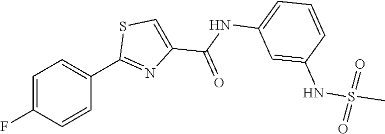 [SMILES: CS(=O)(=O)Nc1cccc(NC(=O)c2csc(-c3ccc(F)cc3)n2)c1] | 24 | 392 | |
| 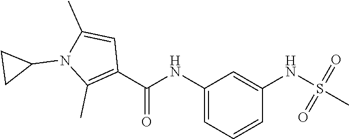 [SMILES: Cc1cc(C(=O)Nc2cccc(NS(C)(=O)=O)c2)c(C)n1C1CC1] | 25 | 348 | |
| 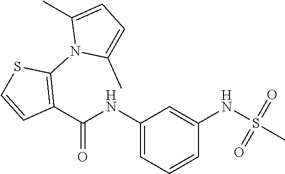 [SMILES: Cc1ccc(C)n1-c1sccc1C(=O)Nc1cccc(NS(C)(=O)=O)c1] | 26 | 390 | |
| 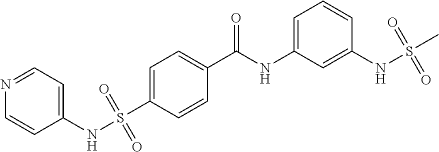 [SMILES: CS(=O)(=O)Nc1cccc(NC(=O)c2ccc(S(=O)(=O)Nc3ccncc3)cc2)c1] | 27 | 447 | |
| 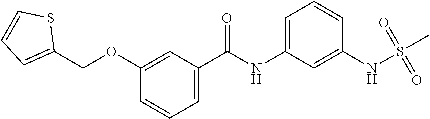 [SMILES: CS(=O)(=O)Nc1cccc(NC(=O)c2cccc(OCc3cccs3)c2)c1] | 28 | 403 | |
| 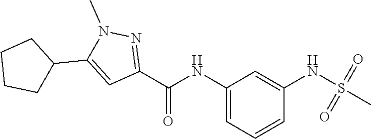 [SMILES: Cn1nc(C(=O)Nc2cccc(NS(C)(=O)=O)c2)cc1C1CCCC1] | 29 | 363 | |
| 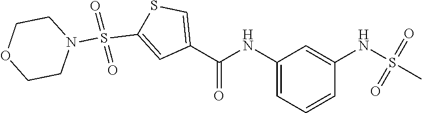 [SMILES: CS(=O)(=O)Nc1cccc(NC(=O)c2csc(S(=O)(=O)N3CCOCC3)c2)c1] | 30 | 446 | |
| 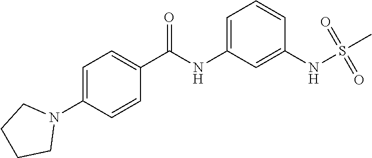 [SMILES: CS(=O)(=O)Nc1cccc(NC(=O)c2ccc(N3CCCC3)cc2)c1] | 31 | | |
| 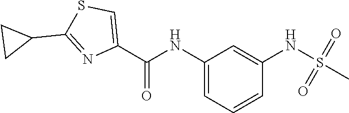 [SMILES: CS(=O)(=O)Nc1cccc(NC(=O)c2csc(C3CC3)n2)c1] | 32 | 338 | |
| 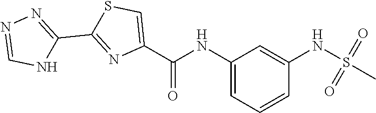 [SMILES: CS(=O)(=O)Nc1cccc(NC(=O)c2csc(-c3nnc[nH]3)n2)c1] | 33 | 365 | |
| 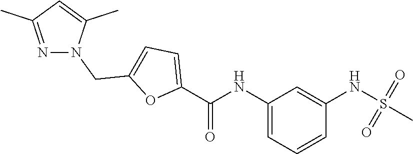 | 34 | 389 | |

### Example 35: 1-benzyl-N-(3-methanesulfonamidophenyl)pyrazole-4-carboxamide

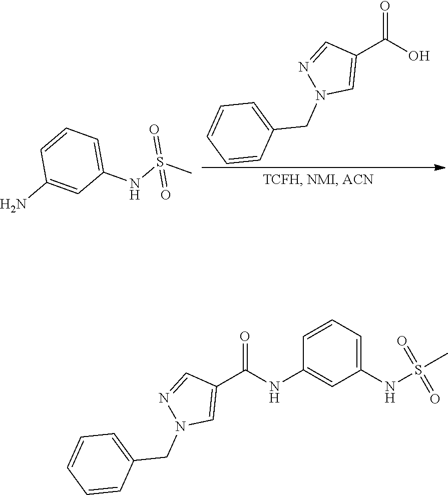

The solution of 1-benzylpyrazole-4-carboxylic acid (130.3 mg, 0.644 mmol, 1.20 equiv), NMI (88.2 mg, 1.074 mmol, 2.00 equiv) in ACN (3 mL) was added TCFH (226 mg, 0.805 mmol, 1.50 equiv) at room temperature. The reaction mixture was stirred for 10 m, then N-(3-aminophenyl)methanesulfonamide (100 mg, 0.537 mmol, 1 equiv) was added and stirred for another one hour. The reaction mixture was purified by HPLC to afford 1-benzyl-N-(3-methanesulfonamidophenyl)pyrazole-4-carboxamide (85.7 mg, 42.96%) as a white solid. LCMS (ESI) [M+H]⁺=˜371. H NMR (300 MHz, DMSO-d6) δ 9.90 (s, 1H), 9.76 (s, 1H), 8.45 (d, J 0.8 Hz, 1H), 8.06 (d, J 0.8 Hz, 1H), 7.64 (t, J 2.1 Hz, 1H), 7.53-7.44 (m, 1H), 7.42-7.23 (m, 6H), 6.90 (ddd, J=8.1, 2.2, 1.0 Hz, 1H), 5.40 (s, 2H), 3.00 (s, 3H). The compounds listed in the following table were prepared using a procedure similar to that described for Example 36:

| | | MS | |
|---|---|---|---|
| | Example | (ESI) | |
| Structure | No. | [M + H]⁺ | ¹H NMR |
| 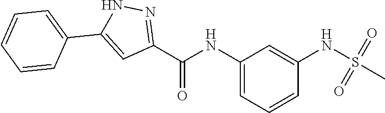 [SMILES: CS(=O)(=O)Nc1cccc(NC(=O)c2cc(-c3ccccc3)[nH]n2)c1] | 36 | 357 | ¹H NMR (400 MHz, DMSO-d6) δ 13.80 (s, 1H), 10.20 (d, J = 58.7 Hz, 1H), 9.78 (s, 1H), 7.93 − 7.64 (m, 3H), 7.62 − 7.11 (m, 6H), 6.93 (d, J = 8.0 Hz, 1H), 3.03 (s, 3H) |
| 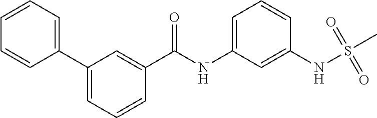 [SMILES: CS(=O)(=O)Nc1cccc(NC(=O)c2cccc(-c3ccccc3)c2)c1] | 37 | 367 | ¹H NMR (300 MHz, DMSO-d6) δ 10.42 (s, 1H), 9.80 (s, 1H), 8.22 (t, J = 1.8 Hz, 1H), 8.01 − 7.86 (m, 2H), 7.82 − 7.72 (m, 3H), 7.63 (t, J = 7.7 Hz, 1H), 7.59 − 7.49 (m, 3H), 7.47 − 7.38 (m, 1H), 7.32 (t, J = 8.1 Hz, 1H), 6.96 (ddd, J = 8.1, 2.2, 1.0 Hz, 1H), 3.02 (s, 3H) |
| 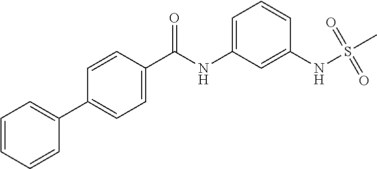 [SMILES: CS(=O)(=O)Nc1cccc(NC(=O)c2ccc(-c3ccccc3)cc2)c1] | 38 | 367 | ¹H NMR (300 MHz, DMSO-d6) δ 10.37 (s, 1H), 9.79 (s, 1H), 8.14 − 7.94 (m, 2H), 7.90 − 7.65 (m, 5H), 7.65 − 7.40 (m, 4H), 7.31 (t, J = 8.1 Hz, 1H), 6.95 (ddd, J = 8.0, 2.2, 1.0 Hz, 1H), 3.03 (s, 3H) |
| 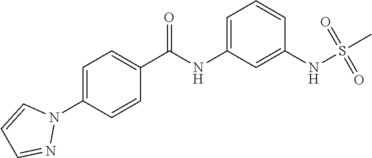 [SMILES: CS(=O)(=O)Nc1cccc(NC(=O)c2ccc(-n3cccn3)cc2)c1] | 39 | 357 | ¹H NMR (300 MHz, DMSO-d6) δ 10.36 (s, 1H), 9.79 (s, 1H), 8.66 (d, J = 2.5 Hz, 1H), 8.11 (d, J = 8.8 Hz, 2H), 8.02 (d, J = 8.7 Hz, 2H), 7.83 (d, J = 1.7 Hz, 1H), 7.76 (s, 1H), 7.55 (d, J = 8.2 Hz, 1H), 7.31 (t, J = 8.0 Hz, 1H), 6.95 (d, J = 8.1 Hz, 1H), 6.66 − 6.55 (m, 1H), 3.02 (d, J = 1.4 Hz, 3H) |
| 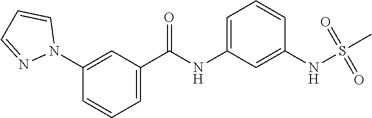 [SMILES: CS(=O)(=O)Nc1cccc(NC(=O)c2cccc(-n3cccn3)c2)c1] | 40 | 357 | ¹H NMR (400 MHz, DMSO-d6) δ 10.53 (s, 1H), 9.23 − 9.04 (m, 1H), 8.70 (dd, J = 3.9, 1.9 Hz, 1H), 8.32 (d, J = 2.0 Hz, 1H), 8.28 (s, 1H), 8.11 − 7.95 (m, 2H), 7.76 (d, J = 2.0 Hz, 4H), 7.16 (dd, J = 8.4, 2.0 Hz, 1H), 7.02 (s, 1H), 6.59 (t, J = 2.2 Hz, |
| | | | 1H), 6.48 (dt, J = 8.4, 2.2 Hz, |
| | | | 1H), 4.63 (d, J = 5.6 Hz, 2H), |
| | | | 3.83 (d, J = 2.0 Hz, 3H), 3.74 (d, |
| | | | J = 2.0 Hz, 3H) |
| 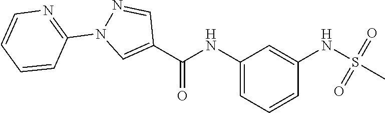 [SMILES: CS(=O)(=O)Nc1cccc(NC(=O)c2cnn(-c3ccccn3)c2)c1] | 41 | 358 | ¹H NMR (300 MHz, DMSO-d6) δ 10.17 (s, 1H), 9.80 (s, 1H), 9.43 (s, 1H), 8.57 (dd, J = 4.6, 1.6 Hz, 1H), 8.31 (s, 1H), 8.19 − 7.90 (m, 2H), 7.71 (t, J = 2.1 Hz, 1H), 7.56 (d, J = 8.2 Hz, 1H), 7.49 − 7.43 (m, 1H), 7.30 (t, J = |
| | | | 8.1 Hz, 1H), 6.93 (dd, J = 7.9, |
| | | | 2.1 Hz, 1H), 3.02 (s, 3H). |
| 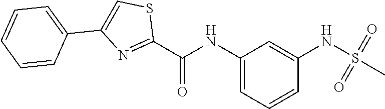 [SMILES: CS(=O)(=O)Nc1cccc(NC(=O)c2nc(-c3ccccc3)cs2)c1] | 42 | 374 | ¹H NMR (300 MHz, DMSO-d6) δ 10.68 (s, 1H), 9.85 (s, 1H), 8.51 (s, 1H), 8.29 − 8.12 (m, 2H), 7.83 (t, J = 2.1 Hz, 1H), 7.65 − 7.58 (m, 1H), 7.57 − 7.47 (m, 2H), 7.47 − 7.39 (m, 1H), 7.35 (t, J = 8.1 Hz, 1H), 7.00 |
| | | | (ddd, J = 8.1, 2.2, 1.0 Hz, 1H), |
| | | | 3.05 (s, 3H). |
| 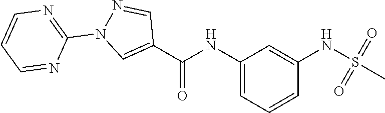 [SMILES: CS(=O)(=O)Nc1cccc(NC(=O)c2cnn(-c3ncccn3)c2)c1] | 43 | 359 | ¹H NMR (400 MHz, DMSO-d6) δ 10.19 (s, 1H), 9.45 (s, 1H), 8.95 (d, J = 4.8 Hz, 2H), 8.32 (s, 1H), 7.69 (d, J = 2.2 Hz, 1H), 7.61 − 7.50 (m, 2H), 7.29 (t, J = 8.1 Hz, 1H), 6.92 (d, J = 8.0 Hz, 1H), 3.00 (s, 3H). |
| 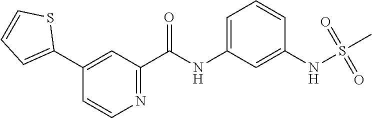 [SMILES: CS(=O)(=O)Nc1cccc(NC(=O)c2cc(-c3cccs3)ccn2)c1] | 44 | 374 | ¹H NMR (400 MHz, DMSO-d6) δ 10.70 (s, 1H), 9.82 (s, 1H), 8.73 (dd, J = 5.2, 0.7 Hz, 1H), 8.38 − 8.26 (m, 1H), 7.97 (dd, J = 3.7, 1.2 Hz, 1H), 7.95 (dd, J = 5.1, 2.0 Hz, 1H), 7.90 (t, J = 2.1 Hz, 1H), 7.83 (dd, J = 5.0, 1.1 Hz, 1H), 7.69 − 7.54 (m, 1H), |
| | | | 7.32 (t, J = 8.1 Hz, 1H), 7.28 (dd, |
| | | | J = 5.0, 3.7 Hz, 1H), 6.95 (ddd, |
| | | | J = 8.1, 2.2, 1.0 Hz, 1H), 3.05 (s, |
| | | | 3H). |

### Example 45: 4-(3-fluorophenyl)-N-(3-(methylsulfonamido)phenyl)thiophene-2-carboxamide

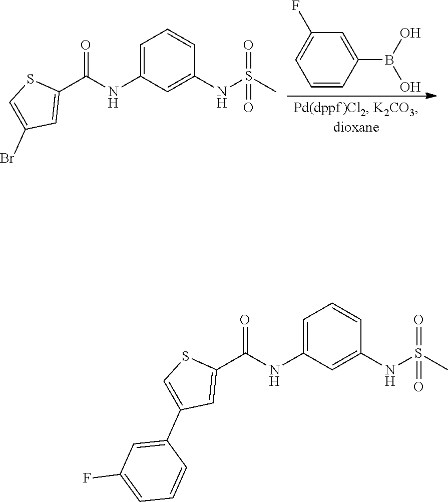

A solution of 4-bromo-N-(3-methanesulfonamidophenyl)thiophene-2-carboxamide (100 mg, 0.266 mmol), Pd(dppf)Cl₂ (19.4 mg, 0.0266 mmol), K₂CO₃ (110 mg, 0.798 mmol) and (3-fluorophenyl)boronic acid (74.4 mg, 0.532 mmol) in dioxane (4 mL) was stirred for 2 h at 80° C. under nitrogen atmosphere. The resulting mixture was concentrated under reduced pressure. The residue was purified by silica gel column chromatography, eluted with Petroleum ether/EtOAc (0-100%) to afford 4-(3-fluorophenyl)-N-(3-methanesulfonamidophenyl)thiophene-2-carboxamide (62.8 mg, 0.160 mmol) as a white solid. LCMS (ESI) [M+H]⁺: 391. ¹H NMR (300 MHz, DMSO-d6) δ 10.32 (s, 1H), 9.83 (s, 1H), 8.52 (d, J=1.5 Hz, 1H), 8.30 (d, J=1.4 Hz, 1H), 7.69 (t, J=2.0 Hz, 1H), 7.66-7.46 (m, 4H), 7.33 (t, J=8.1 Hz, 1H), 7.26-7.13 (m, 1H), 6.96 (dd, J=7.7, 2.2 Hz, 1H), 3.03 (s, 3H).

The compounds listed in the following table were prepared using a procedure similar to that described for example 45:

| | Example | MS (ESI) | |
|---|---|---|---|
| Structure | No. | [M + H]⁺ | ¹H NMR |
| 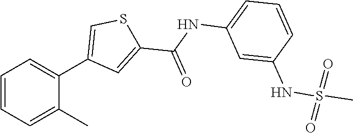 [SMILES: Cc1ccccc1-c1csc(C(=O)Nc2cccc(NS(C)(=O)=O)c2)c1] | 46 | 387 | ¹H NMR (300 MHz, DMSO-d6) δ 10.27 (s, 1H), 9.81 (s, 1H), 8.18 (d, J = 1.4 Hz, 1H), 7.86 (d, J = 1.4 Hz, 1H), 7.67 (t, J = 2.1 Hz, 1H), 7.51 (ddd, J = 8.2, 2.1, 1.0 Hz, 1H), 7.44 − 7.23 (m, 5H), 6.95 (ddd, J = 8.1, |
| | | | 2.2, 1.0 Hz, 1H), 3.01 |
| | | | (s, 3H), 2.37 (s, 3H). |
| 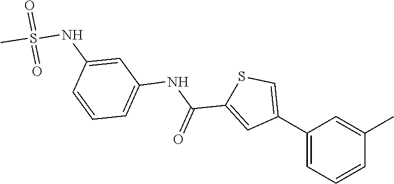 | 47 | 387 | ¹H NMR (300 MHz, DMSO-d6) δ 10.33 (s, 1H), 9.82 (s, 1H), 8.49 (d, J = 1.4 Hz, 1H), 8.17 (d, J = 1.3 Hz, 1H), 7.70 (t, J = 2.1 Hz, 1H), 7.63 − 7.46 (m, 3H), 7.34 (dt, J = 13.5, 7.8 Hz, 2H), 7.18 (d, J = 7.5 Hz, 1H), 6.96 (ddd, J = 8.0, 2.3, 1.0 Hz, 1H), 3.02 (s, 3H), 2.39 (s, 3H). |
| 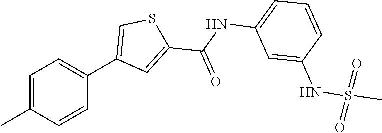 | 48 | 387 | ¹H NMR (400 MHz, DMSO-d6) δ 10.32 (s, 1H), 9.82 (s, 1H), 8.47 (d, J = 1.5 Hz, 1H), 8.13 (d, J = 1.3 Hz, 1H), 7.69 (t, J = 2.1 Hz, 1H), 7.64 (d, J = 7.8 Hz, 2H), 7.52 (dd, J = 8.0, 1.9 Hz, 1H), 7.37 − 7.22 (m, |
| | | | 3H), 6.95 (dd, J = 8.1, |
| | | | 2.0 Hz, 1H), 3.02 (s, |
| | | | 3H), 2.34 (s, 3H). |
| 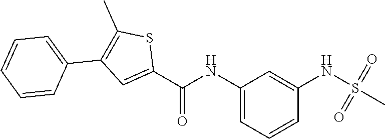 [SMILES: Cc1sc(C(=O)Nc2cccc(NS(C)(=O)=O)c2)cc1-c1ccccc1] | 49 | 387 | ¹H NMR (300 MHz, DMSO-d6) δ 10.20 (s, 1H), 9.79 (s, 1H), 8.10 (s, 1H), 7.67 (t, J = 2.0 Hz, 1H), 7.57 − 7.46 (m, 5H), 7.45 − 7.35 (m, 1H), 7.30 (t, J = 8.1 Hz, 1H), 6.93 (d, J = 8.6 Hz, 1H), 3.01 (s, 3H), 2.54 |
| | | | (s, 3H). |
| 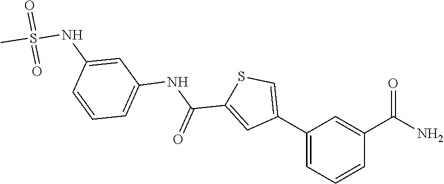 [SMILES: CS(=O)(=O)Nc1cccc(NC(=O)c2cc(-c3cccc(C(N)=O)c3)cs2)c1] | 50 | 416 | ¹H NMR (300 MHz, DMSO-d6) δ 10.42 (s, 1H), 9.83 (s, 1H), 8.58 (d, J = 1.4 Hz, 1H), 8.33 − 8.23 (m, 2H), 8.10 (s, 1H), 7.91 (d, J = 7.8 Hz, 1H), 7.85 (d, J = 7.7 Hz, 1H), 7.70 (t, J = 2.0 Hz, 1H), 7.62 − 7.44 (m, 3H), 7.33 (t, J = 8.1 Hz, 1H), 7.01 − 6.93 (m, 1H), 3.03 (s, 3H). |
| 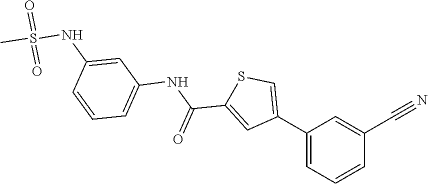 [SMILES: CS(=O)(=O)Nc1cccc(NC(=O)c2cc(-c3cccc(C#N)c3)cs2)c1] | 51 | 398 | ¹H NMR (300 MHz, DMSO-d6) δ 10.33 (s, 1H), 9.84 (s, 1H), 8.56 (d, J = 1.5 Hz, 1H), 8.39 (d, J = 1.4 Hz, 1H), 8.24 (t, J = 1.7 Hz, 1H), 8.10 (dt, J = 8.0, 1.4 Hz, 1H), 7.83 (dt, J = 7.7, 1.3 Hz, 1H), 7.76 − 7.65 (m, 2H), 7.52 (ddd, J = 8.2, 2.0, 1.0 Hz, 1H), 7.33 (t, J = 8.1 Hz, 1H), 6.97 |
| | | | (ddd, J = 8.1, 2.2, 1.0 |
| | | | Hz, 1H), 3.03 (s, 3H). |
| 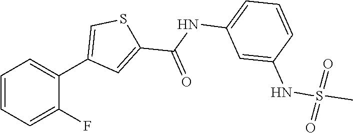 [SMILES: CS(=O)(=O)Nc1cccc(NC(=O)c2cc(-c3ccccc3F)cs2)c1] | 52 | 391 | ¹H NMR (300 MHz, DMSO-d6) δ 10.38 (s, 1H), 9.82 (s, 1H), 8.45 (s, 1H), 8.15 (s, 1H), 7.76 (td, J = 7.7, 1.6 Hz, 1H), 7.69 (t, J = 2.0 Hz, 1H), 7.53 (dd, J = 8.2, 1.7 Hz, 1H), 7.48 − 7.26 (m, 4H), 7.03 − 6.90 (m, |
| | | | 1H), 3.02 (s, 3H). |
| 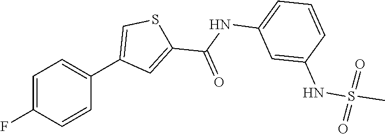 [SMILES: CS(=O)(=O)Nc1cccc(NC(=O)c2cc(-c3ccc(F)cc3)cs2)c1] | 53 | 391 | ¹H NMR (300 MHz, DMSO-d6) δ 10.33 (s, 1H), 9.82 (s, 1H), 8.46 (d, J = 1.5 Hz, 1H), 8.17 (d, J = 1.4 Hz, 1H), 7.88 − 7.74 (m, 2H), 7.69 (t, J = 2.0 Hz, 1H), 7.58 − 7.47 (m, 1H), 7.33 (ddd, J = 9.0, 6.9, 2.3 Hz, |
| | | | 3H), 6.96 (ddd, J = 8.0, |
| | | | 2.2, 1.0 Hz, 1H), 3.02 |
| | | | (s, 3H). |
| 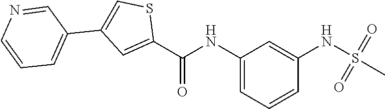 [SMILES: CS(=O)(=O)Nc1cccc(NC(=O)c2cc(-c3cccnc3)cs2)c1] | 54 | 374 | ¹H NMR (300 MHz, DMSO-d6) δ 10.35 (s, 1H), 9.83 (s, 1H), 9.01 (dd, J = 2.4, 0.8 Hz, 1H), 8.63 − 8.48 (m, 2H), 8.35 (d, J = 1.4 Hz, 1H), 8.14 (ddd, J = 8.0, |
| | | | 2.4, 1.6 Hz, 1H), 7.69 (t, |
| | | | J = 2.1 Hz, 1H), 7.59 − |
| | | | 7.47 (m, 2H), 7.33 (t, |
| | | | J = 8.1 Hz, 1H), 6.96 |
| | | | (ddd, J = 8.1, 2.2, 1.0 |
| | | | Hz, 1H), 3.02 (s, 3H). |
| 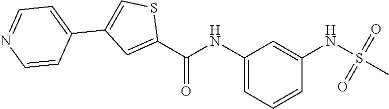 [SMILES: CS(=O)(=O)Nc1cccc(NC(=O)c2cc(-c3ccncc3)cs2)c1] | 55 | 374 | ¹H NMR (300 MHz, DMSO-d6) δ 10.40 (s, 1H), 9.83 (s, 1H), 8.79 − 8.63 (m, 2H), 8.59 (d, J = 1.4 Hz, 1H), 8.51 (d, J = 1.4 Hz, 1H), 7.87 − 7.66 (m, 3H), 7.53 (d, |
| | | | J = 8.1 Hz, 1H), 7.33 (t, |
| | | | J = 8.1 Hz, 1H), 7.05 − |
| | | | 6.91 (m, 1H), 3.03 (s, |
| | | | 3H). |
| 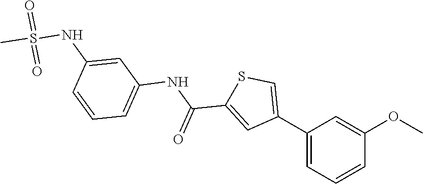 | 56 | 403 | ¹H NMR (300 MHz, DMSO-d6) δ 10.32 (s, 1H), 9.82 (s, 1H), 8.49 (d, J = 1.5 Hz, 1H), 8.22 (d, J = 1.4 Hz, 1H), 7.69 (t, J = 2.1 Hz, 1H), 7.56 − 7.49 (m, 1H), 7.45 − 7.27 (m, 4H), 6.99 − 6.90 (m, 2H), 3.84 (s, 3H), 3.02 (s, 3H). |
| 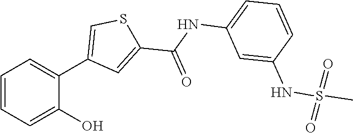 [SMILES: CS(=O)(=O)Nc1cccc(NC(=O)c2cc(-c3ccccc3O)cs2)c1] | 57 | 389 | ¹H NMR (300 MHz, DMSO-d6) δ 10.32 (s, 1H), 9.93 (s, 1H), 9.80 (s, 1H), 8.47 (d, J = 1.4 Hz, 1H), 8.12 (d, J = 1.3 Hz, 1H), 7.70 (t, J = 2.1 Hz, 1H), 7.54 (ddd, J = 6.7, 4.9, 1.7 Hz, 2H), 7.31 (t, J = 8.1 Hz, 1H), |
| | | | 7.21 − 7.13 (m, 1H), |
| | | | 7.02 − 6.87 (m, 3H), |
| | | | 3.02 (s, 3H). |
| 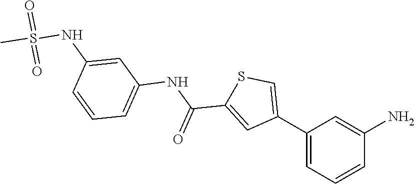 [SMILES: CS(=O)(=O)Nc1cccc(NC(=O)c2cc(-c3cccc(N)c3)cs2)c1] | 58 | 388 | ¹H NMR (300 MHz, DMSO-d6) δ 10.35 (s, 1H), 9.81 (s, 1H), 8.38 (d, J = 1.5 Hz, 1H), 7.98 (d, J = 1.3 Hz, 1H), 7.69 (t, J = 2.0 Hz, 1H), 7.57 − 7.50 (m, 1H), 7.32 (t, J = 8.1 Hz, 1H), 7.13 (t, J = 7.7 Hz, 1H), 6.99 − 6.87 (m, 3H), 6.58 (d, J = 8.2 Hz, 1H), 5.37 (s, 2H), 3.02 (s, 3H). |
| 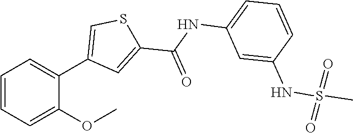 [SMILES: COc1ccccc1-c1csc(C(=O)Nc2cccc(NS(C)(=O)=O)c2)c1] | 59 | 40 Observed molecular ion is [M − H]⁻ | ¹H NMR (300 MHz, DMSO-d6) δ 10.30 (s, 1H), 9.79 (s, 1H), 8.40 (d, J = 1.4 Hz, 1H), 8.06 (d, J = 1.4 Hz, 1H), 7.68 (t, J = 2.1 Hz, 1H), 7.58 (dd, J = 7.6, 1.7 Hz, 1H), 7.55 − 7.48 (m, 1H), 7.41 − 7.26 (m, |
| | | | 2H), 7.15 (dd, J = 8.4, |
| | | | 1.1 Hz, 1H), 7.06 (td, J = |
| | | | 7.5, 1.1 Hz, 1H), 6.95 |
| | | | (ddd, J = 8.0, 2.2, 1.0 |
| | | | Hz, 1H), 3.88 (s, 3H), |
| | | | 3.01 (s, 3H). |
| 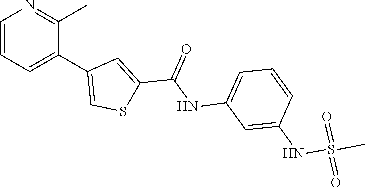 [SMILES: Cc1ncccc1-c1csc(C(=O)Nc2cccc(NS(C)(=O)=O)c2)c1] | 60 | 388 | ¹H NMR (300 MHz, DMSO-d6) δ 10.31 (s, 1H), 9.75 (s, 1H), 8.47 (dd, J = 4.8, 1.7 Hz, 1H), 8.21 (d, J = 1.5 Hz, 1H), 7.97 (d, J = 1.4 Hz, 1H), 7.76 (dd, J = 7.7, 1.8 Hz, 1H), 7.67 (t, J = 2.1 Hz, 1H), 7.60 − 7.45 (m, 1H), 7.43 − 7.20 (m, 2H), 6.95 (ddd, J = 8.1, 2.2, 1.0 Hz, 1H), 3.01 |
| | | | (s, 3H), 2.57 (s, 3H) |
| 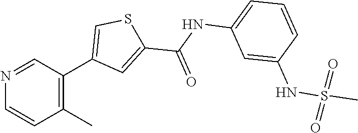 [SMILES: Cc1ccncc1-c1csc(C(=O)Nc2cccc(NS(C)(=O)=O)c2)c1] | 61 | 388 | ¹H NMR (300 MHz, DMSO-d6) δ 10.29 (s, 1H), 9.81 (s, 1H), 8.54 (s, 1H), 8.44 (d, J = 5.0 Hz, 1H), 8.23 (d, J = 1.5 Hz, 1H), 8.01 (d, J = 1.4 Hz, 1H), 7.66 (t, J = 2.1 Hz, 1H), 7.50 (dt, J = 8.3, 1.2 Hz, 1H), 7.40 − |
| | | | 7.24 (m, 2H), 7.06 − |
| | | | 6.80 (m, 1H), 3.01 (s, |
| | | | 3H), 2.40 (s, 3H) |
| 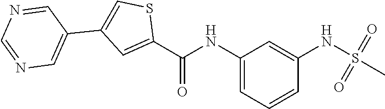 [SMILES: CS(=O)(=O)Nc1cccc(NC(=O)c2cc(-c3cncnc3)cs2)c1] | 62 | 375 | ¹H NMR (300 MHz, DMSO-d6) δ 10.39 (s, 1H), 9.83 (s, 1H), 9.22 (s, 2H), 9.17 (s, 1H), 8.54 (dd, J = 28.5, 1.4 Hz, 2H), 7.69 (t, J = 2.1 Hz, 1H), 7.59 − 7.50 (m, |
| | | | 1H), 7.33 (t, J = 8.1 Hz, |
| | | | 1H), 6.96 (ddd, J = 8.0, |
| | | | 2.2, 0.9 Hz, 1H), 3.02 |
| | | | (s, 3H) |
| 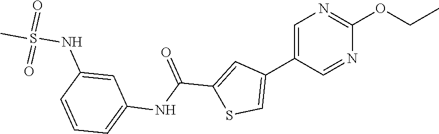 [SMILES: CCOc1ncc(-c2csc(C(=O)Nc3cccc(NS(C)(=O)=O)c3)c2)cn1] | 63 | 419 | ¹H NMR (300 MHz, DMSO-d6) δ 10.35 (s, 1H), 9.83 (s, 1H), 8.98 (s, 2H), 8.47 (d, J = 1.5 Hz, 1H), 8.29 (d, J = 1.4 Hz, 1H), 7.68 (t, J = 2.1 Hz, 1H), 7.58 − 7.43 (m, 1H), 7.33 (t, J = 8.1 Hz, 1H), 6.96 (ddd, J = 8.1, |
| | | | 2.2, 1.0 Hz, 1H), 4.41 |
| | | | (q, J = 7.0 Hz, 2H), 3.02 |
| | | | (s, 3H), 1.37 (t, J = 7.0 |
| | | | Hz, 3H) |
| 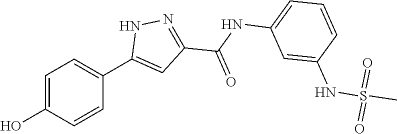 [SMILES: CS(=O)(=O)Nc1cccc(NC(=O)c2cc(-c3ccc(O)cc3)[nH]n2)c1] | 64 | 373 | ¹H NMR (400 MHz, DMSO-d6) δ 13.55 (s, 1H), 10.08 (s, 1H), 9.76 (s, 2H), 7.77 (s, 1H), 7.69 − 7.60 (m, 2H), 7.54 (dt, J = 8.1, 1.4 Hz, 1H), 7.28 (t, J = 8.1 Hz, 1H), 7.02 (s, 1H), 6.97 − 6.91 (m, 1H), 6.85 (d, J = |
| | | | 8.3 Hz, 2H), 3.03 (s, 3H) |
| 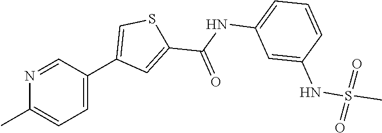 | 65 | 388 | ¹H NMR (300 MHz, DMSO-d6) δ 10.34 (s, 1H), 9.82 (s, 1H), 8.90 − 8.74 (m, 1H), 8.51 (d, J = 1.5 Hz, 1H), 8.27 (d, J = 1.3 Hz, 1H), 8.03 (dd, J = 8.1, 2.4 Hz, 1H), 7.69 (t, J = 2.1 Hz, 1H), 7.56 − 7.47 (m, 1H), |
| | | | 7.40 − 7.18 (m, 2H), |
| | | | 6.96 (ddd, J = 8.0, 2.2, |
| | | | 1.0 Hz, 1H), 3.02 (s, |
| | | | 3H), 2.50 (s, 3H) |
| 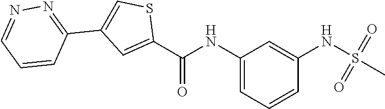 [SMILES: CS(=O)(=O)Nc1cccc(NC(=O)c2cc(-c3cccnn3)cs2)c1] | 66 | 375 | ¹H NMR (300 MHz, DMSO-d6) δ 10.54 (s, 1H), 9.83 (s, 1H), 9.21 (dd, J = 4.9, 1.5 Hz, 1H), 8.95 (d, J = 1.3 Hz, 1H), 8.68 (d, J = 1.3 Hz, 1H), 8.22 (dd, J = 8.7, |
| | | | 1.6 Hz, 1H), 7.82 (dd, |
| | | | J = 8.6, 4.9 Hz, 1H), 7.74 |
| | | | (t, J = 2.1 Hz, 1H), 7.56 |
| | | | (dt, J = 8.4, 1.2 Hz, 1H), |
| | | | 7.32 (t, J = 8.1 Hz, 1H), |
| | | | 7.10 − 6.87 (m, 1H), |
| | | | 3.03 (s, 3H) |
| 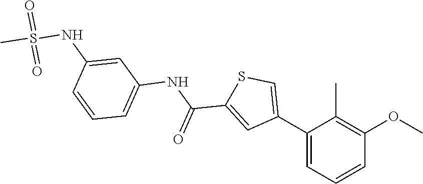 | 67 | 417 | ¹H NMR (400 MHz, DMSO-d6) δ 10.27 (s, 1H), 9.80 (s, 1H), 8.12 (d, J = 1.4 Hz, 1H), 7.80 (d, J = 1.4 Hz, 1H), 7.66 (t, J = 2.1 Hz, 1H), 7.51 (ddd, J = 8.3, 2.1, 1.0 Hz, 1H), 7.28 (dt, J = 21.5, 8.0 Hz, 2H), 7.00 (d, J = 8.2 Hz, 1H), 6.95 (ddd, J = 7.1, 2.9, 1.2 Hz, 2H), 3.84 (s, 3H), |
| | | | 3.01 (s, 3H), 2.18 (s, |
| | | | 3H). |
| 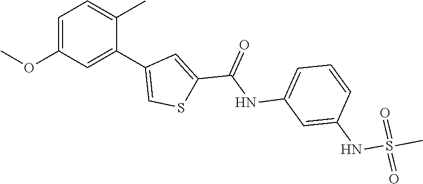 | 68 | 417 | ¹H NMR (400 MHz, DMSO-d6) δ 10.26 (s, 1H), 9.81 (s, 1H), 8.18 (d, J = 1.4 Hz, 1H), 7.86 (d, J = 1.4 Hz, 1H), 7.66 (t, J = 2.1 Hz, 1H), 7.50 (dt, J = 8.1, 1.3 Hz, 1H), 7.30 (t, J = 8.1 Hz, 1H), 7.23 (d, J = 8.4 Hz, 1H), 6.98 − 6.91 (m, 2H), 6.88 (dd, J = 8.3, 2.8 Hz, 1H), 3.77 (s, 3H), |
| | | | 3.01 (s, 3H), 2.29 (s, 3H). |
| 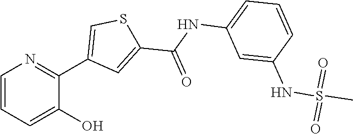 [SMILES: CS(=O)(=O)Nc1cccc(NC(=O)c2cc(-c3ncccc3O)cs2)c1] | 69 | 390 | ¹H NMR (300 MHz, DMSO-d6) δ 10.52 (s, 1H), 8.82 (d, J = 1.4 Hz, 1H), 8.52 (d, J = 1.3 Hz, 1H), 8.15 (dd, J = 4.5, 1.4 Hz, 1H), 7.74 (t, J = 2.2 Hz, 1H), 7.60 − 7.51 (m, 1H), 7.37 (dd, J = 8.2, 1.5 Hz, 1H), 7.30 (t, |
| | | | J = 8.1 Hz, 1H), 7.22 |
| | | | (dd, J = 8.2, 4.5 Hz, |
| | | | 1H), 6.94 (dd, J = 8.0, |
| | | | 2.2 Hz, 1H), 3.02 (s, |
| | | | 3H). |
| 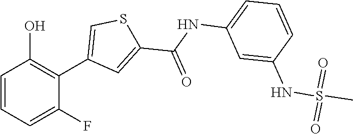 [SMILES: CS(=O)(=O)Nc1cccc(NC(=O)c2cc(-c3c(O)cccc3F)cs2)c1] | 70 | 407 | ¹H NMR (400 MHz, DMSO-d6) δ 10.31 (s, 1H), 10.20 (d, J = 1.6 Hz, 1H), 9.79 (s, 1H), 8.27 (t, J = 1.5 Hz, 1H), 7.93 (t, J = 1.3 Hz, 1H), 7.68 (t, J = 2.1 Hz, 1H), 7.55 − 7.48 (m, 1H), 7.30 (t, J = 8.1 Hz, 1H), |
| | | | 7.20 (td, J = 8.3, 6.7 Hz, |
| | | | 1H), 6.94 (ddd, J = 8.1, |
| | | | 2.2, 1.0 Hz, 1H), 6.86 − |
| | | | 6.80 (m, 1H), 6.76 (ddd, |
| | | | J = 9.6, 8.3, 1.1 Hz, |
| | | | 1H), 3.01 (s, 3H). |
| 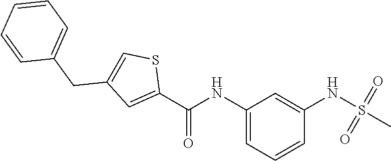 [SMILES: CS(=O)(=O)Nc1cccc(NC(=O)c2cc(Cc3ccccc3)cs2)c1] | 71 | 387 | ¹H NMR (400 MHz, DMSO-d6) δ 10.23 (s, 1H), 9.77 (s, 1H), 7.87 (d, J = 1.4 Hz, 1H), 7.63 (t, J = 2.1 Hz, 1H), 7.54 (s, 1H), 7.47 − 7.43 (m, 1H), 7.36 − 7.19 (m, 6H), 6.91 (dd, J = 7.8, 1.6 Hz, 1H), 3.97 (s, 2H), 2.99 (s, 3H). |

### Example 72: tert-butyl (3-(5-((5-(methylsulfonamido)cyclohex-2-en-1-yl)carbamoyl)thiophen-3-yl)phenyl)carbamate

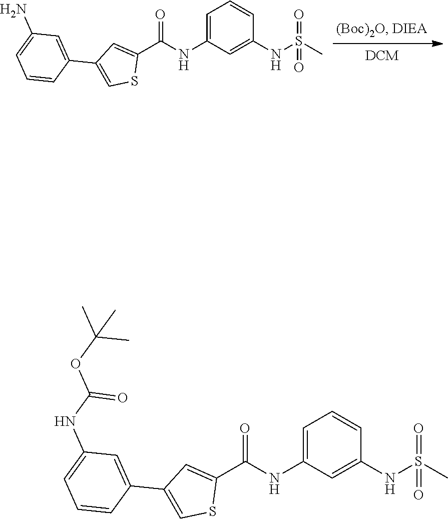 [SMILES: CS(=O)(=O)Nc1cccc(NC(=O)c2cc(-c3cccc(N)c3)cs2)c1 ; CC(C)(C)OC(=O)Nc1cccc(-c2csc(C(=O)Nc3cccc(NS(C)(=O)=O)c3)c2)c1]

A mixture of 4-(3-aminophenyl)-N-(3-methanesulfonamidophenyl) thiophene-2-carboxamide (30 mg, 0.077 mmol, 1 equiv), (BOC)₂O (20.28 mg, 0.092 mmol, 1.2 equiv) and DIEA (30.02 mg, 0.231 mmol, 3 equiv) in DCM (3 mL) was stirred for 1 h at room temperature. The resulting mixture was concentrated under reduced pressure. The residue product was purified by reverse phase flash with the following conditions (Water: ACN=2:3) to afford tert-butyl N-(3-15-[(3-methanesulfonamidophenyl) carbamoyl]thiophen-3-yl}phenyl) carbamate (25.2 mg, 66.75%) as a white solid. LCMS (ESI) [M+H]⁺: 488. ¹H NMR (300 MHz, DMSO-d6) δ 10.42 (s, 1H), 9.81 (s, 1H), 9.46 (s, 1H), 8.39 (d, J=1.4 Hz, 1H), 8.05 (d, J=1.3 Hz, 1H), 7.92 (s, 1H), 7.69 (d, J=2.3 Hz, 1H), 7.53 (d, J=8.0 Hz, 1H), 7.33 (d, J=6.5 Hz, 4H), 6.96 (d, J=7.9 Hz, 1H), 3.02 (s, 3H), 1.50 (s, 9H).

### Example 73: 4-(5-hydroxycyclohexa-2,4-dien-1-yl)-N-(3-(methylsulfonamido)phenyl)thiophene-2-carboxamide

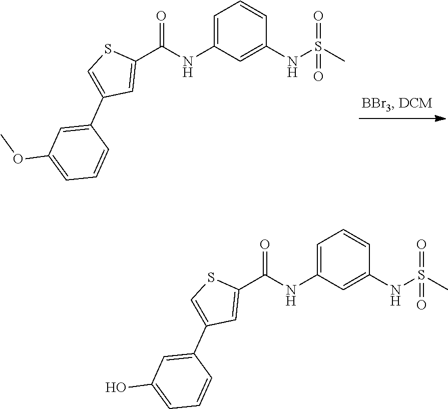

To a stirred solution of N-(3-methanesulfonamidophenyl)-4-(3-methoxyphenyl) thiophene-2-carboxamide (125 mg, 0.310 mmol) in DCM was added BBr₃ (388.53 mg, 1.55 mmol, 5 equiv) dropwise. The reaction was stirred for 3 h at room temperature under nitrogen atmosphere. The mixture was concentrated and purified by reverse phase flash chromatography eluting with ACN/H₂O (5-95%, acidic system) to afford 4-(3-hydroxyphenyl)-N-(3-methanesulfonamidophenyl)thiophene-2-carboxamide (35.5 mg, 99.876%) as a white solid. LCMS (ESI) [M+H]⁺: 389. ¹H NMR (300 MHz, DMSO-d6) δ 10.33 (s, 1H), 9.82 (s, 1H), 9.58 (s, 1H), 8.44 (d, J=1.5 Hz, 1H), 8.11 (d, J=1.3 Hz, 1H), 7.70 (t, J=2.1 Hz, 1H), 7.57-7.48 (m, 1H), 7.30 (dt, J=15.8, 8.0 Hz, 2H), 7.21-7.08 (m, 2H), 7.01-6.91 (m, 1H), 6.77 (ddd, J=7.9, 2.5, 1.0 Hz, 1H), 3.02 (s, 3H).

### Example 74: 4-(3-hydroxy-2-methylphenyl)-N-(3-(methylsulfonamido)phenyl)thiophene-2-carboxamide

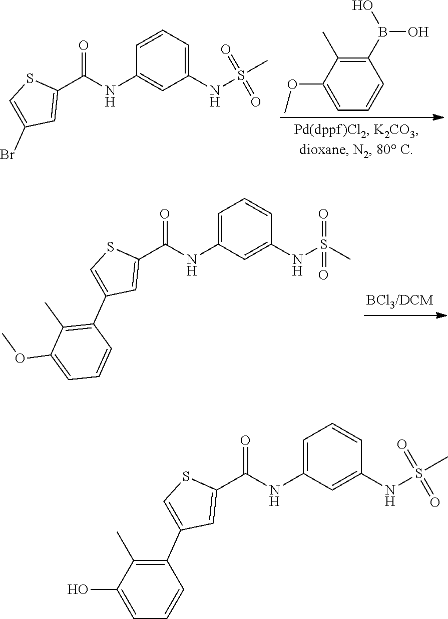

Step 1: To a stirred solution of 4-bromo-N-(3-methanesulfonamidophenyl)thiophene-2-carboxamide (300 mg, 0.799 mmol), (3-methoxy-2-methylphenyl)boronic acid (197 mg, 1.19 mmol) and Pd(dppf)Cl₂ (58.4 m g, 0.07 99 mmol), K₂CO₃ (332 mg, 2.39 mmol) was added dioxane (10 mL). Then the mixture was stirred for 2 h at 80° C. The mixture was concentrated and purified by reverse phase flash chromatography eluting with ACN/H₂O (0-100%, acidic system) to afford N-(3-methan esulfonamidophenyl)-4-(3-methoxy-2-methylphenyl)thiophene-2-carboxamide (87.0 mg, 0.208 mmol) as an off-white solid. LCMS (ESI) [M+H]⁺: 417.09 Step 2: To a stirred solution of N-(3-methanesulfonamidophenyl)-4-(3-methoxy-2-methylphenyl)thiophene-2-carboxamide (87 mg, 0.208 mmol), BCl₃ (72.3 mg, 0.624 mmol) was added DCM (5 mL) dropwise. Then the mixture was stirred for 2 h at room temperature.

The mixture was concentrated and purified by reverse phase flash chromatography eluting with ACN/H₂O (0-100%, acidic system) to afford 4-(3-hydroxy-2-methylphenyl)-N-(3-methanesulfonamidophenyl)thiophene-2-carboxamide (25.7 mg, 0.0629 mmol) as a white solid. LCMS (ESI) [M+H]⁺: 403.07. ¹H NMR (300 MHz, DMSO-d6) δ 10.26 (s, 1H), 9.81 (s, 1H), 9.48 (s, 1H), 8.12 (d, J=1.4 Hz, 1H), 7.77 (d, J=1.3 Hz, 1H), 7.67 (t, J=2.1 Hz, 1H), 7.56-7.46 (m, 1H), 7.31 (t, J=8.1 Hz, 1H), 7.07 (t, J=7.8 Hz, 1H), 6.95 (ddd, J=8.1, 2.2, 1.0 Hz, 1H), 6.82 (ddd, J=11.6, 7.8, 1.3 Hz, 2H), 3.01 (s, 3H), 2.15 (s, 3H).

The compounds listed in the following table were prepared using a procedure similar to that described for Example 74:

| | Example | MS (ESI) | |
|---|---|---|---|
| Structure | No. | [M + H]⁺ | ¹H NMR |
| 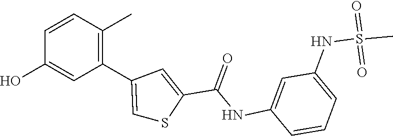 [SMILES: Cc1ccc(O)cc1-c1csc(C(=O)Nc2cccc(NS(C)(=O)=O)c2)c1] | 75 | 403 | ¹H NMR (300 MHz, DMSO-d6) δ 10.26 (s, 1H), 9.79 (s, 1H), 9.32 (s, 1H), 8.16 (d, J = 1.4 Hz, 1H), 7.81 (d, J = 1.3 Hz, 1H), 7.67 (t, J = 2.1 Hz, 1H), 7.54-7.45 (m, 1H), 7.30 (d, J = 8.1 Hz, 1H), 7.10 (d, J = 8.2 Hz, 1H), 7.01- 6.85 (m, 1H), 6.77 (d, J = 2.6 Hz, 1H), 6.70 (dd, J = 8.2, 2.6 Hz, |
| | | | 1H), 3.01 (s, 3H), 2.24 (s, 3H) |

### Example 76: 4-(2,6-difluorophenyl)-N-(3-(methylsulfonamido)phenyl)thiophene-2-carboxamide

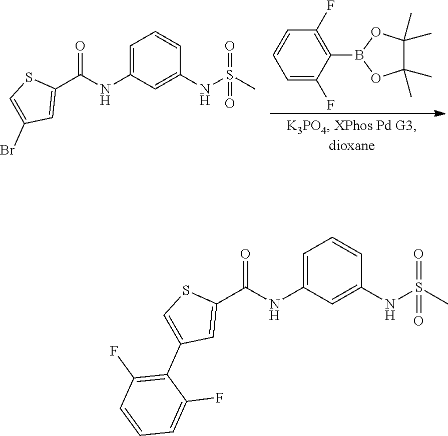

A solution of 4-bromo-N-(3-methanesulfonamidophenyl)thiophene-2-carboxamide (100 mg, 0.266 mmol), XPhos Pd G3 (22.5 mg, 0.0266 mmol), K₃PO₄ (169 mg, 0.798 mmol) and 2-(2,6-difluorophenyl)-4,4,5,5-tetramethyl-1,3,2-dioxaborolane (191 mg, 0.798 mmol) in dioxane (5 mL) was stirred for 2 h at 80° C. under nitrogen atmosphere. The resulting mixture was concentrated under reduced pressure. The residue was purified by silica gel column chromatography, eluted with Petroleum ether/EtOAc (0-100%) to afford 4-(2,6-difluorophenyl)-N-(3-methanesulfonamidophenyl)thiophene-2-carboxamide (69.5 mg, 0.169 mmol) as a white solid. LCMS (ESI) [M+H]⁺: 409. ¹H NMR (300 MHz, DMSO-d6) δ 10.39 (s, 1H), 9.81 (s, 1H), 8.31 (q, J=1.5 Hz, 1H), 8.12 (q, J=1.3 Hz, 1H), 7.68 (t, J=2.0 Hz, 1H), 7.58-7.42 (m, 2H), 7.30 (dt, J=10.7, 8.2 Hz, 3H), 6.96 (ddd, J=8.1, 2.2, 1.0 Hz, 1H), 3.02 (s, 3H).

The compounds listed in the following table were prepared using a procedure similar to that described for Example 76:

| | Example | MS (ESI) | |
|---|---|---|---|
| Structure | No. | [M + H]⁺ | ¹H NMR |
| 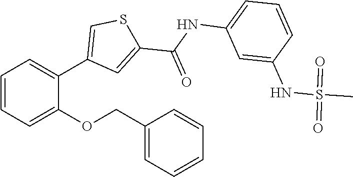 [SMILES: CS(=O)(=O)Nc1cccc(NC(=O)c2cc(-c3ccccc3OCc3ccccc3)cs2)c1] | 77 | 479 | ¹H NMR (300 MHz, DMSO- d6) δ 10.30 (s, 1H), 9.80 (s, 1H), 8.45 (d, J = 1.4 Hz, 1H), 8.07 (d, J = 1.3 Hz, 1H), 7.69 (t, J = 2.1 Hz, 1H), 7.60 (dd, J = 7.6, 1.7 Hz, 1H), 7.50 (t, J = 7.7 Hz, 3H), 7.41-7.27 (m, 5H), 7.23 (d, J = 8.2 Hz, 1H), 7.08 (t, J = 7.6 Hz, 1H), 6.96 (d, J = 8.4 Hz, 1H), 5.24 (s, 2H), 3.02 (s, 3H). |
| 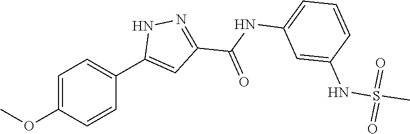 | 78 | 387 | ¹H NMR (300 MHz, DMSO- d6) δ 13.67 (s, 1H), 10.14 (s, 1H), 9.78 (s, 1H), 7.76 (s, 3H), 7.55 (d, J = 8.4 Hz, 1H), 7.35 (s, 1H) 7.28 (t, J = 8.2 Hz, 1H), 7.05 (d, J = 8.2 Hz, 2H), 6.92 (d, J = 8.1 Hz, 1H), 3.81 (s, 3H), 3.03 (s, 3H) |
| 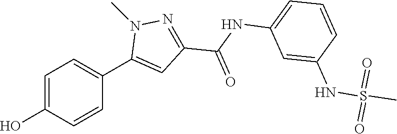 [SMILES: Cn1nc(C(=O)Nc2cccc(NS(C)(=O)=O)c2)cc1-c1ccc(O)cc1] | 79 | 387 | ¹H NMR (400 MHz, DMSO- d6) δ 10.08 (s, 1H), 9.80 (d, J = 32.6 Hz, 2H), 7.78 (t, J = 2.1 Hz, 1H), 7.55 (dt, J = 8.5, 1.2 Hz, 1H), 7.47-7.35 (m, 2H), 7.26 (t, J = 8.1 Hz, 1H), 7.02- 6.84 (m, 3H), 6.80 (s, 1H), 3.93 (s, 3H), 3.03 (s, 3H) |

### Example 80: 5-methyl-N-(3-(methylsulfonamido)phenyl)-4-(pyridin-2-yl)thiophene-2-carboxamide

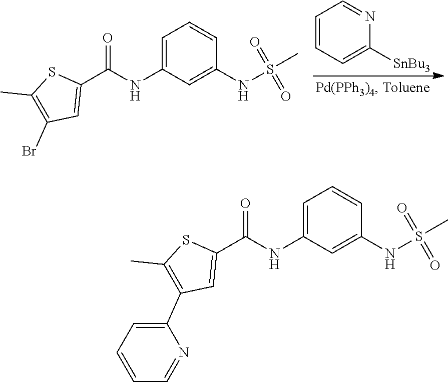

A solution of 4-bromo-N-(3-methanesulfonamidophenyl)-5-methylthiophene-2-carboxamide (300 mg, 0.770 mmol), 2-(tributylstannyl)pyridine (563 mg, 1.53 mmol) and Pd(PPh₃)₄ (177 mg, 0.154 mmol) in Toluene (5 mL) was stirred for 2 h at 120° C. under nitrogen atmosphere. The resulting mixture was concentrated under reduced pressure. The residue was purified by silica gel column chromatography, eluted with Petroleum ether/EtOAc (0-100%) to afford N-(3-methanesulfonamidophenyl)-5-methyl-4-(pyridin-2-yl)thiophene-2-carboxamide (28.8 mg, 0.0740 mmol) as a yellow solid. LCMS (ESI) [M+H]⁺: 388 ¹H NMR (300 MHz, DMSO-d6) δ 10.31 (s, 1H), 9.79 (s, 1H), 8.69 (ddd, J=4.8, 1.9, 0.9 Hz, 1H), 8.40 (s, 1H), 7.93 (td, J=7.7, 1.9 Hz, 1H), 7.75-7.66 (m, 2H), 7.57-7.47 (m, 1H), 7.37 (ddd, J=7.5, 4.8, 1.1 Hz, 1H), 7.30 (t, J=8.1 Hz, 1H), 6.93 (ddd, J=8.0, 2.2, 1.0 Hz, 1H), 3.01 (s, 3H), 2.71 (s, 3H). The compounds listed in the following table were prepared using a procedure similar to that described for Example 80:

| | Example | MS (ESI) | |
|---|---|---|---|
| Structure | No. | [M + H]⁺ | ¹H NMR |
| 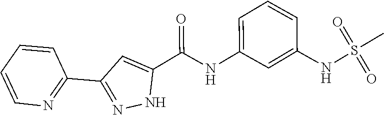 [SMILES: CS(=O)(=O)Nc1cccc(NC(=O)c2cc(-c3ccccn3)n[nH]2)c1] | 81 | 358 | ¹H NMR (400 MHz, DMSO-d6) δ 14.06 (d, J = 47.7 Hz, 1H), 10.26 (d, J = 62.3 Hz, 1H), 9.79 (s, 1H), 8.65 (s, 1H), 8.04-7.81 (m, 2H), 7.79 (s, 1H), 7.56 (d, J = 8.2 Hz, 1H), 7.34 (d, J = 46.0 Hz, 2H), 6.93 (d, J = 8.1 Hz, 1H), |
| | | | 3.03 (s, 3H) |

### Example 82: N-(3-(methylsulfonamido)phenyl)-4-(pyridin-2-yl)thiophene-2-carboxamide

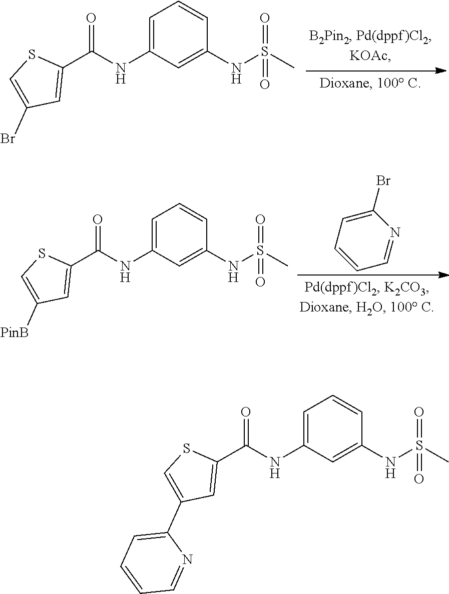

Step 1: A solution of 4-bromo-N-(3-methanesulfonamidophenyl)thiophene-2-carboxamide (400 mg, 1.066 mmol, 1 equiv) and bis(pinacolato)diboron (2706.82 mg, 10.660 mmol, 10 equiv) and Pd(dppf)Cl₂ (77.99 mg, 0.107 mmol, 0.1 equiv) and KOAc (313.84 mg, 3.198 mmol, 3 equiv) in Dioxane (5 mL) was stirred for 2 h at 80° C. under nitrogen atmosphere. The residue was purified by silica gel column chromatography, eluted with Petroleum ether/EtOAc (1:1) to afford N-(3-methanesulfonamidophenyl)-4-(4,4,5,5-tetramethyl-1,3,2-dioxaborolan-2-yl)thiophene-2-carboxamide (352 mg, 78.19%) as a yellow oil. LCMS (ES, m/z): LCMS (ESI) [M+H]⁺: 423.

Step 2: A solution of N-(3-methanesulfonamidophenyl)-4-(4,4,5,5-tetramethyl-1,3,2-dioxaborolan-2-yl)thio phene-2-carboxamide (352 mg, 0.833 mmol, 1.5 equiv) and 2-bromopyridine (87.79 mg, 0.555 mmol, 1 equiv) and Pd(dppf)Cl₂ (40.66 mg, 0.056 mmol, 0.1 equiv) and K₂CO₃ (230.39 mg, 1.666 mmol, 3 equiv) in Dioxane (3 mL) and H₂O (1 mL) was stirred for 2 h at 80° C. under nitrogen atmosphere. The residue was purified by reverse phase flash chromatography with the following conditions: column, silica gel; mobile phase, MeCN in water, 0% to 100% gradient in 20 min; detector, UV 254 nm. This resulted in N-(3-methanesulfonamidophenyl)-4-(pyridin-2-yl)thiophene-2-carboxamide (68.8 mg, 33.15%) as a white solid. LCMS (ES, m/z): LCMS (ESI) [M+H]⁺: 374. ¹H NMR (300 MHz, DMSO-d6) δ 10.48 (s, 1H), 9.81 (s, 1H), 8.75 (d, J=1.4 Hz, 1H), 8.65 (m, J=4.8, 1.4 Hz, 1H), 8.49 (d, J=1.3 Hz, 1H), 7.96-7.88 (m, 2H), 7.73 (t, J=2.0 Hz, 1H), 7.58-7.51 (m, 1H), 7.42-7.28 (m, 2H), 6.95 (m, J=8.1, 2.1, 1.0 Hz, 1H), 3.02 (s, 3H).

The compounds listed in the following table were prepared using a procedure similar to that described for Example 82:

| | Example | MS (ESI) | |
|---|---|---|---|
| Structure | No. | [M + H]⁺ | ¹H NMR |
| 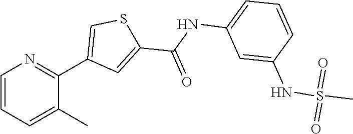 [SMILES: Cc1cccnc1-c1csc(C(=O)Nc2cccc(NS(C)(=O)=O)c2)c1] | 83 | 388 | ¹H NMR (400 MHz, DMSO-d6) δ 10.43 (s, 1H), 9.80 (s, 1H), 8.51 (q, J = 2.3 Hz, 2H), 8.16 (d, J = 1.4 Hz, 1H), 7.80- 7.60 (m, 2H), 7.57- 7.47 (m, 1H), 7.40- 7.22 (m, 2H), 6.94 (ddd, J = 8.0, 2.2, 0.9 Hz, 1H), |
| | | | 3.01 (s, 2H), 2.49 (s, 3H) |
| 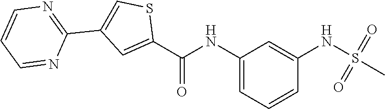 [SMILES: CS(=O)(=O)Nc1cccc(NC(=O)c2cc(-c3ncccn3)cs2)c1] | 84 | 375 | ¹H NMR (300 MHz, DMSO-d6) δ 10.55 (s, 1H), 9.80 (s, 1H), 8.90 (d, J = 4.9 Hz, 2H), 8.84 (d, J = 1.3 Hz, 1H), 8.65 (d, J = 1.3 Hz, 1H), 7.75 (t, J = 2.1 Hz, 1H), 7.56 |
| | | | (ddd, J = 8.3, 2.1, 1.0 |
| | | | Hz, 1H), 7.45 (t, J = 4.9 |
| | | | Hz, 1H), 7.31 (t, J = 8.1 |
| | | | Hz, 1H), 6.95 (ddd, J = |
| | | | 8.1, 2.2, 1.0 Hz, 1H), |
| | | | 3.03 (s, 3H) |
| 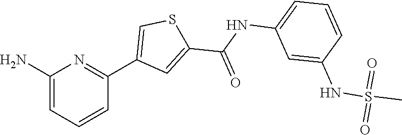 [SMILES: CS(=O)(=O)Nc1cccc(NC(=O)c2cc(-c3cccc(N)n3)cs2)c1] | 85 | 389 | ¹H NMR (300 MHz, Methanol-d4) δ 8.41 (d, J = 1.4 Hz, 1H), 8.15 (d, J = 1.4 Hz, 1H), 7.77 (t, J = 2.0 Hz, 1H), 7.60- 7.43 (m, 2H), 7.35 (t, J = 8.1 Hz, 1H), 7.14-6.85 (m, 2H), 6.54 (dd, J = 8.3, 0.8 Hz, 1H), 3.03 (s, |
| | | | 3H) |
| 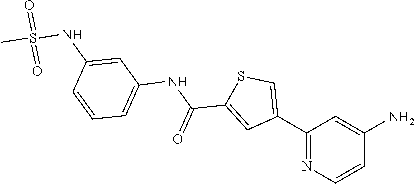 [SMILES: CS(=O)(=O)Nc1cccc(NC(=O)c2cc(-c3cc(N)ccn3)cs2)c1] | 86 | 389 | ¹H NMR (400 MHz, DMSO-d6) δ 10.46 (s, 1H), 9.80 (s, 1H), 8.59 (d, J = 1.4 Hz, 1H), 8.19 (d, J = 1.3 Hz, 1H), 8.05 (d, J = 5.6 Hz, 1H), 7.72 (t, J = 2.1 Hz, 1H), 7.60- 7.47 (m, 1H), 7.30 (t, J = 8.1 Hz, 1H), 6.95- 6.86 (m, 2H), 6.45 (dd, J = 5.6, 2.1 Hz, 1H), 6.12 (s, 2H), 3.02 (s, 3H) |
| 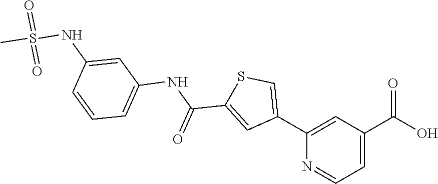 [SMILES: CS(=O)(=O)Nc1cccc(NC(=O)c2cc(-c3cc(C(=O)O)ccn3)cs2)c1] | 87 | 418 | ¹H NMR (300 MHz, DMSO-d6) δ 13.83 (s, 1H), 10.49 (s, 1H), 9.82 (s, 1H), 8.87-8.78 (m, 2H), 8.63 (d, J = 1.3 Hz, 1H), 8.30 (t, J = 1.2 Hz, 1H), 7.82-7.70 (m, 2H), 7.60-7.50 (m, 1H), 7.32 (t, J = 8.1 Hz, 1H), 6.96 (ddd, J = 8.1, 2.2, 1.0 Hz, 1H), 3.03 (s, 3H). |
| 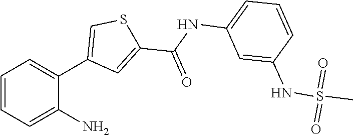 [SMILES: CS(=O)(=O)Nc1cccc(NC(=O)c2cc(-c3ccccc3N)cs2)c1] | 88 | 388 | ¹H NMR (300 MHz, DMSO-d6) δ 10.30 (s, 1H), 9.81 (s, 1H), 8.24 (d, J = 1.4 Hz, 1H), 7.85 (d, J = 1.3 Hz, 1H), 7.68 (t, J = 2.0 Hz, 1H), 7.52 (ddd, J = 8.2, 2.0, 1.0 Hz, 1H), 7.31 (t, J = 8.1 Hz, 1H), 7.16 (dd, J = |
| | | | 7.6, 1.6 Hz, 1H), 7.07 |
| | | | (ddd, J = 8.6, 7.3, 1.6 |
| | | | Hz, 1H), 6.94 (ddd, J = |
| | | | 8.0, 2.2, 1.0 Hz, 1H), |
| | | | 6.79 (dd, J = 8.1, 1.2 Hz, |
| | | | 1H), 6.64 (td, J = 7.4, 1.2 |
| | | | Hz, 1H), 5.03 (s, 2H), |
| | | | 3.01 (s, 3H). |
| 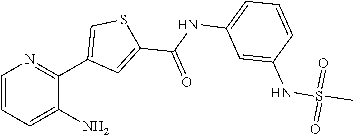 [SMILES: CS(=O)(=O)Nc1cccc(NC(=O)c2cc(-c3ncccc3N)cs2)c1] | 89 | 389 | ¹H NMR (300 MHz, DMSO-d6) δ 10.44 (s, 1H), 9.80 (s, 1H), 8.54 (d, J = 1.4 Hz, 1H), 8.15 (d, J = 1.3 Hz, 1H), 7.93 (dd, J = 4.4, 1.5 Hz, 1H), 7.72 (t, J = 2.0 Hz, 1H), 7.59-7.50 (m, 1H), 7.30 (t, J = 8.1 Hz, 1H), |
| | | | 7.20 (dd, J = 8.1, 1.6 Hz, |
| | | | 1H), 7.09 (dd, J = 8.1, |
| | | | 4.4 Hz, 1H), 6.94 (ddd, |
| | | | J = 8.1, 2.2, 1.0 Hz, 1H), |
| | | | 5.33 (s, 2H), 3.02 (s, |
| | | | 3H). |
| 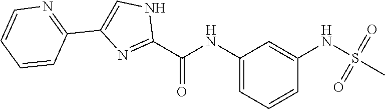 [SMILES: CS(=O)(=O)Nc1cccc(NC(=O)c2nc(-c3ccccn3)c[nH]2)c1] | 90 | 358 | ¹H NMR (300 MHz, DMSO-d6) δ 13.57 (s, 1H), 10.38 (s, 1H), 9.77 (s, 1H),8.57 (d, J = 4.8 Hz, 1H), 8.09 (d, J = 7.9 Hz, 1H), 7.87 (ddd, J = 7.4, 6.2, 2.0 Hz, 3H), |
| | | | 7.67-7.54 (m, 1H), |
| | | | 7.36-7.18 (m, 2H), |
| | | | 7.09-6.82 (m, 1H), |
| | | | 3.06 (s, 3H). |

### Example 91: N-(3-(methylsulfonamido)phenyl)-4-(5-(piperazin-1-yl)pyridin-2-yl)thiophene-2-carboxamide

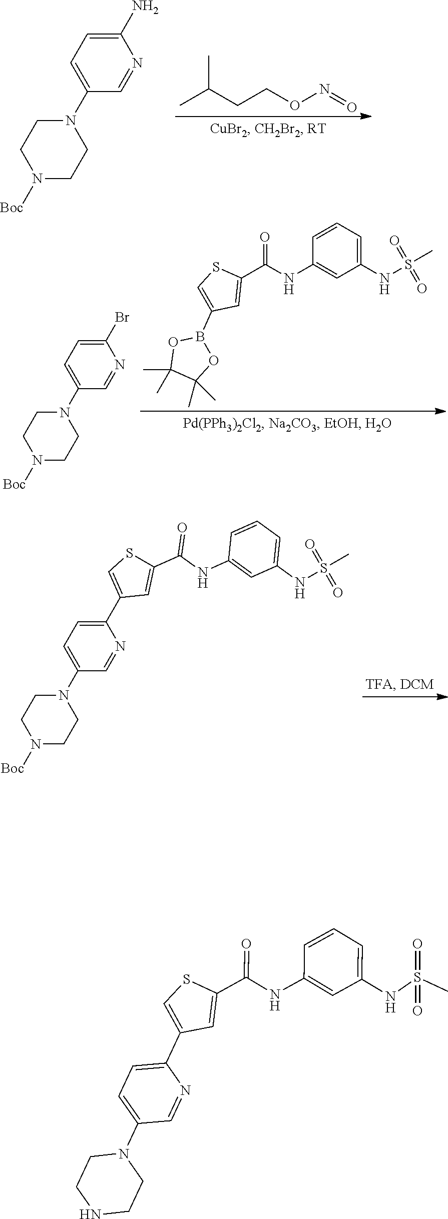

Step 1: To a stirred solution of tert-butyl 4-(6-aminopyridin-3-yl)piperazine-1-carboxylate (300 mg, 1.07 mmol) and CuBr₂ (119 mg, 0.535 mmol) in CH₂Br₂ (5 mL) was added 3-methylbutyl nitrite (137 mg, 1.17 mmol) dropwise at 0° C. Then the mixture was stirred for 5 h at room temperature. The mixture was concentrated and purified by flash chromatography on silica gel eluting with EtOAc/Petroleum ether (0-100%) to afford tert-butyl 4-(6-bromopyridin-3-yl)piperazine-1-carboxylate (188 mg, 0.549 mmol) as a yellow solid. LCMS (ESI) [M+H]⁺: 342.

Step 2: A solution of tert-butyl 4-(6-bromopyridin-3-yl)piperazine-1-carboxylate (190 mg, 0.555 mmol), Pd(PPh₃)₂Cl₂ (77.2 mg, 0.110 mmol), Na₂CO₃ (175 mg, 1.66 mmol) and N-(3-methanesulfonamidophenyl)-4-(4,4,5,5-tetramethyl-1,3,2-dioxaborolan-2-yl)thiophene-2-carboxamide (257 mg, 0.610 mmol) in EtOH (4 mL) and H₂O (1 mL) was stirred for 2 h at 80° C. under nitrogen atmosphere. The resulting mixture was concentrated under reduced pressure. The residue was purified by silica gel column chromatography, eluted with Petroleum ether/EtOAc (0-100%) to afford tert-butyl 4-(6-{5-[(3-methanesulfonamido phenyl)carbamoyl]thiophen-3-yl}pyridin-3-yl)piperazine-1-carboxylate (178 mg, 0.319 mmol) as a yellow solid. LCMS (ESI) [M+H]⁺: 558 Step 3: To a stirred solution of tert-butyl 4-(6-{5-[(3-methanesulfonamidophenyl) carbamoyl]thiophen-3-yl}pyridin-3-yl)piperazine-1-carboxylate (180 mg, 0.322 mmol) in DCM (10 mL) was added TFA (365 mg, 3.21 mmol) dropwise at 0° C. for 1 h. The resulting mixture was concentrated under vacuum. The mixture was basified to pH 9 with NaOH. The mixture was concentrated and purified by flash chromatography on silica gel eluting with EtOAc/Petroleum ether (0-100%) to afford N-(3-methanesulfonamidophenyl)-4-[5-(piperazin-1-yl) pyridin-2-yl]thiophene-2-carboxamide (19.9 mg, 0.0419 mmol) as a yellow solid. LCMS (ESI) [M+H]⁺: 458. ¹H NMR (300 MHz, DMSO-d6) δ 8.61 (d, J=1.4 Hz, 1H), 8.37-8.28 (m, 2H), 8.23 (d, J=1.4 Hz, 1H), 7.80-7.69 (m, 2H), 7.57-7.40 (m, 2H), 7.31 (t, J=8.1 Hz, 1H), 6.94 (dd, J=8.2, 2.3 Hz, 1H), 3.34 (t, J=5.1 Hz, 4H), 3.07 (dd, J=6.3, 3.7 Hz, 4H), 3.01 (s, 3H).

### Example 92: 2-(5-((3-(methylsulfonamido)phenyl)carbamoyl)thiophen-3-yl)-1,4-dihydropyridine-4-carboxamide

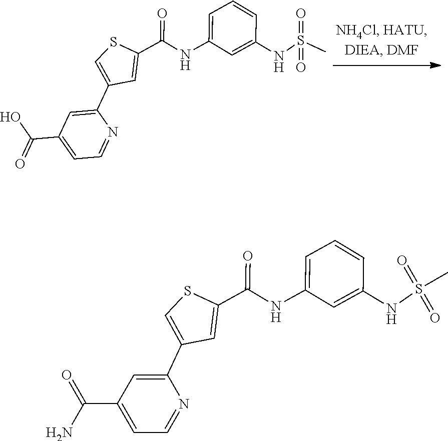 [SMILES: CS(=O)(=O)Nc1cccc(NC(=O)c2cc(-c3cc(C(=O)O)ccn3)cs2)c1 ; CS(=O)(=O)Nc1cccc(NC(=O)c2cc(-c3cc(C(N)=O)ccn3)cs2)c1]

To a stirred solution of 2-{5-[(3-methanesulfonamidophenyl)carbamoyl]thiophen-3-yl}pyridine-4-carboxylic acid (100 mg, 0.240 mmol, 1 equiv) and NH₄Cl (12.81 mg, 0.240 mmol, 1 equiv) in DMF (3 mL) were added DIEA (92.88 mg, 0.720 mmol, 3 equiv) and HATU (136.63 mg, 0.360 mmol, 1.5 equiv) in portions at room temperature. The residue was purified by reverse phase flash chromatography with the following conditions: column, silica gel; mobile phase, MeCN in water, 0% to 100% gradient in 20 min; detector, UV 254 nm. This resulted in 2-{5-[(3-methanesulfonamido phenyl)carbamoyl]thiophen-3-yl}pyridine-4-carboxamide (11.3 mg, 11.33%) as a white solid. LCMS (ES, m/z): LCMS (ESI) [M+H]⁺: 417. ¹H NMR (300 MHz, DMSO-d6) δ 10.51 (s, 1H), 9.76 (s, 1H), 8.84-8.73 (m, 2H), 8.53 (d, J=1.3 Hz, 1H), 8.39-8.26 (m, 2H), 7.83 (s, 1H), 7.73 (m, J=3.3, 1.4 Hz, 2H), 7.55 (m, J=8.4, 1.2 Hz, 1H), 7.31 (t, J=8.1 Hz, 1H), 6.95 (m, J=8.1, 2.2, 1.0 Hz, 1H), 3.02 (s, 3H).

### Example 93: 4-(2-acetamidophenyl)-N-(3-(methylsulfonamido)phenyl)thiophene-2-carboxamide

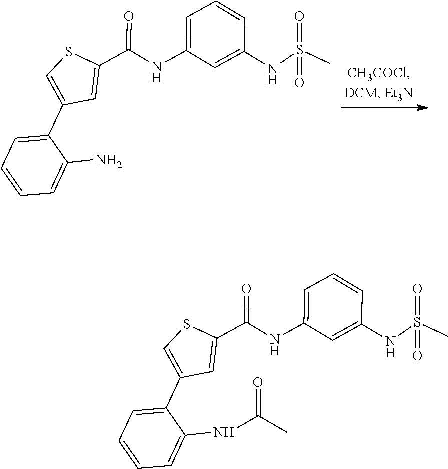 [SMILES: CS(=O)(=O)Nc1cccc(NC(=O)c2cc(-c3ccccc3N)cs2)c1 ; CC(=O)Nc1ccccc1-c1csc(C(=O)Nc2cccc(NS(C)(=O)=O)c2)c1]

To a mixture of 4-(2-aminophenyl)-N-(3-methanesulfonamidophenyl)thiophene-2-carboxamide (80 m g, 0.206 mmol) and Et₃N (62.4 mg, 0.618 mmol) in DCM (1 mL) was added CH₃COCl (19.3 mg, 0.247 mmol). The resulting mixture was stirred for two hours at room temperature. Then it was concentrated, The residue was purified by flash chromatography on silica gel eluting with 35% of ethyl acetate in petroleum ether to afford 4-(2-acetamidophenyl)-N-(3-methanesulfonamidophenyl)thiophene-2-carboxamide (39.5 mg, 44.4%) as a white solid. LCMS [M+H]⁺: 430. ¹H NMR (300 MHz, DMSO-d6) δ 10.34 (s, 1H), 9.82 (s, 1H), 9.39 (s, 1H), 8.27 (d, J=1.4 Hz, 1H), 7.91 (d, J=1.3 Hz, 1H), 7.68 (t, J=2.1 Hz, 1H), 7.61-7.43 (m, 3H), 7.41-7.22 (m, 3H), 6.95 (ddd, J=8.0, 2.2, 1.0 Hz, 1H), 3.02 (s, 3H), 2.00 (s, 3H).

The compounds listed in the following table were prepared using a procedure similar to that described for Example 93:

| | Example | MS (ESI) | |
|---|---|---|---|
| Structure | No. | [M + H]⁺ | ¹H NMR |
| 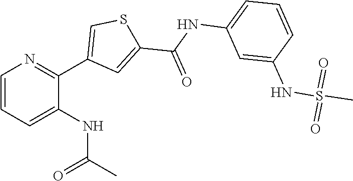 [SMILES: CC(=O)Nc1cccnc1-c1csc(C(=O)Nc2cccc(NS(C)(=O)=O)c2)c1] | 94 | 431 | ¹H NMR (300 MHz, DMSO-d6) δ 10.49 (s, 1H), 9.80 (d, J = 9.3 Hz, 2H), 8.60 (d, J = 1.4 Hz, 1H), 8.49 (dd, J = 4.6, 1.6 Hz, 1H), 8.23 (d, J = 1.3 Hz, 1H), 7.93 (dd, J = 8.2, 1.6 Hz, 1H), 7.72 (t, J = 2.0 Hz, 1H), 7.60- 7.50 (m, 1H), 7.39 (dd, J = 8.1, 4.6 Hz, |
| | | | 1H), 7.31 (t, J = 8.1 |
| | | | Hz, 1H), 6.95 (ddd, |
| | | | J = 8.0, 2.2, 1.0 Hz, |
| | | | 1H), 3.02 (s, 3H), 2.10 |
| | | | (s, 3H). |
| 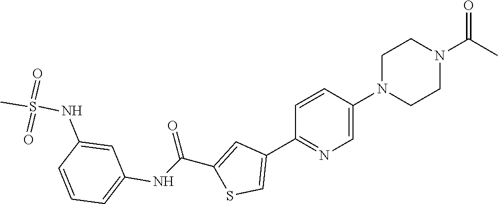 [SMILES: CC(=O)N1CCN(c2ccc(-c3csc(C(=O)Nc4cccc(NS(C)(=O)=O)c4)c3)nc2)CC1] | 95 | 500 | ¹H NMR (300 MHz, DMSO-d6) δ 10.45 (s, 1H), 9.82 (s, 1H), 8.65 (d, J = 1.4 Hz, 1H), 8.36 (d, J = 2.9 Hz, 1H), 8.26 (d, J = 1.3 Hz, 1H), 7.83-7.62 (m, 2H), 7.58-7.40 (m, 2H), 7.31 (t, J = 8.1 Hz, 1H), 7.01- 6.78 (m, 1H), 3.64- 3.49 (m, 4H), 3.30 (d, J = 7.1 Hz, 2H), 3.26- |
| | | | 3.13 (m, 2H), 3.02 (s, |
| | | | 3H), 2.06 (s, 3H) |

### Example 96: N-(3-(ethylsulfonamido)phenyl)-4-phenylthiophene-2-carboxamide

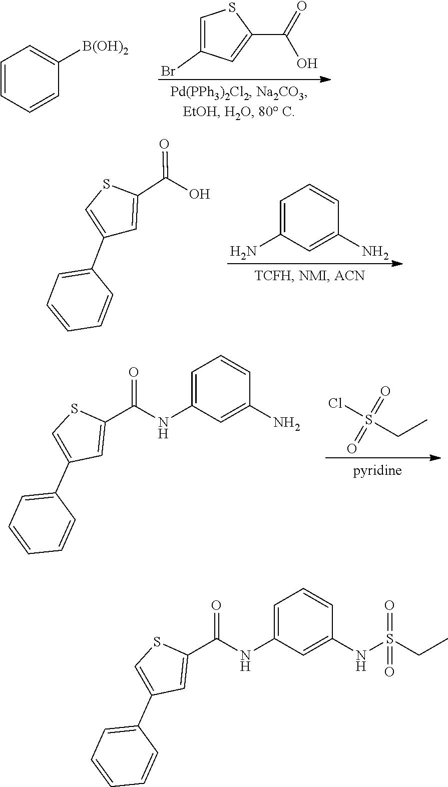

Step 1: A solution of phenyl boronic acid (5 g, 41.007 mmol, 1 equiv) and 4-bromothiophene-2-carboxylic acid (8.49 g, 41.007 mmol, 1 equiv) and Pd(PPh₃)₂Cl₂ (2.88 g, 4.101 mmol, 0.1 equiv) and Na₂CO₃ (13.04 g, 123.021 mmol, 3 equiv) in EtOH (20 mL) and H₂O (5 mL) was stirred for 2 h at 80° C. under nitrogen atmosphere. The mixture was acidified to pH 6 with HCl (aq.). The resulting mixture was concentrated under reduced pressure. The residue was purified by silica gel column chromatography, eluted with Petroleum ether/EtOAc (1:1) to afford 4-phenylthiophene-2-carboxylic acid (4.5 g, 53.73%) as a white solid. LCMS (ES, m/z): LCMS (ESI) [M+H]⁺: 205 Step 2: To a stirred solution of 4-phenylthiophene-2-carboxylic acid (100 mg, 0.490 mmol, 1.0 equiv) and benzene-1,3-diamine (52.95 mg, 0.490 mmol, 1 equiv) in ACN (3 mL) were added TCFH (206.07 mg, 0.735 mmol, 1.5 equiv) and NMI (120.60 mg, 1.470 mmol, 3 equiv) in portions at room temperature. The residue was purified by reverse phase flash chromatography with the following conditions: column, silica gel; mobile phase, MeCN in water, 0% to 100% gradient in 20 min; detector, UV 254 nm. This resulted in N-(3-aminophenyl)-4-phenylthiophene-2-carboxamide (130 mg, 90.20%) as a white solid. LCMS (ES, m/z): LCMS (ESI) [M+H]⁺: 295.

Step 3: A solution of N-(3-aminophenyl)-4-phenylthiophene-2-carboxamide (70 mg, 0.238 mmol, 1.0 equiv) and ethanesulfonyl chloride (36.69 mg, 0.286 mmol, 1.2 equiv) in pyridine (3 mL) was stirred for 1 h at room temperature. The residue was purified by reverse phase flash chromatography with the following conditions: column, silica gel; mobile phase, MeCN in water, 0% to 100% gradient in 20 min; detector, UV 254 nm. This resulted in N-(3-ethanesulfonamidophenyl)-4-phenyl thiophene-2-carboxamide (15.0 mg, 16.32%) as a white solid. LCMS (ES, m/z): LCMS (ESI) [M+H]⁺: 387. ¹H NMR (300 MHz, DMSO-d6) δ 10.33 (s, 1H), 9.86 (s, 1H), 8.50 (d, J=1.5 Hz, 1H), 8.20 (d, J=1.4 Hz, 1H), 7.78-7.73 (m, 2H), 7.70 (t, J=2.1 Hz, 1H), 7.56-7.43 (m, 3H), 7.41-7.21 (m, 2H), 7.01-6.92 (m, 1H), 3.13 (m, J=7.3 Hz, 2H), 1.22 (t, J=7.3 Hz, 3H).

The compounds listed in the following table were prepared using a procedure similar to that described for Example 96, using a synthesized or commercial carboxylic acid:

| | Example | MS (ESI) | |
|---|---|---|---|
| Structure | No. | [M + H]⁺ | ¹H NMR |
| 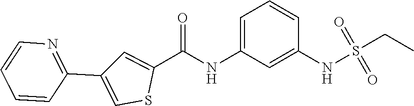 [SMILES: CCS(=O)(=O)Nc1cccc(NC(=O)c2cc(-c3ccccn3)cs2)c1] | 97 | 388 | ¹H NMR (300 MHz, DMSO- d6) δ 10.48 (s, 1H), 9.86 (s, 1H), 8.75 (d, J = 1.3 Hz, 1H), 8.65 (d, J = 4.7 Hz, 1H), 8.49 (d, J = 1.3 Hz, 1H), 7.96- 7.87 (m, 2H), 7.73 (s, 1H), 7.54 (d, J = 8.0 Hz, 1H), 7.38- |
| | | | 7.20 (m, 2H), 6.96 (d, J = |
| | | | 7.9 Hz, 1H), 3.13 (q, J = 7.3 |
| | | | Hz, 2H), 1.22 (t, J = 7.2 Hz, |
| | | | 3H) |

### Example 98: 5-bromo-N-(3-(methylsulfonamido)phenyl)-4-phenylthiophene-2-carboxamide

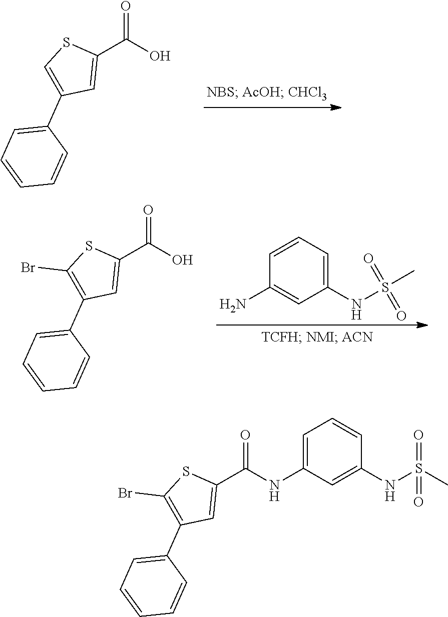 [SMILES: O=C(O)c1cc(-c2ccccc2)cs1 ; O=C(O)c1cc(-c2ccccc2)c(Br)s1 ; CS(=O)(=O)Nc1cccc(N)c1 ; CS(=O)(=O)Nc1cccc(NC(=O)c2cc(-c3ccccc3)c(Br)s2)c1]

Step 1: To a solution of 4-phenylthiophene-2-carboxylic acid (1.5 g, 7.344 mmol, 1 equiv) and NBS (1.57 g, 8.813 mmol, 1.2 equiv) in CHCl₃ (20 mL) was added AcOH (1.32 g, 22.032 mmol, 3 equiv) at room temperature. The resulting solution was stirred for 4 hours at room temperature. The resulting solution was concentrated under vacuum. The residue was purified by flash chromatography on C18 column gel eluting with ACN/water (0.05% FA) (28%) to afford 5-bromo-4-phenylthiophene-2-carboxylic acid (850 mg, 39.04%) as a yellow solid. LCMS: (ES, m/z): [M+H]⁺:283

Step 2: To a solution of 5-bromo-4-phenylthiophene-2-carboxylic acid (850 mg, 3.002 mmol, 1 equiv), NMI (492.9 mg, 6.003 mmol, 2.00 equiv) and TCFH (1.3 g, 4.633 mmol, 1.54 equiv) in ACN (10 mL) at room temperature. The resulting solution was stirred for 10 minus at room temperature. Then N-(3-aminophenyl)methanesulfonamide (838.6 mg, 4.503 mmol, 1.50 equiv) was added and stirred at room temperature for one hours. The residue was purified by flash chromatography on C18 Column gel eluting with ACN/water (0.05% NH₄HCO₃) (56%) to afford 5-bromo-N-(3-methanesulfonamidophenyl)-4-phenylthiophene-2-carboxamide (1.0 g, 67.90%) as a yellow solid and crude product (100 mg) was purify by Pre-HPLC on condition Column: XBridge Prep C18 OBD Column, 19*150 mm, 5 m; Mobile Phase A: Water (10 mmol/L NH4HCO3), Mobile Phase B: ACN; Flow rate: 25 mL/min; Gradient: 41% B to 77% B in 8 min, 77% B; Wave Length: 220 nm; RT1(min): 6.88 to afford 5-bromo-N-(3-methanesulfonamidophenyl)-4-phenylthio phene-2-carboxamide (29.2 mg, 50%) as a yellow solid. LCMS: (ES, m/z): [M+H]⁺: 451. ¹H NMR (400 MHz, DMSO-d6) δ 10.34 (s, 1H), 9.81 (s, 1H), 8.15 (s, 1H), 7.68-7.59 (m, 3H), 7.57-7.41 (m, 4H), 7.31 (t, J=8.1 Hz, 1H), 6.95 (ddd, J=8.0, 2.2, 1.0 Hz, 1H), 3.00 (s, 3H).

### Example 99: N-(3-(methylsulfonamido)phenyl)-4-phenyl-5-(trifluoromethyl)thiophene-2-carboxamide

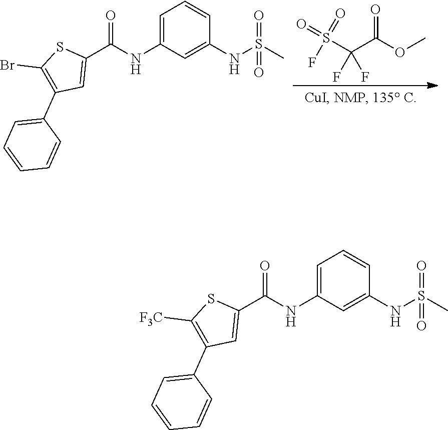

Step 1: To a solution of 5-bromo-N-(3-methanesulfonamidophenyl)-4-phenylthiophene-2-carboxamide (200 mg, 0.443 mmol, 1 equiv), CuI (8.4 mg, 0.044 mmol, 0.10 equiv), NMP (5 mL) in NMP (5 mL) was added methyl 2,2-difluoro-2-sulfoacetate (127.7 mg, 0.665 mmol, 1.50 equiv) at room temperature under nitrogen. The resulting solution was stirred for two hours at 135° C. The residue was purified by Pre-HPLC on condition: Column: Xselect CSH C18 OBD Column 30*150 mm 5 m, n; Mobile Phase A: Water (0.1% FA), Mobile Phase B: ACN; Flow rate: 60 mL/min; Gradient: 50% B to 66% B in 11 min, 66% B; Wave Length: 254/220 nm; RT1(min): 8.47 to afford N-(3-methanesulfonamidophenyl)-4-phenyl-5-(trifluoromethyl) thiophene-2-carboxamide (7.5 mg, 3.82%) as a white solid. LCMS: (ES, m/z): [M+1]=441. ¹H NMR (300 MHz, DMSO-d6) δ 10.55 (s, 1H), 9.86 (s, 1H), 8.12 (s, 1H), 7.69-7.53 (m, 6H), 7.36-7.30 (m, 1H), 6.97 (t, J=8.1 Hz, 1H), 3.02 (s, 3H).

### Example 100: 5-cyano-N-(3-(methylsulfonamido)phenyl)-4-phenylthiophene-2-carboxamide

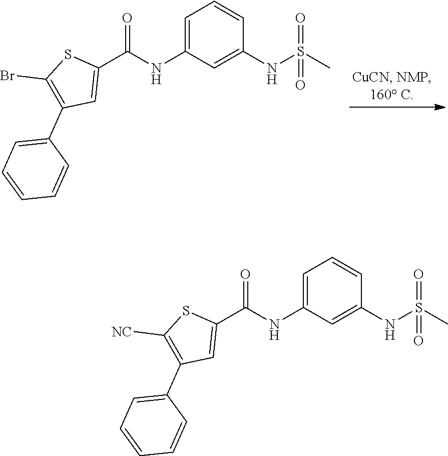 [SMILES: CS(=O)(=O)Nc1cccc(NC(=O)c2cc(-c3ccccc3)c(Br)s2)c1 ; CS(=O)(=O)Nc1cccc(NC(=O)c2cc(-c3ccccc3)c(C#N)s2)c1]

To a solution of 5-bromo-N-(3-methanesulfonamidophenyl)-4-phenylthiophene-2-carboxamide (120 mg, 0.266 mmol, 1 equiv) and CuCN (47.6 mg, 0.531 mmol, 2.00 equiv) in NMP (5 mL) at room temperature under nitrogen. The resulting solution was stirred overnight at 160° C. The residue was purified by Pre-HPLC on condition: Column: YMC-Actus Triart C18 ExRS, 30*150 mm, 5 m; Mobile Phase A: Water (10 mmol/L NH₄HCO₃), Mobile Phase B: ACN; Flow rate: 60 mL/min; Gradient: 52% B to 60% B in 8 min, 60% B; Wave Length: 220 nm; RT1(min): 7.58 to afford 5-cyano-N-(3-methane sulfonamidophenyl)-4-phenylthiophene-2-carboxamide (24.7 mg, 23.31%) as a yellow solid. LCMS: (ES, m/z): [M+1]=398. ¹H NMR (300 MHz, DMSO-d6) δ 10.65 (s, 1H), 9.88 (s, 1H), 8.45 (s, 1H), 7.89-7.73 (m, 2H), 7.71-7.45 (m, 5H), 7.35 (t, J=8.1 Hz, 1H), 6.99 (d, J=8.0 Hz, 1H), 3.02 (s, 3H).

### Example 101: 5-[(3-methanesulfonamidophenyl) carbamoyl]-3-phenylthiophene-2-carboxylic acid

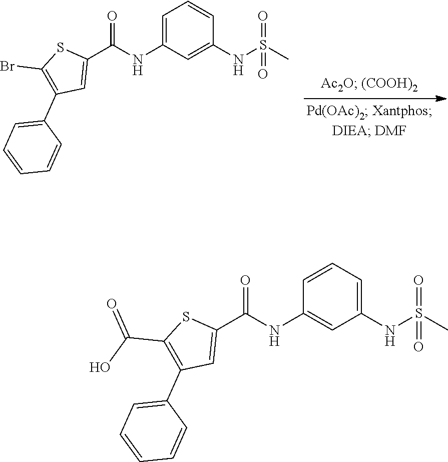 [SMILES: CS(=O)(=O)Nc1cccc(NC(=O)c2cc(-c3ccccc3)c(Br)s2)c1 ; CS(=O)(=O)Nc1cccc(NC(=O)c2cc(-c3ccccc3)c(C(=O)O)s2)c1]

To a solution of 5-bromo-N-(3-methanesulfonamidophenyl)-4-phenylthiophene-2-carboxamide (100 mg, 0.222 mmol, 1 equiv), acetic anhydride (45.3 mg, 0.444 mmol, 2.00 equiv), oxalic acid (39.9 mg, 0.443 mmol, 2.00 equiv), Pd(OAc)₂ (5 mg, 0.022 mmol, 0.10 equiv), and Xantphos (25.6 mg, 0.044 mmol, 0.20 equiv) in DMF (5 mL) was added DIEA (85.9 mg, 0.665 mmol, 3.00 equiv) at room temperature under nitrogen. The resulting solution was stirred for 2 hours at 100° C. The mixture was purified by Prep-HPLC to afford 5-[(3-methanesulfonamidophenyl) carbamoyl]-3-phenylthiophene-2-carboxylic acid (30.5 mg, 33.05%) as a yellow solid.

LCMS (ESI) [M+H]⁺=417

¹H NMR (300 MHz, DMSO-d6) δ 10.42 (s, 1H), 9.83 (s, 1H), 8.09 (s, 1H), 7.68 (t, J=2.1 Hz, 1H), 7.53 (ddd, J=7.2, 6.1, 2.0 Hz, 3H), 7.48-7.39 (m, 3H), 7.32 (t, J=8.1 Hz, 1H), 6.97 (ddd, J=8.0, 2.2, 1.0 Hz, 1H), 3.01 (s, 3H).

### Example 102: N2-(3-(methylsulfonamido)phenyl)-N4-phenylthiophene-2,4-dicarboxamide

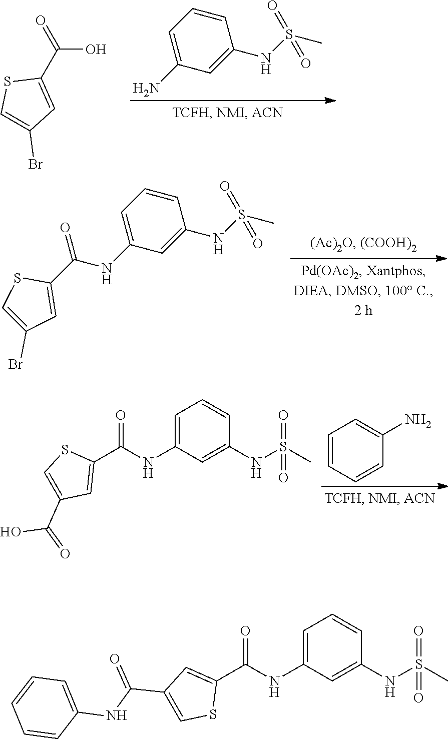

Step 1: To a stirred solution of 4-bromothiophene-2-carboxylic acid (500 mg, 2.41 mmol), TCFH (1.024 g, 3.615 mmol) and NMI (0.598 mg, 7.23 mmol) in ACN (4.00 mL) was added N-(3-aminophenyl)methanesulfonamide (448 mg, 24.1 mmol). Then the mixture was stirred for 4 hours at room temperature. The mixture was concentrated and purified by reverse phase flash chromatography eluting with ACN/H₂O (5-95%, acidic system) to afford 4-bromo-N-(3-methanesulfonamidophenyl)thiophene-2-carboxamide (850 mg, 95%) as a brown solid. Step 2: A solution of 4-bromo-N-(3-methanesulfonamidophenyl)thiophene-2-carboxamide (100 mg, 0.266 mmol, 1 equiv), acetic anhydride (54.41 mg, 0.532 mmol, 2 equiv), Pd(OAc)₂ (11.97 mg, 0.053 mmol, 0.2 equiv), Xantphos (30.84 mg, 0.053 mmol, 0.2 equiv), oxalic acid (47.99 mg, 0.532 mmol, 2 equiv) and DIEA (103.32 mg, 0.798 mmol, 3 equiv) in DMSO (3 mL) was stirred for 2 hours at 100° C. under nitrogen atmosphere. The resulting mixture was concentrated under vacuum. The residue was purified by silica gel column chromatography, eluting with Petroleum ether/EtOAc=2:3, to afford 5-[(3-methanesulfonamidophenyl)carbamoyl]thiophene-3-carboxylic acid (63 mg, 69.46%) as white solid.

Step 3: A solution of 5-[(3-methanesulfonamidophenyl)carbamoyl]thiophene-3-carboxylic acid (50 mg, 0.147 mmol, 1 equiv), aniline (15.05 mg, 0.162 mmol, 1.1 equiv), TCFH (82.43 mg, 0.294 mmol, 2 equiv) and NMI (36.18 mg, 0.441 mmol, 3 equiv) in ACN (2 mL) was stirred for 1 h at room temperature under nitrogen atmosphere. The resulting mixture was concentrated under reduced pressure. The residue was purified by reverse phase Flash Chromatography with the following conditions (water/ACN=2:3) to afford N2-(3-(methylsulfonamido)phenyl)-N4-phenylthiophene-2,4-dicarboxamide (13.2 mg, 21.63%) as a white solid.

LCMS (ESI) [M+H]⁺=416

¹H NMR (300 MHz, DMSO-d6) δ 10.50 (s, 1H), 10.24 (s, 1H), 9.80 (s, 1H), 8.63 (d, J=1.3 Hz, 1H), 8.53 (d, J=1.4 Hz, 1H), 7.81-7.73 (m, 2H), 7.71 (t, J=2.1 Hz, 1H), 7.59-7.51 (m, 1H), 7.38 (t, J=7.9 Hz, 2H), 7.31 (t, J=8.1 Hz, 1H), 7.12 (t, J=7.4 Hz, 1H), 7.00-6.91 (m, 1H), 3.02 (s, 3H).

The compounds listed in the following table were prepared using a procedure similar to that described for Example 102:

| | | MS | |
|---|---|---|---|
| | Example | (ESI) | |
| Structure | No. | [M + H]⁺ | ¹H NMR |
| 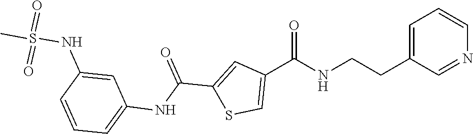 [SMILES: CS(=O)(=O)Nc1cccc(NC(=O)c2cc(C(=O)NCCc3cccnc3)cs2)c1] | 103 | 444 | |
| 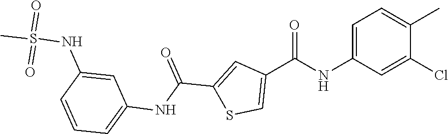 [SMILES: Cc1ccc(NC(=O)c2csc(C(=O)Nc3cccc(NS(C)(=O)=O)c3)c2)cc1Cl] | 104 | 463 | |
| 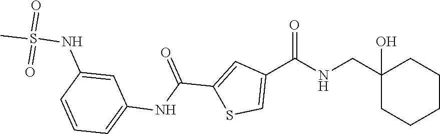 [SMILES: CS(=O)(=O)Nc1cccc(NC(=O)c2cc(C(=O)NCC3(O)CCCCC3)cs2)c1] | 105 | 451 | |
| 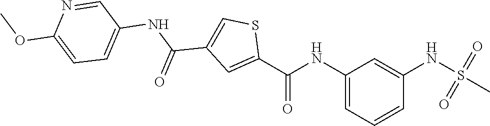 | 106 | 446 | |
| 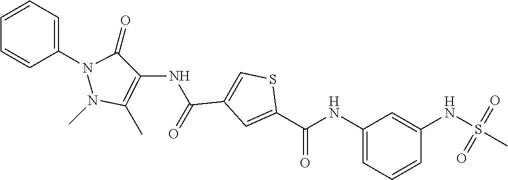 [SMILES: Cc1c(NC(=O)c2csc(C(=O)Nc3cccc(NS(C)(=O)=O)c3)c2)c(=O)n(-c2ccccc2)n1C] | 107 | 526 | |
| 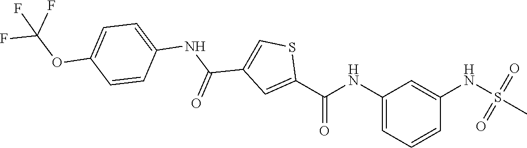 [SMILES: CS(=O)(=O)Nc1cccc(NC(=O)c2cc(C(=O)Nc3ccc(OC(F)(F)F)cc3)cs2)c1] | 108 | 499 | |
| 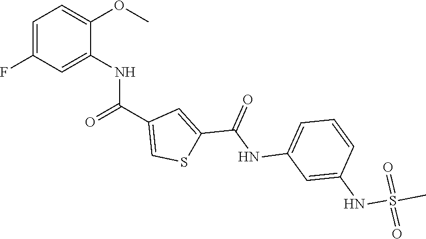 [SMILES: COc1ccc(F)cc1NC(=O)c1csc(C(=O)Nc2cccc(NS(C)(=O)=O)c2)c1] | 109 | 463 | |
| 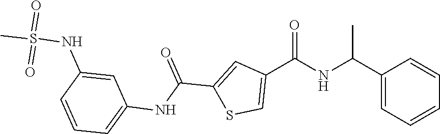 [SMILES: CC(NC(=O)c1csc(C(=O)Nc2cccc(NS(C)(=O)=O)c2)c1)c1ccccc1] | 110 | 443 | |
| 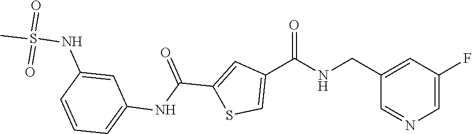 [SMILES: CS(=O)(=O)Nc1cccc(NC(=O)c2cc(C(=O)NCc3cncc(F)c3)cs2)c1] | 111 | 444 | |
| 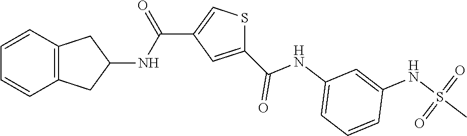 [SMILES: CS(=O)(=O)Nc1cccc(NC(=O)c2cc(C(=O)NC3Cc4ccccc4C3)cs2)c1] | 112 | 455 | |
| 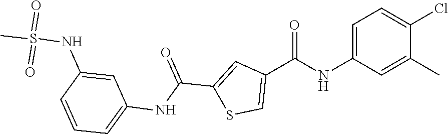 | 113 | 463 | |
| 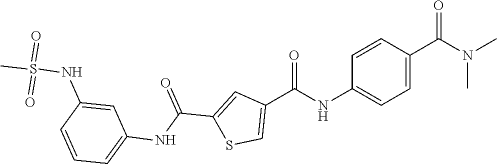 | 114 | 486 | |
| 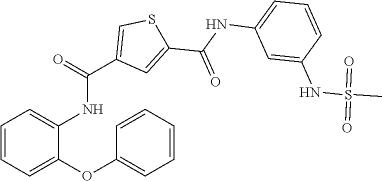 [SMILES: CS(=O)(=O)Nc1cccc(NC(=O)c2cc(C(=O)Nc3ccccc3Oc3ccccc3)cs2)c1] | 115 | 507 | |
| 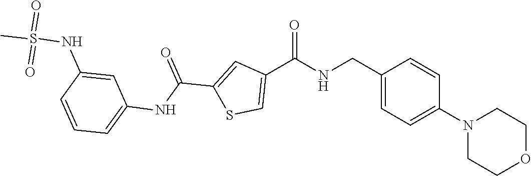 [SMILES: CS(=O)(=O)Nc1cccc(NC(=O)c2cc(C(=O)NCc3ccc(N4CCOCC4)cc3)cs2)c1] | 116 | 514 | |
| 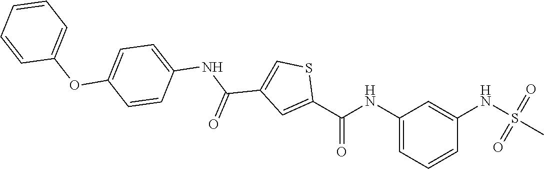 [SMILES: CS(=O)(=O)Nc1cccc(NC(=O)c2cc(C(=O)Nc3ccc(Oc4ccccc4)cc3)cs2)c1] | 117 | 507 | |
| 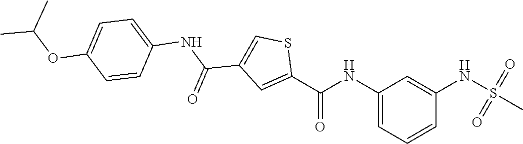 [SMILES: CC(C)Oc1ccc(NC(=O)c2csc(C(=O)Nc3cccc(NS(C)(=O)=O)c3)c2)cc1] | 118 | 473 | |
| 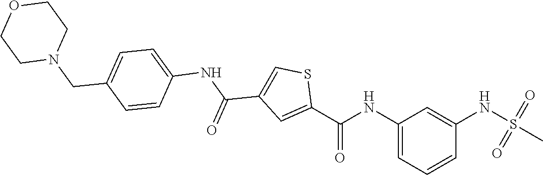 [SMILES: CS(=O)(=O)Nc1cccc(NC(=O)c2cc(C(=O)Nc3ccc(CN4CCOCC4)cc3)cs2)c1] | 119 | 473 | |
| 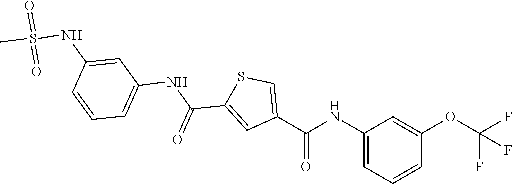 [SMILES: CS(=O)(=O)Nc1cccc(NC(=O)c2cc(C(=O)Nc3cccc(OC(F)(F)F)c3)cs2)c1] | 120 | 499 | |
| 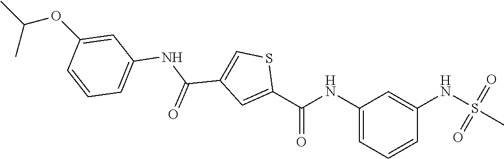 [SMILES: CC(C)Oc1cccc(NC(=O)c2csc(C(=O)Nc3cccc(NS(C)(=O)=O)c3)c2)c1] | 121 | 473 | |
| 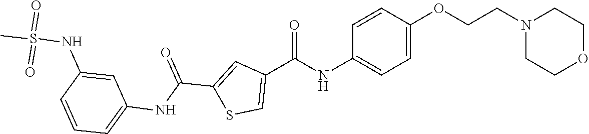 [SMILES: CS(=O)(=O)Nc1cccc(NC(=O)c2cc(C(=O)Nc3ccc(OCCN4CCOCC4)cc3)cs2)c1] | 122 | 544 | |
| 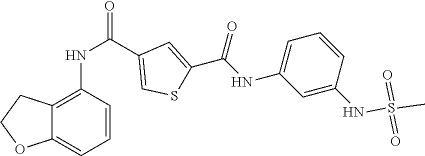 [SMILES: CS(=O)(=O)Nc1cccc(NC(=O)c2cc(C(=O)Nc3cccc4c3CCO4)cs2)c1] | 123 | 457 | |
| 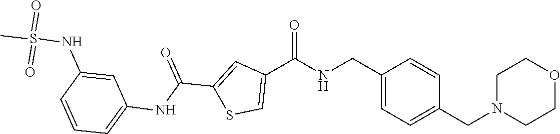 [SMILES: CS(=O)(=O)Nc1cccc(NC(=O)c2cc(C(=O)NCc3ccc(CN4CCOCC4)cc3)cs2)c1] | 124 | 528 | |
| 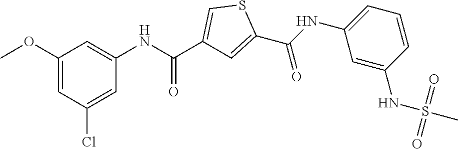 | 125 | 479 | |
| 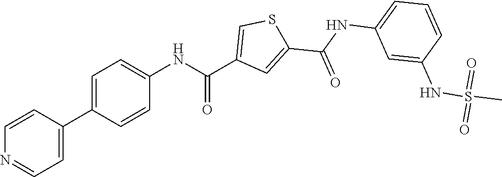 [SMILES: CS(=O)(=O)Nc1cccc(NC(=O)c2cc(C(=O)Nc3ccc(-c4ccncc4)cc3)cs2)c1] | 126 | 479 | |
| 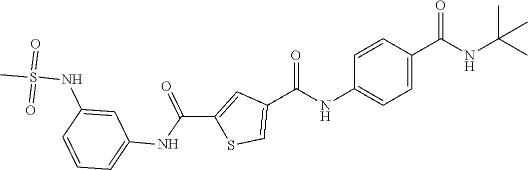 [SMILES: CC(C)(C)NC(=O)c1ccc(NC(=O)c2csc(C(=O)Nc3cccc(NS(C)(=O)=O)c3)c2)cc1] | 127 | 514 | |
| 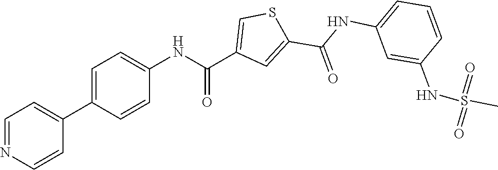 [SMILES: CS(=O)(=O)Nc1cccc(NC(=O)c2cc(C(=O)Nc3ccc(-c4ccncc4)cc3)cs2)c1] | 128 | 492 | |
| 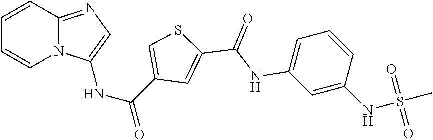 [SMILES: CS(=O)(=O)Nc1cccc(NC(=O)c2cc(C(=O)Nc3cnc4ccccn34)cs2)c1] | 129 | 455 | |
| 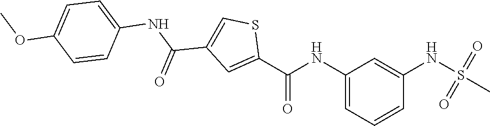 | 130 | 445 | |
| 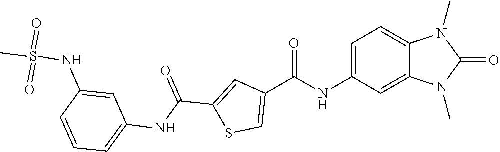 [SMILES: Cn1c(=O)n(C)c2cc(NC(=O)c3csc(C(=O)Nc4cccc(NS(C)(=O)=O)c4)c3)ccc21] | 131 | 500 | |
| 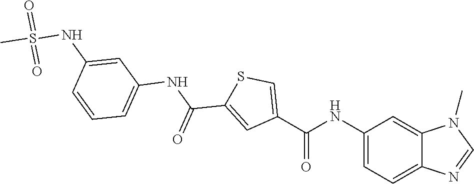 [SMILES: Cn1cnc2ccc(NC(=O)c3csc(C(=O)Nc4cccc(NS(C)(=O)=O)c4)c3)cc21] | 132 | 499 | |
| 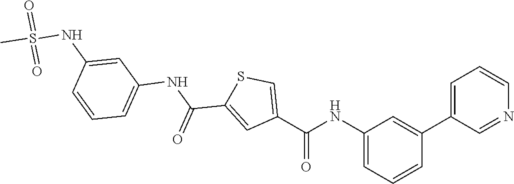 [SMILES: CS(=O)(=O)Nc1cccc(NC(=O)c2cc(C(=O)Nc3cccc(-c4cccnc4)c3)cs2)c1] | 133 | 492 | |
| 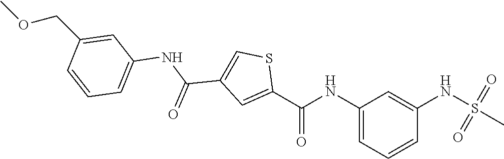 | 134 | 459 | |
| 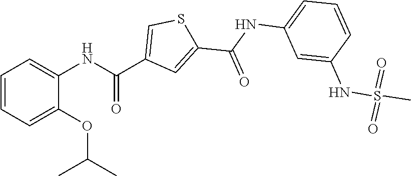 [SMILES: CC(C)Oc1ccccc1NC(=O)c1csc(C(=O)Nc2cccc(NS(C)(=O)=O)c2)c1] | 135 | 473 | |
| 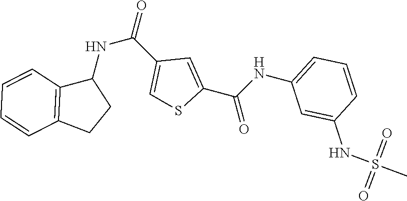 [SMILES: CS(=O)(=O)Nc1cccc(NC(=O)c2cc(C(=O)NC3CCc4ccccc43)cs2)c1] | 136 | 455 | |
| 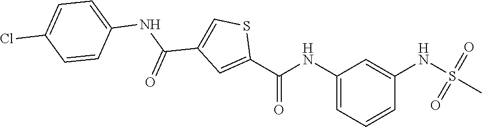 [SMILES: CS(=O)(=O)Nc1cccc(NC(=O)c2cc(C(=O)Nc3ccc(Cl)cc3)cs2)c1] | 137 | 499 | |
| 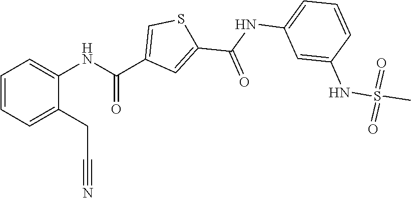 [SMILES: CS(=O)(=O)Nc1cccc(NC(=O)c2cc(C(=O)Nc3ccccc3CC#N)cs2)c1] | 138 | 454 | |
| 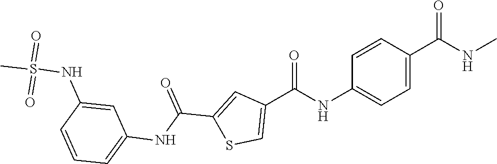 | 139 | 472 | |
| 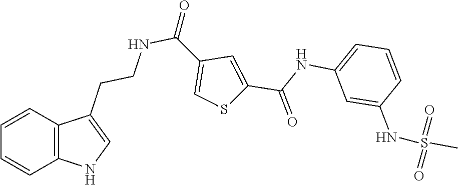 [SMILES: CS(=O)(=O)Nc1cccc(NC(=O)c2cc(C(=O)NCCc3c[nH]c4ccccc34)cs2)c1] | 140 | 483 | |
| 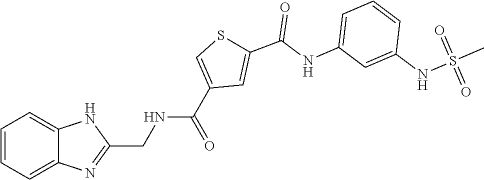 [SMILES: CS(=O)(=O)Nc1cccc(NC(=O)c2cc(C(=O)NCc3nc4ccccc4[nH]3)cs2)c1] | 141 | 469 | |
| 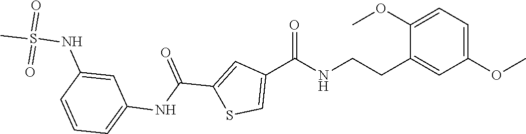 | 142 | 503 | |
| 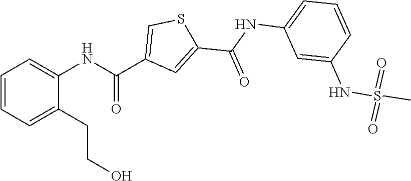 [SMILES: CS(=O)(=O)Nc1cccc(NC(=O)c2cc(C(=O)Nc3ccccc3CCO)cs2)c1] | 143 | 459 | |
| 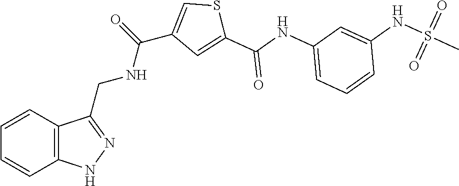 [SMILES: CS(=O)(=O)Nc1cccc(NC(=O)c2cc(C(=O)NCc3n[nH]c4ccccc34)cs2)c1] | 144 | 469 | |
| 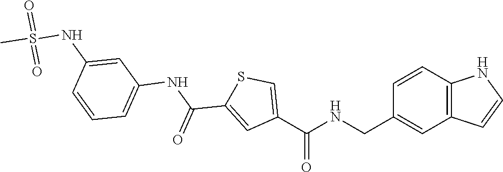 [SMILES: CS(=O)(=O)Nc1cccc(NC(=O)c2cc(C(=O)NCc3ccc4[nH]ccc4c3)cs2)c1] | 145 | 468 | |
| 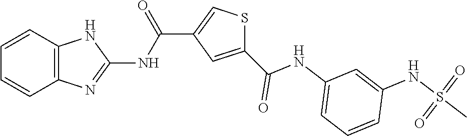 [SMILES: CS(=O)(=O)Nc1cccc(NC(=O)c2cc(C(=O)Nc3nc4ccccc4[nH]3)cs2)c1] | 146 | 455 | |
| 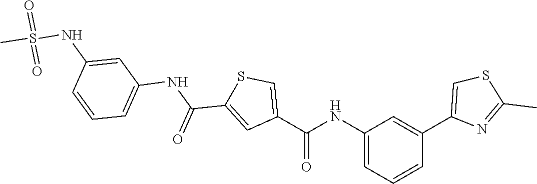 | 147 | 513 | |
| 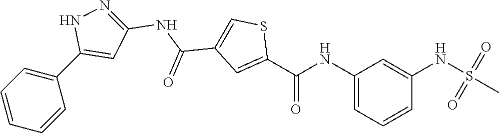 [SMILES: CS(=O)(=O)Nc1cccc(NC(=O)c2cc(C(=O)Nc3cc(-c4ccccc4)[nH]n3)cs2)c1] | 148 | 481 | |
| 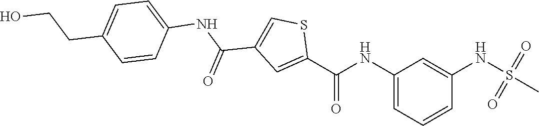 [SMILES: CS(=O)(=O)Nc1cccc(NC(=O)c2cc(C(=O)Nc3ccc(CCO)cc3)cs2)c1] | 149 | 459 | |
| 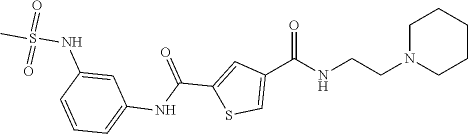 [SMILES: CS(=O)(=O)Nc1cccc(NC(=O)c2cc(C(=O)NCCN3CCCCC3)cs2)c1] | 150 | 450 | |
| 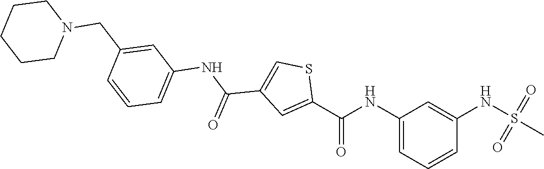 [SMILES: CS(=O)(=O)Nc1cccc(NC(=O)c2cc(C(=O)Nc3cccc(CN4CCCCC4)c3)cs2)c1] | 151 | 469 | |
| 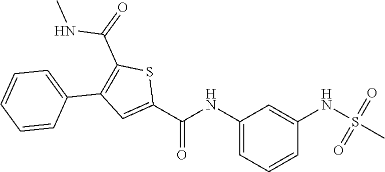 [SMILES: CNC(=O)c1sc(C(=O)Nc2cccc(NS(C)(=O)=O)c2)cc1-c1ccccc1] | 152 | 430 | ¹H NMR (400 MHz, DMSO-d6) δ 10.38 (s, 1H), 9.81 (s, 1H), 8.20 (d, J = 22.8 Hz, 2H), 7.67 (t, J = 2.1 Hz, 1H), 7.55-7.42 (m, 6H), 7.41- 7.36 (m, 1H), 7.30 (d, J = 8.1 Hz, 1H), 6.95 (ddd, J = 8.1, 2.3, 0.9 Hz, 1H), 3.00 (s, 3H), 2.68 (d, J = 4.6 Hz, 3H). |
| 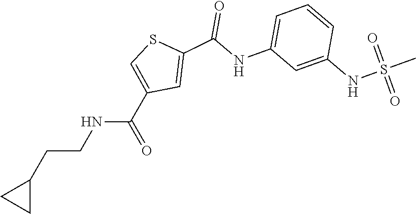 [SMILES: CS(=O)(=O)Nc1cccc(NC(=O)c2cc(C(=O)NCCC3CC3)cs2)c1] | 162 | 407.8 | |

### Example 153: 5-(hydroxymethyl)-N-(3-(methylsulfonamido)phenyl)-4-phenylthiophene-2-carboxamide

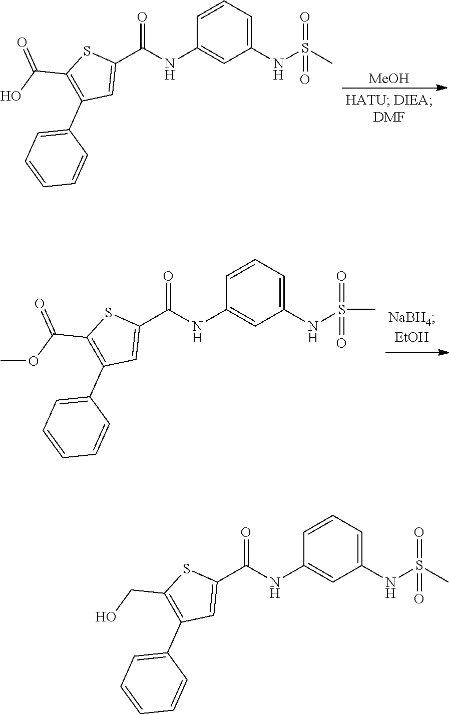

Step 1: To a solution of 5-[(3-methanesulfonamidophenyl)carbamoyl]-3-phenylthiophene-2-carboxylic acid (60 mg, 0.144 mmol, 1 equiv), HATU (82.2 mg, 0.216 mmol, 1.50 equiv) and DIEA (55.8 mg, 0.432 mmol, 3.00 equiv) in DMF (2 mL) was added methanol (9.2 mg, 0.287 mmol, 1.99 equiv) at room temperature. The resulting solution was stirred for two hours at room temperature. The residue was purified by C18 Column on condition: ACN/water (0.05% FA)(45%) to afford methyl 5-[(3-methanesulfonamidophenyl) carbamoyl]-3-phenylthiophene-2-carboxylate (40 mg, 58.37%) as a yellow solid.

Step 2: To a solution of methyl 5-[(3-methanesulfonamidophenyl)carbamoyl]-3-phenylthiophene-2-carboxylate (40 mg, 0.093 mmol, 1 equiv) in EtOH (5 mL, 86.067 mmol, 926.27 equiv) was added NaBH₄ (7.03 mg, 0.186 mmol, 2.00 equiv) at room temperature. The resulting solution was stirred for 2 hours at room temperature. The residue was purified by Pre-HPLC on condition: Column: XBridge Prep C18 OBD Column, 19*150 mm, 5 m; Mobile Phase A: Water (10 mmol/L NH₄HCO₃), Mobile Phase B: ACN; Flow rate: 25 mL/min; Gradient: 29% B to 45% B in 8 min, 45% B; Wave Length: 220 nm; RT1(min): 7.3 to afford 5-(hydroxymethyl)-N-(3-methanesulfonamidophenyl)-4-phenylthiophene-2-carboxamide (8.8 mg, 23.34%) as a yellow solid. LCMS: (ES, m/z): [M+1]=403. ¹H NMR (300 MHz, DMSO-d6) δ 10.24 (s, 1H), 9.79 (s, 1H), 8.14 (s, 1H), 7.69 (t, J=2.1 Hz, 1H), 7.51 (q, J=4.1, 3.1 Hz, 5H), 7.39 (ddd, J=8.6, 5.3, 2.5 Hz, 1H), 7.30 (t, J=8.1 Hz, 1H), 6.94 (dd, J=8.0, 2.1 Hz, 1H), 5.85 (t, J=5.4 Hz, 1H), 4.73 (d, J=5.3 Hz, 2H), 3.01 (s, 3H).

### Example 154: 1-benzyl-N-(3-methane sulfonamidophenyl)-5-methyl-1H-pyrazole-3-carboxamide

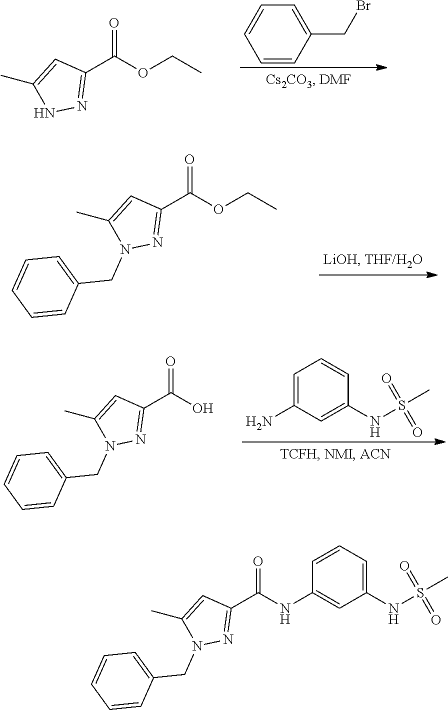 [SMILES: CCOC(=O)c1cc(C)[nH]n1 ; BrCc1ccccc1 ; CCOC(=O)c1cc(C)n(Cc2ccccc2)n1 ; Cc1cc(C(=O)O)nn1Cc1ccccc1 ; CS(=O)(=O)Nc1cccc(N)c1 ; Cc1cc(C(=O)Nc2cccc(NS(C)(=O)=O)c2)nn1Cc1ccccc1]

Step 1: To a stirred solution of ethyl 5-methyl-1H-pyrazole-3-carboxylate (500 mg, 3.24 mmol) and Cs₂CO₃ (3.15 g, 9.72 mmol), (bromomethyl)benzene (554 mg, 3.24 mmol) was added in DMSO (10 mL) dropwise. The mixture was stirred for 2 hours at 80° C. The mixture was concentrated and purified by reverse phase flash chromatography eluting with ACN/H₂O (0-100%, acidic system) to afford ethyl 1-benzyl-5-methyl-1H-pyrazole-3-carboxylate (420 mg, 1.71 mmol) as a yellow solid. LCMS (ESI) [M+H]⁺=245.

Step 2: To a stirred mixture of ethyl 1-benzyl-5-methyl-1H-pyrazole-3-carboxylate (420 mg, 1.71 mmol) and LiOH (123 mg, 5.13 mmol) was added THF (6 mL) and H₂O (2 mL) dropwise. The mixture was stirred for 2 hours at room temperature. The mixture was concentrated and purified by reverse phase flash chromatography eluting with ACN/H₂O (0-100%, acidic system) to afford 1-benzyl-5-methyl-1H-pyrazole-3-carboxylic acid (300 mg, 1.38 mmol) as a yellow solid. LCMS (ESI) [M+H]⁺: 217.

Step 3: To a stirred mixture of 1-benzyl-5-methyl-1H-pyrazole-3-carboxylic acid (60 mg, 0.277 mmol), TCFH (116 mg, 0.415 mmol), N-(3-aminophenyl)methanesulfonamide (51.5 mg, 0.277 mmol) and NMI (113 mg, 1.38 mmol) was added ACN (5 mL). Then the mixture was stirred for 2 hours at room temperature. The mixture was concentrated and purified by reverse phase flash chromatography eluting with ACN/H₂O (0-100%, acidic system) to afford 1-benzyl-N-(3-methane sulfonamidophenyl)-5-methyl-1H-pyrazole-3-carboxamide (58.7 mg, 0.152 mmol) as a white solid. LCMS (ESI) [M+H]⁺: 385. ¹H NMR (300 MHz, DMSO-d6) δ 10.04 (s, 1H), 9.78 (s, 1H), 7.81 (s, 1H), 7.57 (d, J=8.2 Hz, 1H), 7.31 (dq, J=21.2, 7.7 Hz, 4H), 7.17 (d, J=7.4 Hz, 2H), 6.93 (d, J=8.1 Hz, 1H), 6.66 (s, 1H), 5.45 (s, 2H), 3.03 (s, 3H), 2.25 (s, 3H).

### Example 155: 4-(benzylamino)-N-(3-(methylsulfonamido)phenyl)thiophene-2-carboxamide

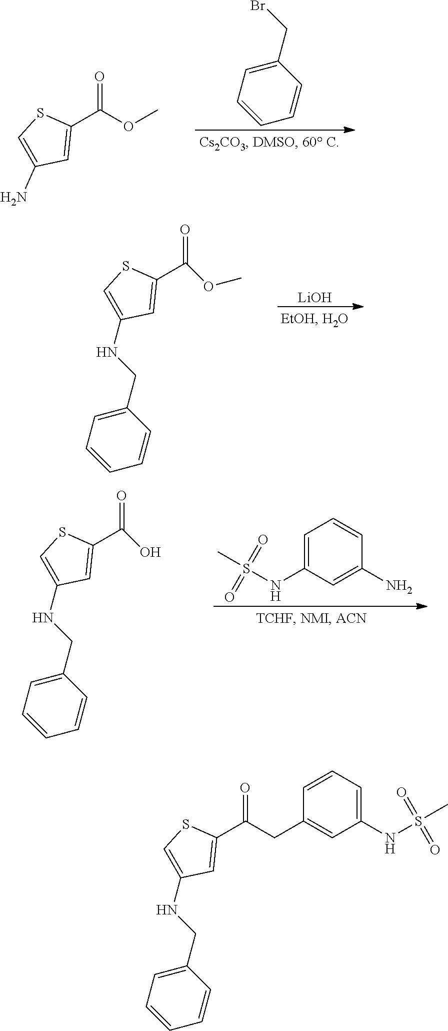

Step 1: A solution of methyl 4-aminothiophene-2-carboxylate (100 mg, 0.636 mmol, 1 equiv) and benzyl bromide (119.69 mg, 0.700 mmol, 1.1 equiv) and Cs₂CO₃ (621.83 mg, 1.908 mmol, 3 equiv) in DMSO (3 mL) was stirred for 1 h at 60° C. The residue was purified by reverse phase flash chromatography with the following conditions: column, silica gel; mobile phase, MeCN in water, 0% to 100% gradient in 20 min; detector, UV 254 nm. This resulted in methyl 4-(benzylamino)thiophene-2-carboxylate (130 mg, 82.63%) as a yellow solid.

LCMS (ESI) [M+H]⁺: 248.

Step 2: A solution of methyl 4-(benzylamino) thiophene-2-carboxylate (130 mg, 0.526 mmol, 1 equiv) an d LiOH (37.77 mg, 1.578 mmol, 3 equiv) in EtOH (3 mL) and H₂O (1 mL) was stirred for 1 h at room temperature. The mixture was acidified to pH 6 with HCl (aq.). The residue was purified by reverse phase flash chromatography with the following conditions: column, silica gel; mobile phase, MeCN in water, 0% to 100% gradient in 20 min; detector, UV 254 nm. This resulted in 4-(benzylamino) thiophene-2-carboxylic acid (90 mg, 73.39%) as a yellow solid. LCMS (ESI) [M+H]⁺: 234.

Step 3: To a stirred solution of 4-(benzylamino) thiophene-2-carboxylic acid (90 mg, 0.386 mmol, 1 equiv) and N-(3-aminophenyl) methanesulfonamide (71.84 mg, 0.386 mmol, 1 equiv) in ACN (3 mL) were added NMI (95.02 mg, 1.158 mmol, 3 equiv) and TCFH (162.37 mg, 0.579 mmol, 1.5 equiv) in portions at room temperature. The residue was purified by reverse phase flash chromatography with the following conditions: column, silica gel; mobile phase, MeCN in water, 0% to 100% gradient in 20 min; detector, UV 254 nm. This resulted in 4-(benzylamino)-N-(3-methanesulfonamidophenyl) thiophene-2-carboxamide (66.2 mg, 42.74%) as a light yellow solid. LCMS (ES, m/z): LCMS (ESI) [M+H]⁺: 402. ¹H NMR (300 MHz, DMSO-d6) δ 10.21 (s, 1H), 9.75 (s, 1H), 7.65 (t, J=2.1 Hz, 1H), 7.56 (d, J=1.7 Hz, 1H), 7.51-7.43 (m, 1H), 7.42-7.20 (m, 6H), 6.92 (m J=8.0, 2.2, 1.0 Hz, 1H), 6.35-6.12 (m, 2H), 4.23 (d, J=6.0 Hz, 2H), 3.01 (s, 3H).

### Example 156: N-(6-methanesulfonamidopyridin-2-yl)-4-phenylthiophene-2-carboxamide

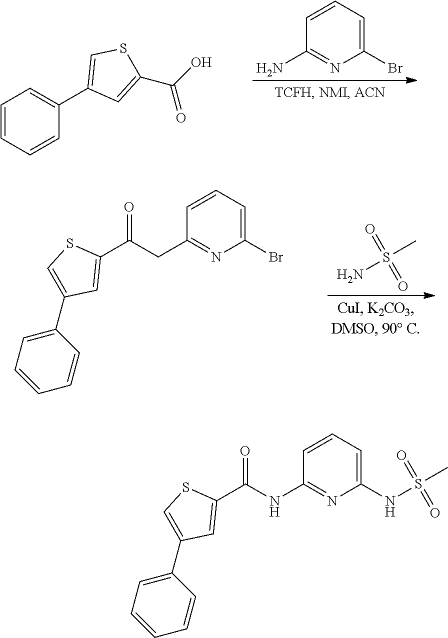

Step 1: To a stirred solution of 4-phenylthiophene-2-carboxylic acid (150 mg, 0.734 mmol), TCFH (309.381 mg, 1.101 mmol) and NMI (180.5 mg, 2.202 mmol) in ACN (10.00 mL) was added 6-bromopyridin-2-amine (126 mg, 0.734 mmol). Then the mixture was stirred for 8 hours at room temperature. The mixture was concentrated and purified by reverse phase flash chromatography eluting with ACN/H₂O (5-95%, acidic system) to afford N-(6-bromopyridin-2-yl)-4-phenylthiophene-2-carboxamide (120 mg, 95%) as a yellow solid. LCMS (ESI) [M+H]⁺: 359.

Step 2: To a stirred solution of N-(6-bromopyridin-2-yl)-4-phenylthiophene-2-carboxamide (100 mg, 0.278 mmol), CuI (10 mg, 0.0556 mmol) and K₂CO₃ (115.092 mg, 0.834 mmol) in DMSO (5.0 mL) was added methanesulfonamide (132 mg, 1.39 mmol) at 0° C. Then the mixture was stirred for 1 hour at room temperature. The mixture was concentrated and purified by flash chromatography on silica gel eluting with EtOAc/Petroleum ether (0-100%) to afford N-(6-methanesulfonamidopyridin-2-yl)-4-phenylthiophene-2-carboxamide (26.8 mg, 98.887%) as a yellow solid. LCMS (ESI) [M+H]⁺: 374. ¹H NMR (400 MHz, DMSO-d6) δ 10.54 (d, J=11.1 Hz, 2H), 8.61 (d, J=1.5 Hz, 1H), 8.24 (d, J=1.5 Hz, 1H), 7.80-7.69 (m, 4H), 7.48 (t, J=7.7 Hz, 2H), 7.40-7.31 (m, 1H), 6.69 (dd, J=7.3, 1.4 Hz, 1H), 3.52 (s, 3H).

### Example 157: N-(3-(methylsulfonoamidimidamido)phenyl)-4-phenylthiophene-2-carboxamide

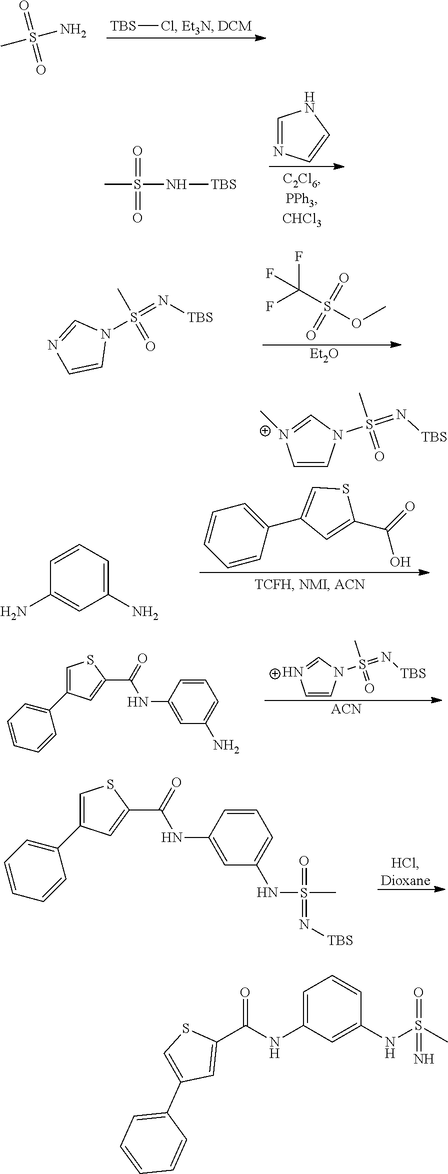

Step 1: To a stirred solution of methanesulfonamide (500 mg, 5.25 mmol) and Et₃N (1.58 g, 15.7 mmol) in DCM (10 mL) was added TBS-Cl (869 mg, 5.77 mmol) dropwise at 0° C. Then the mixture was stirred for 1 h at room temperature. The mixture was concentrated and purified by flash chromatography on silica gel eluting with EtOAc/Petroleum ether (0-100%) to afford N-(tert-butyldimethylsilyl)methanesulfonamide (752 mg, 3.59 mmol) as a yellow solid. LCMS (ESI) [M+H]⁺: 210

Step 2: PPh₃ (962 mg, 3.67 mmol) and C₂Cl₆ (868 mg, 3.67 mmol) were dissolved in dry CHCl3 (10 mL) under argon atmosphere. The resulting solution was heated at 70° C. for 6 h, and the formation of a white suspension was observed. The reaction mixture was cooled to room temperature and Et₃N (439 mg, 4.34 mmol) was added. After stirring for additional 10 min, the formation of a yellow suspension was observed. The reaction mixture was cooled to 0° C. and a solution of N-(tert-butyldimethylsilyl)methanesulfonamide (700 mg, 3.34 mmol) was added dropwise. The mixture was stirred for 30 min and a solution of 1H-imidazole (249 mg, 3.67 mmol) and Et₃N (371 mg, 3.67 mmol) in THF (3 mL) was added dropwise. After stirring at 0° C. for an additional 30 min, the reaction mixture was allowed to warm to room temperature and stirred overnight. The reaction mixture was concentrated under reduced pressure, the obtained solids were extracted with hexanes (200 ml), and the hexane solution was evaporated under vacuum. The residue was dissolved in CH₂Cl₂ (150 mL) and washed with H₂O (3×200 mL). The organic phase was dried over anhydrous Na₂SO₄ and concentrated under reduced pressure. The crude product was purified by column chromatography (EtOAc/hexane (3:2) as eluent, Rf=0.50) to afford (tert-butyldimethylsilyl)[(1H-imidazol-1-yl)(methyl) oxo-λ⁶-sulfanylidene]amine (390 mg, 1.50 mmol) as a yellow solid. LCMS (ESI) [M+H]⁺: 260.

Step 3: A solution of (tert-butyldimethylsilyl)[(1H-imidazol-1-yl)(methyl)oxo-λ⁶-sulfanylidene]amine (370 mg, 1.42 mmol) in dry Et₂O (10 mL) was cooled to 0° C. in an argon atmosphere, methyl trifluoromethanesulfonate (255 mg, 1.56 mmol) was added dropwise and the reaction mixture was stirred at 0° C. for 1 h. The obtained solids were filtered, washed with dry Et₂O (2×200 mL), and dried under vacuum to afford 1-{[(tert-butyldimethylsilyl)imino](methyl)oxo-λ⁶-sulfanyl}-3-methyl-1H-imidazol-3-ium (300 mg, 1.09 mmol) as white solid. LCMS (ESI) [M+H]⁺: 275

Step 4: To a stirred solution of benzene-1,3-diamine (150 mg, 1.38 mmol), TCFH (502 mg, 1.79 mmol) and NMI (339 mg, 4.14 mmol) in ACN (3 mL) was added 4-phenylthiophene-2-carboxylic acid (308 mg, 1.51 mmol). Then the mixture was stirred for 1 h at room temperature. The mixture was concentrated and purified by reverse phase flash chromatography eluting with ACN/H₂O (5-95%, acidic system) to afford N-(3-aminophenyl)-4-phenylthiophene-2-carboxamide (247 mg, 0.840 mmol) as a yellow solid. LCMS (ESI) [M+H]⁺: 295

Step 5: A solution of 1-{[(tert-butyldimethylsilyl)imino](methyl)oxo-λ⁶-sulfanyl}-3-methyl-1H-imidazol-3-ium (300 mg, 1.09 mmol) and N-(3-aminophenyl)-4-phenylthiophene-2-carboxamide (350 mg, 1.19 mmol) in ACN (10 mL) was stirred for 0.5 hours at 80° C. The mixture was concentrated and purified by reverse phase flash chromatography eluting with ACN/H₂O (5-95%, acidic system) to afford N-{3-[N-(tert-butyldimethylsilyl)methanesulfonoimidamido]phenyl}-4-phenylthiophene-2-carboxamide (178 mg, 0.366 mmol) as a white solid. LCMS (ESI) [M+H]⁺: 486 Step 6: HCl (3.08 mmol) in dioxane (5 mL) was added portion-wise to a stirred solution of N-13-[N-(tert-butyldimethylsilyl)methanesulfonoimidamido]phenyl}-4-phenylthiophene-2-carboxamide (150 mg, 0.308 mmol) in dry dioxane (5 mL) at 0° C. The reaction mixture was stirred at 0° C. for an additional 10 min and then it was allowed to warm to room temperature. The obtained mixture was concentrated under reduced pressure. The residue was purified by silica gel column chromatography, eluted with Petroleum ether/EtOAc (0-100%) to afford N-(3-methanesulfonoimidamidophenyl)-4-phenylthiophene-2-carboxamide (26.8 mg, 0.0713 mmol) as a white solid. LCMS (ESI) [M+H]⁺: 372. ¹H NMR (300 MHz, DMSO-d6) δ 10.12 (s, 1H), 8.50 (d, J=1.4 Hz, 1H), 8.17 (d, J=1.4 Hz, 1H), 7.80-7.71 (m, 2H), 7.50-7.23 (m, 5H), 7.14 (t, J=8.0 Hz, 1H), 6.77 (d, J=9.9 Hz, 3H), 3.14 (s, 3H).

### Example 158: N-(3-methyl-5-(methylsulfonamido)phenyl)-4-phenylthiophene-2-carboxamide

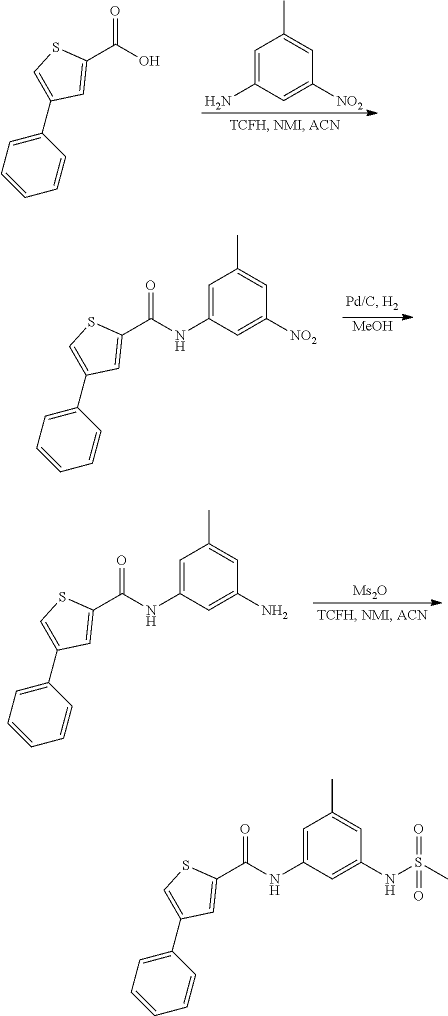

Step 1: To a mixture of 4-phenylthiophene-2-carboxylic acid (110 mg, 0.538 mmol) and 3-methyl-5-nitroaniline (98.1 mg, 0.645 mmol) in CAN (2 mL) was added TCFH (225 mg, 0.807 mmol) and NMI (132 mg, 1.61 mmol) at room temperature for two hours. Then it was concentrated, the residue was purified by reverse phase flash chromatography eluting with 50% of acetonitrile in water (0.1% NH₄HCO₃) to afford N-(3-methyl-5-nitrophenyl)-4-phenylthiophene-2-carboxamide (112 mg, 61.2%) as a white solid. LCMS [M+H]⁺: 339. Step 2: To a mixture of N-(3-methyl-5-nitrophenyl)-4-phenylthiophene-2-carboxamide (110 mg, 0.325 mmol), Pd/C (13.0 mg, 0.325 mmol) was added MeOH (1 mL). The resulting mixture was stirred for 1 hours at room temperature under H₂ atmosphere. The reaction mixture filter and washed with Methanol, then the filtrate was concentrated under reduced pressure. The residue was purified by flash chromatography on silica gel eluting with 35% of ethyl acetate in petroleum ether to afford N-(3-amino-5-methylphenyl)-4-phenylthiophene-2-carboxamide (82 mg, 81.7%) as a light yellow solid. LCMS [M+H]⁺: 309.

Step 3: To a mixture of N-(3-amino-5-methylphenyl)-4-phenylthiophene-2-carboxamide (82 mg, 0.265 mmol) and methanesulfonyl methanesulfonate (55.3 mg, 0.318 mmol) in ACN (1 mL) was added TCFH (111 mg, 0.397 mmol) and NMI (65.1 mg, 0.795 mmol). The resulting mixture was stirred at room temperature for two hours. Then it was concentrated, the residue was purified by reverse phase flash chromatography eluting with 55% of acetonitrile in water (0.1% NH₄HCO₃) to afford N-(3-methanesulfonamido-5-methylphenyl)-4-phenylthiophene-2-carboxamide (34.0 mg, 33.1%) as a white solid. LCMS [M+H]⁺: 387. ¹H NMR (300 MHz, DMSO-d6) δ 10.26 (s, 1H), 9.76 (s, 1H), 8.50 (d, J=1.5 Hz, 1H), 8.19 (d, J=1.4 Hz, 1H), 7.80-7.71 (m, 2H), 7.55-7.43 (m, 3H), 7.42-7.30 (m, 2H), 6.79 (d, J=2.3 Hz, 1H), 3.01 (s, 3H), 2.30 (s, 3H).

### Example 159: N-(3-formyl-5-(methylsulfonamido)phenyl)-4-phenylthiophene-2-carboxamide

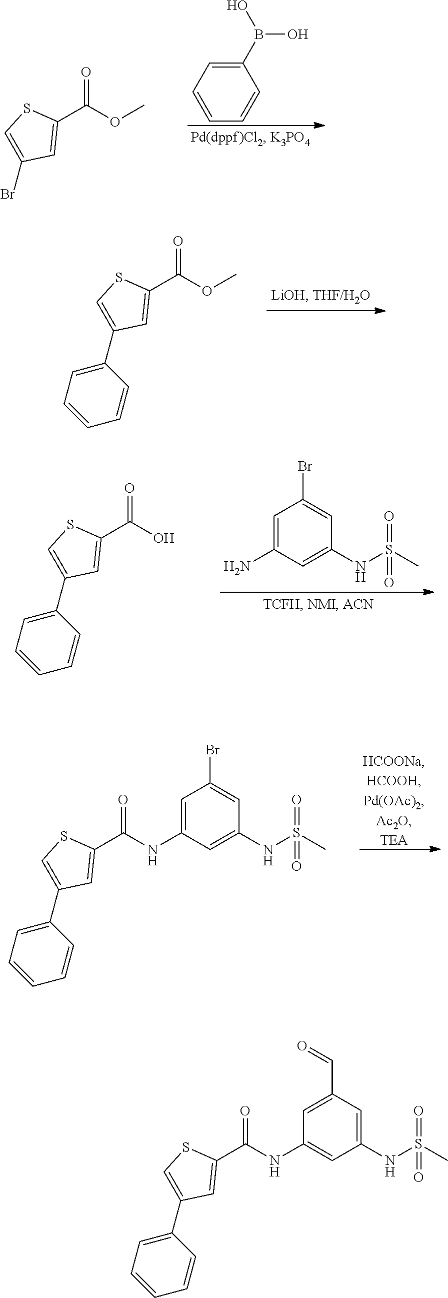

Step 1: To a mixture of methyl 4-bromothiophene-2-carboxylate (30 g, 135 mmol), phenylboronic acid (32.9 g, 270 mmol), Pd(dppf)Cl₂ (11.0 g, 13.5 mmol) and K₃PO₄ (85.8 g, 405 mmol) was added dioxane (300 mL) and H₂O (30 mL). The resulting mixture was stirred for 2 hours at 80° C. under N2 atmosphere. The reaction mixture was extracted with ethyl acetate. The organic phase was concentrated under reduced pressure. The residue was purified by flash chromatography on silica gel eluting with 20% of ethyl acetate in petroleum ether to afford methyl 4-phenylthiophene-2-carboxylate (27.1 g, 124 mmol) as a light yellow solid.

Step 2: To a stirred solution of methyl 4-phenylthiophene-2-carboxylate (26.1 g, 119 mmol) in 260 mL of THF and 260 mL of water was added LiOH (24.9 g, 595 mmol). The resulting mixture was stirred overnight at room temperature. Then it was concentrated, and the pH value of the residue solution was adjusted to 3 with 1M HCl (aq). The product was precipitated from the solution. After filtration, obtained 4-phenylthiophene-2-carboxylic acid (24.4 g, 119 mmol) as a white solid.

Step 3: To a mixture of 4-phenylthiophene-2-carboxylic acid (10.1 g, 49.6 mmol) and N-(3-amino-5-bromophenyl) methanesulfonamide (11 g, 41.4 mmol) in ACN (110 mL) was added TCFH (17.3 g, 62.1 mmol) and NMI (10.1 g, 124 mmol) at room temperature. The resulting mixture was stirred overnight, Then it was concentrated, the residue was purified by reverse phase flash chromatography eluting with 60% of acetonitrile in water (0.1% FA) to afford N-(3-bromo-5-methanesulfonamidophenyl)-4-phenylthiophene-2-carboxamide (11.0519 g, 24.486 mmol) as a light yellow solid. LCMS (ESI) [M−H]⁻: 449. ¹H NMR (300 MHz, DMSO-d6) δ 10.46 (s, 1H), 10.09 (s, 1H), 8.49 (d, J=1.5 Hz, 1H), 8.23 (d, J=1.4 Hz, 1H), 7.96-7.60 (m, 4H), 7.57-7.24 (m, 3H), 7.11 (t, J=1.9 Hz, 1H), 3.08 (s, 3H).

Step 4: A solution of N-(3-bromo-5-methanesulfonamidophenyl)-4-phenylthiophene-2-carboxamide (100 mg, 0.222 mmol, 1 equiv) and HCOOH (20.39 mg, 0.444 mmol, 2 equiv) and Pd(OAc)₂ (9.95 mg, 0.044 mmol, 0.2 equiv) and Ac₂O (45.24 mg, 0.444 mmol, 2 equiv) and HCOONa (75.33 mg, 1.110 mmol, 5 equiv) in Et₃N (44.84 mg, 0.444 mmol, 2 equiv) was stirred overnight at 90° C. under nitrogen atmosphere. The resulting mixture was extracted with EtOAc (3×10 mL). The combined organic layers were washed with brine (20 mL), dried over anhydrous Na₂SO₄. After filtration, the filtrate was concentrated under reduced pressure. The residue was purified by reverse phase flash chromatography with the following conditions: column, C18 silica gel; mobile phase, MeCN in Water (10 mmol/L NH₄HCO₃), 0% to 100% gradient in 10 min; detector, UV 254 nm. This resulted in N-(3-formyl-5-methanesulfonamidophenyl)-4-phenylthiophene-2-carboxamide (11.1 mg, 12.51%) as a white solid. LCMS (ES, m/z): LCMS (ESI) [M+H]⁺: 401. ¹H NMR (300 MHz, DMSO-d6) δ 10.59 (s, 1H), 9.96 (s, 1H), 8.53 (d, J=1.5 Hz, 1H), 8.23 (d, J=1.4 Hz, 1H), 8.06 (s, 1H), 7.98 (s, 1H), 7.81-7.71 (m, 2H), 7.55-7.44 (m, 3H), 7.41-7.34 (m, 1H), 3.07 (d, J=1.2 Hz, 3H).

### Example 160: 5-[5-(3,3-difluoroazetidin-1-yl)pyridin-2-yl]-N-(3-methanesulfonamidophenyl)-1-methylpyrrole-3-carboxamide

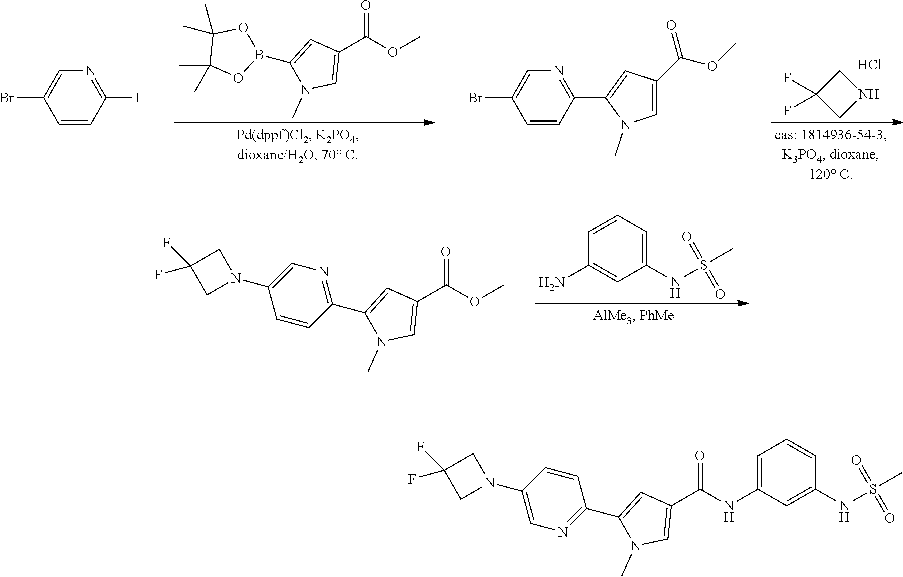

Step 1: To a mixture of 5-bromo-2-iodopyridine (5.1 g, 17.9 mmol), methyl 1-methyl-5-(4,4,5,5-tetramethyl-1,3,2-dioxaborolan-2-yl)-1H-pyrrole-3-carboxylate (5.67 g, 21.4 mmol), Pd(dppf)Cl₂ (1.45 g, 1.78 mmol) and K₃PO₄ (11.3 g, 53.6 mmol) was added 1,4-dioxane (60 ml) and H₂O (6 ml). The resulting mixture was stirred for 2 hours at 70° C. under N2 atmosphere. The reaction mixture was extracted with ethyl acetate. The organic phase was concentrated under reduced pressure. The residue was purified by flash chromatography on silica gel eluting with 15% of ethyl acetate in petroleum ether to afford methyl 5-(5-bromopyridin-2-yl)-1-methyl-1H-pyrrole-3-carboxylate (5.10 g, 96.5%) as a white solid.

LCMS [M+H]⁺: 295

Step 2: To a mixture of methyl 5-(5-bromopyridin-2-yl)-1-methyl-1H-pyrrole-3-carboxylate (3.1 g, 10.5 mmol), 3,3-difluoroazetidine hydrochloride (4.08 g, 31.5 mmol), Cs₂CO₃ (10.28 g, 31.6 mmol) and PEPPSI IHept-Cl (510 mg, 525 μmol) was added dioxane (30 mL). The resulting mixture was stirred for 1 hours at 120° C. under N2 atmosphere. The reaction mixture was extracted with ethyl acetate. The organic phase was concentrated under reduced pressure. The residue was purified by flash chromatography on silica gel eluting with 30% of ethyl acetate in petroleum ether to afford methyl 5-[5-(3,3-difluoroazetidin-1-yl)pyridin-2-yl]-1-methyl-1H-pyrrole-3-carboxylate (2.20 g, 68.3%) as a yellow solid. LCMS [M+H]⁺: 308

Step 3: A solution of methyl 5-[5-(3,3-difluoroazetidin-1-yl)pyridin-2-yl]-1-methylpyrrole-3-carboxylate (140 mg, 0.456 mmol, 1 equiv) and N-(3-aminophenyl)methanesulfonamide (102 mg, 0.547 mmol, 1.2 equiv) in toluene (4 mL) was stirred for 10 min at 0° C. under nitrogen atmosphere. To the above mixture was added AlMe₃ (0.14 mL, 1.368 mmol, 3 equiv) dropwise over 1 min at 0° C. The resulting mixture was stirred for additional 1 hour at 80° C. The mixture was allowed to cool down to room temperature. The reaction was quenched with water at 0° C. The resulting mixture was concentrated under reduced pressure. The residue (120 mg) was purified by Prep-HPLC with the following conditions (Column: XSelect CSH Prep C18 OBD Column, 19*150 mm, 5 μm; Mobile Phase A: Water (0.1% formic acid), Mobile Phase B: ACN; Flow rate: 25 mL/min; Gradient: 21% B to 52% B in 7 min, 52% B; Wave Length: 254/220 nm; RT1(min): 7.20) to afford 5-[5-(3,3-difluoroazetidin-1-yl)pyridin-2-yl]-N-(3-methanesulfonamidophenyl)-1-methylpyrrole-3-carboxamide (104.9 mg, 49.9% yield) as a white solid. LCMS (ESI) [M+H]⁺: 462.15

¹H NMR (400 MHz, DMSO-d₆) δ 9.73 (s, 1H), 9.64 (s, 1H), 7.98 (d, J=2.9 Hz, 1H), 7.69 (d, J=2.0 Hz, 1H), 7.61-7.44 (m, 3H), 7.25 (t, J=8.1 Hz, 1H), 7.14-6.98 (m, 2H), 6.95-6.82 (m, 1H), 4.39 (t, J=12.3 Hz, 4H), 3.92 (s, 3H), 3.00 (s, 3H).

### Example 161: 5-(3,5-difluoropyridin-2-yl)-N-(3-fluoro-5-methanesulfonamidophenyl)-1-methyl-1H-pyrrole-3-carboxamide

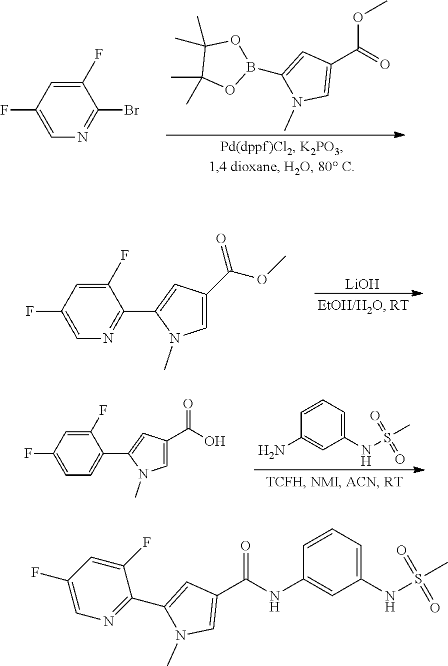

Step 1: A solution of 2-bromo-3,5-difluoropyridine (1.5 g, 7.73 mmol), methyl 1-methyl-5-(4,4,5,5-tetramethyl-1,3,2-dioxaborolan-2-yl)-1H-pyrrole-3-carboxylate (2.04 g, 7.73 mmol), Pd(dppf)Cl₂ (630 mg, 773 μmol) and K₂CO₃ (3.18 g, 23.1 mmol) in dioxane (10 mL) and H₂O (2 mL) was stirred for 1 hour at 80° C. under nitrogen atmosphere. The resulting mixture was concentrated under reduced pressure. The residue product was purified by reverse phase flash with the following conditions (20-80% acetonitrile in water) to afford methyl 5-(3,5-difluoropyridin-2-yl)-1-methyl-1H-pyrrole-3-carboxylate (1.20 g, 4.75 mmol) as white solid.

LCMS (ESI) [M+H]⁺: 253.00.

Step 2: A solution of methyl 5-(3,5-difluoropyridin-2-yl)-1-methyl-1H-pyrrole-3-carboxylate (150 mg, 594 μmol) and lithium hydroxide (113 mg, 4.75 mmol) in H₂O (0.4 mL) and EtOH (2 mL) was stirred for 1 h at room temperature under nitrogen atmosphere. The mixture was acidified to pH 7 with HCl (aq.). The resulting mixture was concentrated under vacuum. The residue was purified by reversed-phase flash chromatography with the following conditions: column, C18 silica gel; mobile phase, MeCN in water (0.1% FA), 10% to 50% gradient in 10 min; detector, UV 254 nm. to afford 5-(3,5-difluoropyridin-2-yl)-1-methyl-1H-pyrrole-3-carboxylic acid (120 mg, 503 μmol) as a white solid. LCMS (ESI) [M+H]⁺: 239.00.

Step 3: A solution of 5-(3,5-difluoropyridin-2-yl)-1-methyl-1H-pyrrole-3-carboxylic acid (120 mg, 503 μmol), N-(3-amino-5-fluorophenyl)methanesulfonamide (102 mg, 503 μmol), TCFH (140 mg, 503 μmol) and NMI (41 mg, 503 μmol) in ACN (2 mL) was stirred for 1 hour at room temperature under nitrogen atmosphere. The resulting mixture was concentrated under vacuum. The residue was purified by reverse flash chromatography with the following conditions: column, C18 silica gel; mobile phase, MeCN in water (0.1% FA), 10% to 50% gradient in 10 min; detector, UV 254 nm. This resulted in 5-(3,5-difluoropyridin-2-yl)-N—(3-fluoro-5-methanesulfonamidophenyl)-1-methyl-1H-pyrrole-3-carboxamide (135.6 mg, 321 μmol) as a light yellow solid. LCMS (ESI) [M+H]⁺: 407.10. ¹H NMR (400 MHz, DMSO-d₆) δ 9.74 (d, J=8.4 Hz, 2H), 8.59 (d, J=2.4 Hz, 1H), 8.08 (ddd, J=11.2, 8.9, 2.5 Hz, 1H), 7.75 (d, J=1.9 Hz, 1H), 7.69 (t, J=2.1 Hz, 1H), 7.52 (dd, J=7.8, 2.2 Hz, 1H), 7.26 (t, J=8.1 Hz, 1H), 7.21 (dd, J=3.7, 1.9 Hz, 1H), 6.92-6.85 (m, 1H), 3.88 (s, 3H), 3.00 (s, 3H).

### Assays

Compounds of the disclosure were assessed for their ability to inhibit DHX9 activity. The inhibitory properties of the compounds of the disclosure described herein can be evidenced by testing in any one of the following assays.

### 1. Biochemical Assay

### Materials and Methods

Recombinant DHX9—Human recombinant DHX9 protein was custom ordered from Viva.

RNA substrate—Oligos for double-stranded RNA substrate were custom ordered from IDT Technologies. (Oligo1—GCCUGGUCCCUGUCCUUGUUAUUUUCCUUGGUUAAUU (SEQ ID NO:1). Oligo2—GAAUUAACCAAGGAAAAUAACAAGGACAGGGACCAGG (SEQ ID NO:2)). Each oligo was reconstituted in RNAse/DNAse-free water to achieve a 100 uM solution. The two oligo solutions were mixed in 1:1 ratio and and annealed by heating at 70° C. for 5 minutes and cooling gradually to room temperature on the benchtop.

Chemicals and Assay Components—Aurintricarboxylic Acid and dimethylsulfoxide were purchased from Sigma-Aldrich (St. Louis, MO). White 384-well assay plates (Catalog #781075) were obtained from Greiner Bio-One (Frickenhausen, Germany). The ADP-Glo™ kinase assay kit from Promega Corporation is composed of ADP-Glo reagent, kinase detection reagent (made by mixing kinase detection buffer with a lyophilized kinase detection substrate), Ultra Pure ATP and ADP.

### ATPase Assay

DHX9 ATPase assay was performed in small-volume, nonbinding, 384-well white plates at a final volume of 10 μL/well. Test compounds (10 mM solution in DMSO; 100 nL/well) were serially diluted on Bravo (Agilent, Santa Clara, CA) and dispensed into wells of columns 3-22 of the plates using an Echo 555 acoustic dispenser (Labcyte, Sunnyvale, CA). 100 nL of Aurintricarboxylic acid was dispensed into low control wells and 100 nL of DMSO was dispensed into high control wells. Then, a Multidrop Combi Reagent Dispenser (Thermo Fisher Scientific, Waltham, MA) was used to add a solution of DHX9 (1.25 nM, 5 μL/well) in assay buffer (40 mM HEPES [pH 7.5], 0.01% Tween 20, 0.01% BSA, 1 mM DTT, 5 mM MgCl₂, 0.004 U/ml RNAseOUT). The reaction was initiated by the addition of 5 μL of substrate solution (30 nM double-stranded RNA substrate, 10 μM Ultra Pure ATP in assay buffer) into the wells. The plates were incubated at room temperature for 1 h. After the indicated incubation times, 10 μL ADP-Glo reagent was added to the reactions and the plate was incubated at room temperature for 40 min. Then, 20 μL of kinase detection reagent was added and after an incubation time of 40 min, luminescence was recorded on Envision plate reader (Perkin-Elmer, Billerica, MA).

### Data Processing and Analysis

All data was analyzed by Accent Therapeutics internal data analysis software developed by Scigilian (Montreal, Canada). The percentage inhibition was calculated based on the high control (DMSO) as 0% inhibition, and low control (10 μM Aurintricarboxylic acid) control as 100% inhibition and used for the calculation of IC₅₀ and IP (inflection point) values by fitting the dose-response curves to a four-parameter logistic model. Assay results reported IP values instead of IC₅₀ values since several compounds did not reach 100% inhibition at higher compound concentrations.

### 2. DHX9 Cellular Proliferation Assay

The CellTiter-Glo® Luminescent Cell Viability Assay is a homogeneous method to determine the number of viable cells in culture based on quantitation of the ATP present, which signals the presence of metabolically active cells.

The following cell lines were obtained from ATCC: HCT116 (CCL-247) and Colo205 (CCL-222). HCT116 cells were grown and assayed in McCoy's 5a growth media supplemented with 10% FBS, and Colo205 cells were grown in RPMI 1640 media supplemented with 10% FBS.

The cells are plated at pre-determined cell densities in 384-well solid white cell culture plates and incubated overnight in 37° C. at 5% CO₂. In a separate plate, reference and test compounds were prepared in DMSO stock solution and serially diluted 3-fold for 10 points. Compound (0.1% final DMSO concentration) was transferred to the cell plate in designated wells, and incubated 37° C. at 5% CO₂ for 120 hours. CellTiter-Glo reagent was prepared fresh according to manufacturer's (Promega) directions and added to each well. The plate is then shaken at 300 rpm for 10 minutes at RT, and then read on an Envision plate reader using a Luminescence protocol.

Data analysis was performed by normalizing the raw data (raw luminescence units or R_{Sample}) to an average of the positive control values for wells containing culture media only (100% cell death or R_{L}C) and the negative control values for 0.1% DMSO (0% cell death or R_{HC}). An IC₅₀ was calculated using a 4-parameter logistic nonlinear regression model in Scigilian Analyzer or GraphPad Prism, constraining the top parameter to 100 and the bottom parameter to 0.

$$\% Inhibition = 100 \% \times ( 1 - \frac{{R}_{Sample} - {R}_{L C}}{{R}_{HC} - {R}_{L C}} )$$

IC₅₀ Fit formula=(A+((B−A)/(1+((x/C)\hat{ }D))))
whereas A: Bottom; B: Top; C: IC₅₀; D: Slope
   3. DHX9 circBRIP1 Cellular Target Engagement SYBR Green Assay

Evaluation of the circular and linear format of BRIP1 mRNA in HCT116 cells by Quantitative Polymerase Chain Reaction (qPCR). Upon DHX9 inhibition circBRIP1 levels are increased and linear BRIP1 mildly decreases or remains unchanged.

HCT116 cells were obtained from ATCC (CCL-247) and were grown and assayed in McCoy's 5a growth media supplemented with 10% FBS.

The cells are plated at pre-determined cell densities in 384-well cell culture plates and incubated overnight in 37° C. at 5% CO₂. In a separate plate, reference and test compounds were prepared in DMSO stock solution and serially diluted 3-fold for 10 points. Compound (0.1% final DMSO concentration) was transferred to the cell plate in designated wells, and incubated 37° C. at 5% CO₂ for 72 hours.

RNA is extracted from the cells using the SYBR Green Fast Advanced Cell-to-CT kit according to manufacturer's (Invitrogen) protocol, and cDNA is reverse transcribed using the High Capacity RNA-to-cDNA Kit according to the manufacturer's (Invitrogen) protocol. Using SYBR Green Master Mix and thermal cycler conditions for the QuantStudio Thermocycler, qPCR is carried out using the below custom primer sequences:

BRIP1-Circular-Forward
(TCT GTG TGC CAG ACT GTG AG (SEQ ID NO: 3))
BRIP1-Circular-Reverse
(ACA CCA AGT TCT GAC GAA AAG G (SEQ ID NO: 4))
BRIP1-Linear-Forward
(GTC CAT CCT GAG GTA GTC GG (SEQ ID NO: 5))
BRIP1-Linear-Reverse
(TTC CCC AGG CTG ACA AGT TC (SEQ ID NO: 6)
GAPDH-Forward
(GTCTCCTCTGACTTCAACAGCG (SEQ ID NO: 7))
GAPDH-Reverse
(ACCACCCTGTTGCTGTAGCCAA (SEQ ID NO: 8))

Note: Forward and Reverse primers are combined per gene, and each sample has to be run on a separate plate for each gene.

Data analysis was performed by using the QuantStudio Thermocycler software to determine threshold Ct values. ΔCt for circBRIP1 or linBRIP1 is calculated by normalizing the target gene Ct to the GAPDH housekeeping gene Ct for each sample. The inhibition activity is calculated by the following formula, whereas:
ΔCts: Mean of Ct(target gene)—Mean of Ct(GAPDH) for each sample
ΔCtc: Mean of Ct(target gene)—Mean of Ct(GAPDH) for DMSO control.

$$\% Inhibition = 100 \% \times \frac{{2}^{Δ Cts}}{{2}^{Δ Ctc}}$$

An EC₅₀ was then calculated using a 4-parameter logistic nonlinear regression model in Scigilian Analyzer or GraphPad Prism, floating both the top and bottom parameters.

EC₅₀ Fit formula=(A+((B−A)/(1+((x/C)\hat{ }D))))
whereas A: Bottom; B: Top; C: IC₅₀; D: Slope
   4. DHX9 circBRIP1 Cellular Target Engagement TaqMan Multiplex Assay:

Evaluation of the circular and linear format of BRIP1 mRNA in HCT116 cells by Quantitative Polymerase Chain Reaction (qPCR). Upon DHX9 inhibition circBRIP1 levels are increased and linear BRIP1 mildly decreases or remains unchanged.

HCT116 cells were obtained from ATCC (CCL-247) and were grown and assayed in McCoy's 5a growth media supplemented with 10% FBS.

The cells are plated at pre-determined cell densities in 384-well cell culture plates and incubated overnight in 37° C. at 5% CO₂. In a separate plate, reference and test compounds were prepared in DMSO stock solution and serially diluted 3-fold for 10 points. Compound (0.1% final DMSO concentration) was transferred to the cell plate in designated wells, and incubated 37° C. at 5% CO₂ for 72 hours.

RNA is extracted from the cells using the TaqMan Gene Expression Cells-to-CT™ kit according to manufacturer's (Invitrogen) protocol, and cDNA is reverse transcribed using the High Capacity RNA-to-cDNA Kit according to the manufacturer's (Invitrogen) protocol. Using TaqMan Multiplex Mastermix and thermal cycler conditions for the QuantStudio Thermocycler, qPCR is carried out using TaqMan Gene Expression Assay with VIC Dye for Linear BRIP1 (Assay ID: Hs00908156_m1), TaqMan GAPDH primer limited assay with JUN dye/QSY probe, and the below custom TaqMan Gene Expression Assay with FAM Dye for circBRIP1:

Forward Primer:
(SEQ ID NO: 9)
GTGAGCCAAGGAATTTTGTGTTT
Reverse Primer:
(SEQ ID NO: 10)
GGTCTGAACTTTGCCATTAATATCTG
Probe:
(SEQ ID NO: 11)
CCATCTTACAAGGCCTTT

Note: All target genes can be combined in one well per sample.

Data analysis was performed by using the QuantStudio Thermocycler software to determine threshold Ct values. ΔCt for circBRIP1 or linBRIP1 is calculated by normalizing the target gene Ct to the GAPDH housekeeping gene Ct for each sample. The inhibition activity is calculated by the following formula, whereas:
ΔCts: Mean of Ct(target gene)—Mean of Ct(GAPDH) for each sample
ΔCtc: Mean of Ct(target gene)—Mean of Ct(GAPDH) for DMSO control.

$$\% Inhibition = 100 \% \times \frac{{2}^{Δ Cts}}{{2}^{Δ Ctc}}$$

An EC₅₀ was then calculated using a 4-parameter logistic nonlinear regression model in Scigilian Analyzer or GraphPad Prism, floating both the top and bottom parameters.

IC50 Fit formula=(A+((B−A)/(1+((x/C)\hat{ }D))))
whereas A: Bottom; B: Top; C: IC50; D: Slope

### 5. DHX9 Cellular Proliferation Assay

The CellTiter-Glo® Luminescent Cell Viability Assay is a homogeneous method to determine the number of viable cells in culture based on quantitation of the ATP present, which signals the presence of metabolically active cells.

The following cell lines were obtained from ATCC: LS411N (CRL-2159) and NCI-H747 (CCL-252). LS411N and NCI-H747 cells were grown in RPMI-1640 media supplemented with 10% FBS. The cells are plated at pre-determined cell densities in 384-well solid white cell culture plates and incubated overnight in 37° C. at 5% CO₂. In a separate plate, reference and test compounds were prepared in DMSO stock solution and serially diluted 3-fold for 10 points. Compound (0.1% final DMSO concentration) was transferred to the cell plate in designated wells, and incubated 37° C. at 5% CO₂ for 120 hours. CellTiter-Glo reagent was prepared fresh according to manufacturer's (Promega) directions and added to each well. The plate is then shaken at 300 rpm for 10 minutes at RT, and then read on an Envision plate reader using a Luminescence protocol. Data analysis was performed by normalizing the raw data (raw luminescence units or RSample) to an average of the positive control values for wells containing culture media only (100% cell death or RLC) and the negative control values for 0.1% DMSO (0% cell death or RHC). An IC₅₀ was calculated using a 4-parameter logistic nonlinear regression model in Scigilian Analyzer or GraphPad Prism, constraining the top parameter to 100 and the bottom parameter to 0.

$$\% Inhibition = 100 \% \times ( 1 - \frac{{R}_{Sample} - {R}_{L C}}{{R}_{HC} - {R}_{L C}} )$$

IC50 Fit formula=(A+((B−A)/(1+((x/C)\hat{ }D))))
whereas A: Bottom; B: Top; C: IC₅₀; D: Slope

### Data for Examples

| | DHX9 | DHX9 | DHX9 | LS411N | H747 |
|---|---|---|---|---|---|
| | Bio- | Bio- | Cellular | (CRC- | (CRC- |
| | chemical | chemical | Target | MSI-H) | MSS) |
| | Inflection | Max | Engagement | Antiprolif- | Antiprolif- |
| Example | Point | Inhibition | EC₅₀ | erative | erative |
| Number | (μM) | (%) | (μM) | IC₅₀ | IC₅₀ |
| 1 | 0.339 | 71.6 | >30 | | |
| 2 | 7.27 | 50.4 | | | |
| 3 | 1.23 | 44.3 | | | |
| 4 | 3.05 | 55.1 | | | |
| 5 | 2.97 | 37.7 | | | |
| 6 | 0.0824 | 79.7 | 16.1 | | |
| 7 | 0.804 | 73.3 | | | |
| 8 | 1.76 | 47.7 | | | |
| 9 | 3.94 | 48.3 | | | |
| 10 | 1.54 | 79.8 | | | |
| 11 | 5.65 | 47.5 | | | |
| 12 | 0.355 | 83.3 | >30 | | |
| 13 | 2.39 | 52.5 | | | |
| 14 | 5.09 | 70.4 | | | |
| 15 | 0.579 | 70.4 | >30 | | |
| 16 | 3.62 | 65.3 | | | |
| 17 | 2.54 | 50.7 | | | |
| 18 | 92.4 | 58.6 | | | |
| 19 | 6.33 | 49.6 | | | |
| 20 | 0.664 | 72 | >30 | | |
| 21 | 3.85 | 72.4 | | | |
| 22 | 1.22 | 71.7 | | | |
| 23 | 0.943 | 51.4 | | | |
| 24 | 1.04 | 62.5 | | | |
| 25 | 2.32 | 67.1 | | | |
| 26 | 56.9 | 60.6 | | | |
| 27 | 1.61 | 63.8 | | | |
| 28 | 4.08 | 76.4 | | | |
| 29 | 1.43 | 81.3 | | | |
| 30 | 34.1 | 69.1 | | | |
| 31 | 0.864 | 80.3 | >30 | | |
| 32 | 5.77 | 67.9 | | | |
| 33 | 1.02 | 65.4 | >30 | | |
| 34 | 4.85 | 70.4 | | | |
| 35 | 1.49 | 69.3 | | | |
| 36 | 1.86 | 56.3 | | | |
| 37 | 1.72 | 67 | | | |
| 38 | 0.345 | 38.6 | | | |
| 39 | 0.994 | 55.3 | | | |
| 40 | 0.371 | 72.8 | >30 | | |
| 41 | 0.285 | 61.2 | 27.4 | | |
| 42 | 0.544 | 71.4 | | | |
| 43 | 0.373 | 68.7 | | | |
| 44 | 6.51 | 47.2 | >30 | | |
| 45 | 0.0989 | 68.7 | 12.8 | | |
| 46 | 0.123 | 73.9 | | | |
| 47 | 0.264 | 67.7 | | | |
| 48 | 0.105 | 67.5 | | | |
| 49 | 0.0406 | 78.4 | 9.04 | | |
| 50 | 0.0458 | 66.5 | >30 | | |
| 51 | 0.335 | 77.3 | 13.4 | | |
| 52 | 0.111 | 68.5 | 20.7 | | |
| 53 | 0.188 | 70.4 | 22.7 | | |
| 54 | 0.0907 | 72.8 | 6.32 | | |
| 55 | 0.0461 | 69.4 | 6.32 | | |
| 56 | 0.148 | 68.2 | 21.4 | | |
| 57 | 0.0419 | 70.7 | 25.3 | | |
| 58 | 0.0558 | 69.2 | 3.12 | | |
| 59 | 0.0543 | 70.8 | 8.13 | | |
| 60 | 0.0766 | 69.2 | 10.9 | | |
| 61 | 0.0746 | 68.7 | 15.1 | | |
| 62 | 0.271 | 60.8 | >30 | | |
| 63 | 0.121 | 66.9 | 18.8 | | |
| 64 | 0.589 | 78.1 | | | |
| 65 | 0.0418 | 66.9 | 8.56 | | |
| 66 | 0.0875 | 71.3 | >30 | | |
| 67 | 0.98 | 70.1 | | | |
| 68 | 0.108 | 70.3 | 13.2 | | |
| 69 | 0.0163 | 75.6 | 4.79 | | |
| 70 | 0.0322 | 71.2 | 4.37 | | |
| 71 | 1.86 | 66.5 | | | |
| 72 | 0.494 | 64.4 | >30 | | |
| 73 | 0.109 | 63.7 | 6.15 | | |
| 74 | 0.0806 | 71.4 | 30 | | |
| 75 | 0.0821 | 70.1 | 8.28 | | |
| 76 | 0.0424 | 75.4 | 6.67 | | |
| 77 | 0.0894 | 72.3 | 2.04 | | |
| 78 | 0.698 | 61.5 | | | |
| 79 | 0.467 | 71.2 | >30 | | |
| 80 | 0.0041 | 80.5 | 1.29 | | |
| 81 | 1.07 | 68.3 | >30 | | |
| 82 | 0.0203 | 79 | 2.11 | | |
| 83 | 0.0173 | 73.1 | 0.917 | | |
| 84 | 0.0125 | 71.4 | 4.84 | | |
| 85 | 0.0195 | 70.7 | 1.39 | | |
| 86 | 0.0453 | 71.1 | 19.5 | | |
| 87 | 0.0226 | 77.2 | 30 | | |
| 88 | 0.0654 | 68.2 | 10.5 | | |
| 89 | 0.0097 | 76.4 | 1.03 | | |
| 90 | 19 | 61.2 | >30 | | |
| 91 | 0.0072 | 74.6 | 7.63 | | |
| 92 | 0.0554 | 75.5 | >30 | | |
| 93 | 0.0399 | 74 | 11.5 | | |
| 94 | 0.125 | 71.2 | >30 | | |
| 95 | 0.0076 | 73.1 | >30 | | |
| 96 | 0.0899 | 59.6 | 22.2 | | |
| 97 | 0.0247 | 70 | 4.32 | | |
| 98 | 0.543 | 77.7 | | | |
| 99 | 0.716 | 35.8 | >30 | | |
| 100 | 0.333 | 65.8 | 28.5 | | |
| 101 | 0.054 | 79.1 | | | |
| 102 | 0.159 | 72.5 | 18.9 | | |
| 103 | 0.126 | 71.7 | | | |
| 104 | 0.504 | 67.3 | | | |
| 105 | 0.21 | 70.3 | | | |
| 106 | 0.0679 | 72.3 | | | |
| 107 | 0.168 | 74.6 | | | |
| 108 | 0.708 | 64.4 | | | |
| 109 | 0.221 | 59 | | | |
| 110 | 0.0819 | 68.9 | | | |
| 111 | 0.0696 | 68.5 | | | |
| 112 | 0.101 | 68.9 | | | |
| 113 | 0.515 | 59.7 | | | |
| 114 | 0.0714 | 73.5 | | | |
| 115 | 0.436 | 75 | | | |
| 116 | 0.0708 | 74.5 | | | |
| 117 | 0.686 | 52.7 | | | |
| 118 | 0.0994 | 66.2 | | | |
| 119 | 0.131 | 67.4 | | | |
| 120 | 0.0505 | 70.4 | | | |
| 121 | 1.07 | 68.9 | | | |
| 122 | 0.196 | 69.7 | | | |
| 123 | 0.0592 | 68.3 | | | |
| 124 | 0.182 | 66.5 | | | |
| 125 | 0.0649 | 71.7 | | | |
| 126 | 0.502 | 67 | | | |
| 127 | 0.115 | 68.3 | | | |
| 128 | 0.146 | 64.6 | | | |
| 129 | 0.0889 | 67.2 | | | |
| 130 | 0.324 | 69.2 | | | |
| 131 | 0.0522 | 65.8 | | | |
| 132 | 0.0738 | 70 | | | |
| 133 | 0.171 | 70.3 | | | |
| 134 | 0.058 | 66.9 | | | |
| 135 | 0.211 | 65.6 | | | |
| 136 | 0.127 | 72.9 | | | |
| 137 | 0.547 | 67.5 | | | |
| 138 | 0.116 | 76.6 | | | |
| 139 | 0.0405 | 68.9 | | | |
| 140 | 0.189 | 70.1 | | | |
| 141 | 0.0926 | 66.3 | | | |
| 142 | 0.167 | 72.6 | | | |
| 143 | 0.0915 | 73.3 | | | |
| 144 | 0.146 | 65.9 | | | |
| 145 | 0.105 | 67.7 | | | |
| 146 | 0.0387 | 71.2 | | | |
| 147 | 0.229 | 64.7 | | | |
| 148 | 5.36 | 58.2 | | | |
| 149 | 0.0507 | 69.8 | | | |
| 150 | 0.102 | 68.6 | | | |
| 151 | 0.0459 | 70.1 | | | |
| 152 | 0.123 | 69.7 | 16.6 | | |
| 153 | 0.0432 | 77.9 | 5.91 | | |
| 154 | 3.89 | 68.4 | | | |
| 155 | 1.01 | 72.3 | | | |
| 156 | 1.92 | 63.7 | | | |
| 157 | 0.205 | 68.6 | >30 | | |
| 158 | 0.223 | 56.3 | 10.9 | | |
| 159 | 0.69 | 50.7 | >10 | | |
| 160 | 0.0284 | 89.6 | 1.41* | 9.4500 | >10 |
| 161 | 0.108 | 68.8 | 3.4* | | |
| 162 | 0.0811 | 68.5 | | | |

*Compounds of examples 160 and 161 were tested using TagMan Multiplex Assay; the rest of the compounds were tested using SYBR Green Assay.

1. A compound represented by Formula (I): 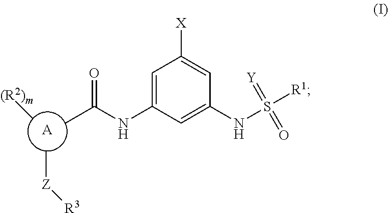
or a pharmaceutically acceptable salt thereof, wherein:
X is H, C₁₋₄alkyl, C₂₋₄haloalkyl, or C(O)R^{x};
R^{x} is H, —OH, —OC₁₋₄alkyl, or C₁₋₄alkyl;
Y is O or NR^{y};
R^{y} is H or C₁₋₄alkyl;
R¹ is C₁₋₄alkyl or C₃₋₆cycloalkyl; wherein the C₁₋₄alkyl or C₃₋₆cycloalkyl are each optionally and independently substituted with 1 to 3 halo;
Ring A is a C₃₋₆cycloalkyl, 4 to 6-membered monocyclic heterocyclyl, 6 to 10 membered bicyclic heterocyclyl, phenyl, 5 to 6-membered monocyclic heteroaryl, or 8 to 10-membered bicyclic heteroaryl;
m is 0 to 3;
each R² is independently selected from C₁₋₄alkyl, C₃₋₆cycloalkyl, halo, OR^{2a}, cyano, —NR^{2b}R^{2c}, —SO₂R^{2a}, —C(O)R^{2d}, and —C(O)NR^{2b}R^{2c}, wherein the C₁₋₄alkyl and C₃₋₆cycloalkyl are optionally substituted with 1 to 4 R^{2e}; or 2 R² together form oxo;
R^{2a} is H, C₁₋₄alkyl, C₁₋₄haloalkyl, C₁₋₄alkoxyC₁₋₄alkyl, or benzyl;
R^{2d} is H, C₁₋₄alkyl, C₃₋₆cycloalkyl; OR^{2a}, phenyl, 4 to 6-membered monocyclic heterocyclyl or 5 to 6-membered monocyclic heteroaryl, wherein the C₁₋₄alkyl is optionally substituted with 1 to 3 halo;
R^{2b} and R^{2e} are each independently selected from H, C₁₋₄alkyl, C₃₋₆cycloalkyl, phenyl, 4 to 6-membered monocyclic heterocyclyl and 5 to 6-membered monocyclic heteroaryl; or R^{2b} and R^{2c}, together with the nitrogen to which they are attached form a 4 to 6 membered monocyclic heterocyclyl or 6 to 10 membered bicyclic heterocyclyl; wherein the 4 to 6 membered monocyclic heterocyclyl or 6 to 10 membered bicyclic heterocyclyl are each optionally substituted with 1 to 3 R^{2d};
each R^{2e} is independently selected from halo, cyano, NR^{2b}R^{2c}, OR^{2a}, phenyl, and 4 to 6 membered monocyclic heterocyclyl;
Z is a bond, —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³;
Z^{a} is H or C₁₋₄alkyl;
provided when X is H and Z is a bond, ring A is phenyl, pyridinyl, thiophenyl, pyrrolyl, pyrazolyl, thiazolyl, imidazolyl, furanyl, or 1,4,5,6-tetrahydrocyclopenta[c]pyrazolyl;
R³ is C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl, wherein the C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl are each optionally and independently substituted with 1 to 4 R⁴;
each R⁴ is independently selected from C₁₋₄alkyl, halo, OR^{4a}, cyano, —NR^{4b}R^{4c}, —C(O)R^{4a}, —C(O)OR^{4a}, —C(O)NR^{4b}R^{4c}, —NR^{4b}C(O)R^{4a}—NR^{4b}C(O)OR^{4a}, —SR^{4a}, —SO₂R^{4a}, —SO₂NR^{4b}R^{4c}, —P(O)R^{4b}R^{4c}, phenyl, 5 to 6-membered monocyclic heteroaryl, 4 to 6-membered monocyclic heterocyclyl, and 6 to 10-membered bicyclic heterocyclyl, wherein the C₁₋₄alkyl is optionally substituted with 1 to 4 R^{4d}, and wherein the phenyl, 5 to 6-membered monocyclic heteroaryl, 4 to 6-membered monocyclic heterocyclyl and 6 to 10-membered bicyclic heterocyclyl are each optionally substituted with 1 to 3 R^{4e}; or two R⁴ together form oxo;
R^{4a} is H, C₁₋₄alkyl optionally substituted with 1 to 4 R^{4d}, C₃₋₆cycloalkyl, 4 to 6-membered monocyclic heterocyclyl, phenyl, or 5 to 6-membered monocyclic heteroaryl; wherein the C₃₋₆cycloalkyl, 4 to 6-membered monocyclic heterocyclyl, phenyl or 5 to 6-membered monocyclic heteroaryl are each optionally and independently substituted with 1 to 3 R^{4e};
R^{4b} and R^{4c} are each independently selected from H, phenyl, 4 to 6-membered monocyclic heterocyclyl, 5 to 6-membered monocyclic heteroaryl, and C₁₋₄alkyl optionally substituted with 1 to 4 R^{4d};
each R^{4d} is independently selected from halo, OR^{4f}, —C(O)C₁₋₄alkyl, —C(O)NR^{4b}R^{4c}, —C(O)C₁₋₄haloalkyl, phenyl, 4- to 6-membered monocyclic heterocyclyl, and 5 to 6-membered monocyclic heteroaryl, wherein the phenyl and 5 to 6-membered monocyclic heteroaryl are each optionally substituted with 1 to 3 R^{4g};
each R^{4e} is independently selected from halo, C₁₋₄alkyl optionally substituted with 1 to 3 halo, cyano, OR^{4f}, —NR^{4b}R^{4c}, —C(O)H, —C(O)C₁₋₄alkyl, —C₁₋₄alkylene-C(O)NR^{4b}R^{4c}, and —C(O)NR^{4b}R^{4c}; or two R^{4e} together form oxo;
R^{4f} is H, C₁₋₄alkyl, C₁₋₄haloalkyl, phenyl or 5 to 6-membered monocyclic heteroaryl, wherein the phenyl and 5 to 6-membered monocyclic heteroaryl are each optionally substituted with one to three halo;
each R^{4g} is independently selected from halo, OR^{4f}, C₁₋₄alkyl, halo, cyano, —NR^{4b}R^{4c}, —C(O)H, and —C(O)OR⁴f;
provided that the compound is not any one of the compounds shown in Table I.

2. The compound of claim 1, wherein the compound is represented by Formula (I): 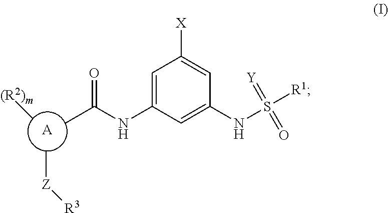
or a pharmaceutically acceptable salt thereof, wherein:
X is H, C₁₋₄alkyl, C₂₋₄haloalkyl, or C(O)R^{x};
R^{x} is H, —OH, —OC₁₋₄alkyl, or C₁₋₄alkyl;
Y is O or NR^{y};
R^{y} is H or C₁₋₄alkyl;
R¹ is C₁₋₄alkyl or C₃₋₆cycloalkyl; wherein the C₁₋₄alkyl or C₃₋₆cycloalkyl are each optionally and independently substituted with 1 to 3 halo;
Ring A is a C₃₋₆cycloalkyl, 4 to 6-membered monocyclic heterocyclyl, 6 to 10 membered bicyclic heterocyclyl, phenyl, 5 to 6-membered monocyclic heteroaryl, or 8 to 10-membered bicyclic heteroaryl;
m is 0 to 3;
each R² is independently selected from C₁₋₄alkyl, C₃₋₆cycloalkyl, halo, OR^{2a}, cyano, —NR^{2b}R^{2c}, —SO₂R^{2a}, —C(O)R^{2d}, and —C(O)NR^{2b}R^{2c}, wherein the C₁₋₄alkyl and C₃₋₆cycloalkyl are optionally substituted with 1 to 4 R^{2e}; or 2 R² together form oxo;
R^{2a} is H, C₁₋₄alkyl, C₁₋₄haloalkyl, C₁₋₄alkoxyC₁₋₄alkyl, or benzyl;
R^{2d} is H, C₁₋₄alkyl, C₃₋₆cycloalkyl; OR^{2a}, phenyl, 4 to 6-membered monocyclic heterocyclyl or 5 to 6-membered monocyclic heteroaryl, wherein the C₁₋₄alkyl is optionally substituted with 1 to 3 halo;
R^{2b} and R^{2c} are each independently selected from H, C₁₋₄alkyl, C₃₋₆cycloalkyl, phenyl, 4 to 6-membered monocyclic heterocyclyl and 5 to 6-membered monocyclic heteroaryl; or R^{2b} and R^{2c}, together with the nitrogen to which they are attached form a 4 to 6 membered monocyclic heterocyclyl or 6 to 10 membered bicyclic heterocyclyl; wherein the 4 to 6 membered monocyclic heterocyclyl or 6 to 10 membered bicyclic heterocyclyl are each optionally substituted with 1 to 3 R^{2d};
each R^{2e} is independently selected from halo, cyano, NR^{2b}R^{2c}, OR^{2a}, phenyl, and 4 to 6 membered monocyclic heterocyclyl;
Z is a bond, —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³;
Z^{a} is H or C₁₋₄alkyl;
provided when X is H and Z is a bond, ring A is phenyl, pyridinyl, thiophenyl, pyrrolyl, pyrazolyl, thiazolyl, imidazolyl, furanyl, or 1,4,5,6-tetrahydrocyclopenta[c]pyrazolyl;
R³ is C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl, wherein the C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl are each optionally and independently substituted with 1 to 4 R⁴;
each R⁴ is independently selected from C₁₋₄alkyl, halo, OR^{4a}, cyano, —NR^{4b}R^{4c}, —C(O)R^{4a}, —C(O)OR^{4a}, —C(O)NR^{4b}R^{4c}, —NR^{4b}C(O)R^{4a}—NR^{4b}C(O)OR^{4a}, —SR^{4a}, —SO₂R^{4a}, —SO₂NR^{4b}R^{4c}, —P(O)R^{4b}R^{4c}, phenyl, 5 to 6-membered monocyclic heteroaryl, 4 to 6-membered monocyclic heterocyclyl, and 6 to 10-membered bicyclic heterocyclyl, wherein the C₁₋₄alkyl is optionally substituted with 1 to 4 R^{4d}, and wherein the phenyl, 5 to 6-membered monocyclic heteroaryl, 4 to 6-membered monocyclic heterocyclyl and 6 to 10-membered bicyclic heterocyclyl are each optionally substituted with 1 to 3 R^{4e}; or two R⁴ together form oxo;
R^{4a} is H, C₁₋₄alkyl optionally substituted with 1 to 4 R^{4d}, C₃₋₆cycloalkyl, 4 to 6-membered monocyclic heterocyclyl, phenyl, or 5 to 6-membered monocyclic heteroaryl; wherein the C₃₋₆cycloalkyl, 4 to 6-membered monocyclic heterocyclyl, phenyl or 5 to 6-membered monocyclic heteroaryl are each optionally and independently substituted with 1 to 3 R^{4e};
R^{4b} and R^{4c} are each independently selected from H, phenyl, 4 to 6-membered monocyclic heterocyclyl, 5 to 6-membered monocyclic heteroaryl, and C₁₋₄alkyl optionally substituted with 1 to 4 R^{4d};
each R^{4d} is independently selected from halo, OR^{4f}, —C(O)C₁₋₄alkyl, —C(O)NR^{4b}R^{4c}, —C(O)C₁₋₄haloalkyl, phenyl, and 5 to 6-membered monocyclic heteroaryl, wherein the phenyl and 5 to 6-membered monocyclic heteroaryl are each optionally substituted with 1 to 3 R^{4g};
each R^{4e} is independently selected from halo, C₁₋₄alkyl optionally substituted with 1 to 3 halo, cyano, OR^{4f}, —NR^{4b}R^{4c}, —C(O)H, —C(O)C₁₋₄alkyl, —C₁₋₄alkylene-C(O)NR^{4b}R^{4c}, and —C(O)NR^{4b}R^{4c}; or two R^{4e} together form oxo;
R^{4f} is H, C₁₋₄alkyl, C₁₋₄haloalkyl, phenyl or 5 to 6-membered monocyclic heteroaryl, wherein the phenyl and 5 to 6-membered monocyclic heteroaryl are each optionally substituted with one to three halo;
each R^{4g} is independently selected from halo, OR^{4f}, C₁₋₄alkyl, halo, cyano, —NR^{4b}R^{4c}, —C(O)H, and —C(O)OR⁴f;
provided that the compound is not any one of the compounds shown in Table I.

3. The compound of claim 1 or 2, wherein R¹ is —CH₃ or —CH₂CH₃.

4. The compound of any one of claims 1 to 3, or a pharmaceutically acceptable salt thereof, wherein X is H.

5. The compound of any one of claims 1 to 3, or a pharmaceutically acceptable salt thereof, wherein X is C₁₋₄alkyl, C₂₋₄haloalkyl, or C(O)R^{x}.

6. The compound of claim 5, or a pharmaceutically acceptable salt thereof, wherein X is —CH₃ or —C(O)H.

7. The compound of any one of claims 1 to 6, or a pharmaceutically acceptable salt thereof, wherein ring A is selected from phenyl, thiophenyl, pyrrolyl, pyrazoyl, furanyl, isothiazoyl, thiazoyl, imidazoyl, cyclobutyl, benzofuranyl, 2-oxo-2,3-dihydro-1H-benzo[d]imidazolyl, imidazo[1,2-a]pyridin-6-yl, 1,4,5,6-tetrahydrocyclopenta[c]pyrazolyl, 2-oxo-2,3-dihydro-1H-imidazolyl, indolizinyl, pyrrolo[1,2-a]pyrimidinyl, pyrrolo[1,2-c]pyrimidinyl, pyrrolo[1,2-a]pyrazinly, 5-oxo-5H-thiazolo[3,2-a]pyridinyl, thieno[3,2-b]pyridinyl, thieno[3,2-c]pyridinyl, thieno[2,3-c]pyridinyl, benzothiophenyl, thieno[3,2-d]pyrimidinyl, pyrrolo[1,2-b]pyridazinyl, pyrrolo[1,2-a]pyrazinyl, pyrrolo[1,2-a]pyrimidinyl, pyrrolo[1,2-b]pyridazinyl, pyrrolo[1,2-c]pyrimidinyl, 1-oxo-1,2-dihydropyrrolo[1,2-a]pyrazinyl, pyrrolo[2,1-f][1,2,4]triazinyl, 4,5,6,7-tetrahydrothieno[2,3-c]pyridinyl, 5,6-dihydro-4H-cyclopenta[b]thiophenyl, and 4,7-dihydro-5H-thieno[2,3-c]pyranyl.

8. The compound of claim 7, or a pharmaceutically acceptable salt thereof, wherein ring A is selected from phenyl, pyridinyl, thiophenyl, pyrrolyl, pyrazolyl, thiazolyl, imidazolyl, furanyl, and 1,4,5,6-tetrahydrocyclopenta[c]pyrazolyl.

9. The compound of any one of claims 1 to 8, or a pharmaceutically acceptable salt thereof, wherein ring A is represented by the following formula: 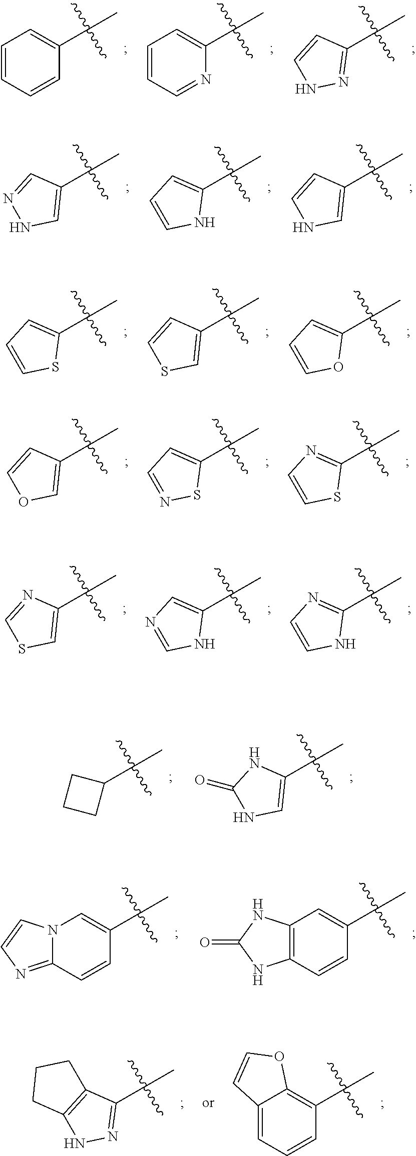
each of which is optionally substituted with 1 to 2 R².

10. The compound of claim 9, or a pharmaceutically acceptable salt thereof, wherein ring
A is represented by the following formula:

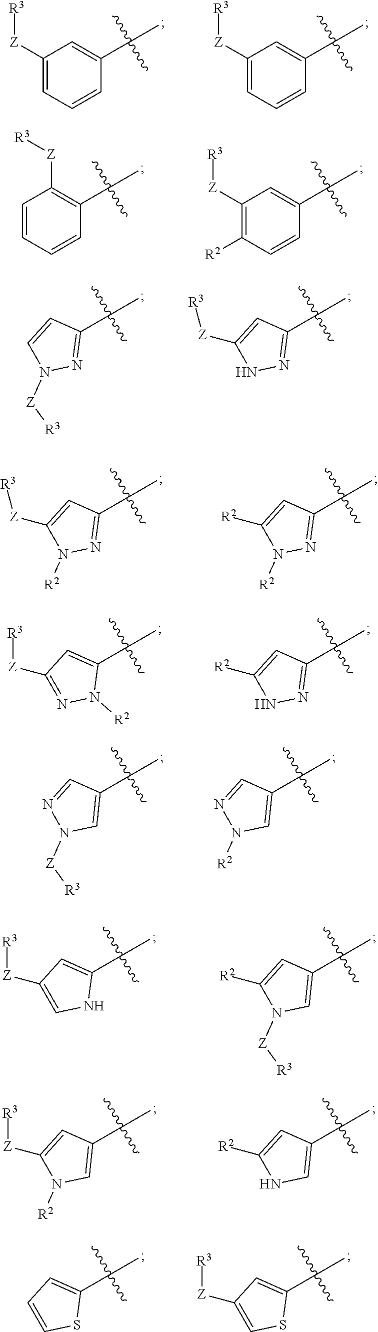

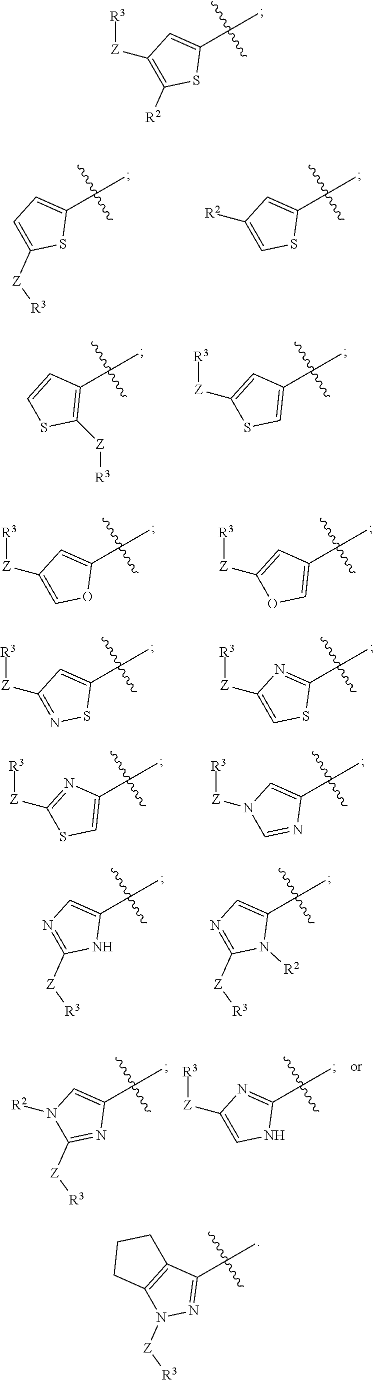

11. The compound of any one of claims 1 to 10, or a pharmaceutically acceptable salt thereof, wherein Z is a bond.

12. The compound of claim 7, or a pharmaceutically acceptable salt thereof, wherein ring A is selected from phenyl, thiophenyl, pyrazolyl, isothiazolyl, and imidazolyl.

13. The compound of claim 12, or a pharmaceutically acceptable salt thereof, wherein ring A is represented by the following formula: 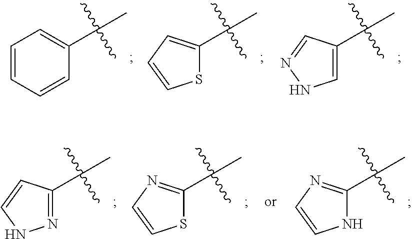 [SMILES: *c1ccccc1 ; *c1cccs1 ; *c1cn[nH]c1 ; *c1cc[nH]n1 ; *c1nccs1 ; *c1ncc[nH]1]
each of which is substituted with 0 to 1 R².

14. The compound of claim 13, or a pharmaceutically acceptable salt thereof, wherein Z is a bond and ring A is represented by the following formula:

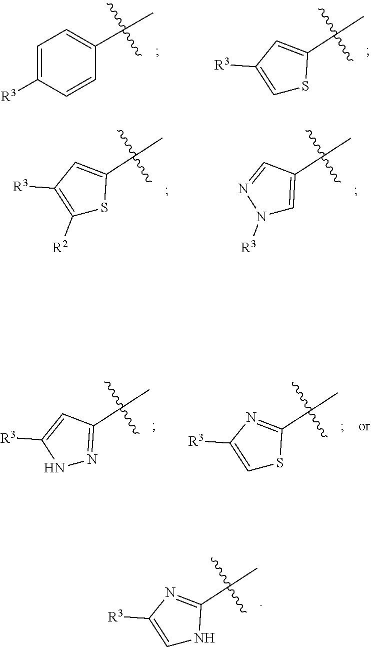

15. The compound of claim 13, or a pharmaceutically acceptable salt thereof, wherein Z is a bond and ring A is represented by the following formula:

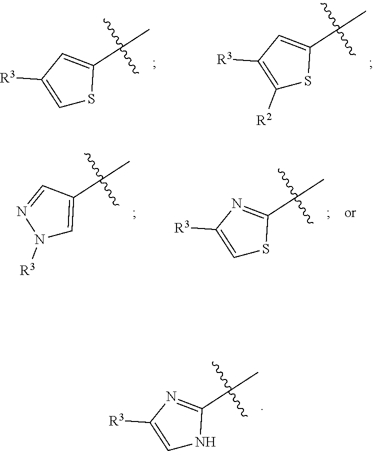

16. The compound of any one of claims 1 to 10, 12, and 13, or a pharmaceutically acceptable salt thereof, wherein Z is —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³.

17. The compound of any one of claims 1 to 10, 12, and 13, or a pharmaceutically acceptable salt thereof, wherein Z is —C(O)NH—* or —C(O)NH—C₁₋₃alkylene-*, wherein * indicates the attachment point to R³.

18. The compound of any one of claims 1 to 10, 12, and 13, or a pharmaceutically acceptable salt thereof, wherein Z is —C(O)NH—*, wherein * indicates the attachment point to R³.

19. The compound of any one of claims 16 to 18, or a pharmaceutically acceptable salt thereof, wherein ring A is represented by the following formula:

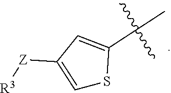

20. The compound of any one of claims 1 to 19, or a pharmaceutically acceptable salt thereof, wherein R² is C₁₋₄alkyl, halo, cyano, —C(O)R^{2d}, or —C(O)NR^{2b}R^{2c}, wherein the C₁₋₄alkyl is optionally substituted with 1 to 4 R^{2e}; R^{2b} and R^{2e} are each independently selected from H or C₁₋₄alkyl optionally substituted with C₁₋₃alkoxy; R^{2d} is OH or C₁₋₃alkoxy; and R^{2e} is halo, —OH or C₁₋₃alkoxy.

21. The compound of claim 20, wherein R² is —CH₃, —CF₃, —CH₂OH, halo, cyano, —C(O)OH, or —C(O)NHCH₃, or R² is represented by the following formula:

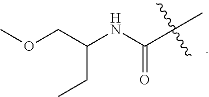 [SMILES: *C(=O)NC(CC)COC]

22. The compound of any one of claims 1 to 21, or a pharmaceutically acceptable salt thereof, wherein R³ is selected from cyclopropyl, cyclobutyl, cyclohexyl, piperidinyl, phenyl, pyrrolyl, pyrazolyl, pyridyl, pyrimidyl, pyrazinyl, pyrazinyl, thiophenyl, tetrahydropyranyl, thiazolyl, triazolyl, tetrazolyl, oxadiazolyl, 3-oxo-2,3-dihydro-1H-pyrazoly, benzamidazolyl, indazolyl, indoyl, 2,3-dihydrobenzofuranyl, imidazo[1,2-a]pyridinyl, 2,3-dihydro-1H-indenyl, 2-oxo-2,3-dihydro-1H-benzo[d]imidazolyl, and 2-azaspiro[3.3]heptanyl, and 2-oxaspiro[3.3]heptanyl, each of which is optionally substituted with 1 to 3 R⁴.

23. The compound of any one of claims 1 to 22, or a pharmaceutically acceptable salt thereof, wherein R³ is represented by the following formula: 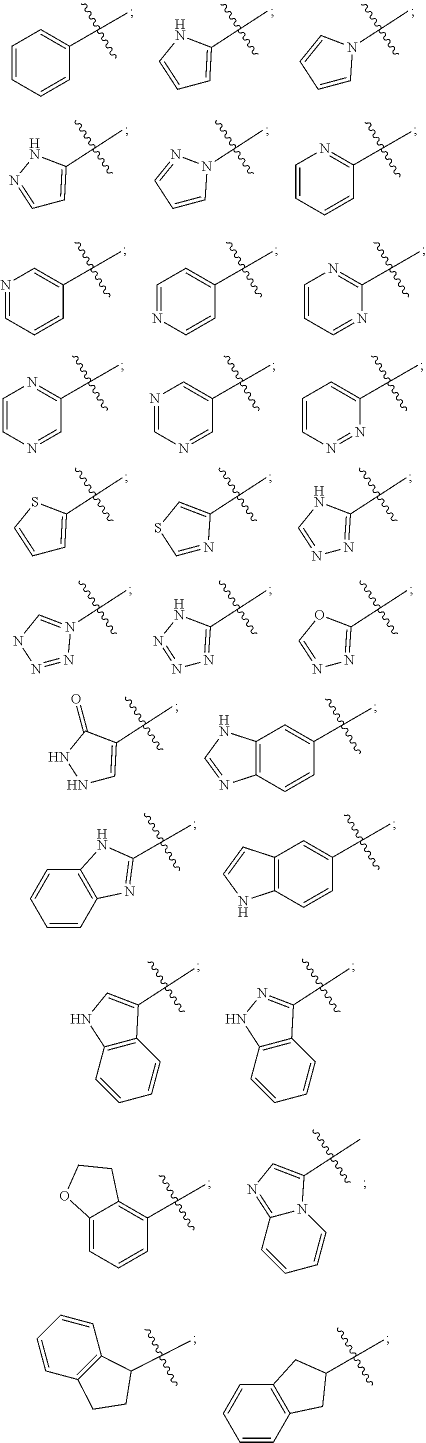 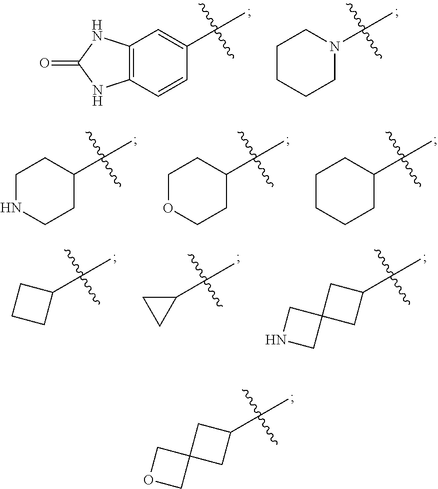
each of which is optionally substituted with 1 to 3 R⁴.

24. The compound of claim 23, or a pharmaceutically acceptable salt thereof, wherein R³ is represented by the following formula:

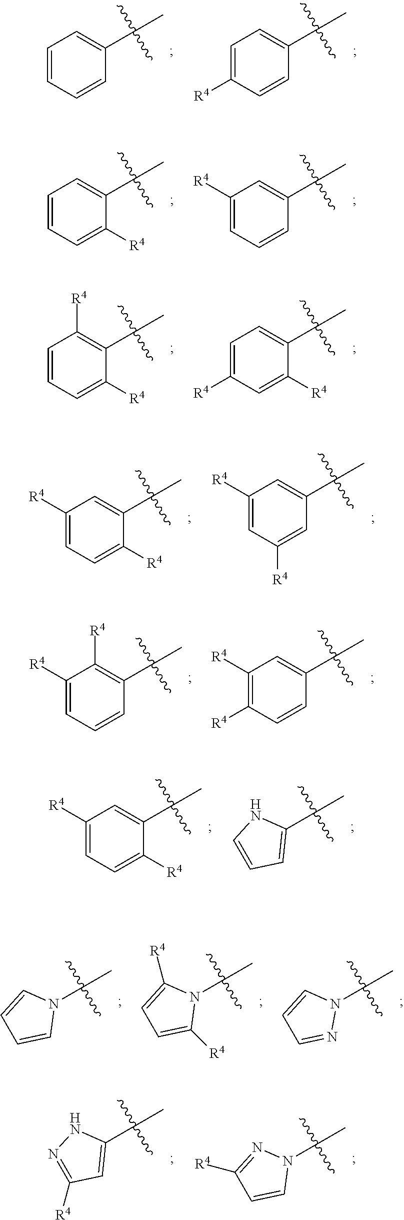

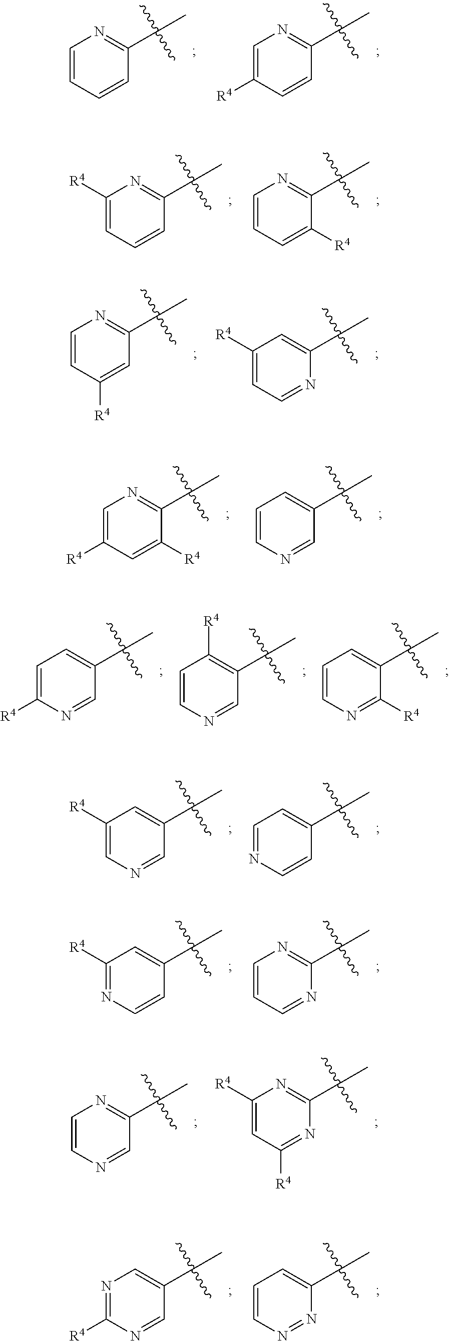

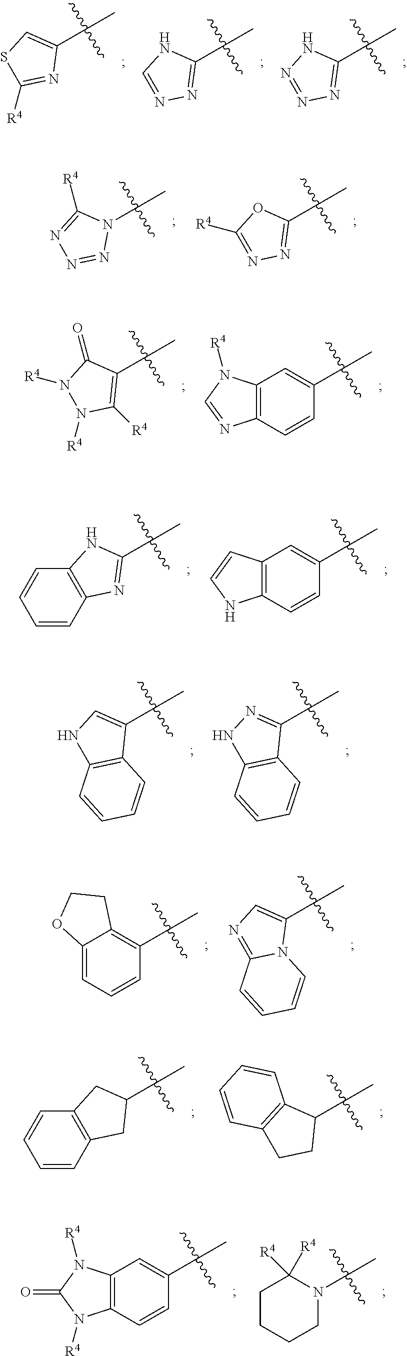

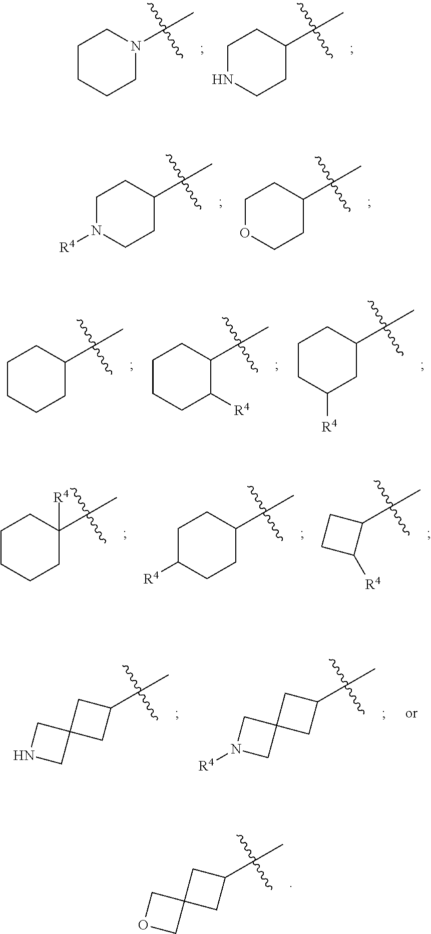

25. The compound of claim 22, or a pharmaceutically acceptable salt thereof, wherein Z is a bond, and R³ is selected from phenyl, pyridyl, pyrimidyl, pyrazinyl, thiophenyl, pyrrolyl, pyrazoyl, isothiazoyl, triazoyl, tetrazoyl, and oxadiazoyl, each of which is optionally substituted with 1 to 2 R⁴.

26. The compound of claim 25, or a pharmaceutically acceptable salt thereof, wherein R³ is represented by the following formula: 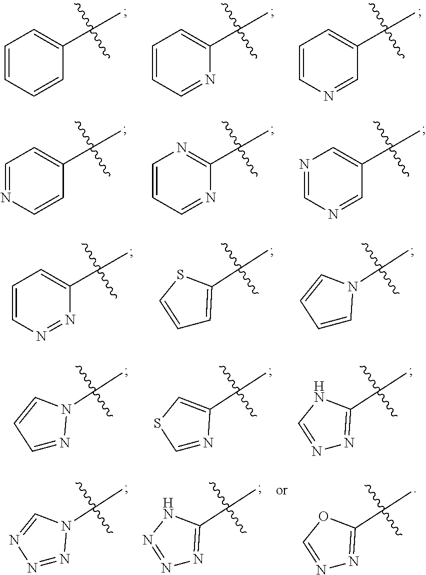
each of which is optionally substituted with 1 to 2 R⁴.

27. The compound of claim 26, or a pharmaceutically acceptable salt thereof, wherein R³ is represented by the following formula:

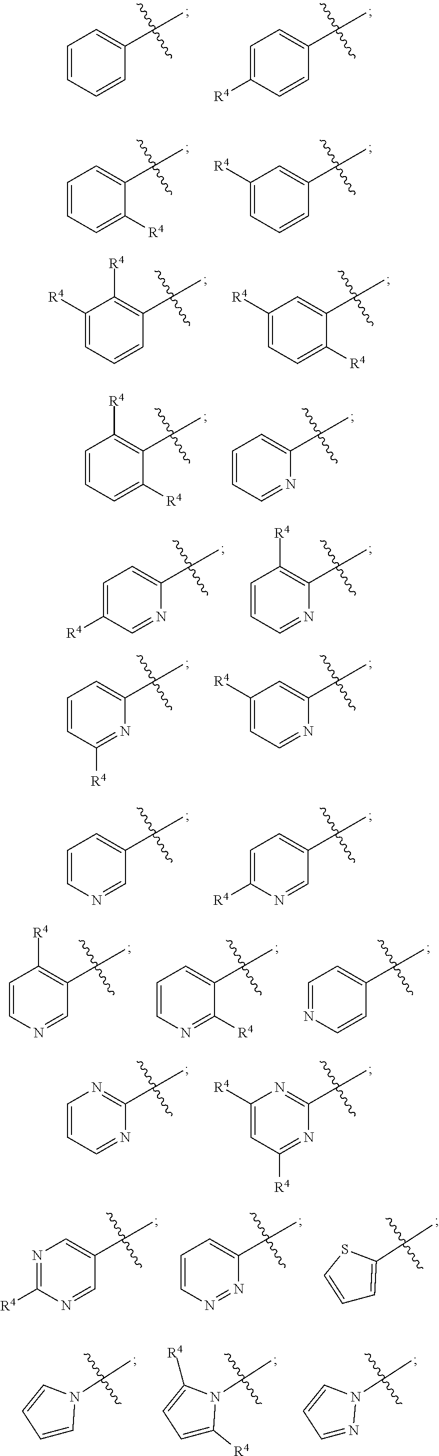

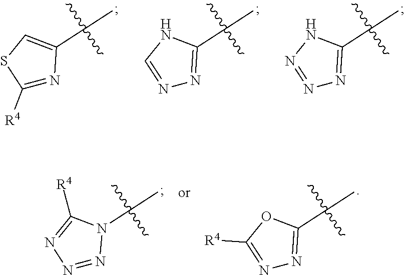

28. The compound of claim 25, or a pharmaceutically acceptable salt thereof, wherein R³ is selected from phenyl, pyridyl, and pyrimidyl, each of which is optionally substituted with 1 to 2 R⁴.

29. The compound of claim 28, or a pharmaceutically acceptable salt thereof, wherein R³ is represented by the following formula: 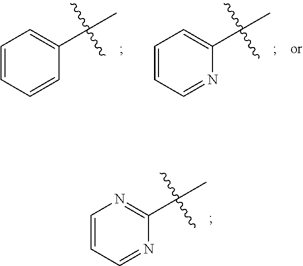 [SMILES: *c1ccccc1 ; *c1ccccn1 ; *c1ncccn1]
each of which is optionally substituted with 1 to 2 R⁴.

30. The compound of claim 29, or a pharmaceutically acceptable salt thereof, wherein R³ is represented by the following formula:

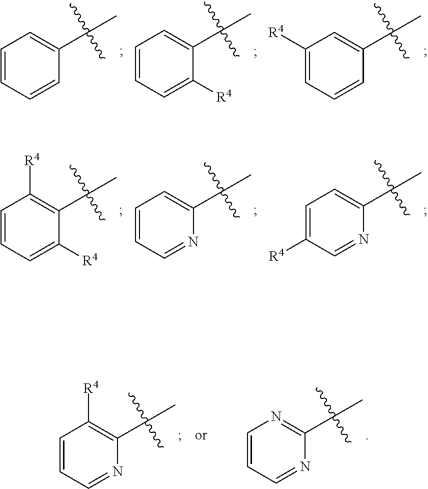

31. The compound of claim 22, or a pharmaceutically acceptable salt thereof, wherein Z is —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³, and wherein R³ is selected from cyclopropyl, cyclohexyl, piperidinyl, phenyl, pyridyl, pyrazoyl, isothiazoyl, 2-oxo-2,3-dihydro-1H-pyrazoly, benzamidazoyl, indazoyl, indoyl, 2,3-dihydrobenzofuranyl, imidazo[1,2-a]pyridinyl, 2,3-dihydro-1H-indenyl, and 2-oxo-2,3-dihydro-1H-benzo[d]imidazolyl, each of which is optionally substituted with 1 to 3 R⁴.

32. The compound of claim 31, or a pharmaceutically acceptable salt thereof, wherein R³ is represented by the following formula: 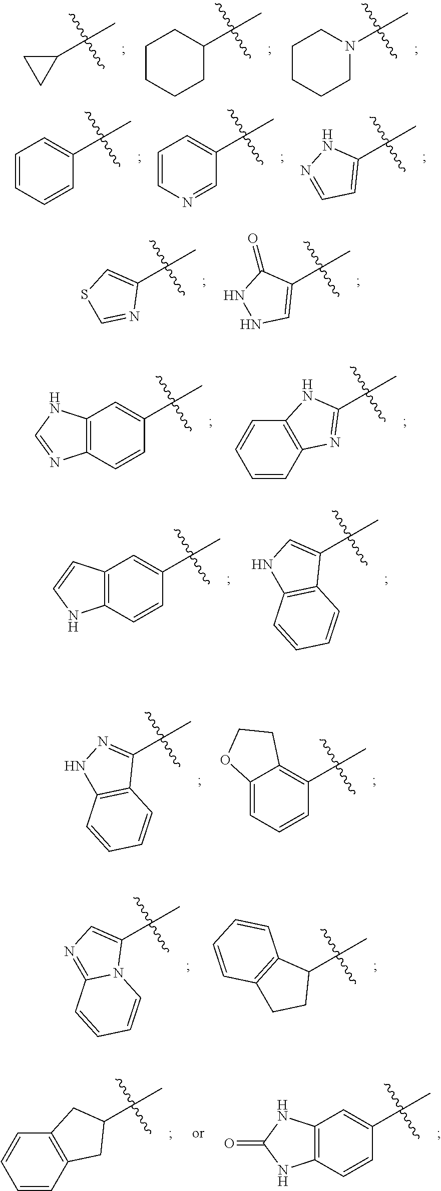
each of which is optionally substituted with 1 to 3 R⁴.

33. The compound of claim 32, or a pharmaceutically acceptable salt thereof, wherein R³ is represented by the following formula:

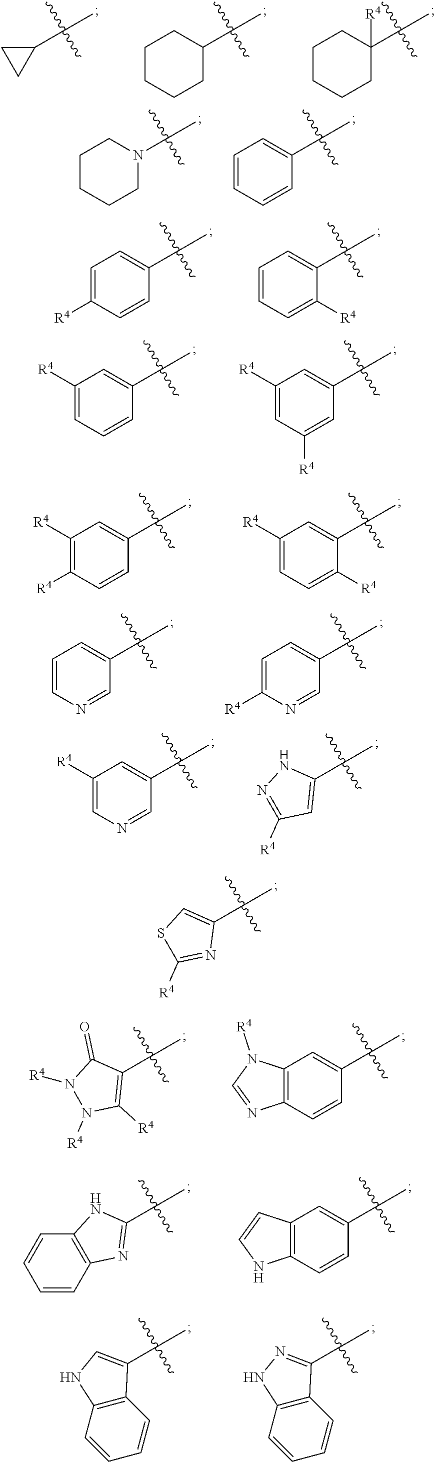

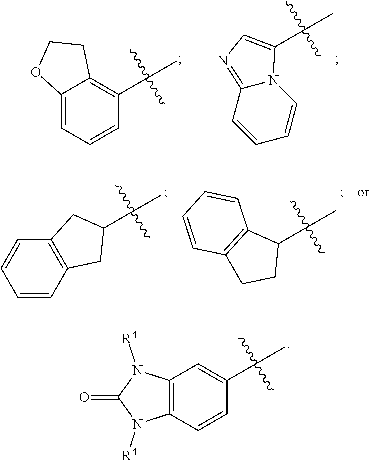

34. The compound of claim 31, or a pharmaceutically acceptable salt thereof, wherein Z is —C(O)NH—*, wherein * indicates the attachment point to R³, and wherein R³ is selected from benzamidazolyl and 2-oxo-2,3-dihydro-1H-benzo[d]imidazolyl, each of which is optionally substituted with 1 to 2 R⁴.

35. The compound of claim 34, or a pharmaceutically acceptable salt thereof, wherein R³ is represented by the following formula: 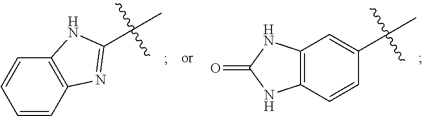 [SMILES: *c1nc2ccccc2[nH]1 ; *c1ccc2[nH]c(=O)[nH]c2c1]
each of which is optionally substituted with 1 to 2 R⁴.

36. The compound of claim 35, or a pharmaceutically acceptable salt thereof, wherein R³ is represented by the following formula:

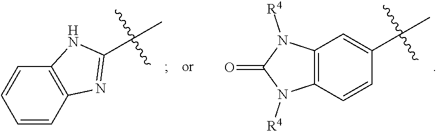

37. The compound of any one of claims 1 to 36, or a pharmaceutically acceptable salt thereof, wherein:
R⁴ is independently selected from C₁₋₄alkyl, halo, OR^{4a}, cyano, —NR^{4b}R^{4c}, —C(O)OH, —C(O)NR^{4b}R^{4c}, —NR^{4b}C(O)R^{4a}, —NR^{4b}C(O)OR^{4a}, 4 to 6-membered monocyclic heterocyclyl, phenyl, and 4 to 6-membered monocyclic heteroaryl, wherein the C₁₋₄alkyl is optionally substituted with 1 to 3 substituents selected from OR^{4f}, cyano, or 4 to 6-membered monocyclic heterocyclyl; and wherein the 4 to 6-membered monocyclic heterocyclyl, phenyl, and to 6-membered monocyclic heteroaryl are each optionally substituted with 1 to 3 R^{4e}; or two R⁴ together form oxo;
R^{4a} is H, phenyl, or C₁₋₄alkyl optionally substituted with 1 to 3 substituents independently selected from halo, phenyl, 5 to 6-membered monocyclic heteroaryl, and 4 to 6-membered monocyclic heterocyclyl, wherein the phenyl, 5 to 6-membered monocyclic heteroaryl, and 4 to 6-membered monocyclic heterocyclyl are each optionally substituted with 1 to 3 R^{4g};
R^{4b} and R^{4c} are each independently selected from H and C₁₋₄alkyl;
each R^{4e} is independently selected from C₁₋₄alkyl, halo, or —C(O)C₁₋₄alkyl;
R^{4f} is H or C₁₋₄alkyl; and
each R^{4g} is independently halo or —OH.

38. The compound of any one of claims 1 to 36, or a pharmaceutically acceptable salt thereof, wherein:
R⁴ is independently selected from C₁₋₄alkyl, halo, OR^{4a}, cyano, —NR^{4b}R^{4c}, —C(O)OH, —C(O)NR^{4b}R^{4c}, —NR^{4b}C(O)R^{4a}, —NR^{4b}C(O)OR^{4a}, 4 to 6-membered monocyclic heterocyclyl, phenyl, and 4 to 6-membered monocyclic heteroaryl, wherein the C₁₋₄alkyl is optionally substituted with 1 to 3 substituents selected from OR^{4f}, cyano, or 4 to 6-membered monocyclic heterocyclyl; and wherein the 4 to 6-membered monocyclic heterocyclyl, phenyl, and to 6-membered monocyclic heteroaryl are each optionally substituted with 1 to 3 R^{4e}; or two R⁴ together form oxo;
R^{4a} is H, phenyl, or C₁₋₄alkyl optionally substituted with 1 to 3 substituents independently selected from halo, phenyl and 4 to 6-membered monocyclic heterocyclyl;
R^{4b} and R^{4c} are each independently selected from H and C₁₋₄alkyl;
each R^{4e} is independently selected from C₁₋₄alkyl or —C(O)C₁₋₄alkyl; and
R^{4f} is H or C₁₋₄alkyl.

39. The compound of any one of claims 1 to 37, or a pharmaceutically acceptable salt thereof, wherein R⁴ is represented by the following structural formula: 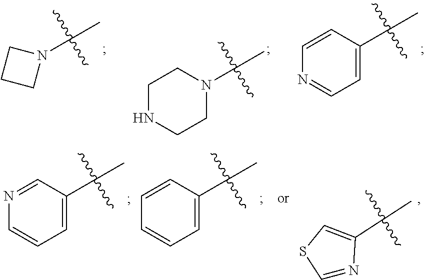
each of which is optionally substituted with 1 to 2 R^{4e}.

40. The compound of any one of claims 1 to 37, or a pharmaceutically acceptable salt thereof, wherein R⁴ is represented by the following structural formula: 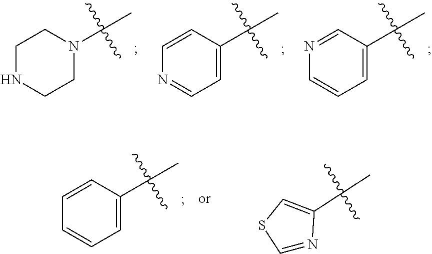 [SMILES: *N1CCNCC1 ; *c1ccncc1 ; *c1cccnc1 ; *c1ccccc1 ; *c1cscn1]
each of which is optionally substituted with 1 to 2 R^{4e}.

41. The compound of claim 39, or a pharmaceutically acceptable salt thereof, wherein R⁴ is represented by the following structural formula:

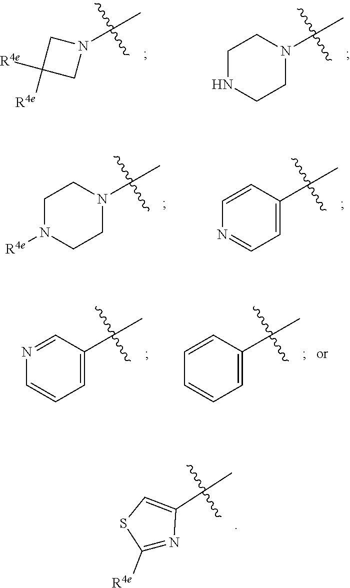

42. The compound of claim 39, or a pharmaceutically acceptable salt thereof, wherein R⁴ is represented by the following structural formula:

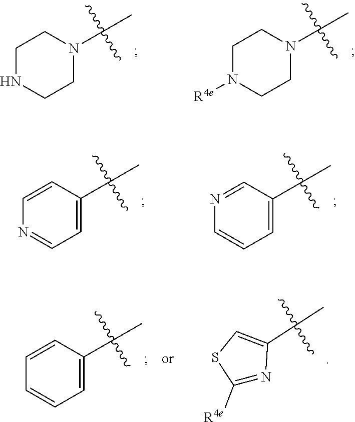

43. The compound of any one of claims 1 to 42, or a pharmaceutically acceptable salt thereof, wherein R^{4e} is —F, —CH₃, or —C(O)CH₃.

44. The compound of any one of claims 1 to 42, or a pharmaceutically acceptable salt thereof, wherein R^{4e} is —CH₃ or —C(O)CH₃.

45. The compound of any one of claims 1 to 38, or a pharmaceutically acceptable salt thereof, wherein R⁴ is —CH₃, —CH₂OCH₃, —CH₂CN, —CH₂CH₂OH, halo, cyano, —OH, —OCH₃, —OCH₂CH₃, —OCH(CH₃)₂, —OCF₃, —NH₂, —NHC(O)CH₃, —C(O)OH, —C(O)NH₂, —C(O)NHCH₃, —C(O)N(CH₃)₂, —C(O)NC(CH₃)₃, or R⁴ is represented by the following formula:

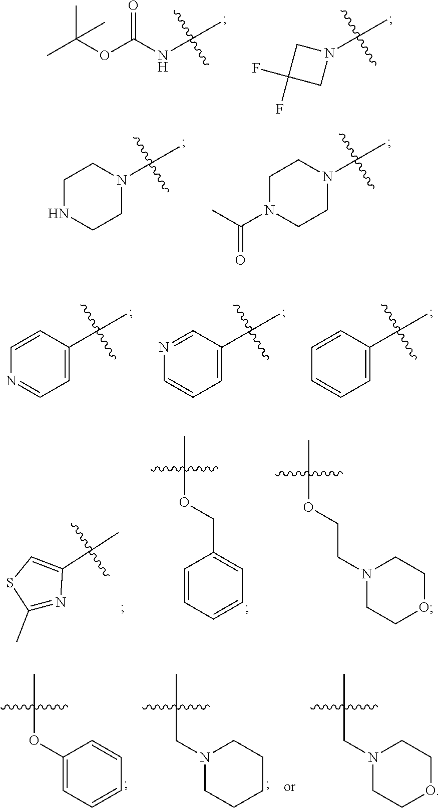

46. The compound of any one of claims 1 to 38, or a pharmaceutically acceptable salt thereof, wherein R⁴ is —CH₃, —CH₂OCH₃, —CH₂CN, —CH₂CH₂OH, halo, cyano, —OH, —OCH₃, —OCH₂CH₃, —OCH(CH₃)₂, —OCF₃, —NH₂, —NHC(O)CH₃, —C(O)OH, —C(O)NH₂, —C(O)NHCH₃, —C(O)N(CH₃)₂, —C(O)NC(CH₃)₃, or R⁴ is represented by the following formula:

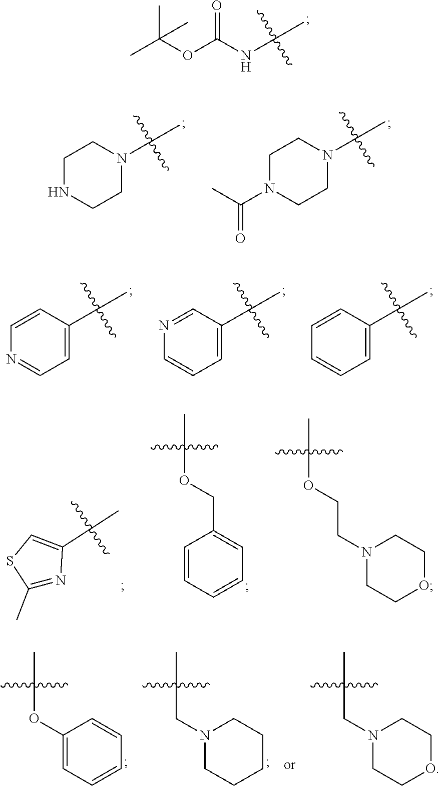

47. The compound of claim 45, or a pharmaceutically acceptable salt thereof, wherein Z is a bond and R⁴ is —CH₃, halo, cyano, —OH, —OCH₃, —OCH₂CH₃, —NH₂, —NHC(O)CH₃, —C(O)OH, —C(O)NH₂, or R⁴ is represented by the following formula:

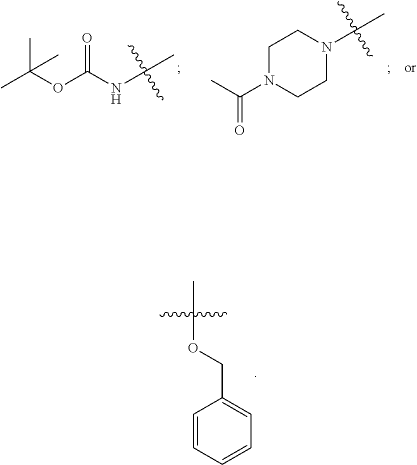

48. The compound of claim 46, or a pharmaceutically acceptable salt thereof, wherein R⁴ is —CH₃, —NH₂, —OH, halo, or —NHC(O)CH₃, or R⁴ is represented by the following formula:

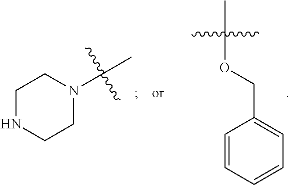 [SMILES: *N1CCNCC1 ; *OCc1ccccc1]

49. The compound of claim 47, or a pharmaceutically acceptable salt thereof, wherein Z is —CH₂—, —OCH₂—*, —S(O)₂, —S(O)₂N(H)—*, —N(H)—, —N(H)CH₂—*, —C(O)NH—*, or —C(O)NH—C₁₋₃alkylene-*, wherein * indicates the attachment point to R³, and wherein R⁴ is —CH₃, —CH₂OCH₃, —CH₂CN, —CH₂CH₂OH, halo, —OCH₃, —OCH(CH₃)₂, —OCF₃, C(O)NHCH₃, —C(O)N(CH₃)₂, —C(O)NC(CH₃)₃, or R⁴ is represented by the following formula:

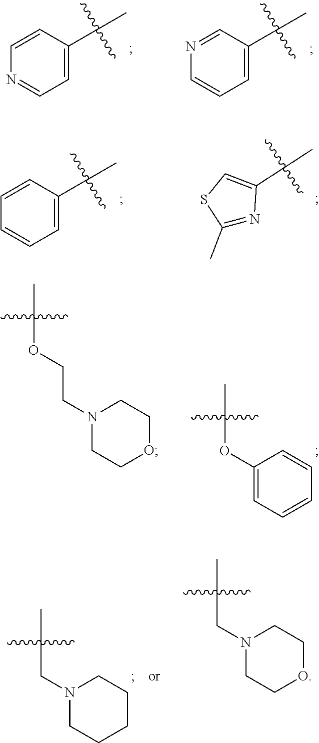

50. The compound of claim 49, or a pharmaceutically acceptable salt thereof, wherein R⁴ is —CH₃.

51. The compound of any one of claims 1 to 6, 20 to 30, and 37 to 48, wherein the compound is represented by Formula (IIA), (IIB) or (IIC): 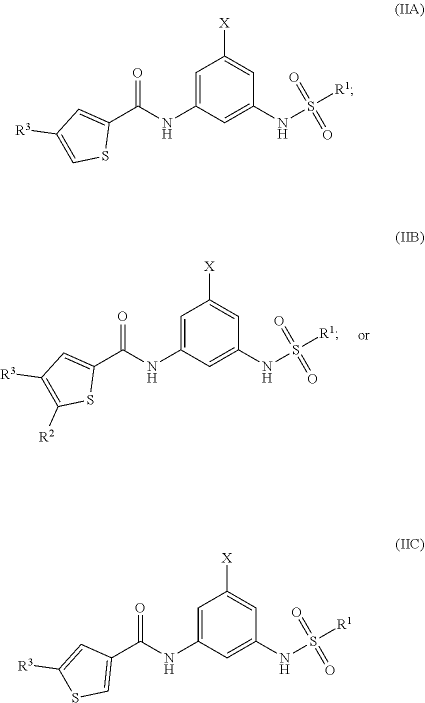
or a pharmaceutically acceptable salt thereof.

52. The compound of any one of claims 1 to 6, 20 to 30, and 37 to 48, wherein the compound is represented by Formula (IIIA) or (IIIB): 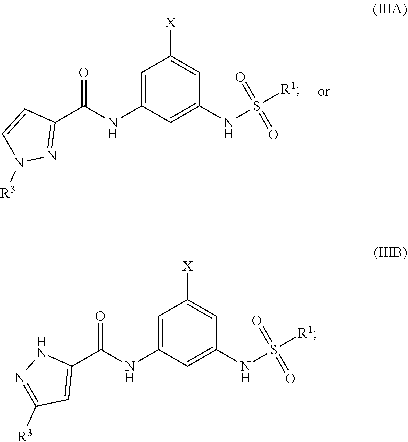
or a pharmaceutically acceptable salt thereof.

53. The compound of any one of claims 1 to 6, 20 to 30, and 37 to 48, wherein the compound is represented by Formula (IVA), (IVB), (IVC), or (IVD): 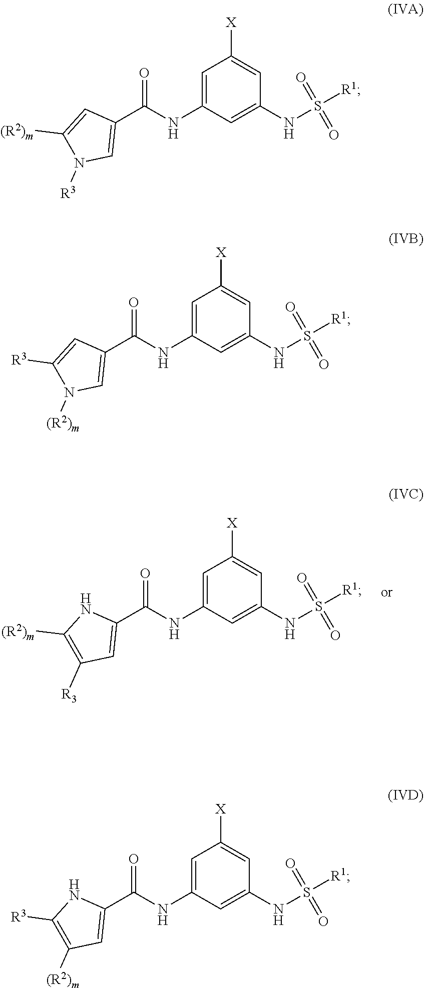
or a pharmaceutically acceptable salt thereof, wherein m is 0 or 1

54. The compound of any one of claims 1 to 6, 16 to 24, 31 to 46, 49, and 50, wherein the compound is represented by Formula (V): 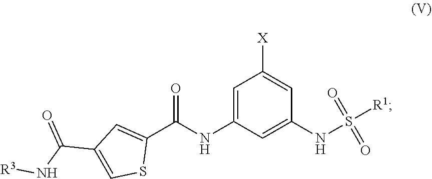
or a pharmaceutically acceptable salt thereof.

55. The compound of claim 1, wherein the compound is represented by Formula (VI): 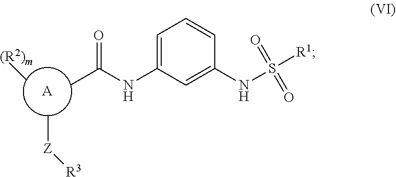
or a pharmaceutically acceptable salt thereof, wherein;
R¹ is C₁₋₂alkyl;
Ring A is selected from phenyl, thiophenyl, pyrazolyl, isothiazolyl, and imidazolyl;
m is 0 to 1;
each R² is independently selected from C₁₋₄alkyl and —C(O)OH;
Z is a bond or —C(O)NH—*, wherein * indicates the attachment point to R³;
R³ is selected from phenyl, pyridyl, pyrimidyl, benzimidazolyl and 2-oxo-2,3-dihydro-1H-benzo[d]imidazolyl, each of which is optionally substituted with 1 to 2 R⁴;
each R⁴ is independently selected from C₁₋₄alkyl, halo, —OH, —O-benzyl, cyano, —NR^{4b}R^{4c}, —C(O)NR^{4b}R^{4c}, or 4 to 6-membered monocyclic heterocyclyl;
R^{4b} and R^{4c} are each independently selected from H and C₁₋₄alkyl.

56. The compound of claim 55, or a pharmaceutically acceptable salt thereof, wherein Z is a bond and wherein ring A is represented by the following formula: 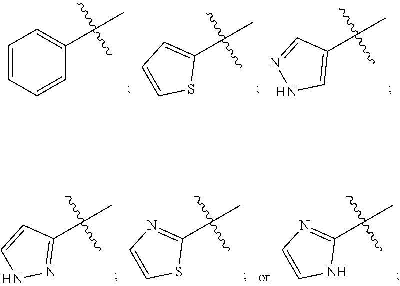 [SMILES: *c1ccccc1 ; *c1cccs1 ; *c1cn[nH]c1 ; *c1cc[nH]n1 ; *c1nccs1 ; *c1ncc[nH]1]
each of which is substituted with 1 R³ and optionally substituted with 1 R².

57. The compound of claim 56, or a pharmaceutically acceptable salt thereof, wherein ring A is represented by the following formula:

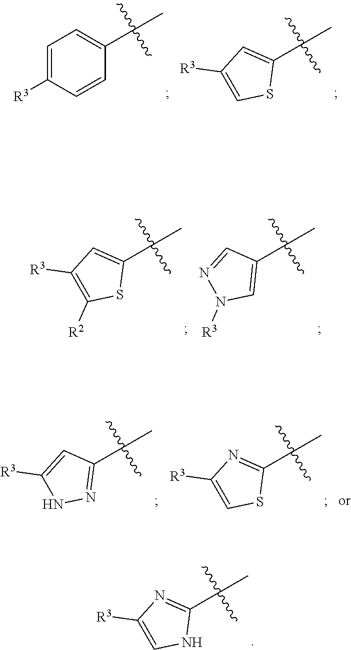

58. The compound of claim 56, or a pharmaceutically acceptable salt thereof, wherein ring A is represented by the following formula:

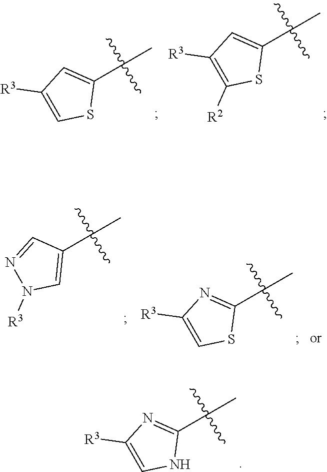

59. The compound of any one of claims 55 to 58, or a pharmaceutically acceptable salt thereof, wherein R² is —CH₃ or —C(O)OH.

60. The compound of any one of claims 55 to 59, or a pharmaceutically acceptable salt thereof, wherein R³ is selected from phenyl, pyridyl, and pyrimidyl, each of which is optionally substituted with 1 to 2 R⁴.

61. The compound of claim 60, or a pharmaceutically acceptable salt thereof, wherein R³ is represented by the following formula: 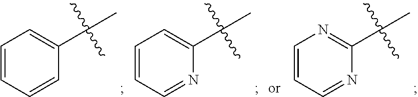 [SMILES: *c1ccccc1 ; *c1ccccn1 ; *c1ncccn1]
each of which is optionally substituted with 1 to 2 R⁴.

62. The compound of claim 61, or a pharmaceutically acceptable salt thereof, wherein R³ is represented by the following formula:

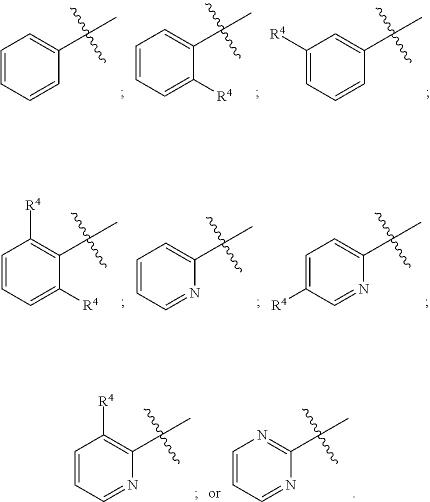

63. The compound of any one of claims 55 to 62, or a pharmaceutically acceptable salt thereof, wherein R⁴ is —CH₃, —NH₂, —OH, halo, or —NHC(O)CH₃, or R⁴ is represented by the following formula:

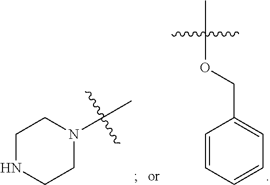 [SMILES: *N1CCNCC1 ; *OCc1ccccc1]

64. The compound of claim 55, or a pharmaceutically acceptable salt thereof, wherein Z is —C(O)NH—*, wherein * indicates the attachment point to R³; and wherein ring A is represented by the following formula: 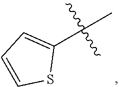 [SMILES: *c1cccs1]
which is substituted with 1 R³.

65. The compound of claim 64, or a pharmaceutically acceptable salt thereof, wherein ring A is represented by the following formula:

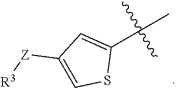

66. The compound of any one of claims 55, 64, or 65, or a pharmaceutically acceptable salt thereof, wherein R³ is selected from benzamidazolyl and 2-oxo-2,3-dihydro-1H-benzo[d]imidazolyl, each of which is optionally substituted with 1 to 2 R⁴.

67. The compound of claim 66, or a pharmaceutically acceptable salt thereof, wherein R³ is represented by the following formula:

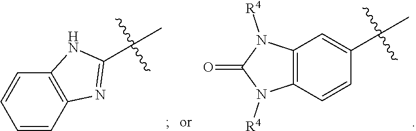

68. The compound of any of claims 55 and 64 to 67, or a pharmaceutically acceptable salt thereof, wherein R⁴ is —CH₃.

69. The compound of claim 1, wherein the compound is selected from:
N-(3-(methylsulfonamido)phenyl)-1-phenyl-1H-pyrazole-4-carboxamide;
N-(3-(methylsulfonamido)phenyl)-4-phenylpicolinamide;
4-(benzyloxy)-N-(3-(methylsulfonamido)phenyl)benzamide;
3-(benzyloxy)-N-(3-(methylsulfonamido)phenyl)benzamide;
N-(3-(methylsulfonamido)phenyl)-5-phenylthiophene-2-carboxamide;
N-(3-(methylsulfonamido)phenyl)-4-phenylthiophene-2-carboxamide;
4-(5-methyl-1,3,4-oxadiazol-2-yl)-N-(3-(methylsulfonamido)phenyl)benzamide;
N-(3-(methylsulfonamido)phenyl)-4-(1H-tetrazol-5-yl)benzamide;
N-(3-(methylsulfonamido)phenyl)-2-(4H-1,2,4-triazol-3-yl)thiazole-4-carboxamide;
1-benzyl-N-(3-(methylsulfonamido)phenyl)-1H-pyrazole-4-carboxamide;
N-(3-(methylsulfonamido)phenyl)-[1,1′-biphenyl]-3-carboxamide;
N-(3-(methylsulfonamido)phenyl)-[1,1′-biphenyl]-4-carboxamide;
N-(3-(methylsulfonamido)phenyl)-4-(1H-pyrazol-1-yl)benzamide;
N-(3-(methylsulfonamido)phenyl)-3-(1H-pyrazol-1-yl)benzamide;
N-(3-(methylsulfonamido)phenyl)-1-(pyridin-2-yl)-1H-pyrazole-4-carboxamide;
N-(3-(methylsulfonamido)phenyl)-4-phenylthiazole-2-carboxamide;
N-(3-(methylsulfonamido)phenyl)-1-(pyrimidin-2-yl)-1H-pyrazole-4-carboxamide;
N-(3-(methylsulfonamido)phenyl)-4-(thiophen-2-yl)picolinamide;
4-(3-fluorophenyl)-N-(3-(methylsulfonamido)phenyl)thiophene-2-carboxamide;
N-(3-(methylsulfonamido)phenyl)-4-(o-tolyl)thiophene-2-carboxamide;
N-(3-(methylsulfonamido)phenyl)-4-(m-tolyl)thiophene-2-carboxamide;
N-(3-(methylsulfonamido)phenyl)-4-(p-tolyl)thiophene-2-carboxamide;
5-methyl-N-(3-(methylsulfonamido)phenyl)-4-phenylthiophene-2-carboxamide;
4-(3-carbamoylphenyl)-N-(3-(methylsulfonamido)phenyl)thiophene-2-carboxamide;
4-(3-cyanophenyl)-N-(3-(methylsulfonamido)phenyl)thiophene-2-carboxamide;
4-(2-fluorophenyl)-N-(3-(methylsulfonamido)phenyl)thiophene-2-carboxamide;
4-(4-fluorophenyl)-N-(3-(methylsulfonamido)phenyl)thiophene-2-carboxamide;
N-(3-(methylsulfonamido)phenyl)-4-(pyridin-3-yl)thiophene-2-carboxamide;
N-(3-(methylsulfonamido)phenyl)-4-(pyridin-4-yl)thiophene-2-carboxamide;
4-(3-methoxyphenyl)-N-(3-(methylsulfonamido)phenyl)thiophene-2-carboxamide;
4-(2-hydroxyphenyl)-N-(3-(methylsulfonamido)phenyl)thiophene-2-carboxamide;
4-(3-aminophenyl)-N-(3-(methylsulfonamido)phenyl)thiophene-2-carboxamide;
4-(2-methoxyphenyl)-N-(3-(methylsulfonamido)phenyl)thiophene-2-carboxamide;
4-(2-methylpyridin-3-yl)-N-(3-(methylsulfonamido)phenyl)thiophene-2-carboxamide;
4-(4-methylpyridin-3-yl)-N-(3-(methylsulfonamido)phenyl)thiophene-2-carboxamide;
N-(3-(methylsulfonamido)phenyl)-4-(pyrimidin-5-yl)thiophene-2-carboxamide;
4-(2-ethoxypyrimidin-5-yl)-N-(3-(methylsulfonamido)phenyl)thiophene-2-carboxamide;
5-(4-hydroxyphenyl)-N-(3-(methylsulfonamido)phenyl)-1H-pyrazole-3-carboxamide;
4-(6-methylpyridin-3-yl)-N-(3-(methylsulfonamido)phenyl)thiophene-2-carboxamide;
N-(3-(methylsulfonamido)phenyl)-4-(pyridazin-3-yl)thiophene-2-carboxamide;
4-(3-methoxy-2-methylphenyl)-N-(3-(methylsulfonamido)phenyl)thiophene-2-carboxamide;
4-(5-methoxy-2-methylphenyl)-N-(3-(methylsulfonamido)phenyl)thiophene-2-carboxamide;
4-(3-hydroxypyridin-2-yl)-N-(3-(methylsulfonamido)phenyl)thiophene-2-carboxamide;
4-(2-fluoro-6-hydroxyphenyl)-N-(3-(methylsulfonamido)phenyl)thiophene-2-carboxamide;
4-benzyl-N-(3-(methylsulfonamido)phenyl)thiophene-2-carboxamide;
tert-butyl (3-(5-((3-(methylsulfonamido)phenyl)carbamoyl)thiophen-3-yl)phenyl)carbamate;
4-(3-hydroxyphenyl)-N-(3-(methylsulfonamido)phenyl)thiophene-2-carboxamide;
4-(3-hydroxy-2-methylphenyl)-N-(3-(methylsulfonamido)phenyl)thiophene-2-carboxamide;
4-(5-hydroxy-2-methylphenyl)-N-(3-(methylsulfonamido)phenyl)thiophene-2-carboxamide;
4-(2,6-difluorophenyl)-N-(3-(methylsulfonamido)phenyl)thiophene-2-carboxamide;
4-(2-(benzyloxy)phenyl)-N-(3-(methylsulfonamido)phenyl)thiophene-2-carboxamide;
5-(4-methoxyphenyl)-N-(3-(methylsulfonamido)phenyl)-1H-pyrazole-3-carboxamide;
5-(4-hydroxyphenyl)-1-methyl-N-(3-(methylsulfonamido)phenyl)-1H-pyrazole-3-carboxamide;
5-methyl-N-(3-(methylsulfonamido)phenyl)-4-(pyridin-2-yl)thiophene-2-carboxamide;
N-(3-(methylsulfonamido)phenyl)-3-(pyridin-2-yl)-1H-pyrazole-5-carboxamide;
N-(3-(methylsulfonamido)phenyl)-4-(pyridin-2-yl)thiophene-2-carboxamide;
4-(3-methylpyridin-2-yl)-N-(3-(methylsulfonamido)phenyl)thiophene-2-carboxamide;
N-(3-(methylsulfonamido)phenyl)-4-(pyrimidin-2-yl)thiophene-2-carboxamide;
4-(6-aminopyridin-2-yl)-N-(3-(methylsulfonamido)phenyl)thiophene-2-carboxamide;
4-(4-aminopyridin-2-yl)-N-(3-(methylsulfonamido)phenyl)thiophene-2-carboxamide;
2-(5-((3-(methylsulfonamido)phenyl)carbamoyl)thiophen-3-yl)isonicotinic acid;
4-(2-aminophenyl)-N-(3-(methylsulfonamido)phenyl)thiophene-2-carboxamide;
4-(3-aminopyridin-2-yl)-N-(3-(methylsulfonamido)phenyl)thiophene-2-carboxamide;
N-(3-(methylsulfonamido)phenyl)-4-(pyridin-2-yl)-1H-imidazole-2-carboxamide;
N-(3-(methylsulfonamido)phenyl)-4-(5-(piperazin-1-yl)pyridin-2-yl)thiophene-2-carboxamide;
2-(5-((3-(methylsulfonamido)phenyl)carbamoyl)thiophen-3-yl)isonicotinamide;
4-(2-acetamidophenyl)-N-(3-(methylsulfonamido)phenyl)thiophene-2-carboxamide;
4-(3-acetamidopyridin-2-yl)-N-(3-(methylsulfonamido)phenyl)thiophene-2-carboxamide;
4-(5-(4-acetylpiperazin-1-yl)pyridin-2-yl)-N-(3-(methylsulfonamido)phenyl)thiophene-2-carboxamide;
N-(3-(ethylsulfonamido)phenyl)-4-phenylthiophene-2-carboxamide;
N-(3-(ethylsulfonamido)phenyl)-4-(pyridin-2-yl)thiophene-2-carboxamide;
5-bromo-N-(3-(methylsulfonamido)phenyl)-4-phenylthiophene-2-carboxamide;
N-(3-(methylsulfonamido)phenyl)-4-phenyl-5-(trifluoromethyl)thiophene-2-carboxamide;
5-cyano-N-(3-(methylsulfonamido)phenyl)-4-phenylthiophene-2-carboxamide;
5-((3-(methylsulfonamido)phenyl)carbamoyl)-3-phenylthiophene-2-carboxylic acid;
N2-(3-(methylsulfonamido)phenyl)-N4-phenylthiophene-2,4-dicarboxamide;
N2-(3-(methylsulfonamido)phenyl)-N4-(2-(pyridin-3-yl)ethyl)thiophene-2,4-dicarboxamide;
N4-(3-chloro-4-methylphenyl)-N2-(3-(methylsulfonamido)phenyl)thiophene-2,4-dicarboxamide;
N4-((1-hydroxycyclohexyl)methyl)-N2-(3-(methylsulfonamido)phenyl)thiophene-2,4-dicarboxamide;
N4-(6-methoxypyridin-3-yl)-N2-(3-(methylsulfonamido)phenyl)thiophene-2,4-dicarboxamide;
N4-(1,5-dimethyl-3-oxo-2-phenyl-2,3-dihydro-1H-pyrazol-4-yl)-N2-(3-(methylsulfonamido)phenyl)thiophene-2,4-dicarboxamide;
N2-(3-(methylsulfonamido)phenyl)-N4-(4-(trifluoromethoxy)phenyl)thiophene-2,4-dicarboxamide;
N4-(5-fluoro-2-methoxyphenyl)-N2-(3-(methylsulfonamido)phenyl)thiophene-2,4-dicarboxamide;
N2-(3-(methylsulfonamido)phenyl)-N4-(1-phenylethyl)thiophene-2,4-dicarboxamide;
N4-((5-fluoropyridin-3-yl)methyl)-N2-(3-(methylsulfonamido)phenyl)thiophene-2,4-dicarboxamide;
N4-(2,3-dihydro-1H-inden-2-yl)-N2-(3-(methylsulfonamido)phenyl)thiophene-2,4-dicarboxamide;
N4-(4-chloro-3-methylphenyl)-N2-(3-(methylsulfonamido)phenyl)thiophene-2,4-dicarboxamide;
N4-(4-(dimethylcarbamoyl)phenyl)-N2-(3-(methylsulfonamido)phenyl)thiophene-2,4-dicarboxamide;
N2-(3-(methylsulfonamido)phenyl)-N4-(2-phenoxyphenyl)thiophene-2,4-dicarboxamide;
N2-(3-(methylsulfonamido)phenyl)-N4-(4-morpholinobenzyl)thiophene-2,4-dicarboxamide;
N2-(3-(methylsulfonamido)phenyl)-N4-(4-phenoxyphenyl)thiophene-2,4-dicarboxamide;
N4-(4-isopropoxyphenyl)-N2-(3-(methylsulfonamido)phenyl)thiophene-2,4-dicarboxamide;
N4-(cyclopropylmethyl)-N2-(3-(methylsulfonamido)phenyl)thiophene-2,4-dicarboxamide;
N2-(3-(methylsulfonamido)phenyl)-N4-(4-(morpholinomethyl)phenyl)thiophene-2,4-dicarboxamide;
N2-(3-(methylsulfonamido)phenyl)-N4-(3-(trifluoromethoxy)phenyl)thiophene-2,4-dicarboxamide;
N4-(3-isopropoxyphenyl)-N2-(3-(methylsulfonamido)phenyl)thiophene-2,4-dicarboxamide;
N2-(3-(methylsulfonamido)phenyl)-N4-(4-(2-morpholinoethoxy)phenyl)thiophene-2,4-dicarboxamide;
N4-(2,3-dihydrobenzofuran-4-yl)-N2-(3-(methylsulfonamido)phenyl)thiophene-2,4-dicarboxamide;
N2-(3-(methylsulfonamido)phenyl)-N4-(4-(morpholinomethyl)benzyl)thiophene-2,4-dicarboxamide;
N4-(3-chloro-5-methoxyphenyl)-N2-(3-(methylsulfonamido)phenyl)thiophene-2,4-dicarboxamide;
N4-(4-(tert-butylcarbamoyl)phenyl)-N2-(3-(methylsulfonamido)phenyl)thiophene-2,4-dicarboxamide;
N2-(3-(methylsulfonamido)phenyl)-N4-(4-(pyridin-4-yl)phenyl)thiophene-2,4-dicarboxamide;
N4-(imidazo[1,2-a]pyridin-3-yl)-N2-(3-(methylsulfonamido)phenyl)thiophene-2,4-dicarboxamide;
N4-(4-methoxyphenyl)-N2-(3-(methylsulfonamido)phenyl)thiophene-2,4-dicarboxamide;
N4-(1,3-dimethyl-2-oxo-2,3-dihydro-1H-benzo[d]imidazol-5-yl)-N2-(3-(methylsulfonamido)phenyl)thiophene-2,4-dicarboxamide;
N4-(1-methyl-1H-benzo [d]imidazol-6-yl)-N2-(3-(methylsulfonamido)phenyl)thiophene-2,4-dicarboxamide;
N2-(3-(methylsulfonamido)phenyl)-N4-(3-(pyridin-3-yl)phenyl)thiophene-2,4-dicarboxamide;
N4-(3-(methoxymethyl)phenyl)-N2-(3-(methylsulfonamido)phenyl)thiophene-2,4-dicarboxamide;
N4-(2-isopropoxyphenyl)-N2-(3-(methylsulfonamido)phenyl)thiophene-2,4-dicarboxamide;
N4-(2,3-dihydro-1H-inden-1-yl)-N2-(3-(methylsulfonamido)phenyl)thiophene-2,4-dicarboxamide;
N4-(4-chlorophenyl)-N2-(3-(methylsulfonamido)phenyl)thiophene-2,4-dicarboxamide;
N4-(2-(cyanomethyl)phenyl)-N2-(3-(methylsulfonamido)phenyl)thiophene-2,4-dicarboxamide;
N4-(4-(methylcarbamoyl)phenyl)-N2-(3-(methylsulfonamido)phenyl)thiophene-2,4-dicarboxamide;
N4-(2-(1H-indol-3-yl)ethyl)-N2-(3-(methylsulfonamido)phenyl)thiophene-2,4-dicarboxamide;
N4-((1H-benzo[d]imidazol-2-yl)methyl)-N2-(3-(methylsulfonamido)phenyl)thiophene-2,4-dicarboxamide;
N4-(2,5-dimethoxyphenethyl)-N2-(3-(methylsulfonamido)phenyl)thiophene-2,4-dicarboxamide;
N4-(2-(2-hydroxyethyl)phenyl)-N2-(3-(methylsulfonamido)phenyl)thiophene-2,4-dicarboxamide;
N4-((1H-indazol-3-yl)methyl)-N2-(3-(methylsulfonamido)phenyl)thiophene-2,4-dicarboxamide;
N4-((1H-indol-5-yl)methyl)-N2-(3-(methylsulfonamido)phenyl)thiophene-2,4-dicarboxamide;
N4-(1H-benzo[d]imidazol-2-yl)-N2-(3-(methylsulfonamido)phenyl)thiophene-2,4-dicarboxamide;
N2-(3-(methylsulfonamido)phenyl)-N4-(3-(2-methylthiazol-4-yl)phenyl)thiophene-2,4-dicarboxamide;
N2-(3-(methylsulfonamido)phenyl)-N4-(5-phenyl-1H-pyrazol-3-yl)thiophene-2,4-dicarboxamide;
N4-(4-(2-hydroxyethyl)phenyl)-N2-(3-(methylsulfonamido)phenyl)thiophene-2,4-dicarboxamide;
N2-(3-(methylsulfonamido)phenyl)-N4-(2-(piperidin-1-yl)ethyl)thiophene-2,4-dicarboxamide;
N2-(3-(methylsulfonamido)phenyl)-N4-(3-(piperidin-1-ylmethyl)phenyl)thiophene-2,4-dicarboxamide;
N2-methyl-N5-(3-(methylsulfonamido)phenyl)-3-phenylthiophene-2,5-dicarboxamide;
5-(hydroxymethyl)-N-(3-(methylsulfonamido)phenyl)-4-phenylthiophene-2-carboxamide;
1-benzyl-5-methyl-N-(3-(methylsulfonamido)phenyl)-1H-pyrazole-3-carboxamide;
4-(benzylamino)-N-(3-(methylsulfonamido)phenyl)thiophene-2-carboxamide;
N-(6-(methylsulfonamido)pyridin-2-yl)-4-phenylthiophene-2-carboxamide;
N-(3-(methylsulfonoamidimidamido)phenyl)-4-phenylthiophene-2-carboxamide;
N-(3-methyl-5-(methylsulfonamido)phenyl)-4-phenylthiophene-2-carboxamide;
N-(3-formyl-5-(methylsulfonamido)phenyl)-4-phenylthiophene-2-carboxamide;
5-(5-(3,3-difluoroazetidin-1-yl)pyridin-2-yl)-1-methyl-N-(3-(methylsulfonamido)phenyl)-1H-pyrrole-3-carboxamide;
5-(3,5-difluoropyridin-2-yl)-1-methyl-N-(3-(methylsulfonamido)phenyl)-1H-pyrrole-3-carboxamide; and
N4-(2-cyclopropylethyl)-N2-(3-(methylsulfonamido)phenyl)thiophene-2,4-dicarboxamide;
or a pharmaceutically acceptable salt thereof.

70. A pharmaceutical composition comprising a compound of any one of claims 1 to 69, or a pharmaceutically acceptable salt thereof, and a pharmaceutically acceptable excipient.

71. A method of treating a DHX9 mediated disease or disorder in a subject, comprising administering to the subject a compound, or pharmaceutically acceptable salt thereof, of any one of claims 1 to 69, or the pharmaceutical composition of claim 70.

72. A method of treating a DHX9 mediated disease or disorder in a subject, comprising administering to the subject a compound represented by Formula (I): 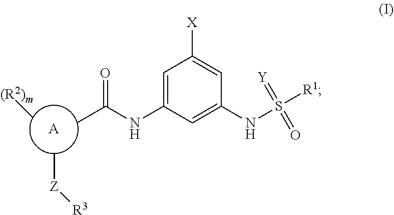
or a pharmaceutically acceptable salt thereof, wherein:
X is H, C₁₋₄alkyl, C₂₋₄haloalkyl, or C(O)R^{x};
R^{x} is H, —OH, —OC₁₋₄alkyl, or C₁₋₄alkyl;
Y is O or NR^{y};
R^{y} is H or C₁₋₄alkyl;
R¹ is C₁₋₄alkyl or C₃₋₆cycloalkyl; wherein the C₁₋₄alkyl or C₃₋₆cycloalkyl are each optionally and independently substituted with 1 to 3 halo;
Ring A is a C₃₋₆cycloalkyl, 4 to 6-membered monocyclic heterocyclyl, 6 to 10 membered bicyclic heterocyclyl, phenyl, 5 to 6-membered monocyclic heteroaryl, or 8 to 10-membered bicyclic heteroaryl;
m is 0 to 3;
each R² is independently selected from C₁₋₄alkyl, C₃₋₆cycloalkyl, halo, OR^{2a}, cyano, —NR^{2b}R^{2c}, —SO₂R^{2a}, —C(O)R^{2d}, and —C(O)NR^{2b}R^{2c}, wherein the C₁₋₄alkyl and C₃₋₆cycloalkyl are optionally substituted with 1 to 4 R^{2e}; or 2 R² together form oxo;
R^{2a} is H, C₁₋₄alkyl, C₁₋₄haloalkyl, C₁₋₄alkoxyC₁₋₄alkyl, or benzyl;
R^{2d} is H, C₁₋₄alkyl, C₃₋₆cycloalkyl; OR^{2a}, phenyl, 4 to 6-membered monocyclic heterocyclyl or 5 to 6-membered monocyclic heteroaryl, wherein the C₁₋₄alkyl is optionally substituted with 1 to 3 halo;
R^{2b} and R^{2c} are each independently selected from H, C₁₋₄alkyl, C₃₋₆cycloalkyl, phenyl, 4 to 6-membered monocyclic heterocyclyl and 5 to 6-membered monocyclic heteroaryl; or R^{2b} and R^{2c}, together with the nitrogen to which they are attached form a 4 to 6 membered monocyclic heterocyclyl or 6 to 10 membered bicyclic heterocyclyl; wherein the 4 to 6 membered monocyclic heterocyclyl or 6 to 10 membered bicyclic heterocyclyl are each optionally substituted with 1 to 3 R^{2d};
each R^{2e} is independently selected from halo, cyano, NR^{2b}R^{2c}, OR^{2a}, phenyl, and 4 to 6 membered monocyclic heterocyclyl;
Z is a bond, —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, or —C(O)N(Z^{a})—C₁₋₃alkylene-*, wherein * indicates the attachment point to R³;
Z^{a} is H or C₁₋₄alkyl;
provided when X is H and Z is a bond, ring A is phenyl, pyridinyl, thiophenyl, pyrrolyl, pyrazolyl, thiazolyl, imidazolyl, furanyl, or 1,4,5,6-tetrahydrocyclopenta[c]pyrazolyl;
R³ is C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl, wherein the C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl are each optionally and independently substituted with 1 to 4 R⁴;
each R⁴ is independently selected from C₁₋₄alkyl, halo, OR^{4a}, cyano, —NR^{4b}R^{4c}, —C(O)R^{4a}, —C(O)OR^{4a}, —C(O)NR^{4b}R^{4c}, —NR^{4b}C(O)R^{4a}—NR^{4b}C(O)OR^{4a}, —SR^{4a}, —SO₂R^{4a}, —SO₂NR^{4b}R^{4c}, —P(O)R^{4b}R^{4c}, phenyl, 5 to 6-membered monocyclic heteroaryl, 4 to 6-membered monocyclic heterocyclyl, and 6 to 10-membered bicyclic heterocyclyl, wherein the C₁₋₄alkyl is optionally substituted with 1 to 4 R^{4d}, and wherein the phenyl, 5 to 6-membered monocyclic heteroaryl, 4 to 6-membered monocyclic heterocyclyl and 6 to 10-membered bicyclic heterocyclyl are each optionally substituted with 1 to 3 R^{4e}; or two R⁴ together form oxo;
R^{4a} is H, C₁₋₄alkyl optionally substituted with 1 to 4 R^{4d}, C₃₋₆cycloalkyl, 4 to 6-membered monocyclic heterocyclyl, phenyl, or 5 to 6-membered monocyclic heteroaryl; wherein the C₃₋₆cycloalkyl, 4 to 6-membered monocyclic heterocyclyl, phenyl or 5 to 6-membered monocyclic heteroaryl are each optionally and independently substituted with 1 to 3 R^{4e};
R^{4b} and R^{4c} are each independently selected from H, phenyl, 4 to 6-membered monocyclic heterocyclyl, 5 to 6-membered monocyclic heteroaryl, and C₁₋₄alkyl optionally substituted with 1 to 4 R^{4d};
each R^{4d} is independently selected from halo, OR^{4f}, —C(O)C₁₋₄alkyl, —C(O)NR^{4b}R^{4c}, —C(O)C₁₋₄haloalkyl, phenyl, 4- to 6-membered monocyclic heterocyclyl, and 5 to 6-membered monocyclic heteroaryl, wherein the phenyl and 5 to 6-membered monocyclic heteroaryl are each optionally substituted with 1 to 3 R^{4g};
each R^{4e} is independently selected from halo, C₁₋₄alkyl optionally substituted with 1 to 3 halo, cyano, OR^{4f}, —NR^{4b}R^{4c}, —C(O)H, —C(O)C₁₋₄alkyl, —C₁₋₄alkylene-C(O)NR^{4b}R^{4c}, and —C(O)NR^{4b}R^{4c}; or two R^{4e} together form oxo;
R^{4f} is H, C₁₋₄alkyl, C₁₋₄haloalkyl, phenyl or 5 to 6-membered monocyclic heteroaryl, wherein the phenyl and 5 to 6-membered monocyclic heteroaryl are each optionally substituted with one to three halo;
each R^{4g} is independently selected from halo, OR^{4f}, C₁₋₄alkyl, halo, cyano, —NR^{4b}R^{4c}, —C(O)H, and —C(O)OR^{4f}.

73. The method of claim 72, wherein:
X is H, C₁₋₄alkyl, C₂₋₄haloalkyl, or C(O)R^{x};
R^{x} is H, —OH, —OC₁₋₄alkyl, or C₁₋₄alkyl;
Y is O or NR^{y};
R^{y} is H or C₁₋₄alkyl;
R¹ is C₁₋₄alkyl or C₃₋₆cycloalkyl; wherein the C₁₋₄alkyl or C₃₋₆cycloalkyl are each optionally and independently substituted with 1 to 3 halo;
Ring A is a C₃₋₆cycloalkyl, 4 to 6-membered monocyclic heterocyclyl, 6 to 10 membered bicyclic heterocyclyl, phenyl, 5 to 6-membered monocyclic heteroaryl, or 8 to 10-membered bicyclic heteroaryl;
m is 0 to 3;
each R² is independently selected from C₁₋₄alkyl, C₃₋₆cycloalkyl, halo, OR^{2a}, cyano, —NR^{2b}R^{2c}, —SO₂R^{2a}, —C(O)R^{2d}, and —C(O)NR^{2b}R^{2c}, wherein the C₁₋₄alkyl and C₃₋₆cycloalkyl are optionally substituted with 1 to 4 R^{2e}; or 2 R² together form oxo;
R^{2a} is H, C₁₋₄alkyl, C₁₋₄haloalkyl, C₁₋₄alkoxyC₁₋₄alkyl, or benzyl;
R^{2d} is H, C₁₋₄alkyl, C₃₋₆cycloalkyl; OR^{2a}, phenyl, 4 to 6-membered monocyclic heterocyclyl or 5 to 6-membered monocyclic heteroaryl, wherein the C₁₋₄alkyl is optionally substituted with 1 to 3 halo;
R^{2b} and R^{2c} are each independently selected from H, C₁₋₄alkyl, C₃₋₆cycloalkyl, phenyl, 4 to 6-membered monocyclic heterocyclyl and 5 to 6-membered monocyclic heteroaryl; or R^{2b} and R^{2c}, together with the nitrogen to which they are attached form a 4 to 6 membered monocyclic heterocyclyl or 6 to 10 membered bicyclic heterocyclyl; wherein the 4 to 6 membered monocyclic heterocyclyl or 6 to 10 membered bicyclic heterocyclyl are each optionally substituted with 1 to 3 R^{2d};
each R^{2e} is independently selected from halo, cyano, NR^{2b}R^{2c}, OR^{2a}, phenyl, and 4 to 6 membered monocyclic heterocyclyl;
Z is a bond, —CH₂—, —O—, —O—C₁₋₄alkylene-*, —C₁₋₄alkylene-O—*, —C(O)—, —C(O)O—*, —OC(O)—*, —S(O)₂—, —S(O)₂N(Z^{a})—*, —N(Z^{a})S(O)₂—*, —N(Z^{a})—, —N(Z^{a})—C₁₋₄alkylene-*, —C₁₋₄alkylene-N(Z^{a})—*, —C(O)N(Z^{a})—*, —N(Z^{a})C(O)—*, or —C(O)N(Z^{a})—C₁₋₃alkylene-*, wherein * indicates the attachment point to R³;
Z^{a} is H or C₁₋₄alkyl;
provided when X is H and Z is a bond, ring A is phenyl, pyridinyl, thiophenyl, pyrrolyl, pyrazolyl, thiazolyl, imidazolyl, furanyl, or 1,4,5,6-tetrahydrocyclopenta[c]pyrazolyl;
R³ is C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl, wherein the C₃₋₆cycloalkyl, 7 to 10-membered bicyclic carbocyclyl, 4 to 6-membered monocyclic heterocyclyl, 6 to 10-membered bicyclic heterocyclyl, phenyl, 5 to 6-membered monocyclic heteroaryl, or 8 to 10-membered bicyclic heteroaryl are each optionally and independently substituted with 1 to 4 R⁴;
each R⁴ is independently selected from C₁₋₄alkyl, halo, OR^{4a}, cyano, —NR^{4b}R^{4c}, —C(O)R^{4a}, —C(O)OR^{4a}, —C(O)NR^{4b}R^{4c}, —NR^{4b}C(O)R^{4a} —NR^{4b}C(O)OR^{4a}, —SR^{4a}, —SO₂R^{4a}, —SO₂NR^{4b}R^{4c}, —P(O)R^{4b}R^{4c}, phenyl, 5 to 6-membered monocyclic heteroaryl, 4 to 6-membered monocyclic heterocyclyl, and 6 to 10-membered bicyclic heterocyclyl, wherein the C₁₋₄alkyl is optionally substituted with 1 to 4 R^{4d}, and wherein the phenyl, 5 to 6-membered monocyclic heteroaryl, 4 to 6-membered monocyclic heterocyclyl and 6 to 10-membered bicyclic heterocyclyl are each optionally substituted with 1 to 3 R^{4e}; or two R⁴ together form oxo;
R^{4a} is H, C₁₋₄alkyl optionally substituted with 1 to 4 R^{4d}, C₃₋₆cycloalkyl, 4 to 6-membered monocyclic heterocyclyl, phenyl, or 5 to 6-membered monocyclic heteroaryl; wherein the C₃₋₆cycloalkyl, 4 to 6-membered monocyclic heterocyclyl, phenyl or 5 to 6-membered monocyclic heteroaryl are each optionally and independently substituted with 1 to 3 R^{4e};
R^{4b} and R^{4c} are each independently selected from H, phenyl, 4 to 6-membered monocyclic heterocyclyl, 5 to 6-membered monocyclic heteroaryl, and C₁₋₄alkyl optionally substituted with 1 to 4 R^{4d};
each R^{4d} is independently selected from halo, OR^{4f}, —C(O)C₁₋₄alkyl, —C(O)NR^{4b}R^{4c}, —C(O)C₁₋₄haloalkyl, phenyl, and 5 to 6-membered monocyclic heteroaryl, wherein the phenyl and 5 to 6-membered monocyclic heteroaryl are each optionally substituted with 1 to 3 R^{4g};
each R^{4e} is independently selected from halo, C₁₋₄alkyl optionally substituted with 1 to 3 halo, cyano, OR^{4f}, —NR^{4b}R^{4c}, —C(O)H, —C(O)C₁₋₄alkyl, —C₁₋₄alkylene-C(O)NR^{4b}R^{4c}, and —C(O)NR^{4b}R^{4c}; or two R^{4e} together form oxo;
R^{4f} is H, C₁₋₄alkyl, C₁₋₄haloalkyl, phenyl or 5 to 6-membered monocyclic heteroaryl, wherein the phenyl and 5 to 6-membered monocyclic heteroaryl are each optionally substituted with one to three halo;
each R^{4g} is independently selected from halo, OR^{4f}, C₁₋₄alkyl, halo, cyano, —NR^{4b}R^{4c}, —C(O)H, and —C(O)OR^{4f}.

74. The method of any one of claims 71 to 73, wherein the disease or disorder is cancer, viral infection or autoimmune disease.

75. The method of claim 74, wherein the disease or disorder is cancer.

76. The method of claim 75, wherein the cancer is selected from colorectal, endometrial, ovarian, gastric, hematopoietic, breast, brain, skin, lung, blood, prostate, head and neck, pancreatic, bladder, bone, soft-tissue, kidney and liver cancer.

77. The method of claim 75, wherein the cancer is selected from colorectal, endometrial, ovarian, gastric, hematopoietic, breast, brain, skin, lung, blood, prostate, head and neck, pancreatic, and liver cancer.

78. The method of claim 77, wherein the cancer is selected from colorectal, endometrial, ovarian, hematopoietic, and gastric cancer.

79. The method of any one of claims 75 to 78, wherein the cancer is a microsatellite instability (MSI) cancer.

80. The method of any one of claims 75 to 78, wherein the cancer has mutations or defects in DNA mis-match repair (MMR), and/or mutations or defects in RNA splicing and the kinetochore complex.

81. The method of claim 79, wherein the cancer is a microsatellite instability-high (MSI-H) colorectal cancer.

82. The method of claim 76, wherein the cancer is Ewing's Sarcoma.


SEQUENCE LISTING (11 sequences)

SEQ ID NO: 1 - RNA, 37 nt, synthetic construct
  misc_feature 1..37: note = Synthetic: Oligo1
  source 1..37: mol_type = other RNA
gcctggtccctgtccttgttattttccttggttaatt

SEQ ID NO: 2 - RNA, 37 nt, synthetic construct
  misc_feature 1..37: note = Synthetic: Oligo2
  source 1..37: mol_type = other RNA
gaattaaccaaggaaaataacaaggacagggaccagg

SEQ ID NO: 3 - DNA, 20 bp, synthetic construct
  misc_feature 1..20: note = Synthetic: BRIP1-Circular-Forward
  source 1..20: mol_type = other DNA
tctgtgtgccagactgtgag

SEQ ID NO: 4 - DNA, 22 bp, synthetic construct
  misc_feature 1..22: note = Synthetic: BRIP1-Circular-Reverse
  source 1..22: mol_type = other DNA
acaccaagttctgacgaaaagg

SEQ ID NO: 5 - DNA, 20 bp, synthetic construct
  misc_feature 1..20: note = Synthetic: BRIP1-Linear-Forward
  source 1..20: mol_type = other DNA
gtccatcctgaggtagtcgg

SEQ ID NO: 6 - DNA, 20 bp, synthetic construct
  misc_feature 1..20: note = Synthetic: BRIP1-Linear-Reverse
  source 1..20: mol_type = other DNA
ttccccaggctgacaagttc

SEQ ID NO: 7 - DNA, 22 bp, synthetic construct
  misc_feature 1..22: note = Synthetic: GAPDH-Forward
  source 1..22: mol_type = other DNA
gtctcctctgacttcaacagcg

SEQ ID NO: 8 - DNA, 22 bp, synthetic construct
  misc_feature 1..22: note = Synthetic: GAPDH-Reverse
  source 1..22: mol_type = other DNA
accaccctgttgctgtagccaa

SEQ ID NO: 9 - DNA, 23 bp, synthetic construct
  misc_feature 1..23: note = Synthetic: Forward Primer
  source 1..23: mol_type = other DNA
gtgagccaaggaattttgtgttt

SEQ ID NO: 10 - DNA, 26 bp, synthetic construct
  misc_feature 1..26: note = Synthetic: Reverse Primer
  source 1..26: mol_type = other DNA
ggtctgaactttgccattaatatctg

SEQ ID NO: 11 - DNA, 18 bp, synthetic construct
  misc_feature 1..18: note = Synthetic: Probe
  source 1..18: mol_type = other DNA
ccatcttacaaggccttt